(19) United States
(12) Patent Application Publication
(10) Pub. No.: US 2006/0088852 A1
(43) Pub. Date: Apr. 27, 2006

(54) Method for determining the homeostasis of hairy skin

(76) Inventors: Dirk Petersohn, Cologne (DE); Kordula Schlotmann, Duesseldorf (DE); Thomas Gassenmeier, Duesseldorf (DE); Olaf Holtkoetter, Huerth (DE); Marcus Conradt, Berlin (DE); Kay Hofmann, Cologne (DE)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/158,209

(22) Filed: Jun. 20, 2005

Related U.S. Application Data
(63) Continuation of application No. PCT/EP03/14070, filed on Dec. 11, 2003.

(30) Foreign Application Priority Data
Dec. 20, 2002 (DE) 102 60 931.4

(51) Int. Cl. C12Q 1/68; G01N 33/53

(52) U.S. Cl. 435/6; 435/7.1

(57) ABSTRACT

The invention relates to a method for determining the homeostasis of hairy skin in vitro, to test kits and biochips for determining markers of hairy skin, in addition to the use of proteins, mRNA molecules or fragments thereof as markers of hairy skin. The invention also relates to a test method for detecting the effectiveness of cosmetic or pharmaceutical active substances for treating hairy skin in addition to a screening method for identifying cosmetic or pharmaceutical active substances for treating hairy skin and to a method for producing a cosmetic or pharmaceutical preparation for treating hairy skin.

### CROSS REFERENCE TO RELATED APPLICATIONS

This application is a §365 (c) continuation application of PCT/EP2003/014070 filed 11 Dec. 2003, which in turn claims priority to DE Application 102 60 931.4 filed 20 Dec. 2002, each of the foregoing applications is incorporated herein by reference.

### FIELD OF THE INVENTION

The present invention relates to a method for determining the homeostasis of hairy skin in vitro, to test kits and biochips for determining genetic and protein markers of hairy skin, and to the use of proteins, mRNA molecules or fragments thereof as molecular markers of hairy skin. Also provided is a test method for determining the efficacy of cosmetic or pharmaceutically active ingredients for the treatment of hairy skin, and a method for producing a cosmetic or pharmaceutical preparation so identified.

### BACKGROUND OF THE INVENTION

Every living cell responds to signals from its surroundings. The responses of cells are implemented by an ordered regulation of gene expression such that cellular metabolism is a dynamic, rather than static state. The human genome comprises, according to the most recent estimates, between 30,000 and 140,000 genes. However, each cell uses only a small portion of this genetic information which is specific for that particular cell. Thus, gene expression patterns differ from cell type to cell type. Exogenous signals often trigger complex signal transduction cascades which effect changes in gene expression patterns. In this way, signals from cellular surroundings give rise to alterations in cellular metabolism.

Besides these relatively short-lived changes in gene expression patterns, each living cell is subject to an aging phenomenon, a process which is associated with a slow change in gene expression.

The human skin is complex in structure and is the largest organ of the human body. The skin forms the body's interface with its surroundings and is comprised of many different cell types. Most cells of the skin are found in the epidermis and dermis.

Analysis of gene expression is crucially important for understanding the general biological responses of the skin and in particular the modulation of cutaneous structure formation in response to exogenous stimuli. Cutaneous macromolecular structures or appendages, including, without limitation, hair follicles, sebaceous glands, sweat glands etc. are formed by a relatively small proportion of specialized cells (e.g., fewer than 5% of skin cells are involved in hair follicle structure). Accordingly, analyzing the cells which contribute to these structures (e.g., by gene expression profiling) is performed only with difficulty.

Isolation of cutaneous structures is technically difficult and very time-consuming. Removal of cells from their natural biological state tends to inhibit realistic visualization of biochemical processes in the skin or its appendages. Every manipulation of tissue (e.g., to isolate or concentrate particular structures) perturbs the cells, resulting in an alteration of gene expression patterns. This state no longer reflects the natural cellular state and thus cannot be regarded as representative.

Hairs are present on most regions of human skin, with the exception of the lips, the palms of the hands and the soles of the feet. Inapparent vellus hairs differ macroscopically from the cosmetically relevant cephalic hairs on the head. Microscopic differentiation is possible for both types of hair follicles and for the hairs themselves. However, the biochemical and molecular biological mechanisms underlying these differences are substantially unknown.

A relevant feature of hairs and their follicles is that with increasing age, the cells lose their ability to maintain the homeostasis of the organ. Thus, for example, over time the number of hair follicles per unit area decreases. There is likewise a change in the structure of hairs in that, for example, the hair diameter becomes smaller. Frequently pigment producing cells in the hair follicles lose this ability with increasing age resulting the graying of the hair. The molecular mechanisms underlying this development are as yet substantially unclear.

Effective cosmetic or pharmaceutical hair products should provide beneficial cosmetic and/or therapeutic effects on a variety of molecular processes in the hair follicle. However, to date, only a few molecular reaction mechanisms in the hair follicle have been described, thereby limiting the number of suitable targets for new cosmetic hair products.

Every cell type in the skin and its appendages expresses about 15,000 different genes and synthesizes therefrom a corresponding number of proteins. However, it is as yet substantially unclear which particular genes are important in hair follicles.

The skin consists of a plurality of different cell types (e.g. fibroblasts, keratinocytes in various states of differentiation, melanocytes, Merkel cells, Langerhans cells, a large number of different cells of the hair follicle or other cutaneous appendages) so that the complexity of genes expressed in the skin is very great. To date, it has not been possible to identify the particular genes or markers associated with the hair follicle from this complex mixture. An additional difficulty is that mRNA molecules occur in the cell in widely ranging concentrations (e.g., between a few and several hundred copies). Thus, weakly expressed genes are difficult to assess with currently available analytical techniques but may very well be of crucial importance in the hair follicle.

The transcriptome of the human hair follicle, that is the totality of all transcribed genes therein, has not been described to date.

Transcriptome analyses of the skin by various methods, including SAGE™ analysis reflects the current state of the art. However, previously described approaches employed isolated keratinocytes (in vitro) or epidermis explants, but as explained above, these artificial models are not representative of the naturally occurring complex events in the skin.

DE-A-101 00 127.4-41 to the present inventors discloses subjecting skin cells to a SAGE™ analysis in order to characterize the complete transcriptome of the skin. DE-A-101 00 121.5-41 of the applicant discloses the identification of markers of stressed or aged skin on the basis of a comparative SAGE™ analysis between stressed and aged skin and unstressed and young skin, respectively. However, information about specific markers of hairy skin is not to be found in these publications.

J Invest Dermatol 2002 July; 119(1):3-13; “A serial analysis of gene expression in sun-damaged human skin”; Urschitz J. et al.; discloses the determination of markers of sun-damaged skin by means of a comparative SAGE™ analysis of full-thickness skin explants taken from in front of the oracle (sun-damage) and behind the oracle (protected from the sun's rays). It is likewise impossible to obtain any information about specific markers of hairy skin from this publication.

There is thus a need to identify as many as possible, and preferably all, of the genes which contribute to the hairiness of the skin.

### SUMMARY OF THE INVENTION

It is an object of the present invention to identify a large portion, if not all, of the genes important for the hairiness of skin. Also encompassed by the invention is the use of this genetic information in methods for determining the homeostasis of hairy skin.

According to a first object of the invention, a method is provided for identifying differentially expressed genes which modulate the hairiness of skin in humans in vitro. An exemplary method comprises:
a) providing a first sample of genetically encoded factors which are expressed, i.e. transcribed and, optionally, also translated, in hairy human skin (Thus, the first sample may comprise mRNA molecules and/or proteins and/or fragments thereof which are obtained from hairy human skin, preferably from hairy scalp);
b) providing a second sample of genetically encoded factors which are expressed, i.e. transcribed and, optionally translated in hairless human skin (Thus, the second sample may comprise mRNA molecules and/or proteins and/or fragments thereof obtained from hairless human skin, preferably from hairless facial skin); and
c) subjecting the samples obtained in steps a) and b) to serial analysis of gene expression (SAGE™) thereby identifying differentially expressed genes and gene products present in hairy and hairless skin.

For the purposes of the present invention “hairless” also means hairiness comprising the fine and scarcely visible vellus hairs.

The method of the invention facilitates an understanding of the complex processes involved in the development of hair and the causal relationships of the changes in hair. Only with this knowledge, is it possible to develop novel targets for cosmetic hair products which exert their effect on the variety of genes expressed in the hair follicle. The present inventors, employing SAGE™ analysis have identified for the first time the differential gene expression patterns present in hairy (with cephalic hair) scalp skin and essentially hairless skin.

According to a second object of the present invention, a method is provided for determining the homeostasis of hairy skin in humans, especially in women, in vitro. An exemplary method entails
a) performing SAGE™ analysis in accordance with the first object of the invention to identity differentially expressed gene products which are associated with hairy skin thereby identifying a sample of hairy-skin associated molecules, e.g., proteins, and/or mRNA molecules and/or fragments thereof;
b) obtaining a skin sample from a patient and determining the presence and optionally, the amount of the hairy-skin associated molecules which are differentially expressed in hairy and hairless skin; and
c) designating the sample of step b) as
   i) healthy or homeostasis-undergoing hairy skin if it comprises predominantly proteins, and/or mRNA molecules or fragments thereof which are expressed at higher levels in hairy skin relative to hairless skin, or as
   ii) diseased or homeostasis-impaired hairy skin if it comprises predominantly proteins, and/or mRNA molecules or fragments thereof which are expressed at higher levels in hairless skin relative to hairy skin.

A third object of the invention relates to yet another embodiment for determining the homeostasis of hairy skin in humans. An exemplary method entails performing SAGE analysis as described above to identify molecules characteristic of healthy hairy human skin. At least two molecules (e.g., mRNA or proteins) identified as important in hairy skin are quantified and the expression ratios relative one to another determined, thereby providing an expression quotient. A hairy skin sample is obtained from a patient and the sample is assessed for the expression ratios of the at least two molecules, the expression quotients in the patient sample are then compared with those provided in Tables 3-12, columns 3 and 5, the patient sample is then designated as healthy hairy skin (homeostasis-undergoing) if the expression ratios of the sample correspond to the expression ratios in hairy skin, or designated as diseased (homeostasis-impaired) skin if the expression ratios of the patient skin sample correspond to the expression ratios of hairless skin.

Examples of disorders or impairments of homeostasis of hairy skin are: pili torti (corkscrew hairs, twisted hairs), monilethrix (beaded hair), wooly hairs (crinkled hair), hair shaft changes with breaks [trichorrhexis nodosa, trichorrhexis invaginata, trichoschisis, trichoptilosis (hair splitting)], hair shaft changes associated with metabolic disorders, pili recurvati, ingrowing hairs, changes in hair color [heterochromia, albinism, poliosis (acquired focal lack of hair pigment), canitis (physiological graying)], hypertrichoses, hirsutism, alopecias (irreversible alopecia: e.g. androgenic alopecia of men and women); reversible alopecia: e.g. symptomatic diffuse alopecias due to infections, noxious chemicals and drugs, hormonal disorders, diseases, etc.) and alopecia greata.

The present invention also provides a kits for performing the methods described above. Also provided in the invention is a biochip which facilitates the practice of the present invention. An exemplary biochip comprises
i. a solid, i.e. rigid or flexible support and
ii. probes immobilized thereon which are capable of specific binding to at least one of the proteins, mRNA molecules or fragments thereof which are defined by their UniGene accession number in column 7 in Tables 3 to 12.

A biochip which is particularly preferred according to the invention includes probes which are selected from those capable of specific binding to at least one of the proteins, mRNA molecules or fragments thereof which are defined in column 1 in Tables 3 to 6 by the following consecutive serial number: 2, 4, 9, 12, 14, 16, 22, 25, 29, 31, 35, 36, 38, 39, 40, 42, 43, 44, 46, 59, 62, 63, 65, 67, 68, 69, 74.

The present invention also relates to a method for screening pharmaceutically and/or cosmetically active ingredients for efficacy in the treatment of disorders or impairment of homeostasis of hairy skin in vitro. The method entails incubating the hairy skin sample in the presence and absence of the ingredient and determining whether the homeostasis of hairy skin using the methods set forth above is improved. Ingredients which improve the state of the hairy skin can then be isolated and formulated with cosmetically and/or pharmaceutically suitable and acceptable carriers.

### DETAILED DESCRIPTION OF THE INVENTION

Cosmetic hair products available on the market usually exert their effects on the hair shaft (e.g. hair colorations). In accordance with the present invention, it is now possible for the first time to gain an understanding of the complex genetic and biological processes in the hair follicle. Identification of suitable markers present in the hair follicle thus permits a targeted search for substances or combinations of substances having a broad range of effects on gene expression in the hair follicle. It has not been possible until the present time to develop products of this type, because a large number of the hair follicle markers were as yet, unknown.

The technique employed to establish the transcriptome of hairy skin was “serial analysis of gene expression” (SAGE™). This technique permits simultaneous identification and quantification of all genes expressed in hairy skin. It is true that gene expression can also be analyzed by quantifying specific mRNA molecules (e.g. Northern blotting, RNase protection experiments). However, often these techniques measure only a relatively limited number of genes. It would theoretically be possible for the techniques of MPSS (massive parallel signature sequencing) or techniques based on differential display to replace the SAGE™ analysis. However, in practice, the SAGE™ technique is faster and more reliable than alternative methods and is thus preferred.

Comparison of the transcriptome of hairy skin with the transcriptome of hairless skin permits differentiation between genes relevant and not relevant for the hairiness of the skin.

Human skin from healthy female donors was used for the SAGE™ analysis. The SAGE™ analysis was carried out as described in EP-A-0 761 822 and by Velculescu, V. E. et al., 1995 Science 270, 484-487. This technique permits simultaneous identification and quantification of the genes expressed in the hairy scalp. Comparison of the transcriptome of hairy scalp with the transcriptome of facial skin permits the identification of relevant genes for hairy scalp. These may be genes which are highly expressed in hairy scalp or alternatively, genes which are characterized in that their expression is diminished when compared with hairless facial skin. SAGE™ analysis is a technique with particular sensitivity and even surprisingly reveals interindividual differences in gene expression profiles. For description of the transcriptome of hairy skin, therefore, comparison with the transcriptome of hairless skin is especially effective when the analyzed tissues are derived from one individual, that is one donor. Thus, interindividual differences in gene expression do not apply. Hairy tissues from the region above the ear and the region behind the ear are obtained from a patient during plastic surgery operations such as, for example, “lower facelifts”. At the same time, cutaneous tissue that—at least in female donors—bears only vellus hair is removed in front of the ear. Analysis of such tissue samples therefore permits description of the transcriptomes of hairy scalp and hairless (or only having vellus hair) facial skin while avoiding interindividual differences in gene expression.

Human skin from a healthy female donor (65 years old) was used for the SAGE™ analysis. The SAGE™ analysis was carried out as described in EP-A-0 761 822 and by Velculescu, V. E. et al., 1995 Science 270, 484-487. Two SAGE™ libraries from a patient's skin samples from various locations were analyzed. The first sample was derived from a removal point above the ear and provided skin with hair of the head. The second sample was derived from the same operation, was taken in front of the ear and provided skin with only vellus hair. For further analysis, the two SAGE™ libraries were normalized to the average number of tags. The two libraries were compared with one another in order to identify genes with hair-specific regulation. As expected for two libraries of the same tissue type, the tag repertoire of the two skin libraries is substantially similar. Despite the similarity of the tissues and the relatively small number of tags, 74 tags show a significantly differential expression at a significance level of p>0.05.

For hairy skin, it is possible to show an increased number of tags for genes which are expressed in the hair follicle or in hair appendages. Besides an increased level of hair- and hair follicle-typical keratins, hairy skin also shows, for example, elevated FGF7 levels, a transcription factor involved in hair development. Dermcidin and cystatin, both involved in the defense against bacterial and viral invasions in the hair shaft, likewise show significantly increased expression levels in hairy skin. Expression of differentiation-dependent genes of the interfollicular epidermis is increased in hairless skin as reflected in the results of the investigation.

The differentially expressed genes are led by keratin 10, a typical marker for the differentiation of stratified epithelia. Keratin 1, the partner of keratin 10, does not show these differences in expression. Analysis of further differentiation-dependently expressed genes likewise confirms increased expression thereof in hairless skin (See Table 2). Another keratin, keratin 2e, is according to the literature, expressed mainly in hairless skin, and this is likewise observed in the analysis carried out. Expression of keratins 5, 14 and 15, which are expressed in the basal layer of the epidermis and in the hair follicles, is, as expected, virtually identical in the two samples (Table. 2).

Table 1 lists markers which are indicated in the literature to be differentially expressed in the hair follicle compared with the remainder of the skin (interfollicular skin). They serve as positive controls for the experiment.
+H=with hair (main source above the ear)
−H=without hair (main source in front of the ear)

Table 2 shows differentiation-dependent genes of the interfollicular epidermis.
HF=hair follicle
ORS=outer root sheath
IRS=inner root sheath
+H=with hair (main source above the ear)
−H=without hair (main source in front of the ear)

Further SAGE™ analyses of human skin were taken into account for describing and verifying subsignificant differences in gene expression (Tables 7 to 12). Thus, low expression of a hair-specific gene is to be expected not only in hairless facial skin but also in other hairless body skin. The subsignificant differences from the comparison of the transcriptome of hairy scalp with the transcriptome of hairless facial skin were therefore compared with the expression profile of hairless breast skin. Breast skin from a healthy female donor (69 years old) was used for the SAGE™ analysis of breast skin. It was possible thereby to confirm some significant differences in gene expression.

Tables 3 to 12 contain a detailed listing of genes differentially expressed in hairy and hairless skin identified with the aid of the method of the invention, and include:
a consecutive SEQ ID NO: in column 1,
the tag sequence used in column 2,
the quotient of the measured relative expression frequency in hairy scalp (scalp) and the measured relative expression frequency in hairless facial skin (face) in column 3,
the significance of the values mentioned in column 3 in column 4,
the quotient of the measured relative expression frequency in hairy scalp (scalp) and the measured relative expression frequency in hairless body skin (breast) in column 5,
the significance of the values mentioned in column 5 in column 6,
the UniGene accession number in column 7,
a brief description of the gene or gene product in column 8 and
in Tables 8, 10 and 12 the quotient (scalp/face)/(scalp/breast) in column 9.

The quotient in column 3 indicates the strength of differential expression, i.e. the factor by which the particular gene is more highly or strongly expressed in hairy scalp (scalp) than in hairless facial skin (face), or vice versa.

The quotient in column 5 indicates the strength of differential expression, i.e. the factor by which the particular gene is more strongly expressed in hairy scalp (scalp) than in hairless body skin (breast), or vice versa.

The respective genes and gene products are disclosed under their UniGene accession number in the database of the National Center for Biotechnology Information (NCBI). This database can be accessed in the internet under the following address:
www.ncbi.nlm.nih.gov/.

The genes and gene products can additionally be accessed directly under the internet addresses
www.ncbi.nlm.nih.gov/UniGene/Hs.Home or
www.ncbi.nlm.nih.gov/genome/guide.

Table 3 lists all the genes which exhibit at least a 10-fold differential in expression levels in hairy scalp (scalp) when compared with hairless facial skin (face) with a p value of p>0.05 (signif>1.3).

Table 4 lists all the genes which exhibit at least a 5-fold differential in expression levels in hairy scalp (scalp) when compared with hairless facial skin (face) with a p value of p>0.05 (signif>1.3)

Table 5 lists all the genes which exhibit at least a 3-fold differential in expression levels in hairy scalp (scalp) when compared with hairless facial skin (face) with a p value of p>0.05 (signif>1.3)

Table 6 lists all the genes which exhibit at least a 1.9-fold differential in expression levels in hairy scalp (scalp) when compared with hairless facial skin (face) with a p value of p>0.05 (signif>1.3)

Table 7 lists all the genes which exhibit at least a 5-fold differential in expression levels in hairy scalp (scalp) when compared with hairless facial skin (face) with a p value of p<0.05 (signif<1.3) and which exhibit at least a 5-fold differential in expression levels in hairy scalp (scalp) when compared with hairless body skin (breast) with a p value of p>0.05 (signif>1.3). Comparison of the subsignificant scalp/face data with independent SAGE™ experiments (scalp/breast) confirms the differential gene expression and validates the markers of hairy skin.

Table 8 lists all the genes which exhibit at least a 5-fold differential in expression levels in hairy scalp (scalp) when compared with hairless facial skin (face) with a p value of p<0.05 (signif<1.3) and which exhibit at least a 5-fold differential in expression levels in hairy scalp (scalp) when compared with hairless body skin (breast) with a p value of p<0.05 (signif<1.3) whose expression differs by less than one power of 10, i.e. the quotient (scalp/face)/(scalp/breast) is less than 10 or greater than 0.1. Comparison of the subsignificant scalp/face data with independent SAGE™ experiments (scalp/breast) confirms the differential gene expression and validates the markers of hairy skin.

Table 9 lists all the genes which exhibit at least a 3-fold differential in expression levels in hairy scalp (scalp) when compared with hairless facial skin (face) with a p value of p<0.05 (signif<1.3) and which exhibit at least a 3-fold differential in expression level in hairy scalp (scalp) when compared with hairless body skin (breast) with a p value of p>0.05 (signif>1.3). Comparison of the subsignificant scalp/face data with independent SAGE™ experiments (scalp/breast) confirms the differential gene expression and validates the markers of hairy skin.

Table 10 lists all genes which exhibit at least a 3-fold differential in expression level when expressed in hairy scalp (scalp) when compared with hairless facial skin (face) with a p value of p<0.05 (signif<1.3) and exhibit at least a 3-fold differential in expression level in hairy scalp (scalp) when compared with hairless body skin (breast) with a p value of p<0.05 (signif<1.3) whose expression differs by less than one power of 10, i.e. the quotient (scalp/face)/(scalp/breast) is less than 10 or greater than 0.1. Comparison of the subsignificant scalp/face data with independent SAGE™ experiments (scalp/breast) confirms the differential gene expression and validates the markers of hairy skin.

Table 11 lists all genes which exhibit at least a 1.9-fold differential in expression levels in hairy scalp (scalp) when compared with hairless facial skin (face) with a p value of p<0.05 (signif<1.3) and which exhibit at least a 1.9-fold differential in expression level in hairy scalp (scalp) when compared with hairless body skin (breast) with a p value of p>0.05 (signif>1.3). Comparison of the subsignificant scalp/face data with independent SAGE™ experiments (scalp/breast) confirms the differential gene expression and validates the markers of hairy skin.

Table 12 lists all genes which exhibit at least a 1.9-fold differential in expression level in hairy scalp (scalp) when compared with hairless facial skin (face) with a p value of p<0.05 (signif<1.3) and which exhibit at least a 1.9-fold differential in expression level in hairy scalp (scalp) when compared with hairless body skin (breast) with a p value of p<0.05 (signif<1.3) whose expression differs by less than one power of 10, i.e. the quotient (scalp/face)/(scalp/breast) is less than 10 or greater than 0.1. Comparison of the subsignificant scalp/face data with independent SAGE™ experiments (scalp/breast) confirms the differential gene expression and validates the markers of hairy skin.

According to a second object of the present invention, a method is provided for determining the homeostasis of hairy skin in humans, especially in women, in vitro. An exemplary method entails
a) performing SAGE analysis in accordance with the first object of the invention to identity differentially expressed gene products which are associated with hairy skin thereby identifying a mixture of hairy-skin associated molecules, e.g., proteins, and/or mRNA molecules or fragments thereof;
b) obtaining a skin sample from a patient and determining the presence and optionally, the amount of the hairy-skin associated molecules which are differentially expressed in hairy and hairless skin; and
c) designating the sample of step b) as
   i) healthy or homeostasis-undergoing hairy skin if it comprises predominantly proteins, and/or mRNA molecules or fragments thereof which are expressed at higher levels in hairy skin relative to hairless skin,
      or as
   ii) diseased or homeostasis-impaired hairy skin if it comprises predominantly proteins, and/or mRNA molecules or fragments thereof which are expressed at higher levels in hairless skin relative to hairy skin.

Examples of disorders or impairments of homeostasis of hairy skin are: pili torti (corkscrew hairs, twisted hairs), monilethrix (beaded hair), wooly hairs (crinkled hair), hair shaft changes with breaks [trichorrhexis nodosa, trichorrhexis invaginata, trichoschisis, trichoptilosis (hair splitting)], hair shaft changes associated with metabolic disorders, pili recurvati, ingrowing hairs, changes in hair color [heterochromia, albinism, poliosis (acquired focal lack of hair pigment), canitis (physiological graying)], hypertrichoses, hirsutism, alopecias (irreversible alopecia: e.g. androgenic alopecia of men and women); reversible alopecia: e.g. symptomatic diffuse alopecias due to infections, chem. noxae and drugs, hormonal disorders, diseases, etc.) and alopecia greata.

The sample of step b) of the method of the invention for determining the homeostasis of hairy skin in a patient may be obtained from full-thickness skin samples, hairy skin equivalents, isolated hair follicles, hair follicle equivalents or cells of hairy skin.

It may be sufficient in step c) of the method to investigate the obtained sample for the presence of at least one of the proteins, mRNA molecules or fragments thereof which are identified by serial analysis of gene expression (SAGE) as being exclusively expressed in hairy or exclusively in hairless skin. In all other cases (e.g., genes which are differentially expressed in both tissue types), the amount of the differentially expressed molecules must also be investigated in step b), i.e. the expression must be quantified.

In step c) of the method for determining the homeostasis of hairy skin, the sample investigated in b) is designated as healthy hairy skin if it comprises predominantly proteins, mRNA molecules or fragments of proteins or mRNA molecules which are expressed at higher levels in hairy skin when compared to hairless skin, i.e. that the sample either comprises a greater number of molecules (e.g., proteins or mRNA) typically expressed in hairy skin as compared to those typically expressed in hairless skin (qualitative differentiation), or comprises more copies of molecules typically expressed in hairy skin than those typically present in hairless skin (quantitative differentiation). A complementary procedure is used to assign to diseased or homeostasis-impaired hairy skin.

A preferred embodiment of the method of the invention for determining the homeostasis of hairy skin comprises determining whether at least one of the proteins, mRNA molecules or fragments of proteins or mRNA molecules which are defined by their UniGene accession number in column 7 in Tables 11 and 12 are present in the sample, and further comparing the expression quotients indicated in column 3 and column 5 in Tables 11 and 12. In step c) the sample is assigned to healthy hairy skin if it comprises predominantly proteins, mRNA molecules or fragments thereof which are expressed at approximately 1.9-fold higher levels in healthy hairy skin as compared to hairless skin. Alternatively the sample is designated as diseased or homeostasis-impaired hairy skin if it comprises predominantly proteins, mRNA molecules or thereof which are expressed at approximately 1.9-fold higher levels in hairless skin as compared to hairy skin.

A further preferred embodiment of the method of the invention for determining the homeostasis of hairy skin comprises determining the presence and, where appropriate, the amount of at least one of the proteins, mRNA molecules or fragments thereof as defined by their UniGene accession number in column 7 in Tables 9 and 10 in the sample, and comparing the results with the expression quotients indicated in column 3 and column 5 in Tables 9 and 10. The sample is designated as healthy hairy skin if it comprises predominantly proteins, mRNA molecules or fragments thereof which are expressed at least 3-fold higher in healthy hairy skin as compared to hairless skin. Alternatively, the sample is designated as diseased or homeostasis-impaired hairy skin if it comprises predominantly proteins, mRNA molecules or fragments thereof which are expressed at least 3-fold higher in hairless skin as compared to hairy skin.

A further preferred embodiment of the method of the invention for determining the homeostasis of hairy skin comprises assessing the sample for the presence and where appropriate, the amount of at least one of the proteins, mRNA molecules or fragments thereof as defined by their UniGene accession number in column 7 in Tables 7 and 8, comparing the expression quotients indicated in column 3 and column 5 in Tables 7 and 8, and designating the sample as healthy hairy skin if it comprises predominantly proteins, mRNA molecules or fragments thereof which are expressed at least 5-fold higher in healthy hairy skin when compared to hairless skin. Alternatively, the sample is designated as diseased or homeostasis-impaired hairy skin if it comprises predominantly proteins, mRNA molecules or fragments thereof which are expressed at least 5-fold higher in hairless skin when compared to hairy skin.

A particularly preferred embodiment of the method of the invention for determining the homeostasis of hairy skin comprises assessing the patient sample for the presence and where appropriate, the amount of at least one of the proteins, mRNA molecules or fragments thereof as defined by their UniGene accession number in column 7 in Table 6, comparing the results with the expression quotients indicated in column 3 and column 5 in Table 6, and designating the sample as healthy hairy skin if it comprises predominantly proteins, mRNA molecules or fragments thereof which are expressed at a level at least 1.9-fold higher in healthy hairy skin when compared to hairless skin. Alternatively, the sample is designated as diseased or homeostasis-impaired hairy skin if it comprises predominantly proteins, mRNA molecules or fragments thereof proteins which are expressed at a level at least 1.9-fold higher in hairless skin when compared to hairy skin.

A further particularly preferred embodiment of the method of the invention for determining the homeostasis of hairy skin comprises assessing the sample for the presence and where appropriate, the amount of at least one of the proteins, mRNA molecules or fragments thereof as defined by their UniGene accession number in column 7 in Table 5, comparing the results with the expression quotients indicated in column 3 and column 5 in Table 5, and designating the sample as healthy hairy skin if it comprises predominantly proteins, mRNA molecules or fragments thereof which are expressed at levels at least 3-fold higher in healthy hairy skin when compared to hairless skin. Alternatively, the sample is designated as diseased or homeostasis-impaired hairy skin if it comprises predominantly proteins, mRNA molecules or fragments thereof proteins which are expressed at levels at least 3-fold higher in hairless skin when compared to hairy skin.

A further particularly preferred embodiment of the method of the invention for determining the homeostasis of hairy skin comprises assessing the sample for the presence and where appropriate, the amount of at least one of the proteins, mRNA molecules or fragments thereof as defined by their UniGene accession number in column 7 in Table 4, comparing the results with the expression quotients indicated in column 3 and column 5 in Table 4, and designating the sample as healthy hairy skin if it comprises predominantly proteins, mRNA molecules or fragments thereof which are expressed at least 5-fold higher in healthy hairy skin when compared to hairless skin. Alternatively, the sample is designated as diseased or homeostasis-impaired hairy skin if it comprises predominantly proteins, mRNA molecules or fragments thereof which are expressed at least 5-fold higher in hairless skin when compared to hairy skin.

A very particularly preferred embodiment of the method of the invention for determining the homeostasis of hairy skin comprises assessing the sample for the presence and where appropriate, the amount of at least one of the proteins, mRNA molecules or fragments thereof as defined by their UniGene accession number in column 7 in Table 3, comparing the results with the expression quotients indicated in column 3 and column 5 in Table 3, and designating the sample as healthy hairy skin if it comprises predominantly proteins, mRNA molecules or fragments thereof which are expressed at least 10-fold higher in healthy hairy skin when compared to hairless skin. Alternatively, the sample is designated diseased or homeostasis-impaired hairy skin if it comprises predominantly proteins, mRNA molecules or fragments thereof which are expressed at least 10-fold higher in hairless skin when compared to hairy skin.

The state of the skin can also be described by quantifying a plurality of expression products of genes important for hairy skin (e.g., markers) which must then be active among themselves in a characteristic ratio in order to represent healthy (homeostasis-undergoing) hairy skin, or must be active in the characteristic ratio differing therefrom in order to represent diseased (homeostasis-impaired) skin.

The third object of the present invention therefore further relates to yet another embodiment of a method for determining the homeostasis of hairy skin in humans, especially in women, in vitro.

An exemplary method entails
a) performing SAGE analysis on hairy and hairless skin as set forth in the first object of the invention, thereby identifying a plurality of molecules which are important for hairy skin and obtaining a patient sample of hairy skin.
b) quantifying at least two of the proteins, mRNA molecules or fragments thereof characteristic of hairy skin identified by SAGE analysis;
c) determining the expression ratios of the at least two proteins, mRNA molecules or fragments thereof relative one to another, thereby providing an expression quotient,
d) comparing the expression ratios from c) with the expression ratios typically present in hairy and in hairless skin for the molecules quantified in b), in particular with the expression ratios provided in Tables 3 to 12, columns 3 and 5, and
e) designating the patient sample as healthy (homeostasis-undergoing) hairy skin if the expression ratios of the sample correspond to the expression ratios in hairy skin, or designating the sample obtained in step a) as diseased (homeostasis-impaired) skin if the expression ratios of the patient skin sample correspond to the expression ratios of hairless skin.

The mixture in step a) of the method of the invention for determining the homeostasis of hairy skin is preferably obtained from a skin sample, in particular from a full-thickness skin sample or from an epidermis sample. In this connection, the full-thickness skin sample facilitates a more comprehensive comparison if the SAGE libraries which are likewise obtained from full-thickness skin. The epidermis sample is, on the other hand, easier to obtain, for example by applying an adhesive tape to the skin and peeling it off, as described in WO 00/10579, which is incorporated herein by reference.

In a further embodiment of the method of the invention for determining the homeostasis of hairy skin, the mixture is obtained in step a) by microdialysis. The technique of microdialysis is described for example in “Microdialysis: A method for measurement of local tissue metabolism”, Nielsen P S, Winge K, Petersen L M; Ugeskr Laeger 1999 Mar. 22 161:12 1735-8; and in “Cutaneous microdialysis for human in vivo dermal absorption studies”, Anderson, C. et al.; Drugs Pharm. Sci., 1998, 91, 231-244; and also in the internet under www.microdialysis.se/techniqu which are incorporated herein by reference.

When microdialysis is used, typically a probe is introduced into the skin and a suitable carrier solution is used to rinse the probe slowly. After the acute reactions following the puncture have subsided, the microdialysis provides proteins, mRNA molecules or fragments of proteins or mRNA molecules which occur in the extracellular space and which can then be isolated, for example by fractionation of the carrier liquid, and analyzed in vitro. Microdialysis is less invasive than removal of a full-thickness skin sample; however, a disadvantage is that it is confined to obtaining molecules occurring in the extracellular space.

A further preferred embodiment of the methods of the second and third objects of the invention for determining the homeostasis of hairy skin comprises assessing the sample(s) for the presence and, where appropriate, the amount of at least one of the proteins or fragments thereof. Such determinations can be carried out by a method selected from
i. one- or two-dimensional gel electrophoresis
ii. affinity chromatography
iii. protein-protein complexation in solution
iv. mass spectrometry, especially matrix-assisted laser desorption ionization (MALDI) and, in particular,
v. use of protein chips,
or by suitable combinations of these methods.

These methods which can be employed according to the invention are described in the review article by Akhilesh Pandey and Matthias Mann: “Proteomics to study genes and genomes”, Nature, Volume 405, Number 6788, 837-846 (2000), and the references indicated therein, which are incorporated herein by reference.

2D gel electrophoresis is described for example in L. D. Adams, Two-dimensional Gel Electrophoresis using the Isodalt System or in L. D. Adams & S. R. Gallagher, Two-dimensional Gel Electrophoresis using the O'Farrell System; both in Current Protocols in Molecular Biology (1997, Eds. F. M. Ausubel et al.), Unit 10.3.1-10.4.13; or in 2-D Electrophoresis Manual; T. Berkelman, T. Senstedt; Amersham Pharmacia Biotech, 1998 (Order No. 80-6429-60).

Characterization of the proteins or protein fragments by mass spectrometry takes place in a manner known to those skilled in the art, for example as described in the following references: Methods in Molecular Biology, 1999; Vol. 112; 2-D Proteome Analysis Protocols; Editor: A. J. Link; Humana Press; Totowa; N.J. Especially: Courchesne, P. L. and Patterson, S. D.; pp. 487-512 therein and Carr, S. A. and Annan, R. S.; 1997; in: Current Protocols in Molecular Biology; Editor: Ausubel, F. M. et al.; John Wiley and Sons, Inc. 10.2.1-10.21.27.

A further preferred embodiment of the methods of the second and third objects of the invention for determining the homeostasis of hairy skin comprises assessing the sample(s) for the presence and, where appropriate, the amount of at least one of mRNA molecule and/or fragments thereof. Such determinations can be carried out by a method selected from
i. Northern blots
ii. reverse transcriptase polymerase chain reaction (RT-PCR),
iii. RNase protection experiments,
iv. dot blots,
v. cDNA sequencing,
vi. clone hybridization,
vii. differential display,
viii. subtractive hybridization,
ix. cDNA fragment fingerprinting,
x. total gene expression analysis (TOGA),
xi. serial analysis of gene expression (SAGE),
xii. massively parallel signature sequencing (MPSS®) and, in particular,
xiii. use of nucleic acid chips,
or by suitable combinations of these methods.

These methods which can be employed according to the invention are described in the review articles by Akhilesh Pandey and Matthias Mann: “Proteomics to study genes and genomes”, Nature, Volume 405, Number 6788, 837-846 (2000), and “Genomics, gene expression and DNA arrays”, Nature, Volume 405, Number 6788, 827-836 (2000), and the references indicated therein, which are incorporated herein by reference.

The TOGA method is described in “J. Gregor Sutcliffe et al, TOGA: An automated parsing technology for analyzing expression of nearly all genes, Proceedings of the National Academy of Sciences of the United States of America (PNAS), Vol. 97, No. 5, pp. 1976-1981 (2000)”, which is incorporated herein by reference.

The MPSS® method is described in U.S. Pat. No. 6,013,445, which is incorporated herein by reference.

However, it is also possible according to the invention to employ other methods known to the skilled worker for investigating for the presence and, where appropriate, the amount of at least one of the proteins, mRNA molecules or fragments thereof in a sample.

A further preferred embodiment of the method of the invention for determining the homeostasis of hairy skin entails assessing the sample of step b) for the presence and, optionally, the amount of from 1 to about 5000, preferably from 1 to about 1000, in particular from about 10 to about 500, preferably from about 10 to about 250, particularly preferably from about 10 to about 100 and very particularly preferably from about 10 to about 50 of the proteins, mRNA molecules or fragments thereof which are defined by their UniGene accession number in column 7 in Tables 3 to 12.

The present invention further relates to a test kit for determining the homeostasis of hairy skin in humans in vitro, including means for carrying out the methods of the invention for determining the homeostasis of hairy skin.

The present invention also relates to a biochip for determining the homeostasis of hairy skin in humans in vitro, including
i. a solid, i.e. rigid or flexible support and
ii. probes immobilized thereon which are capable of specific binding to at least one of the proteins, mRNA molecules or fragments thereof which are defined by their UniGene accession number in column 7 in Tables 3 to 12.

A biochip which is particularly preferred according to the invention includes probes which are selected from those capable of specific binding to at least one of the proteins, mRNA molecules or fragments thereof which are defined in column 1 in Tables 3 to 6 by the following SEQ ID NOS: 2, 4, 9, 12, 14, 16, 22, 25, 29, 31, 35, 36, 38, 39, 40, 42, 43, 44, 46, 59, 62, 63, 65, 67, 68, 69, 74.

A biochip is a miniaturized functional element having molecules, in particular biomolecules, which can act as specific interaction partners and are immobilized on a surface. The structure of these functional elements frequently comprises rows and columns; the term used is then chip arrays. Since thousands of biological or biochemical functional elements can be arranged on one chip, they must generally be fabricated by microengineering methods. Particularly suitable biological and biochemical functional elements are: DNA, RNA, PNA (nucleic acids and their chemical derivatives may be for example in the form of single strands, triplex structures or combinations thereof), saccharides, peptides, proteins (e.g. antibodies, antigens, receptors) and derivatives of combinatorial chemistry (e.g. organic molecules).

Biochips generally have a 2D basic area for coating with biologically or biochemically functional materials. The basic areas may for example also be formed by walls of one or more capillaries or by channels. Reference may be made to the following publications for example for the prior art: Nature Genetics, Vol. 21, supplement (complete), January 1999 (BioChips); Nature Biotechnology, Vol. 16, pp. 981-983, October 1998 (BioChips); Trends in Biotechnology, Vol. 16, pp. 301-306, July 1998 (BioChips) and the previously mentioned review articles by Akhilesh Pandey and Matthias Mann: “Proteomics to study genes and genomes”, Nature, Volume 405, Number 6788, 837-846 (2000), and “Genomics, gene expression and DNA arrays”, Nature, Volume 405, Number 6788, 827-836 (2000), and the references cited therein, which are incorporated herein by reference.

A clear description of the practical methods for using DNA chip technology is provided by the books “DNA Microarrays: A Practical Approach” (editor: Mark Schena, 1999, Oxford University Press) and “Microarray Biochip Technology” (editor: Mark Schena, 2000, Eaton Publishing), which are incorporated herein by reference.

The DNA chip technology which is particularly preferred for the purposes of the present invention is based on the ability of nucleic acids to enter into complementary base pairings. This technical principle, which is referred to as hybridization, has been employed for some years in Southern blotting and Northern blotting analyses. Compared with these conventional methods, in which only a few genes are analyzed, DNA chip technology permits parallel investigation of some hundreds and up to several ten thousands of genes. A DNA chip consists essentially of a support material (e.g. glass or plastic) on which single-stranded, gene-specific probes are immobilized in high density at a defined site (spot). Problems associated therewith are assessed as being the technique of probe application and the chemistry of probe immobilization.

In the current state of the art, several ways for immobilizing probes are implemented:

E. M. Southern (E. M. Southern et al (1992), Nucleic Acid Research 20, 1679-1684 and E. M. Southern et al. (1997), Nucleic Acid Research 25, 1155-1161) describes the production of oligonucleotide arrays by direct synthesis on a glass surface which has been derivatized with 3-glycidoxypropyltrimethoxysilane and then with a glycol.

A similar method achieves the in situ synthesis of oligonucleotides by means of a photosensitive, combinatorial chemistry which can be compared with photolithographic techniques (Pease, A. C. et al. (1994), Proc. Natl. Acad Sci USA 91, 5022-5026).

Besides these techniques which are based on the in situ synthesis of oligonucleotides, it is likewise possible for already present DNA molecules to be bound to surfaces of support material.

P. O. Brown (DeRisi et al. (1997), Science 278, 680-686) describes the immobilization of DNA on polylysine-coated glass surfaces.

The publication by L. M. Smith (Guo, Z. et al. (1994), Nucleic Acid Research 22, 5456-5465) discloses a similar method: oligonucleotides having a 5′ terminal amino group can be bound to a glass surface which has been treated with 3-aminopropyltrimethoxysilane and then with 1,4-phenyl diisothiocyanate.

The DNA probes can be applied to a support with a so-called pin spotter. For this purpose, thin metal needles with, for example, a diameter of 250 μm are dipped into probe solutions and then transfer the pendant sample material with defined volumes to the support material of the DNA chip.

The probes are preferably applied by means of a piezo-controlled nanodispenser which, similar to an inkjet printer, applies probe solutions with a volume of 100 picoliters without contact to the surface of the support material.

The probes are immobilized for example as described in EP-A-0 965 647. In this case, DNA probes are generated by PCR using a sequence-specific primer pair, one primer being modified at the 5′ end and having a linker with a free amino group. This ensures that a defined strand of the PCR products can be bound to a glass surface which has been treated with 3-aminopropyltrimethoxysilane and then with 1,4-phenyl diisothiocyanate. The gene-specific PCR products should ideally have a defined nucleic acid sequence in a length of 200-400 bp and not include redundant sequences. After the immobilization of the PCR products via the derivatized primer, the complementary strand of the PCR product is removed by incubation at 96° C. for 10 min.

In a typical application for DNA chips, mRNA is isolated from the two cell populations to be compared. The isolated mRNAs are converted into cDNA by reverse transcription using, for example, fluorescence-labeled nucleotides. In this case, the samples to be compared are labeled with, for example, red or green fluorescent nucleotides. The cDNAs are then hybridized with gene probes immobilized on the DNA chip and subsequently the bound fluorescences are quantified.

The analysis chips mentioned in DE-A-100 28 257.1-52 and in DE-A-101 02 063.5-52 are very particularly preferred for producing small biochips (including up to about 500 probes). These analysis chips have an electrically addressable structure which allows electrofocusing of the samples. This advantageously makes it possible for samples to be focused and immobilized irrespective of their viscosity with the aid of electrodes at defined points in a grid of points (array). The focusability simultaneously results in an increase in the local concentration of the samples and thus a higher specificity. During the analysis itself it is possible to address the test material on the individual positions of the array. It is thus potentially possible for all the information investigated to be traced with the highest possible sensitivity. Cross-contamination by adjacent spots is virtually precluded.

The biochip of the invention preferably includes from 1 to about 5000, more preferably from 1 to about 1000, in particular from about 10 to about 500, preferably from about 10 to about 250, particularly preferably from about 10 to about 100 and very particularly preferably from about 10 to about 50 mutually different probes. The mutually different probes may in each case be present in more than one copy on the chip.

The biochip of the invention preferably includes nucleic acid probes, especially RNA or PNA probes, particularly preferably DNA probes. The nucleic acid probes preferably have a length from about 10 to about 1000, in particular from about 10 to about 800, preferably from about 100 to about 600, particularly preferably from about 200 to about 400 nucleotides.

In a further preferred form, the biochip of the invention includes peptide or protein probes, in particular antibodies.

The present invention further relates to the use of the proteins, mRNA molecules or fragments thereof defined by their UniGene accession number in column 7 in Tables 3 to 12 as markers of hairy skin in humans.

In this connection, some of the genes and gene products listed in Tables 3 to 12 are of particular interest, for example the protein CDT6, whose role as a hair cycle marker is particularly surprising. It was anticipated that VEGF or angiopoietin would be more appropriate markers in this regard. The same applies to proteins of the 14-3-3 family. Although these are expressed ubiquitously, it was not easy to predict which isoforms are involved in the hair cycle, nor which expression pattern these isoforms will possess.

In the overrepresented groups, especially the DPPIV family and the DNA helicases are noteworthy. The skilled worker would have been unable to predict the involvement of either of these gene families in the hair cycle.

Mention should also be made of the genes of melanin biosynthesis. It is unexpected and surprising that the genes of melanin biosynthesis are more strongly expressed in the catagenic than in the anagenic hair follicle.

The present invention further relates to a test method for detecting the efficacy of cosmetic or pharmaceutical active ingredients for disorders or impairments of homeostasis of hairy skin in vitro. An exemplary method entails
a) determining the skin status of hairy skin by a method of the invention
   for determining the homeostasis of hairy skin, or by means of a test kit of the invention for determining the homeostasis of hairy skin, or by means of a biochip of the invention,
b) applying an active ingredient for disorders or impairments of homeostasis of hairy skin at least once to the hairy skin,
c) reassessing the skin status of hairy skin by a method of the invention for determining the homeostasis of hairy skin, or by means of a test kit of the invention for determining the homeostasis of hairy skin, or by means of a biochip of the invention, and
d) measuring the efficacy of the active ingredient by comparing the results from a) and c).

The test method of the invention can be carried out with full-thickness skin samples, hairy skin equivalents, isolated hair follicles, hair follicle equivalents or cells of hairy skin.

The present invention further relates to a test kit for detecting the efficacy of cosmetic or pharmaceutically active ingredients for disorders or impairments of homeostasis of hairy skin, including means for carrying out the test method of the invention.

The present invention further relates to the use of the proteins, mRNA molecules or fragments thereof which are defined by their UniGene accession number in column 7 in Tables 3 to 12 for detecting the efficacy of cosmetic or pharmaceutical active ingredients for disorders or impairments of homeostasis of hairy skin.

The present invention further relates to a screening method for identifying cosmetic or pharmaceutically active ingredients for disorders or impairments of homeostasis of hairy skin in vitro. An exemplary method entails
a) determining the skin status of hairy skin by a method of the invention for determining the homeostasis of hairy skin, or by means of a test kit of the invention for determining the homeostasis of hairy skin, or by means of a biochip of the invention,
b) applying a potential active ingredient for disorders or impairments of homeostasis of hairy skin at least once to the skin,
c) determining the skin status of hairy skin by a method of the invention for determining the homeostasis of hairy skin, or by means of a test kit of the invention for determining the homeostasis of hairy skin, or by means of a biochip of the invention, and
d) identifying effective active ingredients by comparing the results from a) and c).

The present invention further relates to the use of the proteins, mRNA molecules or fragments thereof which are defined by their UniGene accession number in column 7 in Tables 3 to 12 for identifying cosmetic or pharmaceutical active ingredients for disorders or impairments of homeostasis of hairy skin.

The present invention further relates to a method for producing a cosmetic or pharmaceutical preparation for disorders or impairments of homeostasis of hairy skin. An exemplary method entails
a) identifying effective active ingredients in accordance with the screening method of the invention, or of the use for identifying cosmetic or pharmaceutical active ingredients for disorders or impairments of homeostasis of hairy skin, and
b) mixing the active ingredients so identified with cosmetically and pharmacologically suitable and acceptable carriers.

Tables:

TABLE 1

| | | | | | | Localization/ |
|---|---|---|---|---|---|---|
| Gene 1.1. | Tag | Quotient | +Hair | −Hair | Signif. | function |
| hHa Keratin 1 | GCTGGAGTTGC | 6.00 | 3.02 | 0.00 | 0.91 | HF, matrix cells |
| hHa Keratin | GTCTGGAAGGA | 4.00 | 2.02 | 0.00 | 0.61 | HF, matrix cells |
| hHb Keratin 6 | TGCTTTTGCCT | 2.00 | 1.01 | 0.00 | 0.31 | HF |
| Keratin 6a | AAAGCACAAGT | 2.00 | 1.01 | 0.00 | 0.31 | HF, ORS |
| Keratin 6b | CGAATGTCCTT | 2.03 | 2.02 | 0.99 | 0.21 | HF, ORS |
| Keratin 17 | AAAGCATCCCT | 1.52 | 58.44 | 38.78 | 1.33 | HF, basal cells |
| Trichohyalin | TTGGGGGTGTA | 1.39 | 11.08 | 7.96 | 0.31 | HF, IRS |
| Nexin-1 | AAAAGGTTATG | 1.35 | 4.03 | 2.98 | 0.15 | HF, |
| FGF7 (KGF) | CTTTTTTCCTA | 8.00 | 4.03 | 0.00 | 1.22 | HF, |
| Dermcidin | ACGTTAAAGAC | 2.59 | 69.47 | 26.82 | 4.96 | sweat glands |
| Cystatin | GTGGAGGGCAC | 1.43 | 305.06 | 213.56 | 4.24 | hair shaft |

TABLE 2

| | | | | | | Localization/ |
|---|---|---|---|---|---|---|
| 1.1.1.1.1.1.1.Gene | Tag | Quotient | +Hair | −Hair | Signif. | function |
| Keratin 10 | GAAAACAAAGT | 1.91 | 118.8 | 226.47 | 8.26 | suprabasal |
| Keratin 2e | CCTCTTTGCAT | 7.90 | 1.01 | 7.96 | 1.64 | suprabasal |
| Periplakin | AAAATAAACCT | 1.27 | 7.05 | 8.95 | 0.19 | suprabasal |
| Profilaggrin | GAGAGCTAACT | 2.58 | 13.09 | 33.77 | 2.61 | suprabasal |
| Loricrin | AGAAAAAAAAA | 1.44 | 46.35 | 66.63 | 1.24 | suprabasal |
| small proline-rich | GGCTTCTAACA | 1.97 | 6.05 | 11.93 | 0.75 | suprabasal |
| protein | | | | | | |
| IGFBP 7 | CATATCATTAA | 7.90 | 29.22 | 40.77 | 0.77 | terminal |
| | | | | | | differentiation |
| Keratin 1 | ACATTTCAAAG | −1.08 | 61.46 | 56.68 | 0.18 | suprabasal |
| Keratin 5 | GCCCCTGCTGA | 1.42 | 53.40 | 75.58 | 1.64 | basal, HF |
| Keratin 14 | GATGTGCACGA | 1.18 | 10.08 | 11.93 | 0.15 | basal, HF |
| Keratin 15 | TAATAAAGAAT | −1.06 | 23.17 | 21.88 | 0.07 | basal, HF |

TABLE 3

| | | Scalp/ | | Scalp/ | | | |
|---|---|---|---|---|---|---|---|
| No. | Tag sequence | face | Sign. | breast | Sign. | Annotation | Description |
| 1. | GTTTGCAAGTG | 18.00 | 2.74 | 3.67 | 1.07 | Hs.151787 | U5 snRNP-specific |
| | | | | | | | protein, 116 kD [Swis |
| 2. | ATGCGAAAGGC | 14.00 | 2.13 | 11.40 | 1.77 | Hs.89466 | dodecenoyl-Coenzyme A |
| | | | | | | | delta Isomerase (3 |
| 3. | CTTATGGTTGA | 14.00 | 2.13 | 2.85 | 0.70 | Hs.14084 | ring finger protein 7 |
| | | | | | | | [Swissprot: sp\|Q9 |
| 4. | TTGGGGAAACA | 14.00 | 2.13 | 5.70 | 1.11 | Hs.81029 | biliverdin reductase A |
| | | | | | | | [Swissprot: sp\|P |
| 5. | GGCCGTGCTGC | 14.00 | 2.13 | 11.40 | 1.77 | Hs.131034 | ESTs, Weakly similar to |
| | | | | | | | I78885 serine/th |
| 6. | TGCGCCTTTAT | 12.16 | 2.77 | 1.22 | 0.16 | Hs.133230 | (Manual assignment) |
| | | | | | | | probably reverse tag |
| 7. | TCATTGTAATG | 12.00 | 1.83 | 4.89 | 0.89 | Hs.283722 | (Manual assignment) |
| | | | | | | | GTT1, START domain p |
| 8. | AGGAACACAAA | 12.00 | 1.83 | 4.89 | 0.89 | Hs.334437 | hypothetical protein |
| | | | | | | | MGC4248 [Swissprot |
| 9. | TAGTTGCAAAT | 12.00 | 1.83 | 9.77 | 1.51 | Hs.77311 | BTG family, member 3 |
| | | | | | | | [Swissprot: sp\|P78 |
| 10. | TTTGGGGCTGG | 10.00 | 1.52 | 4.07 | 0.68 | Hs.7476 | ATPase, H+ transporting, |
| | | | | | | | lysosomal (vacu |
| 11. | AATGGAATGGA | 10.00 | 1.52 | 8.15 | 1.25 | Hs.321130 | hypothetical protein |
| | | | | | | | MGC2771 [Swissprot |
| 12. | CTCTTCAGGAG | 10.00 | 1.52 | 8.15 | 1.25 | Hs.30954 | phosphomevalonate |
| | | | | | | | kinase [Swissprot: sp |
| 13. | GGAGGCCGAGC | 10.00 | 1.52 | 8.15 | 1.25 | Hs.301342 | hypothetical protein |
| | | | | | | | MGC4342 [Swissprot |
| 14. | TTTTAACAAAA | 10.00 | 1.52 | 4.07 | 0.68 | Hs.169370 | FYN oncogene related to |
| | | | | | | | SRC, FGR, YES [ |
| 15. | GGCAGGATGAT | 10.00 | 1.52 | 2.04 | 0.35 | Hs.274319 | hypothetical protein |
| | | | | | | | FLJ10509 [Swisspro |
| 16. | TGACTGTATTA | 10.00 | 1.52 | 8.15 | 1.25 | Hs.198241 | amine oxidase, copper |
| | | | | | | | containing 3 (vasc |
| 17. | TGAAAAGCTTA | −11.84 | 1.79 | −2.00 | 0.39 | Hs.2384 | tumor protein D52 |
| | | | | | | | [Swissprot: sp\|P55327 |
| 18. | AAGCACAAAAA | −11.84 | 1.79 | −8.00 | 1.44 | Hs.9963 | TYRO protein tyrosine |
| | | | | | | | kinase binding pro |
| 19. | CCTAAGGCTAA | −11.84 | 1.79 | −4.00 | 0.74 | Hs.108371 | E2F transcription factor 4, |
| | | | | | | | p107/p130-bi |
| 20. | GGAGTCTAACT | −11.84 | 1.79 | −2.00 | 0.39 | Hs.240170 | hypothetical protein |
| | | | | | | | MGC2731 [Swissprot |
| 21. | CAGGAGGAAAG | −13.81 | 2.08 | −8.00 | 1.44 | Hs.177425 | (Manual assignment) |
| | | | | | | | DLGAP4 SAP90/PSD-95- |
| 22. | TTTTACTCACA | −13.81 | 2.08 | −4.00 | 0.74 | Hs.183706 | adducin 1 (alpha) |
| | | | | | | | [Swissprot: sp\|P35611 |

TABLE 4

| | | Scalp/ | | Scalp/ | | | |
|---|---|---|---|---|---|---|---|
| No. | Tag sequence | face | Sign. | breast | Sign. | Annotation | Description |
| 23. | GGTGAAGACAA | 9.12 | 1.96 | 1.22 | 0.13 | Hs.26951 | KIAA0375 gene product |
| | | | | | | | [Swissprot: sp\|O1 |
| 24. | TGTGCTAATAT | 8.11 | 1.69 | 3.26 | 0.88 | Hs.183037 | protein kinase, cAMP- |
| | | | | | | | dependent, regulato |
| 25. | CCACTCCTCCA | 8.11 | 1.69 | 2.17 | 0.57 | Hs.82890 | defender against cell |
| | | | | | | | death 1 [Swisspro |
| 26. | CACCATTCAGT | 8.11 | 1.69 | 13.03 | 2.03 | Hs.108530 | hypothetical protein |
| | | | | | | | FLJ10856 [Swisspro |
| 27. | GAGGACCCAAC | 8.11 | 1.69 | 13.03 | 2.03 | Hs.77313 | cyclin-dependent kinase |
| | | | | | | | (CDC2-like) 10 |
| 28. | CTGCCTCCTTA | 7.10 | 1.43 | 1.90 | 0.41 | Hs.179666 | uncharacterized |
| | | | | | | | hypothalamus protein |
| | | | | | | | HSM |
| 29. | TTCTCTCCACA | 7.10 | 1.43 | 5.70 | 1.11 | Hs.94446 | polyamine-modulated |
| | | | | | | | factor 1 [Swissprot |
| 30. | GGGCTTACTGT | 7.10 | 1.43 | 5.70 | 1.11 | Hs.5472 | hypothetical protein |
| | | | | | | | FLJ22679 [Swisspro |
| 31. | ATTTAGCAAGC | 6.59 | 2.41 | 10.59 | 2.46 | Hs.83213 | fatty acid binding protein |
| | | | | | | | 4, adipocyte |
| 32. | GTAGACTTGTC | 6.08 | 2.16 | 4.89 | 1.66 | Hs.25213 | hypothetical protein |
| | | | | | | | [Swissprot: sp\|Q96 |
| 33. | CAGAAGAGGCT | 5.07 | 1.68 | 16.29 | 2.55 | Hs.347285 | DiGeorge syndrome |
| | | | | | | | critical region gene 6 |
| 34. | CGGTTACTGTG | −5.43 | 1.86 | −1.23 | 0.09 | Hs.49767 | NADH dehydrogenase |
| | | | | | | | (ubiquinone) Fe—S pro |
| 35. | ACATTCCAAGT | −6.91 | 1.39 | −1.23 | 0.07 | Hs.245188 | tissue inhibitor of |
| | | | | | | | metalloproteinase 3 |
| 36. | CCTCTTTGCAT | −7.89 | 1.64 | −27.01 | 6.56 | Hs.707 | keratin 2A (epidermalichthyosis |
| | | | | | | | bullosa |
| 37. | GGAAGATGAAC | −9.87 | 1.49 | −2.00 | 0.39 | Hs.300906 | hypothetical protein |
| | | | | | | | FLJ10656 [Swisspro |
| 38. | CAGGGAGCGCC | −9.87 | 1.49 | −4.00 | 0.74 | Hs.8657 | PC2 (positive cofactor 2, |
| | | | | | | | multiprotein c |
| 39. | CCCCCACCTAA | −9.87 | 2.17 | −9.82 | 2.05 | Hs.77422 | proteolipid protein 2 |
| | | | | | | | (colonic epitheliu |
| 40. | TTGTTATTGCC | −9.87 | 1.49 | −2.00 | 0.39 | Hs.78637 | annexin A7 [Swissprot: |
| | | | | | | | sp\|P20073;] |
| 41. | GCTGAACGCGT | −9.87 | 1.49 | −8.00 | 1.44 | Hs.99029 | CCAAT/enhancer binding |
| | | | | | | | protein (C/EBP), |
| 42. | AACTGCTTCAA | −9.87 | 1.49 | −10.00 | 1.79 | Hs.11538 | actin related protein 2/3 |
| | | | | | | | complex, subun |

TABLE 5

| | | Scalp/ | | Scalp/ | | | |
|---|---|---|---|---|---|---|---|
| No. | Tag sequence | face | Sign. | breast | Sign. | Annotation | Description |
| 43. | CGCACCATTGC | 4.56 | 1.44 | 3.67 | 1.07 | Hs.94672 | GCN5 (general control of |
| | | | | | | | amino-acid synt |
| 44. | TGGCCTCTCTG | 4.56 | 1.44 | 7.33 | 1.55 | Hs.299629 | peroxisomal long-chain |
| | | | | | | | acyl-coA thioeste |
| 45. | TGGCTGTGTGG | 3.72 | 1.48 | 2.99 | 1.06 | Hs.250465 | H. sapiens mRNA; cDNA |
| | | | | | | | DKFZp434E2023 (f |
| 46. | GTGCTGATTCT | 3.72 | 1.48 | 4.48 | 1.46 | Hs.1640 | collagen, type VII, alpha 1 |
| | | | | | | | (epidermolys |
| 47. | TGCTTTGGGAT | 3.65 | 2.22 | 7.33 | 2.94 | Hs.84344 | CGI-135 protein |
| | | | | | | | [Swissprot: sp\|Q9BTP3; s |
| 48. | ACTGGGTCTAT | 3.55 | 1.75 | 3.80 | 1.61 | Hs.275163 | (Manual assignment) |
| | | | | | | | NME2 Nucleoside diph |
| 49. | AAGGTAATATG | 3.29 | 1.54 | 21.18 | 3.32 | Hs.81874 | microsomal glutathione S- |
| | | | | | | | transferase 2 |
| 50. | GGGCCCTTCCT | 3.04 | 1.34 | 9.77 | 2.23 | Hs.168073 | DKFZP727M231 protein |
| | | | | | | | [Swissprot: sp\|Q96 |
| 51. | AAGGCAATTTA | −4.44 | 1.39 | −2.46 | 0.52 | Hs.244621 | (Manual assignment) |
| | | | | | | | probably reverse tag |
| 52. | TGTGGGTGCTG | −4.44 | 1.39 | −1.23 | 0.09 | Hs.306339 | H. sapiens mRNA; cDNA |
| | | | | | | | DKFZp586N2022 (f |
| 53. | GCTGCCCTGGG | −4.93 | 1.62 | −6.75 | 2.38 | Hs.286084 | (Manual assignment) |
| | | | | | | | Centaurin gamma3 (MR |

TABLE 6

| | | Scalp/ | | Scalp/ | | | |
|---|---|---|---|---|---|---|---|
| No. | Tag sequence | face | Sign. | breast | Sign. | Annotation | Description |
| 54. | CGCACCATTGC | 4.56 | 1.44 | 3.67 | 1.07 | Hs.94672 | GCN5 (general control of |
| | | | | | | | amino-acid synt |
| 55. | TGGCCTCTCTG | 4.56 | 1.44 | 7.33 | 1.55 | Hs.299629 | peroxisomal long-chain |
| | | | | | | | acyl-coA thioeste |
| 56. | TGGCTGTGTGG | 3.72 | 1.48 | 2.99 | 1.06 | Hs.250465 | H. sapiens mRNA; cDNA |
| | | | | | | | DKFZp434E2023 (f |
| 57. | GTGCGCTGAGC | 2.87 | 1.68 | 2.31 | 1.13 | Hs.277477 | major histocompatibility |
| | | | | | | | complex, class |
| 58. | AGAACCTTAAA | 2.87 | 1.68 | 4.62 | 2.19 | Hs.181165 | eukaryotic translation |
| | | | | | | | elongation factor |
| 59. | CAGTTTGTACA | 2.84 | 1.41 | 11.40 | 2.69 | Hs.1023 | pyruvate dehydrogenase |
| | | | | | | | (lipoamide) alpha |
| 60. | GGTGTCTGTGG | 2.70 | 2.09 | 9.77 | 4.28 | Hs.334305 | GS1999full [Swissprot: |
| | | | | | | | sp\|Q96BS0; sp\|Q96 |
| 61. | CAGTTCTCTGT | 2.59 | 1.92 | 6.24 | 3.42 | Hs.279921 | HSPC035 protein |
| | | | | | | | [Swissprot: sp\|Q96BY9; s |
| 62. | TTGATGTACAG | 2.53 | 1.31 | 24.44 | 3.84 | Hs.11482 | splicing factor, |
| | | | | | | | arginine/serine-rich 11 |
| 63. | TACATCAGTAA | 2.48 | 1.74 | 5.97 | 3.21 | Hs.65029 | growth arrest-specific 1 |
| | | | | | | | [Swissprot: sp |
| 64. | GGAGGCTGAGG | 2.28 | 2.33 | 1.22 | 0.34 | Hs.118793 | hypothetical protein |
| | | | | | | | FLJ10688 [Swisspro |
| 65. | TAACCAATCAG | 2.12 | 1.43 | 37.47 | 5.91 | Hs.479 | RAB5C, member RAS |
| | | | | | | | oncogene family [Swis |
| 66. | GACTCTGGTGC | 1.91 | 2.09 | 6.38 | 6.73 | Hs.343665 | ribosomal protein S15a |
| | | | | | | | [Swissprot: sp\|P |
| 67. | GATCTCTTGGG | 1.90 | 1.45 | 6.11 | 4.32 | Hs.38991 | S100 calcium-binding |
| | | | | | | | protein A2 [Swissp |
| 68. | GAAAACAAAGT | −1.91 | 8.26 | −3.45 | 34.71 | Hs.99936 | keratin 10 (epidermolytic |
| | | | | | | | hyperkeratosis |
| 69. | TTTGGTTTTCC | −1.92 | 1.81 | −1.29 | 0.39 | Hs.179573 | collagen, type I, alpha 2 |
| | | | | | | | [Swissprot: s |
| 70. | GCAAAAAAAAA | −2.47 | 1.58 | −1.99 | 0.92 | Hs.4746 | hypothetical protein |
| | | | | | | | FLJ21324 [Swisspro |
| 71. | GAGAGCTAACT | −2.58 | 2.61 | −1.13 | 0.00 | Hs.73995 | (Manual assignment) FLG |
| | | | | | | | Profilaggrin (fi |
| 72. | ACATTCTTTTT | −2.63 | 1.99 | −1.77 | 0.74 | Hs.82226 | glycoprotein |
| | | | | | | | (transmembrane) nmb |
| | | | | | | | [Swiss |
| 73. | CTTCTTGCCCC | −2.80 | 1.61 | −1.84 | 0.61 | Hs.347939 | hemoglobin, alpha 2 |
| | | | | | | | [Swissprot: sp\|P019 |
| 74. | TCACCTTAGGT | −2.80 | 1.61 | −1.84 | 0.61 | Hs.239625 | integral membrane protein |
| | | | | | | | 2B [Swissprot |

TABLE 7

| | | Scalp/ | | Scalp/ | | | |
|---|---|---|---|---|---|---|---|
| No. | Tag sequence | face | Sign. | breast | Sign. | Annotation | Description |
| 75. | GGAGGTGGAGG | −7.89 | 1.19 | −12.00 | 2.13 | Hs.93871 | hypothetical protein |
| | | | | | | | FLJ10783 [Swisspro |
| 76. | GGGGGTGGGTG | 6.08 | 1.17 | 9.77 | 1.51 | Hs.57904 | mago-nashi (Drosophila) |
| | | | | | | | homolog, prolife |
| 77. | GGCTATGTTAA | 6.08 | 1.17 | 9.77 | 1.51 | Hs.5464 | thyroid hormone receptor |
| | | | | | | | coactivating pr |
| 78. | CTAGATTCCCT | 6.08 | 1.17 | 9.77 | 1.51 | Hs.46783 | Homo sapiens cDNA: |
| | | | | | | | FLJ22382 fis, clone H |
| 79. | AGGTGGACAGG | 6.08 | 1.17 | 9.77 | 1.51 | Hs.127007 | potassium channel, |
| | | | | | | | subfamily K, member 5 |
| 80. | GCATAGTGTTT | 6.08 | 1.17 | 9.77 | 1.51 | Hs.11050 | F-box only protein 9 |
| | | | | | | | [Swissprot: sp\|O75 |
| 81. | GATGTGAAAAG | 6.08 | 1.17 | 9.77 | 1.51 | Hs.106148 | Homo sapiens mRNA; |
| | | | | | | | cDNA DKFZp434G0972 [symbol] |

TABLE 8

| | | | | | | | | Quotient |
|---|---|---|---|---|---|---|---|---|
| | | | | | | | | (scalp/ |
| | | | | | | | | face)/ |
| | | Scalp/ | | Scalp/ | | | | (scalp/ |
| No. | Tag sequence | face | Sign. | breast | Sign. | Annotation | Description | breast) |
| 82. | AGGTCTAAGGC | 5.07 | 0.92 | 8.15 | 1.25 | Hs.93868 | KIAA0662 gene | 0.62 |
| | | | | | | | product | |
| | | | | | | | [Swissprot: | |
| | | | | | | | sp\|O7 | |
| 83. | GTCTCATTTGA | 5.07 | 0.92 | 8.15 | 1.25 | Hs.92381 | nudix | 0.62 |
| | | | | | | | (nucleoside | |
| | | | | | | | diphosphate | |
| | | | | | | | linked mol | |
| 84. | TCTGGGGAAAT | 5.07 | 0.92 | 8.15 | 1.25 | Hs.87417 | cathepsin L2 | 0.62 |
| | | | | | | | [Swissprot: | |
| | | | | | | | sp\|O60911;] | |
| 85. | TTGTCTGCTTT | 5.07 | 0.92 | 8.15 | 1.25 | Hs.350511 | [Swissprot: | 0.62 |
| | | | | | | | sp\|Q9BYT5;] | |
| 86. | GTTCTTGTATC | 5.07 | 0.92 | 8.15 | 1.25 | Hs.307012 | keratin | 0.62 |
| | | | | | | | associated | |
| | | | | | | | protein 9.3 | |
| | | | | | | | [Swisspr | |
| 87. | CTTCTGGATAA | 5.07 | 0.92 | 8.15 | 1.25 | Hs.274368 | MSTP032 | 0.62 |
| | | | | | | | protein | |
| | | | | | | | [Swissprot: | |
| | | | | | | | sp\|Q9H3F2;] | |
| 88. | AAAATAAACAA | 5.07 | 0.92 | 8.15 | 1.25 | Hs.25035 | chloride | 0.62 |
| | | | | | | | intracellular | |
| | | | | | | | channel 4 | |
| | | | | | | | [Swiss | |
| 89. | TTACTTCCCCC | 5.07 | 0.92 | 8.15 | 1.25 | Hs.184641 | fatty acid | 0.62 |
| | | | | | | | desaturase 2 | |
| | | | | | | | [Swissprot: sp\| | |
| 90. | CTTTTTGTGCC | 5.07 | 0.92 | 8.15 | 1.25 | Hs.182238 | GW128 protein | 0.62 |
| | | | | | | | [Swissprot: | |
| | | | | | | | sp\|Q9Y649;] | |
| 91. | TGGGTCTGAAC | 5.07 | 0.92 | 8.15 | 1.25 | Hs.173484 | hypothetical | 0.62 |
| | | | | | | | protein | |
| | | | | | | | FLJ10337 | |
| | | | | | | | [Swisspro | |
| 92. | TGCCAATTAAG | 5.07 | 0.92 | 8.15 | 1.25 | Hs.165337 | ESTs | 0.62 |
| | | | | | | | [Swissprot: | |
| | | | | | | | none] | |
| 93. | AAGGGGGCAAG | −7.89 | 1.19 | −6.00 | 1.09 | Hs.85266 | integrin, beta 4 | 1.32 |
| | | | | | | | [Swissprot: | |
| | | | | | | | sp\|P16144; | |
| 94. | TCTGCAAAGGA | −7.89 | 1.19 | −6.00 | 1.09 | Hs.80684 | high-mobility | 1.32 |
| | | | | | | | group | |
| | | | | | | | (nonhistone | |
| | | | | | | | chromoso | |
| 95. | ACGAAACCCCG | −7.89 | 1.19 | −6.00 | 1.09 | Hs.331088 | EST [Swissprot: | 1.32 |
| | | | | | | | none] | |
| 96. | CATCCTGCTGC | 8.00 | 1.22 | 6.52 | 0.99 | Hs.74619 | proteasome | 1.23 |
| | | | | | | | (prosome, | |
| | | | | | | | macropain) 26S | |
| | | | | | | | subu | |
| 97. | CTGTTGCTGGG | 8.00 | 1.22 | 6.52 | 0.99 | Hs.61976 | Homo sapiens | 1.23 |
| | | | | | | | cDNA FLJ12947 | |
| | | | | | | | fis, clone NT | |
| 98. | GACAAAGAGAG | 8.00 | 1.22 | 6.52 | 0.99 | Hs.46735 | deafness locus | 1.23 |
| | | | | | | | associated | |
| | | | | | | | putative guani | |
| 99. | TGGATGTACAT | 8.00 | 1.22 | 6.52 | 0.99 | Hs.43773 | ESTs, Weakly | 1.23 |
| | | | | | | | similar to | |
| | | | | | | | T2D3_HUMAN | |
| | | | | | | | TRANS | |
| 100. | TGTGAGGGAAG | 8.00 | 1.22 | 6.52 | 0.99 | Hs.3849 | hypothetical | 1.23 |
| | | | | | | | protein | |
| | | | | | | | FLJ22041 | |
| | | | | | | | similar to | |
| 101. | ATGTTCAGGCT | 8.00 | 1.22 | 6.52 | 0.99 | Hs.326494 | Homo sapiens, | 1.23 |
| | | | | | | | Similar to | |
| | | | | | | | deoxyguanosine | |
| 102. | TATGAGATAGA | 8.00 | 1.22 | 6.52 | 0.99 | Hs.323914 | translocase of | 1.23 |
| | | | | | | | inner | |
| | | | | | | | mitochondrial | |
| | | | | | | | membr | |
| 103. | TACCAAAATAA | 8.00 | 1.22 | 6.52 | 0.99 | Hs.307016 | keratin | 1.23 |
| | | | | | | | associated | |
| | | | | | | | protein 4.12 | |
| | | | | | | | [Swissp | |
| 104. | TGCTGCCCCCC | 8.00 | 1.22 | 6.52 | 0.99 | Hs.29797 | ribosomal | 1.23 |
| | | | | | | | protein L10 | |
| | | | | | | | [Swissprot: | |
| | | | | | | | sp\|O9 | |
| 105. | TTTTATTGGAA | 8.00 | 1.22 | 6.52 | 0.99 | Hs.296242 | recombination | 1.23 |
| | | | | | | | protein REC14 | |
| | | | | | | | [Swissprot: | |
| 106. | CTCGGTGATGT | 8.00 | 1.22 | 6.52 | 0.99 | Hs.279903 | Ras homolog | 1.23 |
| | | | | | | | enriched in brain | |
| | | | | | | | 2 [Swissp | |
| 107. | CAAATAAACCT | 8.00 | 1.22 | 6.52 | 0.99 | Hs.279663 | Pirin [Swissprot: | 1.23 |
| | | | | | | | sp\|O00625;] | |
| 108. | GCAAATCAGAT | 8.00 | 1.22 | 6.52 | 0.99 | Hs.279477 | ESTs | 1.23 |
| | | | | | | | [Swissprot: | |
| | | | | | | | none] | |
| 109. | TAACTAACAAA | 8.00 | 1.22 | 6.52 | 0.99 | Hs.23488 | KIAA0107 gene | 1.23 |
| | | | | | | | product | |
| | | | | | | | [Swissprot: | |
| | | | | | | | sp\|Q1 | |
| 110. | GTGGCCTGTGC | 8.00 | 1.22 | 6.52 | 0.99 | Hs.209346 | ESTs, Weakly | 1.23 |
| | | | | | | | similar to | |
| | | | | | | | ALU1_HUMAN | |
| | | | | | | | ALU S | |
| 111. | GGGGCTGGGGT | 8.00 | 1.22 | 6.52 | 0.99 | Hs.183698 | ribosomal | 1.23 |
| | | | | | | | protein L29 | |
| | | | | | | | [Swissprot: | |
| | | | | | | | sp\|P4 | |
| 112. | TAATAAAGAAC | 8.00 | 1.22 | 6.52 | 0.99 | Hs.17893 | rab interacting | 1.23 |
| | | | | | | | lysosomal | |
| | | | | | | | protein [Swis | |
| 113. | CTTTTTTCCTA | 8.00 | 1.22 | 6.52 | 0.99 | Hs.164568 | fibroblast growth | 1.23 |
| | | | | | | | factor 7 | |
| | | | | | | | (keratinocyte | |
| 114. | GGGGTGGGGCC | 8.00 | 1.22 | 6.52 | 0.99 | Hs.154868 | carbamoyl- | 1.23 |
| | | | | | | | phosphate | |
| | | | | | | | synthetase 2, | |
| | | | | | | | aspart | |
| 115. | AAACTCTATAT | 8.00 | 1.22 | 6.52 | 0.99 | Hs.13619 | ESTs | 1.23 |
| | | | | | | | [Swissprot: | |
| | | | | | | | none] | |
| 116. | GCCCAGTGATA | 8.00 | 1.22 | 6.52 | 0.99 | Hs.12702 | Homo sapiens | 1.23 |
| | | | | | | | mRNA; cDNA | |
| | | | | | | | DKFZp586N012 | |
| | | | | | | | (fr | |
| 117. | CTAAGGACCAA | 8.00 | 1.22 | 6.52 | 0.99 | Hs.119021 | DKFZP434N061 | 1.23 |
| | | | | | | | protein | |
| | | | | | | | [Swissprot: | |
| | | | | | | | sp\|Q9B | |
| 118. | CACGAAGATGC | 8.00 | 1.22 | 6.52 | 0.99 | Hs.10247 | activated | 1.23 |
| | | | | | | | leucocyte cell | |
| | | | | | | | adhesion | |
| | | | | | | | molecu | |
| 119. | GTATTTAACAT | 6.08 | 1.17 | 4.89 | 0.89 | Hs.9006 | VAMP (vesicle- | 1.24 |
| | | | | | | | associated | |
| | | | | | | | membrane protei | |
| 120. | GACTGAATGTA | 6.08 | 1.17 | 4.89 | 0.89 | Hs.343953 | Homo sapiens | 1.24 |
| | | | | | | | clone | |
| | | | | | | | TCBAP0774 | |
| | | | | | | | mRNA sequen | |
| 121. | TACAGATCACA | 6.08 | 1.17 | 4.89 | 0.89 | Hs.173859 | frizzled | 1.24 |
| | | | | | | | (Drosophila) | |
| | | | | | | | homolog 7 | |
| | | | | | | | [Swissp | |
| 122. | CCCTATCACAA | 6.08 | 1.17 | 4.89 | 0.89 | Hs.150826 | CATX-8 protein | 1.24 |
| | | | | | | | [Swissprot: | |
| | | | | | | | sp\|P57735; sp | |
| 123. | GACATAAATCC | 6.08 | 1.17 | 4.89 | 0.89 | Hs.109281 | Nef-associated | 1.24 |
| | | | | | | | factor 1 | |
| | | | | | | | [Swissprot: sp\| | |
| 124. | TGCTTCATCTG | −5.92 | 1.13 | −4.91 | 0.86 | Hs.10842 | RAN, member | 1.21 |
| | | | | | | | RAS oncogene | |
| | | | | | | | family [Swissp | |
| 125. | CAGTGTTGGGG | −7.89 | 1.19 | −4.00 | 0.74 | Hs.296847 | spastic | 1.97 |
| | | | | | | | paraplegia 7, | |
| | | | | | | | paraplegin (pure a | |
| 126. | AAGTTTCCAAT | −7.89 | 1.19 | −4.00 | 0.74 | Hs.2903 | protein | 1.97 |
| | | | | | | | phosphatase 4 | |
| | | | | | | | (formerly X), | |
| | | | | | | | cata | |
| 127. | TTAAATAGCAC | −7.89 | 1.19 | −4.00 | 0.74 | Hs.172928 | collagen, type I, | 1.97 |
| | | | | | | | alpha 1 | |
| | | | | | | | [Swissprot: s | |
| 128. | TTTTTCACTTA | 6.00 | 0.91 | 4.89 | 0.73 | Hs.99472 | ESTs | 1.23 |
| | | | | | | | [Swissprot: | |
| | | | | | | | none] | |
| 129. | CCTATGGCTTT | 6.00 | 0.91 | 4.89 | 0.73 | Hs.98855 | hypothetical | 1.23 |
| | | | | | | | protein | |
| | | | | | | | FLJ20909 | |
| | | | | | | | [Swisspro | |
| 130. | GCATTGATGTG | 6.00 | 0.91 | 4.89 | 0.73 | Hs.95870 | PTD015 protein | 1.23 |
| | | | | | | | [Swissprot: | |
| | | | | | | | sp\|Q9H7C9; sp | |
| 131. | GACGTCCAGAG | 6.00 | 0.91 | 4.89 | 0.73 | Hs.95262 | nuclear factor | 1.23 |
| | | | | | | | related to kappa | |
| | | | | | | | B bindin | |
| 132. | GTTGTAAATAA | 6.00 | 0.91 | 4.89 | 0.73 | Hs.92033 | integrin-linked | 1.23 |
| | | | | | | | kinase- | |
| | | | | | | | associated | |
| | | | | | | | serine | |
| 133. | CTGATTTGTAC | 6.00 | 0.91 | 4.89 | 0.73 | Hs.91390 | poly (ADP- | 1.23 |
| | | | | | | | ribose) | |
| | | | | | | | glycohydrolase | |
| | | | | | | | [Swiss | |
| 134. | CCCTGCTCCTC | 6.00 | 0.91 | 4.89 | 0.73 | Hs.9081 | phenylalanyl- | 1.23 |
| | | | | | | | tRNA synthetase | |
| | | | | | | | beta-subuni | |
| 135. | TGGCGTGGCCG | 6.00 | 0.91 | 4.89 | 0.73 | Hs.8854 | pvt-1 (murine) | 1.23 |
| | | | | | | | oncogene | |
| | | | | | | | homolog, MYC | |
| | | | | | | | act | |
| 136. | CTGGATCTGGG | 6.00 | 0.91 | 4.89 | 0.73 | Hs.75658 | phosphorylase, | 1.23 |
| | | | | | | | glycogen; brain | |
| | | | | | | | [Swisspr | |
| 137. | CAGATCGAAAA | 6.00 | 0.91 | 4.89 | 0.73 | Hs.75613 | CD36 antigen | 1.23 |
| | | | | | | | (collagen type I | |
| | | | | | | | receptor, | |
| 138. | CAGGTTGTGAG | 6.00 | 0.91 | 4.89 | 0.73 | Hs.75589 | acid | 1.23 |
| | | | | | | | phosphatase 2, | |
| | | | | | | | lysosomal | |
| | | | | | | | [Swisspro | |
| 139. | AAATAAAAGGT | 6.00 | 0.91 | 4.89 | 0.73 | Hs.7535 | COBW-like | 1.23 |
| | | | | | | | protein | |
| | | | | | | | [Swissprot: | |
| | | | | | | | sp\|O60748 | |
| 140. | GTTACCGAGGA | 6.00 | 0.91 | 4.89 | 0.73 | Hs.75339 | inositol | 1.23 |
| | | | | | | | polyphosphate | |
| | | | | | | | phosphatase- | |
| | | | | | | | like | |
| 141. | ATTTCAAAAAA | 6.00 | 0.91 | 4.89 | 0.73 | Hs.7378 | Homo sapiens | 1.23 |
| | | | | | | | mRNA; cDNA | |
| | | | | | | | DKFZp434G227 | |
| | | | | | | | (fr | |
| 142. | GTTATGGCTGG | 6.00 | 0.91 | 4.89 | 0.73 | Hs.687 | cytochrome | 1.23 |
| | | | | | | | P450, subfamily | |
| | | | | | | | IVB, polypept | |
| 143. | TTCCCTGTGAG | 6.00 | 0.91 | 4.89 | 0.73 | Hs.621 | lectin, | 1.23 |
| | | | | | | | galactoside- | |
| | | | | | | | binding, soluble, 3 | |
| 144. | GAAGATGAATA | 6.00 | 0.91 | 4.89 | 0.73 | Hs.54982 | Homo sapiens | 1.23 |
| | | | | | | | cDNA FLJ14014 | |
| | | | | | | | fis, clone HE | |
| 145. | ACTACTAAATA | 6.00 | 0.91 | 4.89 | 0.73 | Hs.5437 | Tax1 (human T- | 1.23 |
| | | | | | | | cell leukemia | |
| | | | | | | | virus type I | |
| 146. | GAGAAAAAGTG | 6.00 | 0.91 | 4.89 | 0.73 | Hs.50450 | hypothetical | 1.23 |
| | | | | | | | protein | |
| | | | | | | | MGC12928 | |
| | | | | | | | [Swisspro | |
| 147. | GGCGGGTCGGG | 6.00 | 0.91 | 4.89 | 0.73 | Hs.48938 | hypothetical | 1.23 |
| | | | | | | | protein | |
| | | | | | | | FLJ21802 | |
| | | | | | | | [Swisspro | |
| 148. | AAACCAGAGGT | 6.00 | 0.91 | 4.89 | 0.73 | Hs.4291 | golgi peripheral | 1.23 |
| | | | | | | | membrane | |
| | | | | | | | protein p65 [ | |
| 149. | GCCAGGTTGCC | 6.00 | 0.91 | 4.89 | 0.73 | Hs.42824 | hypothetical | 1.23 |
| | | | | | | | protein | |
| | | | | | | | FLJ10718 | |
| | | | | | | | [Swisspro | |
| 150. | GCTGGAGTTGC | 6.00 | 0.91 | 4.89 | 0.73 | Hs.41696 | keratin, hair, | 1.23 |
| | | | | | | | acidic, 1 | |
| | | | | | | | [Swissprot: sp\| | |
| 151. | ACAGGTGTTTA | 6.00 | 0.91 | 4.89 | 0.73 | Hs.40588 | ESTs | 1.23 |
| | | | | | | | [Swissprot: | |
| | | | | | | | none] | |
| 152. | TTTCAGATTGG | 6.00 | 0.91 | 4.89 | 0.73 | Hs.349507 | [Swissprot: | 1.23 |
| | | | | | | | sp\|P53999; sp\|Q96L29;] | |
| 153. | TGTGACACTGA | 6.00 | 0.91 | 4.89 | 0.73 | Hs.340959 | Ts translation | 1.23 |
| | | | | | | | elongation | |
| | | | | | | | factor, mitoch | |
| 154. | AAGTTGGTGCT | 6.00 | 0.91 | 4.89 | 0.73 | Hs.339808 | hypothetical | 1.23 |
| | | | | | | | protein | |
| | | | | | | | FLJ10120 | |
| | | | | | | | [Swisspro | |
| 155. | CTTTATGTGAT | 6.00 | 0.91 | 4.89 | 0.73 | Hs.3352 | histone | 1.23 |
| | | | | | | | deacetylase 2 | |
| | | | | | | | [Swissprot: | |
| | | | | | | | sp\|Q9 | |
| 156. | GGTTGTCTAAT | 6.00 | 0.91 | 4.89 | 0.73 | Hs.334691 | splicing factor | 1.23 |
| | | | | | | | 3a, subunit 1, | |
| | | | | | | | 120 kD [S | |
| 157. | CTGGAGTGCAG | 6.00 | 0.91 | 4.89 | 0.73 | Hs.327202 | ESTs | 1.23 |
| | | | | | | | [Swissprot: | |
| | | | | | | | none] | |
| 158. | ATTATCCTAAT | 6.00 | 0.91 | 4.89 | 0.73 | Hs.307025 | keratin | 1.23 |
| | | | | | | | associated | |
| | | | | | | | protein 3.3 | |
| | | | | | | | [Swisspr | |
| 159. | GCAATAAGTGT | 6.00 | 0.91 | 4.89 | 0.73 | Hs.285976 | longevity | 1.23 |
| | | | | | | | assurance | |
| | | | | | | | (LAG1, S. cerevisiae | |
| 160. | TTGTAATAAAA | 6.00 | 0.91 | 4.89 | 0.73 | Hs.283429 | SMC (mouse) | 1.23 |
| | | | | | | | homolog, X | |
| | | | | | | | chromosome | |
| | | | | | | | [Swis | |
| 161. | GAAAGCATACC | 6.00 | 0.91 | 4.89 | 0.73 | Hs.28298 | adaptor-related | 1.23 |
| | | | | | | | protein complex | |
| | | | | | | | 4, beta | |
| 162. | AAGGAGATTAT | 6.00 | 0.91 | 4.89 | 0.73 | Hs.279832 | hypothetical | 1.23 |
| | | | | | | | protein | |
| | | | | | | | FLJ10488 | |
| | | | | | | | [Swisspro | |
| 163. | GCTGTATAATT | 6.00 | 0.91 | 4.89 | 0.73 | Hs.279009 | matrix Gla | 1.23 |
| | | | | | | | protein | |
| | | | | | | | [Swissprot: | |
| | | | | | | | sp\|P0849 | |
| 164. | GTTGGATAGGG | 6.00 | 0.91 | 4.89 | 0.73 | Hs.27184 | growth factor, | 1.23 |
| | | | | | | | erv1 (S. cerevisiae)- | |
| | | | | | | | like | |
| 165. | CAGAAATATAT | 6.00 | 0.91 | 4.89 | 0.73 | Hs.262823 | hypothetical | 1.23 |
| | | | | | | | protein | |
| | | | | | | | FLJ10326 | |
| | | | | | | | [Swisspro | |
| 166. | GAGGTGAAGGG | 6.00 | 0.91 | 4.89 | 0.73 | Hs.26216 | LOC50627 | 1.23 |
| | | | | | | | [Swissprot: | |
| | | | | | | | none] | |
| 167. | TTACTCTTTCT | 6.00 | 0.91 | 4.89 | 0.73 | Hs.2533 | aldehyde | 1.23 |
| | | | | | | | dehydrogenase | |
| | | | | | | | 9 family, | |
| | | | | | | | member | |
| 168. | TAAACTGTAAA | 6.00 | 0.91 | 4.89 | 0.73 | Hs.244621 | ribosomal | 1.23 |
| | | | | | | | protein S14 | |
| | | | | | | | [Swissprot: | |
| | | | | | | | sp\|P0 | |
| 169. | CGTTTAATCAT | 6.00 | 0.91 | 4.89 | 0.73 | Hs.239527 | RAP2B, member | 1.23 |
| | | | | | | | of RAS | |
| | | | | | | | oncogene family | |
| | | | | | | | [S | |
| 170. | GTCATAAGAGG | 6.00 | 0.91 | 4.89 | 0.73 | Hs.23518 | hypothetical | 1.23 |
| | | | | | | | protein from | |
| | | | | | | | BCRA2 region | |
| 171. | CTGAACTCTTC | 6.00 | 0.91 | 4.89 | 0.73 | Hs.22268 | Homo sapiens, | 1.23 |
| | | | | | | | Similar to RIKEN | |
| | | | | | | | cDNA 2600 | |
| 172. | CAGGCTGTAGA | 6.00 | 0.91 | 4.89 | 0.73 | Hs.22157 | hypothetical | 1.23 |
| | | | | | | | protein | |
| | | | | | | | FLJ22353 | |
| | | | | | | | [Swisspro | |
| 173. | TTCTGGTATTA | 6.00 | 0.91 | 4.89 | 0.73 | Hs.198689 | KIAA0728 | 1.23 |
| | | | | | | | protein | |
| | | | | | | | [Swissprot: | |
| | | | | | | | sp\|O94833; | |
| 174. | AGGGTGGGGGT | 6.00 | 0.91 | 4.89 | 0.73 | Hs.19405 | caspase | 1.23 |
| | | | | | | | recruitment | |
| | | | | | | | domain 4 | |
| | | | | | | | [Swissprot | |
| 175. | TGAAGTGCTTG | 6.00 | 0.91 | 4.89 | 0.73 | Hs.184343 | hypothetical | 1.23 |
| | | | | | | | protein | |
| | | | | | | | FLJ12517 | |
| | | | | | | | [Swisspro | |
| 176. | TTAATAGTGGG | 6.00 | 0.91 | 4.89 | 0.73 | Hs.18271 | golgi | 1.23 |
| | | | | | | | phosphoprotein | |
| | | | | | | | 3 (coat-protein) [ | |
| 177. | ATTCTGTCATA | 6.00 | 0.91 | 4.89 | 0.73 | Hs.182217 | succinate-CoA | 1.23 |
| | | | | | | | ligase, ADP- | |
| | | | | | | | forming, beta | |
| 178. | AAATATGTGTT | 6.00 | 0.91 | 4.89 | 0.73 | Hs.173840 | HUEL (C4orf1)- | 1.23 |
| | | | | | | | interacting | |
| | | | | | | | protein [Swis | |
| 179. | TGCACTCCTTC | 6.00 | 0.91 | 4.89 | 0.73 | Hs.16081 | chromodomain | 1.23 |
| | | | | | | | protein, Y | |
| | | | | | | | chromosome- | |
| | | | | | | | like | |
| 180. | AGCCCTGGCTG | 6.00 | 0.91 | 4.89 | 0.73 | Hs.15896 | pericentrin 2 | 1.23 |
| | | | | | | | (kendrin) | |
| | | | | | | | [Swissprot: sp\| | |
| 181. | AGCTCCTTGAG | 6.00 | 0.91 | 4.89 | 0.73 | Hs.15744 | SH2-B homolog | 1.23 |
| | | | | | | | [Swissprot: | |
| | | | | | | | sp\|Q96FK3; sp\| | |
| 182. | TGACTTGTAAT | 6.00 | 0.91 | 4.89 | 0.73 | Hs.157426 | KIAA1504 | 1.23 |
| | | | | | | | protein | |
| | | | | | | | [Swissprot: | |
| | | | | | | | sp\|O60362; | |
| 183. | TATCCAAGTAA | 6.00 | 0.91 | 4.89 | 0.73 | Hs.132554 | ESTs | 1.23 |
| | | | | | | | [Swissprot: | |
| | | | | | | | none] | |
| 184. | AGTCCCCAACC | 6.00 | 0.91 | 4.89 | 0.73 | Hs.130881 | B-cell | 1.23 |
| | | | | | | | CLL/lymphoma | |
| | | | | | | | 11A (zinc finger | |
| | | | | | | | pro | |
| 185. | GAAACAGACGG | 6.00 | 0.91 | 4.89 | 0.73 | Hs.12124 | elaC (E. coli) | 1.23 |
| | | | | | | | homolog 2 | |
| | | | | | | | [Swissprot: sp | |
| 186. | GAGAGCCTCAG | 6.00 | 0.91 | 4.89 | 0.73 | Hs.12040 | STE20-like | 1.23 |
| | | | | | | | kinase | |
| | | | | | | | [Swissprot: | |
| | | | | | | | sp\|Q9HC79 | |
| 187. | TGTACTACTTA | 6.00 | 0.91 | 4.89 | 0.73 | Hs.117950 | multifunctional | 1.23 |
| | | | | | | | polypeptide | |
| | | | | | | | similar to S | |
| 188. | GTGGGACCATT | 6.00 | 0.91 | 4.89 | 0.73 | Hs.11774 | protein (peptidyl- | 1.23 |
| | | | | | | | prolyl cis/transisome | |
| 189. | GTTCTTCTGTT | 6.00 | 0.91 | 4.89 | 0.73 | Hs.106432 | Homo sapiens | 1.23 |
| | | | | | | | cDNA FLJ13410 | |
| | | | | | | | fis, clone PL | |
| 190. | ACTGAAGGCGC | 5.07 | 0.92 | 4.07 | 0.68 | Hs.92208 | a disintegrin and | 1.25 |
| | | | | | | | metalloproteinase | |
| | | | | | | | doma | |
| 191. | GGTTTGATTAC | 5.07 | 0.92 | 4.07 | 0.68 | Hs.69559 | KIAA1096 | 1.25 |
| | | | | | | | protein | |
| | | | | | | | [Swissprot: | |
| | | | | | | | sp\|Q9NSM8; | |
| 192. | CCGTAGTGCCT | 5.07 | 0.92 | 4.07 | 0.68 | Hs.6891 | splicing factor, | 1.25 |
| | | | | | | | arginine/serine- | |
| | | | | | | | rich 6 | |
| 193. | TGGGGATTACC | 5.07 | 0.92 | 4.07 | 0.68 | Hs.57813 | zinc ribbon | 1.25 |
| | | | | | | | domain | |
| | | | | | | | containing, 1 | |
| | | | | | | | [Swiss | |
| 194. | CGATATTCCCC | 5.07 | 0.92 | 4.07 | 0.68 | Hs.37616 | Human D9 | 1.25 |
| | | | | | | | splice variant A | |
| | | | | | | | mRNA, complete | |
| 195. | TTGGTGCTTGG | 5.07 | 0.92 | 4.07 | 0.68 | Hs.282960 | hypothetical | 1.25 |
| | | | | | | | protein | |
| | | | | | | | MGC10870 | |
| | | | | | | | [Swisspro | |
| 196. | GTCCCTGCCTT | 5.07 | 0.92 | 4.07 | 0.68 | Hs.279837 | glutathione S- | 1.25 |
| | | | | | | | transferase M2 | |
| | | | | | | | (muscle) [ | |
| 197. | TATTCTCAATA | 5.07 | 0.92 | 4.07 | 0.68 | Hs.181311 | asparaginyl- | 1.25 |
| | | | | | | | tRNA synthetase | |
| | | | | | | | [Swissprot: | |
| 198. | TGTGGCAAAGT | 6.08 | 1.17 | 2.44 | 0.52 | Hs.7243 | ubiquitin specific | 2.49 |
| | | | | | | | protease 24 | |
| | | | | | | | [Swisspr | |
| 199. | CCCCATACTAC | 6.08 | 1.17 | 2.44 | 0.52 | Hs.57652 | cadherin, EGF | 2.49 |
| | | | | | | | LAG seven-pass | |
| | | | | | | | G-type rece | |
| 200. | GTGTTGGGGGT | 6.08 | 1.17 | 2.44 | 0.52 | Hs.55016 | hypothetical | 2.49 |
| | | | | | | | protein | |
| | | | | | | | FLJ21935 | |
| | | | | | | | [Swisspro | |
| 201. | GCCAGCTGACA | 6.08 | 1.17 | 2.44 | 0.52 | Hs.118913 | ESTs, | 2.49 |
| | | | | | | | Moderately | |
| | | | | | | | similar to | |
| | | | | | | | T46371 hypot | |
| 202. | CTGAGGCCTGG | 8.00 | 1.22 | 3.26 | 0.48 | Hs.82109 | syndecan 1 | 2.45 |
| | | | | | | | [Swissprot: | |
| | | | | | | | sp\|P18827; sp\|Q96 | |
| 203. | GGCGCCTCCTT | 8.00 | 1.22 | 3.26 | 0.48 | Hs.77290 | transaldolase 1 | 2.45 |
| | | | | | | | [Swissprot: | |
| | | | | | | | sp\|P37837; s | |
| 204. | AAGAAGCAAGA | 8.00 | 1.22 | 3.26 | 0.48 | Hs.343665 | ribosomal | 2.45 |
| | | | | | | | protein S15a | |
| | | | | | | | [Swissprot: sp\|P | |
| 205. | GCGGGGTACCC | 8.00 | 1.22 | 3.26 | 0.48 | Hs.322466 | Homo sapiens | 2.45 |
| | | | | | | | cDNA: | |
| | | | | | | | FLJ23491 fis, | |
| | | | | | | | clone L | |
| 206. | ATTTTGTCGTG | 8.00 | 1.22 | 3.26 | 0.48 | Hs.305953 | zinc finger | 2.45 |
| | | | | | | | protein 83 | |
| | | | | | | | (HPF1) | |
| | | | | | | | [Swisspro | |
| 207. | TGGCTTATTAA | 8.00 | 1.22 | 3.26 | 0.48 | Hs.18021 | hypothetical | 2.45 |
| | | | | | | | protein | |
| | | | | | | | FLJ20446 | |
| | | | | | | | [Swisspro | |
| 208. | GCACTTCAAAC | −7.89 | 1.19 | −2.00 | 0.39 | Hs.66191 | Homo sapiens | 3.95 |
| | | | | | | | clone 24675 | |
| | | | | | | | mRNA sequence | |
| 209. | TATATGGATGT | −7.89 | 1.19 | −2.00 | 0.39 | Hs.42758 | Homo sapiens, | 3.95 |
| | | | | | | | clone | |
| | | | | | | | IMAGE: 3354845, | |
| | | | | | | | mRNA, | |
| 210. | TCAGTTTGGAG | −7.89 | 1.19 | −2.00 | 0.39 | Hs.3873 | palmitoyl-protein | 3.95 |
| | | | | | | | thioesterase 1 | |
| | | | | | | | (ceroid | |
| 211. | AAAATAAAGCT | −7.89 | 1.19 | −2.00 | 0.39 | Hs.293818 | Homo sapiens, | 3.95 |
| | | | | | | | Similar to | |
| | | | | | | | hypothetical pr | |
| 212. | CACAAAATCTC | −7.89 | 1.19 | −2.00 | 0.39 | Hs.12372 | tripartite motif- | 3.95 |
| | | | | | | | containing 2 | |
| | | | | | | | [Swisspro | |
| 213. | CTGAAAAAAAA | −7.89 | 1.19 | −2.00 | 0.39 | Hs.12142 | WD repeat | 3.95 |
| | | | | | | | domain 13 | |
| | | | | | | | [Swissprot: | |
| | | | | | | | sp\|Q9BU | |
| 214. | AATGGAAGGTG | −7.89 | 1.19 | −2.00 | 0.39 | Hs.11260 | hypothetical | 3.95 |
| | | | | | | | protein | |
| | | | | | | | FLJ11264 | |
| | | | | | | | [Swisspro | |
| 215. | GCTCATTAAAG | −7.89 | 1.19 | −2.00 | 0.39 | Hs.112237 | ESTs | 3.95 |
| | | | | | | | [Swissprot: | |
| | | | | | | | none] | |
| 216. | ATGGCCTCCTC | 5.07 | 0.92 | 2.04 | 0.35 | Hs.83734 | syntaxin 4A | 2.49 |
| | | | | | | | (placental) | |
| | | | | | | | [Swissprot: spl | |
| 217. | TTTTCTGCTGG | −5.92 | 1.13 | −2.46 | 0.32 | Hs.204041 | chromosome 14 | 2.41 |
| | | | | | | | open reading | |
| | | | | | | | frame 3 [Swi | |
| 218. | ATTTGTGTGTA | 6.00 | 0.91 | 2.44 | 0.29 | Hs.94499 | ESTs | 2.46 |
| | | | | | | | [Swissprot: | |
| | | | | | | | none] | |
| 219. | TGTGTGTGTGT | 6.00 | 0.91 | 2.44 | 0.29 | Hs.9291 | hypothetical | 2.46 |
| | | | | | | | protein | |
| | | | | | | | FLJ13511 | |
| | | | | | | | [Swisspro | |
| 220. | TCTGTTAATAA | 6.00 | 0.91 | 2.44 | 0.29 | Hs.89434 | drebrin 1 | 2.46 |
| | | | | | | | [Swissprot: | |
| | | | | | | | sp\|Q16643; sp\|Q9UF | |
| 221. | AACAACTGGCT | 6.00 | 0.91 | 2.44 | 0.29 | Hs.75243 | bromodomain- | 2.46 |
| | | | | | | | containing 2 | |
| | | | | | | | [Swissprot: sp | |
| 222. | TGTCAGAGATG | 6.00 | 0.91 | 2.44 | 0.29 | Hs.73957 | RAB5A, member | 2.46 |
| | | | | | | | RAS oncogene | |
| | | | | | | | family [Swis | |
| 223. | TGGGACTCCAG | 6.00 | 0.91 | 2.44 | 0.29 | Hs.59384 | hypothetical | 2.46 |
| | | | | | | | protein | |
| | | | | | | | MGC3047 | |
| | | | | | | | [Swissprot | |
| 224. | GAAACTAGATC | 6.00 | 0.91 | 2.44 | 0.29 | Hs.37883 | hypothetical | 2.46 |
| | | | | | | | protein PNAS- | |
| | | | | | | | 131 [Swisspro | |
| 225. | GTGGCGCGCAC | 6.00 | 0.91 | 2.44 | 0.29 | Hs.346741 | EST [Swissprot: | 2.46 |
| | | | | | | | none] | |
| 226. | TGGGCTCTGAA | 6.00 | 0.91 | 2.44 | 0.29 | Hs.323567 | CD36 antigen | 2.46 |
| | | | | | | | (collagen type I | |
| | | | | | | | receptor. | |
| 227. | TACCAAGCCAG | 6.00 | 0.91 | 2.44 | 0.29 | Hs.31388 | ESTs | 2.46 |
| | | | | | | | [Swissprot: | |
| | | | | | | | none] | |
| 228. | CTGAAAACCAC | 6.00 | 0.91 | 2.44 | 0.29 | Hs.285582 | ESTs, Weakly | 2.46 |
| | | | | | | | similar to | |
| | | | | | | | ubiquitous TPRm | |
| 229. | AATTGCCACTG | 6.00 | 0.91 | 2.44 | 0.29 | Hs.287863 | hypothetical | 2.46 |
| | | | | | | | protein | |
| | | | | | | | FLJ12475 | |
| | | | | | | | [Swisspro | |
| 230. | TTCAGGGCTTC | 6.00 | 0.91 | 2.44 | 0.29 | Hs.286236 | KIAA1856 | 2.46 |
| | | | | | | | protein | |
| | | | | | | | [Swissprot: | |
| | | | | | | | sp\|P55010; | |
| 231. | AGCTTTUCCAAT | 6.00 | 0.91 | 2.44 | 0.29 | Hs.274402 | heat shock 70 kD | 2.46 |
| | | | | | | | protein 1B | |
| | | | | | | | [Swissprot: | |
| 232. | TTGGGAGTGAG | 6.00 | 0.91 | 2.44 | 0.29 | Hs.26285 | imidazoline | 2.46 |
| | | | | | | | receptor | |
| | | | | | | | candidate | |
| | | | | | | | [Swisspr | |
| 233. | TGGTTCTATAT | 6.00 | 0.91 | 2.44 | 0.29 | Hs.26213 | Homo sapiens | 2.46 |
| | | | | | | | mRNA for TdT | |
| | | | | | | | binding protei | |
| 234. | GGTGTGCACCT | 6.00 | 0.91 | 2.44 | 0.29 | Hs.24587 | signal | 2.46 |
| | | | | | | | transduction | |
| | | | | | | | protein (SH3 | |
| | | | | | | | contain | |
| 235. | AGTGGCTGCCC | 6.00 | 0.91 | 2.44 | 0.29 | Hs.24435 | Homo sapiens | 2.46 |
| | | | | | | | clone | |
| | | | | | | | CDABP0028 | |
| | | | | | | | mRNA sequen | |
| 236. | CAGGGCGGGTT | 6.00 | 0.91 | 2.44 | 0.29 | Hs.23978 | scaffold | 2.46 |
| | | | | | | | attachment | |
| | | | | | | | factor B | |
| | | | | | | | [Swissprot | |
| 237. | TAAACATTGTC | 6.00 | 0.91 | 2.44 | 0.29 | Hs.23060 | DKFZP564F052 | 2.46 |
| | | | | | | | 2 protein | |
| | | | | | | | [Swissprot: | |
| | | | | | | | sp\|Q9 | |
| 238. | GCAGCTGACGG | 6.00 | 0.91 | 2.44 | 0.29 | Hs.227751 | lectin, | 2.46 |
| | | | | | | | galactoside- | |
| | | | | | | | binding, soluble, 1 | |
| 239. | AATAATCCTGG | 6.00 | 0.91 | 2.44 | 0.29 | Hs.188361 | Homo sapiens | 2.46 |
| | | | | | | | cDNA FLJ12807 | |
| | | | | | | | fis, clone NT | |
| 240. | ACTGCACCACT | 6.00 | 0.91 | 2.44 | 0.29 | Hs.185910 | ESTs, Weakly | 2.46 |
| | | | | | | | similar to | |
| | | | | | | | ALU1_HUMAN | |
| | | | | | | | ALU S | |
| 241. | TGGCTGCATAG | 6.00 | 0.91 | 2.44 | 0.29 | Hs.164478 | hypothetical | 2.46 |
| | | | | | | | protein | |
| | | | | | | | FLJ21939 | |
| | | | | | | | similar to | |
| 242. | GGCTTGCTGAC | 6.00 | 0.91 | 2.44 | 0.29 | Hs.1369 | decay | 2.46 |
| | | | | | | | accelerating | |
| | | | | | | | factor for | |
| | | | | | | | complement | |
| 243. | TTTCTGGAGGT | 6.00 | 0.91 | 2.44 | 0.29 | Hs.129943 | KIAA0545 | 2.46 |
| | | | | | | | protein | |
| | | | | | | | [Swissprot: | |
| | | | | | | | sp\|O60292; | |
| 244. | TTGGGAGGCTG | 6.00 | 0.91 | 2.44 | 0.29 | Hs.118269 | ESTs, Weakly | 2.46 |
| | | | | | | | similar to | |
| | | | | | | | A46010 X-linked | |
| 245. | AAGATATTCTC | 6.00 | 0.91 | 2.44 | 0.29 | Hs.103804 | heterogeneous | 2.46 |
| | | | | | | | nuclear | |
| | | | | | | | ribonucleoprotein | |
| 246. | CCTTGTCCAGC | 6.00 | 0.91 | 2.44 | 0.29 | Hs.101067 | GCN5 (general | 2.46 |
| | | | | | | | control of amino- | |
| | | | | | | | acid synt | |
| 247. | GCTTTTATTCA | 6.08 | 1.17 | 1.63 | 0.27 | Hs.31819 | HT014 | 3.73 |
| | | | | | | | [Swissprot: | |
| | | | | | | | sp\|Q9GZP4; sp\| | |
| | | | | | | | Q9NRI8;] | |
| 248. | TTTGTTAATTC | 8.00 | 1.22 | 1.63 | 0.20 | Hs.278857 | heterogeneous | 4.91 |
| | | | | | | | nuclear | |
| | | | | | | | ribonucleoprotein | |
| 249. | GGGGCAACAGC | 8.00 | 1.22 | 1.63 | 0.20 | Hs.276770 | CDW52 antigen | 4.91 |
| | | | | | | | (CAMPATH-1 | |
| | | | | | | | antigen) [Swis | |
| 250. | CTGGCGCCGAT | 8.00 | 1.22 | 1.63 | 0.20 | Hs.183180 | anaphase | 4.91 |
| | | | | | | | promoting | |
| | | | | | | | complex subunit | |
| | | | | | | | 11 (y | |
| 251. | CATCGTTACAT | 8.00 | 1.22 | 1.63 | 0.20 | Hs.173802 | KIAA0603 gene | 4.91 |
| | | | | | | | product | |
| | | | | | | | [Swissprot: | |
| | | | | | | | sp\|O6 | |
| 252. | TTGTCCAGAGG | 8.00 | 1.22 | 1.63 | 0.20 | Hs.14839 | polymerase | 4.91 |
| | | | | | | | (RNA) II (DNA | |
| | | | | | | | directed) polyp | |
| 253. | GACAATGCCAG | 6.08 | 1.17 | 1.22 | 0.10 | Hs.155433 | ATP synthase, | 4.98 |
| | | | | | | | H+ transporting, | |
| | | | | | | | mitochond | |
| 254. | TACATTCTGTG | −5.92 | 1.13 | −1.23 | 0.07 | Hs.86386 | myeloid cell | 4.81 |
| | | | | | | | leukemia | |
| | | | | | | | sequence 1 | |
| | | | | | | | (BCL2-r | |
| 255. | ATAGACATAAA | −5.92 | 1.13 | −1.23 | 0.07 | Hs.78614 | complement | 4.81 |
| | | | | | | | component 1, q | |
| | | | | | | | subcomponent b | |
| 256. | GTGGATGGACT | −5.92 | 1.13 | −1.23 | 0.07 | Hs.350752 | [Swissprot: | 4.81 |
| | | | | | | | sp\|Q96AJ6; sp\|Q9P2R4;] | |
| 257. | CCTTCCAAATT | −5.92 | 1.13 | −1.23 | 0.07 | Hs.343521 | malate | 4.81 |
| | | | | | | | dehydrogenase | |
| | | | | | | | 2, NAD | |
| | | | | | | | (mitochondri | |
| 258. | ACAAAAAAAAA | −5.92 | 1.13 | −1.23 | 0.07 | Hs.326583 | Homo sapiens | 4.81 |
| | | | | | | | mRNA; cDNA | |
| | | | | | | | DKFZp434A1520 | |
| | | | | | | | (f | |
| 259. | CAAGCAAAATA | 6.00 | 0.91 | 1.22 | 0.05 | Hs.9908 | nitrogen fixation | 4.92 |
| | | | | | | | cluster-like | |
| | | | | | | | [Swisspr | |
| 260. | TATTTCAATTG | 6.00 | 0.91 | 1.22 | 0.05 | Hs.79507 | KIAA0582 | 4.92 |
| | | | | | | | protein | |
| | | | | | | | [Swissprot: | |
| | | | | | | | sp\|O60326; | |
| 261. | TCATAGCCTTG | 6.00 | 0.91 | 1.22 | 0.05 | Hs.78846 | heat shock 27 kD | 4.92 |
| | | | | | | | protein 2 | |
| | | | | | | | [Swissprot: s | |
| 262. | TTGGTGGAGGT | 6.00 | 0.91 | 1.22 | 0.05 | Hs.76294 | CD63 antigen | 4.92 |
| | | | | | | | (melanoma 1 | |
| | | | | | | | antigen) [Swis | |
| 263. | TCTTGAACAGC | 6.00 | 0.91 | 1.22 | 0.05 | Hs.72249 | par-3 | 4.92 |
| | | | | | | | (partitioning | |
| | | | | | | | defective 3, | |
| | | | | | | | C. elega | |
| 264. | TCAGACAAAAG | 6.00 | 0.91 | 1.22 | 0.05 | Hs.66881 | dynein, | 4.92 |
| | | | | | | | cytoplasmic, | |
| | | | | | | | intermediate | |
| | | | | | | | polype | |
| 265. | TACCCCCAAAC | 6.00 | 0.91 | 1.22 | 0.05 | Hs.345694 | ESTs | 4.92 |
| | | | | | | | [Swissprot: | |
| | | | | | | | none] | |
| 266. | GACCCTTTTGG | 6.00 | 0.91 | 1.22 | 0.05 | Hs.312705 | hypothetical | 4.92 |
| | | | | | | | protein | |
| | | | | | | | FLJ21019 | |
| | | | | | | | [Swisspro | |
| 267. | TTGGCATTGTC | 6.00 | 0.91 | 1.22 | 0.05 | Hs.306117 | KIAA0306 | 4.92 |
| | | | | | | | protein | |
| | | | | | | | [Swissprot: | |
| | | | | | | | sp\|P49840; | |
| 268. | TGAAAGTAACA | 6.00 | 0.91 | 1.22 | 0.05 | Hs.256583 | interleukin | 4.92 |
| | | | | | | | enhancer | |
| | | | | | | | binding factor 3, 9 | |
| 269. | GAGGAGTGGGT | 6.00 | 0.91 | 1.22 | 0.05 | Hs.206770 | zinc finger | 4.92 |
| | | | | | | | protein 297 | |
| | | | | | | | [Swissprot: sp\| | |
| 270. | GCAAGACCCCA | 6.00 | 0.91 | 1.22 | 0.05 | Hs.170861 | ESTs, Weakly | 4.92 |
| | | | | | | | similar to | |
| | | | | | | | Z195_HUMAN | |
| | | | | | | | ZINC | |
| 271. | TGCTTGACAAG | 6.00 | 0.91 | 1.22 | 0.05 | Hs.106127 | RNA polymerase | 4.92 |
| | | | | | | | I 16 kDa subunit | |
| | | | | | | | [Swissp | |
| 272. | TGTGGTGGTGT | 5.07 | 0.92 | 1.02 | 0.00 | Hs.83422 | MLN51 protein | 4.97 |
| | | | | | | | [Swissprot: | |
| | | | | | | | sp\|O15234;] | |
| 273. | AAGCCTTGCTG | 5.07 | 0.92 | 1.02 | 0.00 | Hs.6289 | hypothetical | 4.97 |
| | | | | | | | protein | |
| | | | | | | | FLJ20886 | |
| | | | | | | | [Swisspro | |

TABLE 9

| | | Scalp/ | | Scalp/ | | | |
|---|---|---|---|---|---|---|---|
| No. | Tag sequence | face | Sign. | breast | Sign. | Annotation | Description |
| 274. | CACGCAATGCT | −3.95 | 0.65 | −13.50 | 2.99 | Hs.21907 | histone acetyltransferase |
| | | | | | | | [Swissprot: s |
| 275. | GCTAACCCCTG | −3.95 | 0.65 | −12.28 | 2.68 | Hs.279772 | brain specific protein |
| | | | | | | | [Swissprot: sp\|Q |
| 276. | TGTGGCGTATA | 3.04 | 1.06 | 14.66 | 2.29 | Hs.288965 | Homo sapiens cDNA: |
| | | | | | | | FLJ22300 fis, clone H |
| 277. | GACTCTTCAGT | 4.05 | 1.21 | 13.03 | 2.03 | Hs.234726 | serine (or cysteine) |
| | | | | | | | proteinase inhibito |
| 278. | GATGAACTGAA | 4.05 | 1.21 | 13.03 | 2.03 | Hs.30035 | splicing factor, |
| | | | | | | | arginine/serine-rich (t |
| 279. | CTGCCAAGTTG | −3.29 | 1.22 | −4.50 | 1.91 | Hs.75873 | zyxin [Swissprot: |
| | | | | | | | sp\|Q15942; sp\|Q28617; s |
| 280. | AATGAACAATA | 3.55 | 0.98 | 11.40 | 1.77 | Hs.11342 | ninjurin 1 [Swissprot: |
| | | | | | | | sp\|Q92982;] |
| 281. | GTTGTCTTTGA | 3.55 | 0.98 | 11.40 | 1.77 | Hs.258798 | hypothetical protein |
| | | | | | | | FLJ20003 [Swisspro |
| 282. | GATTGAACCTC | 3.04 | 1.06 | 7.33 | 1.55 | Hs.239926 | sterol-C4-methyl oxidase- |
| | | | | | | | like [Swisspro |
| 283. | AGCTCTTGGAG | 3.04 | 1.06 | 7.33 | 1.55 | Hs.334841 | selenium binding protein 1 |
| | | | | | | | [Swissprot: |
| 284. | TGATGTGGAAT | 3.04 | 1.06 | 7.33 | 1.55 | Hs.5687 | protein phosphatase 1B |
| | | | | | | | (formerly 2C), ma |
| 285. | TCACAAAAGAG | 3.04 | 0.76 | 9.77 | 1.51 | Hs.12646 | hypothetical protein |
| | | | | | | | FLJ22693 [Swisspro |
| 286. | TAGATGTGATT | 3.04 | 0.76 | 9.77 | 1.51 | Hs.131740 | Homo sapiens cDNA: |
| | | | | | | | FLJ22562 fis, clone H |
| 287. | GCATCTGTTTA | 3.04 | 0.76 | 9.77 | 1.51 | Hs.250175 | homolog of yeast long |
| | | | | | | | chain polyunsatura |
| 288. | GCTCTGTAAGC | 3.04 | 0.76 | 9.77 | 1.51 | Hs.268149 | putative methyltransferase |
| | | | | | | | [Swissprot: |
| 289. | TGAAGTTATAC | 3.04 | 0.76 | 9.77 | 1.51 | Hs.287797 | integrin, beta 1 (fibronectin |
| | | | | | | | receptor, |
| 290. | TTATGGATCTC | 3.04 | 0.76 | 9.77 | 1.51 | Hs.5662 | guanine nucleotide binding |
| | | | | | | | protein (G pr |
| 291. | GTGAGACCCCA | −3.95 | 0.65 | −7.37 | 1.45 | Hs.198671 | ESTs [Swissprot: none] |
| 292. | CCAGTGCACTC | −3.95 | 0.65 | −7.37 | 1.45 | Hs.317309 | EST [Swissprot: none] |
| 293. | TTGTAAAAGGA | 4.05 | 1.21 | 6.52 | 1.33 | Hs.106357 | valosin-containing protein |
| | | | | | | | [Swissprot: |
| 294. | TAAGTTTAATT | 4.05 | 1.21 | 6.52 | 1.33 | Hs.75760 | sterol carrier protein 2 |
| | | | | | | | [Swissprot: sp |

TABLE 10

| | | | | | | | | Quotient |
|---|---|---|---|---|---|---|---|---|
| | | | | | | | | (scalp/ |
| | | | | | | | | face)/ |
| | | Scalp/ | | Scalp/ | | | | (scalp/ |
| No. | Tag sequence | face | Sign. | breast | Sign. | Annotation | Description | breast) |
| 295. | CAAAATCAGGA | −4.93 | 0.89 | −6.14 | 1.15 | Hs.79933 | cyclin I | 0.80 |
| | | | | | | | [Swissprot: | |
| | | | | | | | sp\|Q14094;] | |
| 296. | ATGTGTAACGA | −4.93 | 0.89 | −6.14 | 1.15 | Hs.81256 | S100 calcium- | 0.80 |
| | | | | | | | binding protein | |
| | | | | | | | A4 (calcium | |
| 297. | GCAGTCGCTTG | 3.55 | 0.98 | 5.70 | 1.11 | Hs.100002 | HSPC162 | 0.62 |
| | | | | | | | protein | |
| | | | | | | | [Swissprot: | |
| | | | | | | | sp\|Q96IV3; s | |
| 298. | GGGCAGAATAT | 3.55 | 0.98 | 5.70 | 1.11 | Hs.168670 | peroxisomal | 0.62 |
| | | | | | | | farnesylated | |
| | | | | | | | protein [Swiss | |
| 299. | CAGTTCCCTCC | 4.05 | 0.67 | 6.52 | 0.99 | Hs.104800 | hypothetical | 0.62 |
| | | | | | | | protein | |
| | | | | | | | FLJ10134 | |
| | | | | | | | [Swisspro | |
| 300. | TCGGGTGTGGG | 4.05 | 0.67 | 6.52 | 0.99 | Hs.104938 | hypothetical | 0.62 |
| | | | | | | | protein | |
| | | | | | | | MGC15906 | |
| | | | | | | | [Swisspro | |
| 301. | TTAAAATACAG | 4.05 | 0.67 | 6.52 | 0.99 | Hs.11356 | ESTs | 0.62 |
| | | | | | | | [Swissprot: | |
| | | | | | | | none] | |
| 302. | CTCAGAACACT | 4.05 | 0.67 | 6.52 | 0.99 | Hs.1227 | aminolevulinate, | 0.62 |
| | | | | | | | delta-, | |
| | | | | | | | dehydratase [S | |
| 303. | CCTGCACCAAC | 4.05 | 0.67 | 6.52 | 0.99 | Hs.124747 | ESTs | 0.62 |
| | | | | | | | [Swissprot: | |
| | | | | | | | none] | |
| 304. | TTGTTTGTAAA | 4.05 | 0.67 | 6.52 | 0.99 | Hs.15020 | homolog of | 0.62 |
| | | | | | | | mouse quaking | |
| | | | | | | | QKI (KH domain | |
| 305. | TGTTTATATTG | 4.05 | 0.67 | 6.52 | 0.99 | Hs.154151 | protein tyrosine | 0.62 |
| | | | | | | | phosphatase, | |
| | | | | | | | receptor I | |
| 306. | GCACCTCCTAG | 4.05 | 0.67 | 6.52 | 0.99 | Hs.155218 | E1B-55 kDa- | 0.62 |
| | | | | | | | associated | |
| | | | | | | | protein 5 | |
| | | | | | | | [Swisspr | |
| 307. | TAAATCTGAAG | 4.05 | 0.67 | 6.52 | 0.99 | Hs.15702 | ESTs | 0.62 |
| | | | | | | | [Swissprot: | |
| | | | | | | | none] | |
| 308. | CATAACCTTCC | 4.05 | 0.67 | 6.52 | 0.99 | Hs.167460 | splicing factor, | 0.62 |
| | | | | | | | arginine/serine- | |
| | | | | | | | rich 3 | |
| 309. | GGGGGGGTCTC | 4.05 | 0.67 | 6.52 | 0.99 | Hs.171734 | protein | 0.62 |
| | | | | | | | phosphatase 2, | |
| | | | | | | | regulatory | |
| | | | | | | | subuni | |
| 310. | TGTCTGGTTGT | 4.05 | 0.67 | 6.52 | 0.99 | Hs.180450 | ribosomal | 0.62 |
| | | | | | | | protein S24 | |
| | | | | | | | [Swissprot: | |
| | | | | | | | sp\|P1 | |
| 311. | TCTGGTCTGGG | 4.05 | 0.67 | 6.52 | 0.99 | Hs.184488 | flotillin 2 | 0.62 |
| | | | | | | | [Swissprot: | |
| | | | | | | | sp\|Q14254; sp\|Q9 | |
| 312. | AGTCAGCTGGA | 4.05 | 0.67 | 6.52 | 0.99 | Hs.2132 | epidermal | 0.62 |
| | | | | | | | growth factor | |
| | | | | | | | receptor | |
| | | | | | | | pathway | |
| 313. | AAATGACTTAT | 4.05 | 0.67 | 6.52 | 0.99 | Hs.225977 | nuclear receptor | 0.62 |
| | | | | | | | coactivator 3 | |
| | | | | | | | [Swisspr | |
| 314. | TAGAGTGTAAA | 4.05 | 0.67 | 6.52 | 0.99 | Hs.25063 | PRO0461 | 0.62 |
| | | | | | | | protein | |
| | | | | | | | [Swissprot: | |
| | | | | | | | sp\|Q9UI25;] | |
| 315. | TCAGTGTGTGA | 4.05 | 0.67 | 6.52 | 0.99 | Hs.27099 | likely ortholog of | 0.62 |
| | | | | | | | mouse ADP- | |
| | | | | | | | ribosylatio | |
| 316. | ACACCTCTAAA | 4.05 | 0.67 | 6.52 | 0.99 | Hs.273230 | hypothetical | 0.62 |
| | | | | | | | protein | |
| | | | | | | | FLJ10830 | |
| | | | | | | | [Swisspro | |
| 317. | GAGGCTCAATC | 4.05 | 0.67 | 6.52 | 0.99 | Hs.278554 | chromobox | 0.62 |
| | | | | | | | homolog 3 | |
| | | | | | | | (Drosophila HP1 | |
| | | | | | | | gamm | |
| 318. | CTGAACTGTGA | 4.05 | 0.67 | 6.52 | 0.99 | Hs.282990 | putative protein | 0.62 |
| | | | | | | | tyrosine kinase | |
| | | | | | | | [Swiss | |
| 319. | CACCTAGCATA | 4.05 | 0.67 | 6.52 | 0.99 | Hs.29403 | hypothetical | 0.62 |
| | | | | | | | protein | |
| | | | | | | | FLJ22060 | |
| | | | | | | | [Swisspro | |
| 320. | AAGTTTTCTAA | 4.05 | 0.67 | 6.52 | 0.99 | Hs.30022 | ESTs, Weakly | 0.62 |
| | | | | | | | similar to | |
| | | | | | | | NAH6_HUMAN | |
| | | | | | | | SODIU | |
| 321. | CTAAAAGGAGA | 4.05 | 0.67 | 6.52 | 0.99 | Hs.334612 | small nuclear | 0.62 |
| | | | | | | | ribonucleoprotein | |
| | | | | | | | polypept | |
| 322. | ATTCCTTTAAT | 4.05 | 0.67 | 6.52 | 0.99 | Hs.334762 | hypothetical | 0.62 |
| | | | | | | | protein | |
| | | | | | | | FLJ14735 | |
| | | | | | | | [Swisspro | |
| 323. | GCTTAATGTGT | 4.05 | 0.67 | 6.52 | 0.99 | Hs.334885 | mitochondrial | 0.62 |
| | | | | | | | GTP binding | |
| | | | | | | | protein [Swis | |
| 324. | CTATGGTTGCA | 4.05 | 0.67 | 6.52 | 0.99 | Hs.33724 | CGI-40 protein | 0.62 |
| | | | | | | | [Swissprot: | |
| | | | | | | | sp\|Q9Y357;] | |
| 325. | CTTTTCATCAT | 4.05 | 0.67 | 6.52 | 0.99 | Hs.3726 | x 003 protein | 0.62 |
| | | | | | | | [Swissprot: | |
| | | | | | | | sp\|Q969U7; sp\| | |
| 326. | GTCTTTCTGGG | 4.05 | 0.67 | 6.52 | 0.99 | Hs.49753 | uveal | 0.62 |
| | | | | | | | autoantigen with | |
| | | | | | | | coiled-coil domal | |
| 327. | TGAGTTTTACA | 4.05 | 0.67 | 6.52 | 0.99 | Hs.58373 | ESTs | 0.62 |
| | | | | | | | [Swissprot: | |
| | | | | | | | none] | |
| 328. | TCTACAAATAT | 4.05 | 0.67 | 6.52 | 0.99 | Hs.6019 | Homo sapiens | 0.62 |
| | | | | | | | cDNA: | |
| | | | | | | | FLJ21288 fis, | |
| | | | | | | | clone C | |
| 329. | CTGAGTTAGGT | 4.05 | 0.67 | 6.52 | 0.99 | Hs.72980 | Protein P3 | 0.62 |
| | | | | | | | [Swissprot: | |
| | | | | | | | sp\|P09131; sp\|P11 | |
| 330. | GGAAGGGGAGG | 4.05 | 0.67 | 6.52 | 0.99 | Hs.73090 | nuclear factor of | 0.62 |
| | | | | | | | kappa light | |
| | | | | | | | polypeptid | |
| 331. | GAGATGTCTGA | 4.05 | 0.67 | 6.52 | 0.99 | Hs.74861 | activated RNA | 0.62 |
| | | | | | | | polymerase II | |
| | | | | | | | transcriptio | |
| 332. | GTTACAAACTA | 4.05 | 0.67 | 6.52 | 0.99 | Hs.75248 | topoisomerase | 0.62 |
| | | | | | | | (DNA) II beta | |
| | | | | | | | (180 kD) [Sw | |
| 333. | TCTGGTTTGTC | 4.05 | 0.67 | 6.52 | 0.99 | Hs.76293 | thymosin, beta | 0.62 |
| | | | | | | | 10 [Swissprot: | |
| | | | | | | | sp\|P13472 | |
| 334. | TTCTAATCATT | 4.05 | 0.67 | 6.52 | 0.99 | Hs.79732 | fibulin 1 | 0.62 |
| | | | | | | | [Swissprot: | |
| | | | | | | | sp\|O60822; sp\|P231 | |
| 335. | TAGTTTCAACT | 4.05 | 0.67 | 6.52 | 0.99 | Hs.8127 | KIAA0144 gene | 0.62 |
| | | | | | | | product | |
| | | | | | | | [Swissprot: | |
| | | | | | | | sp\|Q1 | |
| 336. | GAGGTTCTTCC | 4.05 | 0.67 | 6.52 | 0.99 | Hs.831 | 3- | 0.62 |
| | | | | | | | hydroxymethyl- | |
| | | | | | | | 3-methylglutaryl- | |
| | | | | | | | Coenzym | |
| 337. | AGGGCTGCAGG | 4.05 | 0.67 | 6.52 | 0.99 | Hs.89839 | EphA1 | 0.62 |
| | | | | | | | [Swissprot: | |
| | | | | | | | sp\|P21709;] | |
| 338. | GTGTAATAAGA | 3.38 | 1.27 | 2.72 | 0.89 | Hs.232400 | heterogeneous | 1.24 |
| | | | | | | | nuclear | |
| | | | | | | | ribonucleoprotein | |
| 339. | GGATACAACCT | 3.04 | 0.76 | 4.89 | 0.89 | Hs.173993 | RNA binding | 0.62 |
| | | | | | | | motif protein 6 | |
| | | | | | | | [Swissprot: | |
| 340. | AAGAATCAAAA | 3.04 | 0.76 | 4.89 | 0.89 | Hs.183435 | NADH | 0.62 |
| | | | | | | | dehydrogenase | |
| | | | | | | | (ubiquinone) 1 | |
| | | | | | | | beta s | |
| 341. | TGAGCCTCGTG | 3.04 | 0.76 | 4.89 | 0.89 | Hs.254105 | enolase 1, | 0.62 |
| | | | | | | | (alpha) | |
| | | | | | | | [Swissprot: | |
| | | | | | | | sp\|P0673 | |
| 342. | ACTGTTTGTTT | 3.04 | 0.76 | 4.89 | 0.89 | Hs.814 | major | 0.62 |
| | | | | | | | histocompatibility | |
| | | | | | | | complex, class | |
| 343. | CCACTGCAGTC | −3.95 | 0.65 | −4.91 | 0.86 | Hs.324405 | ESTs, Weakly | 0.80 |
| | | | | | | | similar to | |
| | | | | | | | ALU1_HUMAN | |
| | | | | | | | ALU S | |
| 344. | CTGCTATGGTC | 3.04 | 0.44 | 4.89 | 0.73 | Hs.103291 | neuritin | 0.62 |
| | | | | | | | [Swissprot: | |
| | | | | | | | sp\|Q9NPD7;] | |
| 345. | TGAGGCCTCTC | 3.04 | 0.44 | 4.89 | 0.73 | Hs.108115 | hypothetical | 0.62 |
| | | | | | | | protein | |
| | | | | | | | FLJ10101 | |
| | | | | | | | [Swisspro | |
| 346. | GCTGTAGGGGC | 3.04 | 0.44 | 4.89 | 0.73 | Hs.111611 | ribosomal | 0.62 |
| | | | | | | | protein L27 | |
| | | | | | | | [Swissprot: | |
| | | | | | | | sp\|P0 | |
| 347. | TTGGCAACATT | 3.04 | 0.44 | 4.89 | 0.73 | Hs.11463 | UMP-CMP | 0.62 |
| | | | | | | | kinase | |
| | | | | | | | [Swissprot: | |
| | | | | | | | sp\|P30085; sp | |
| 348. | ATAGTAGCTTC | 3.04 | 0.44 | 4.89 | 0.73 | Hs.118400 | singed | 0.62 |
| | | | | | | | (Drosophila)-like | |
| | | | | | | | (sea urchin fas | |
| 349. | GGGCTACGTCC | 3.04 | 0.44 | 4.89 | 0.73 | Hs.123107 | kallikrein 1, | 0.62 |
| | | | | | | | renal/pancreas/salivary [ | |
| 350. | GAAAGCCCCTA | 3.04 | 0.44 | 4.89 | 0.73 | Hs.12526 | Homo sapiens | 0.62 |
| | | | | | | | clone 23903 | |
| | | | | | | | mRNA sequence | |
| 351. | ACCCAGCGGGC | 3.04 | 0.44 | 4.89 | 0.73 | Hs.126705 | ESTs | 0.62 |
| | | | | | | | [Swissprot: | |
| | | | | | | | none] | |
| 352. | AGAGCTCACTA | 3.04 | 0.44 | 4.89 | 0.73 | Hs.13845 | solute carrier | 0.62 |
| | | | | | | | family 25 | |
| | | | | | | | (carnitine/acyl | |
| 353. | CTTCTAGGGAG | 3.04 | 0.44 | 4.89 | 0.73 | Hs.13999 | KIAA0700 | 0.62 |
| | | | | | | | protein | |
| | | | | | | | [Swissprot: | |
| | | | | | | | sp\|O75182; | |
| 354. | CCTAAGGGAGA | 3.04 | 0.44 | 4.89 | 0.73 | Hs.153022 | TATA box | 0.62 |
| | | | | | | | binding protein | |
| | | | | | | | (TBP)-associate | |
| 355. | ACTAACTGTGT | 3.04 | 0.44 | 4.89 | 0.73 | Hs.16003 | retinoblastoma- | 0.62 |
| | | | | | | | binding protein 4 | |
| | | | | | | | [Swiss | |
| 356. | TGTAAGAAAAG | 3.04 | 0.44 | 4.89 | 0.73 | Hs.16340 | sulfite oxidase | 0.62 |
| | | | | | | | [Swissprot: | |
| | | | | | | | sp\|P51687;] | |
| 357. | TTAAATCCCAT | 3.04 | 0.44 | 4.89 | 0.73 | Hs.170121 | protein tyrosine | 0.62 |
| | | | | | | | phosphatase, | |
| | | | | | | | receptor t | |
| 358. | TAAACTTCTGA | 3.04 | 0.44 | 4.89 | 0.73 | Hs.172108 | nucleoporin | 0.62 |
| | | | | | | | 88 kD | |
| | | | | | | | [Swissprot: | |
| | | | | | | | sp\|Q99567; | |
| 359. | CAAGTAACTAG | 3.04 | 0.44 | 4.89 | 0.73 | Hs.172199 | adenylate | 0.62 |
| | | | | | | | cyclase 7 | |
| | | | | | | | [Swissprot: | |
| | | | | | | | sp\|P518 | |
| 360. | ACATAGACCGA | 3.04 | 0.44 | 4.89 | 0.73 | Hs.173594 | serine (or | 0.62 |
| | | | | | | | cysteine) | |
| | | | | | | | proteinase | |
| | | | | | | | inhibitor | |
| 361. | AGCTGTCTCTT | 3.04 | 0.44 | 4.89 | 0.73 | Hs.177744 | ESTs | 0.62 |
| | | | | | | | [Swissprot: | |
| | | | | | | | none] | |
| 362. | TGGGAGAAGTG | 3.04 | 0.44 | 4.89 | 0.73 | Hs.184544 | Homo sapiens, | 0.62 |
| | | | | | | | clone | |
| | | | | | | | IMAGE: 3355383, | |
| | | | | | | | mRNA, | |
| 363. | CCATTGCATTC | 3.04 | 0.44 | 4.89 | 0.73 | Hs.185156 | ESTs | 0.62 |
| | | | | | | | [Swissprot: | |
| | | | | | | | none] | |
| 364. | TATAGACCTCA | 3.04 | 0.44 | 4.89 | 0.73 | Hs.19851 | peroxisomal | 0.62 |
| | | | | | | | biogenesis | |
| | | | | | | | factor 14 [Swiss | |
| 365. | TGCCCTCAGGA | 3.04 | 0.44 | 4.89 | 0.73 | Hs.204238 | lipocalin 2 | 0.62 |
| | | | | | | | (oncogene | |
| | | | | | | | 24p3) | |
| | | | | | | | [Swissprot: | |
| 366. | GTCTTTAGGAA | 3.04 | 0.44 | 4.89 | 0.73 | Hs.2057 | uridine | 0.62 |
| | | | | | | | monophosphate | |
| | | | | | | | synthetase | |
| | | | | | | | (orotat | |
| 367. | GAAAAAAATGG | 3.04 | 0.44 | 4.89 | 0.73 | Hs.21189 | DnaJ (Hsp40) | 0.62 |
| | | | | | | | homolog, | |
| | | | | | | | subfamily A, | |
| | | | | | | | membe | |
| 368. | GGAGAGTACAC | 3.04 | 0.44 | 4.89 | 0.73 | Hs.225939 | sialyltransferase | 0.62 |
| | | | | | | | 9 (CMP- | |
| | | | | | | | NeuAc: lactosylc | |
| 369. | AATTGAAAAGG | 3.04 | 0.44 | 4.89 | 0.73 | Hs.251664 | insulin-like | 0.62 |
| | | | | | | | growth factor 2 | |
| | | | | | | | (somatomedi | |
| 370. | TTGTCCTCTGG | 3.04 | 0.44 | 4.89 | 0.73 | Hs.264190 | vacuolar protein | 0.62 |
| | | | | | | | sorting 35 (yeast | |
| | | | | | | | homol | |
| 371. | CTGCTAAGATG | 3.04 | 0.44 | 4.89 | 0.73 | Hs.26510 | vacuolar protein | 0.62 |
| | | | | | | | sorting 33B | |
| | | | | | | | (yeast homo | |
| 372. | CAGAACTAGAC | 3.04 | 0.44 | 4.89 | 0.73 | Hs.266483 | dynein light | 0.62 |
| | | | | | | | chain-A | |
| | | | | | | | [Swissprot: | |
| | | | | | | | sp\|Q9Y | |
| 373. | TTTTGATGAGA | 3.04 | 0.44 | 4.89 | 0.73 | Hs.271411 | beta-site APP- | 0.62 |
| | | | | | | | cleaving enzyme | |
| | | | | | | | 2 [Swissp | |
| 374. | GCTAGTCTGTG | 3.04 | 0.44 | 4.89 | 0.73 | Hs.27860 | Homo sapiens | 0.62 |
| | | | | | | | mRNA; cDNA | |
| | | | | | | | DKFZp586M0723 | |
| | | | | | | | (f | |
| 375. | GGCACAGAAGG | 3.04 | 0.44 | 4.89 | 0.73 | Hs.279843 | mutL (E. coli) | 0.62 |
| | | | | | | | homolog 3 | |
| | | | | | | | [Swissprot: sp | |
| 376. | TTTTTCTTAAA | 3.04 | 0.44 | 4.89 | 0.73 | Hs.279860 | tumor protein, | 0.62 |
| | | | | | | | translationally- | |
| | | | | | | | controlle | |
| 377. | GCCAAAGTGTT | 3.04 | 0.44 | 4.89 | 0.73 | Hs.288880 | PAN2 protein | 0.62 |
| | | | | | | | [Swissprot: | |
| | | | | | | | sp\|Q9HBH5;] | |
| 378. | TATGAACAAAA | 3.04 | 0.44 | 4.89 | 0.73 | Hs.289008 | Homo sapiens | 0.62 |
| | | | | | | | cDNA; | |
| | | | | | | | FLJ21814 fis, | |
| | | | | | | | clone H | |
| 379. | CATTCACCATA | 3.04 | 0.44 | 4.89 | 0.73 | Hs.293678 | hypothetical | 0.62 |
| | | | | | | | protein | |
| | | | | | | | TCBAP0758 | |
| | | | | | | | [Swisspr | |
| 380. | ATAGGTCAGAA | 3.04 | 0.44 | 4.89 | 0.73 | Hs.29665 | KIAA0911 | 0.62 |
| | | | | | | | protein | |
| | | | | | | | [Swissprot: | |
| | | | | | | | sp\|O94985; | |
| 381. | AAGAACTAAAA | 3.04 | 0.44 | 4.89 | 0.73 | Hs.300631 | hypothetical | 0.62 |
| | | | | | | | protein | |
| | | | | | | | [Swissprot: | |
| | | | | | | | sp\|Q9B | |
| 382. | TGATATTAGGG | 3.04 | 0.44 | 4.89 | 0.73 | Hs.301002 | hypothetical | 0.62 |
| | | | | | | | protein | |
| | | | | | | | FLJ20871 | |
| | | | | | | | similar to | |
| 383. | GTTTCCCCAGA | 3.04 | 0.44 | 4.89 | 0.73 | Hs.302130 | CIG30 gene | 0.62 |
| | | | | | | | [Swissprot: | |
| | | | | | | | sp\|Q9HB03;] | |
| 384. | CAGATAAGTTT | 3.04 | 0.44 | 4.89 | 0.73 | Hs.306798 | Homo sapiens | 0.62 |
| | | | | | | | cDNA: | |
| | | | | | | | FLJ21655 fis, | |
| | | | | | | | clone C | |
| 385. | TAAAAGGAGGT | 3.04 | 0.44 | 4.89 | 0.73 | Hs.3069 | heat shock 70 kD | 0.62 |
| | | | | | | | protein 9B | |
| | | | | | | | (mortalin-2) | |
| 386. | CAATGGAGCTT | 3.04 | 0.44 | 4.89 | 0.73 | Hs.30925 | hypothetical | 0.62 |
| | | | | | | | protein | |
| | | | | | | | FLJ10199 | |
| | | | | | | | [Swisspro | |
| 387. | TATGTATGTTG | 3.04 | 0.44 | 4.89 | 0.73 | Hs.343477 | hypothetical | 0.62 |
| | | | | | | | protein | |
| | | | | | | | PRO2975 | |
| | | | | | | | [Swissprot | |
| 388. | TCCAGAATAAA | 3.04 | 0.44 | 4.89 | 0.73 | Hs.343589 | exosome | 0.62 |
| | | | | | | | component | |
| | | | | | | | Rrp41 | |
| | | | | | | | [Swissprot: sp\| | |
| 389. | TTTATCTGCTG | 3.04 | 0.44 | 4.89 | 0.73 | Hs.343598 | Homo sapiens, | 0.62 |
| | | | | | | | clone | |
| | | | | | | | IMAGE: 3875308, | |
| | | | | | | | mRNA, | |
| 390. | CCAATAAAAGA | 3.04 | 0.44 | 4.89 | 0.73 | Hs.348425 | hypothetical | 0.62 |
| | | | | | | | protein | |
| | | | | | | | FLJ10975 | |
| | | | | | | | [Swisspro | |
| 391. | AGCCGGGCTTT | 3.04 | 0.44 | 4.89 | 0.73 | Hs.57079 | Homo sapiens | 0.62 |
| | | | | | | | cDNA FLJ13267 | |
| | | | | | | | fis, clone OV | |
| 392. | CAAAACTGTTT | 3.04 | 0.44 | 4.89 | 0.73 | Hs.61638 | myosin X | 0.62 |
| | | | | | | | [Swissprot: | |
| | | | | | | | sp\|O94893; sp\|Q9HD6 | |
| 393. | TATTTTTACTG | 3.04 | 0.44 | 4.89 | 0.73 | Hs.6582 | Rho guanine | 0.62 |
| | | | | | | | exchange factor | |
| | | | | | | | (GEF) 12 [S | |
| 394. | ATACATACTGT | 3.04 | 0.44 | 4.89 | 0.73 | Hs.74313 | KIAA1265 | 0.62 |
| | | | | | | | protein | |
| | | | | | | | [Swissprot: | |
| | | | | | | | sp\|Q9ULF5; | |
| 395. | GCCAACAACGA | 3.04 | 0.44 | 4.89 | 0.73 | Hs.76669 | nicotinamide N- | 0.62 |
| | | | | | | | methyltransferase | |
| | | | | | | | [Swiss | |
| 396. | TGTTTATCCTA | 3.04 | 0.44 | 4.89 | 0.73 | Hs.78888 | diazepam | 0.62 |
| | | | | | | | binding inhibitor | |
| | | | | | | | (GABA recepto | |
| 397. | CGAAGGCTGTA | 3.04 | 0.44 | 4.89 | 0.73 | Hs.79334 | nuclear factor, | 0.62 |
| | | | | | | | interleukin 3 | |
| | | | | | | | regulated | |
| 398. | GCCATTATAAG | 3.04 | 0.44 | 4.89 | 0.73 | Hs.8262 | lysosomal- | 0.62 |
| | | | | | | | associated | |
| | | | | | | | membrane | |
| | | | | | | | protein 2 | |
| 399. | ATGTTTGCCCT | 3.04 | 0.44 | 4.89 | 0.73 | Hs.84928 | nuclear | 0.62 |
| | | | | | | | transcription | |
| | | | | | | | factor Y, beta [S | |
| 400. | TATGCTTAGTA | 3.04 | 0.44 | 4.89 | 0.73 | Hs.86041 | CGG triplet | 0.62 |
| | | | | | | | repeat binding | |
| | | | | | | | protein 1 [S | |
| 401. | TTTTACAGTAC | 3.04 | 0.44 | 4.89 | 0.73 | Hs.86347 | hypothetical | 0.62 |
| | | | | | | | protein | |
| | | | | | | | [Swissprot: | |
| | | | | | | | sp\|Q96 | |
| 402. | TCTGTAAAAAA | 3.04 | 0.44 | 4.89 | 0.73 | Hs.90960 | ESTs | 0.62 |
| | | | | | | | [Swissprot: | |
| | | | | | | | none] | |
| 403. | GAAAAAATAAA | 3.04 | 0.44 | 4.89 | 0.73 | Hs.94925 | dihydroorotate | 0.62 |
| | | | | | | | dehydrogenase | |
| | | | | | | | [Swissprot | |
| 404. | TTTGCCTGGAT | 3.04 | 0.44 | 4.89 | 0.73 | Hs.95260 | family with | 0.62 |
| | | | | | | | sequence | |
| | | | | | | | similarity B, | |
| | | | | | | | membe | |
| 405. | TTACTGTGTAA | 3.04 | 0.44 | 4.89 | 0.73 | Hs.9587 | Homo sapiens | 0.62 |
| | | | | | | | cDNA: | |
| | | | | | | | FLJ22290 fis, | |
| | | | | | | | clone H | |
| 406. | AATAAAGGCTA | 3.38 | 1.27 | 2.04 | 0.61 | Hs.179735 | ras homolog | 1.66 |
| | | | | | | | gene family, | |
| | | | | | | | member C | |
| | | | | | | | [Swis | |
| 407. | CCATTGTACTC | −3.95 | 0.65 | −3.68 | 0.58 | Hs.288771 | DKFZP586A0522 | 1.07 |
| | | | | | | | protein | |
| | | | | | | | [Swissprot: | |
| | | | | | | | sp\|Q9 | |
| 408. | AAACTCGAGCA | −4.93 | 0.89 | −3.68 | 0.58 | Hs.278378 | karyopherin beta | 1.34 |
| | | | | | | | 2b, transportin | |
| | | | | | | | [Swiss | |
| 409. | GTGACTGCCAC | 4.05 | 1.21 | 2.17 | 0.57 | H5.84183 | diptheria toxin | 1.87 |
| | | | | | | | resistance | |
| | | | | | | | protein requi | |
| 410. | GGGCAAGCCAG | 3.04 | 0.76 | 2.44 | 0.52 | Hs.110849 | estrogen-related | 1.25 |
| | | | | | | | receptor alpha | |
| | | | | | | | [Swissp | |
| 411. | ATACTGTCAGT | 3.04 | 0.76 | 2.44 | 0.52 | Hs.11441 | chromosome 1 | 1.25 |
| | | | | | | | open reading | |
| | | | | | | | frame 8 [Swis | |
| 412. | ATGGGGGTGAT | 3.04 | 0.76 | 2.44 | 0.52 | Hs.19210 | hypothetical | 1.25 |
| | | | | | | | protein | |
| | | | | | | | MGC11308 | |
| | | | | | | | [Swisspro | |
| 413. | GAAAAAATGTT | 3.04 | 0.76 | 2.44 | 0.52 | Hs.194329 | hypothetical | 1.25 |
| | | | | | | | protein | |
| | | | | | | | FLJ21174 | |
| | | | | | | | [Swisspro | |
| 414. | GAATAAATGTT | 3.04 | 0.76 | 2.44 | 0.52 | Hs.302749 | FK506-binding | 1.25 |
| | | | | | | | protein 9 (63 kD) | |
| | | | | | | | [Swissp | |
| 415. | GTAGGAGCTGG | 3.04 | 0.76 | 2.44 | 0.52 | Hs.81728 | unc119 | 1.25 |
| | | | | | | | (C. elegans) | |
| | | | | | | | homolog | |
| | | | | | | | [Swissprot: | |
| 416. | GTGTATCTTTT | −3.45 | 0.94 | −2.46 | 0.52 | Hs.73965 | splicing factor, | 1.40 |
| | | | | | | | arginine/serine- | |
| | | | | | | | rich 2 | |
| 417. | CTTTTGTTCTG | 4.05 | 0.67 | 3.26 | 0.48 | Hs.106823 | hypothetical | 1.24 |
| | | | | | | | protein | |
| | | | | | | | MGC14797 | |
| | | | | | | | [Swisspro | |
| 418. | GGGCTTGGTAT | 4.05 | 0.67 | 3.26 | 0.48 | Hs.107882 | hypothetical | 1.24 |
| | | | | | | | protein | |
| | | | | | | | FLJ10659 | |
| | | | | | | | [Swisspro | |
| 419. | CACACCATTGT | 4.05 | 0.67 | 3.26 | 0.48 | Hs.136644 | CS box- | 1.24 |
| | | | | | | | containing WD | |
| | | | | | | | protein | |
| | | | | | | | [Swissprot | |
| 420. | GAGTGAGTGAG | 4.05 | 0.67 | 3.26 | 0.48 | Hs.193264 | hypothetical | 1.24 |
| | | | | | | | protein | |
| | | | | | | | MGC3234 | |
| | | | | | | | [Swissprot | |
| 421. | TTGCTATGAAA | 4.05 | 0.67 | 3.26 | 0.48 | Hs.26549 | KIAA1708 | 1.24 |
| | | | | | | | protein | |
| | | | | | | | [Swissprot: | |
| | | | | | | | sp\|Q9C0F5; | |
| 422. | AGAAGTACTGA | 4.05 | 0.67 | 3.26 | 0.48 | Hs.2934 | ribonucleotide | 1.24 |
| | | | | | | | reductase M1 | |
| | | | | | | | polypeptide | |
| 423. | GCTTAATGTTT | 4.05 | 0.67 | 3.26 | 0.48 | Hs.76359 | catalase | 1.24 |
| | | | | | | | [Swissprot: | |
| | | | | | | | sp\|P04040; sp\|Q9BWT | |
| 424. | TGACTGCTGCT | 4.05 | 0.67 | 3.26 | 0.48 | Hs.90063 | neurocalcin | 1.24 |
| | | | | | | | delta | |
| | | | | | | | [Swissprot: | |
| | | | | | | | sp\|Q96G57 | |
| 425. | TCTCAAGAAGC | 4.00 | 0.61 | 3.26 | 0.48 | Hs.100555 | DEAD/H (Asp- | 1.23 |
| | | | | | | | Glu-Ala-Asp/His) | |
| | | | | | | | box polypep | |
| 426. | CTCCCGGAAAT | 4.00 | 0.61 | 3.26 | 0.48 | Hs.103291 | neuritin | 1.23 |
| | | | | | | | [Swissprot: | |
| | | | | | | | sp\|Q9NPD7;] | |
| 427. | TGTGAGGGCAT | 4.00 | 0.61 | 3.26 | 0.48 | Hs.103808 | hypothetical | 1.23 |
| | | | | | | | protein | |
| | | | | | | | FLJ20602 | |
| | | | | | | | [Swisspro | |
| 428. | GTGAATGTATG | 4.00 | 0.61 | 3.26 | 0.48 | Hs.103839 | erythrocyte | 1.23 |
| | | | | | | | membrane | |
| | | | | | | | protein band | |
| | | | | | | | 4.1-II | |
| 429. | AATTTAGAGCA | 4.00 | 0.61 | 3.26 | 0.48 | Hs.104222 | hypothetical | 1.23 |
| | | | | | | | protein | |
| | | | | | | | FLJ10702 | |
| | | | | | | | [Swisspro | |
| 430. | CCTGTTCTCCT | 4.00 | 0.61 | 3.26 | 0.48 | Hs.109798 | G8 protein | 1.23 |
| | | | | | | | [Swissprot: | |
| | | | | | | | sp\|Q9BW21; sp\| | |
| | | | | | | | Q9U | |
| 431. | AAGTTGTGTGC | 4.00 | 0.61 | 3.26 | 0.48 | Hs.109854 | ESTs, Weakly | 1.23 |
| | | | | | | | similar to | |
| | | | | | | | ALU1_HUMAN | |
| | | | | | | | ALU S | |
| 432. | TTGCAGAGGGG | 4.00 | 0.61 | 3.26 | 0.48 | Hs.110373 | ESTs, Highly | 1.23 |
| | | | | | | | similar to | |
| | | | | | | | T42626 secreted | |
| 433. | TTGGCAGTATT | 4.00 | 0.61 | 3.26 | 0.48 | Hs.110708 | sarcoglycan, | 1.23 |
| | | | | | | | epsilon | |
| | | | | | | | [Swissprot: | |
| | | | | | | | sp\|O43 | |
| 434. | TTTGTTAAAAA | 4.00 | 0.61 | 3.26 | 0.48 | Hs.111244 | hypothetical | 1.23 |
| | | | | | | | protein | |
| | | | | | | | [Swissprot: | |
| | | | | | | | sp\|Q9H | |
| 435. | ACCCACTGTGC | 4.00 | 0.61 | 3.26 | 0.48 | Hs.112751 | KIAA0892 | 1.23 |
| | | | | | | | protein | |
| | | | | | | | [Swissprot: | |
| | | | | | | | sp\|Q9UFX8; | |
| 436. | TTGTTTCTACT | 4.00 | 0.61 | 3.26 | 0.48 | Hs.11638 | fatty-acid- | 1.23 |
| | | | | | | | Coenzyme A | |
| | | | | | | | ligase, long- | |
| | | | | | | | chain | |
| 437. | AAGACTATGTT | 4.00 | 0.61 | 3.26 | 0.48 | Hs.116796 | ESTs | 1.23 |
| | | | | | | | [Swissprot: | |
| | | | | | | | none] | |
| 438. | TCAAATGTGAA | 4.00 | 0.61 | 3.26 | 0.48 | Hs.11805 | ESTs | 1.23 |
| | | | | | | | [Swissprot: | |
| | | | | | | | none] | |
| 439. | TGAATGAATGG | 4.00 | 0.61 | 3.26 | 0.48 | Hs.118797 | ubiquitin- | 1.23 |
| | | | | | | | conjugating | |
| | | | | | | | enzyme E2D 3 | |
| | | | | | | | (homo | |
| 440. | TTTTGTGTTGG | 4.00 | 0.61 | 3.26 | 0.48 | Hs.118797 | ubiquitin- | 1.23 |
| | | | | | | | conjugating | |
| | | | | | | | enzyme E2D 3 | |
| | | | | | | | (homo | |
| 441. | GACTCCCAGGA | 4.00 | 0.61 | 3.26 | 0.48 | Hs.119004 | KIAA0665 gene | 1.23 |
| | | | | | | | product | |
| | | | | | | | [Swissprot: | |
| | | | | | | | sp\|O7 | |
| 442. | GACCGCAGGAG | 4.00 | 0.61 | 3.26 | 0.48 | Hs.119129 | collagen, type | 1.23 |
| | | | | | | | IV, alpha 1 | |
| | | | | | | | [Swissprot: | |
| 443. | GCTTTGGGATG | 4.00 | 0.61 | 3.26 | 0.48 | Hs.119394 | ESTs | 1.23 |
| | | | | | | | [Swissprot: | |
| | | | | | | | none] | |
| 444. | AGCACATTCTT | 4.00 | 0.61 | 3.26 | 0.48 | Hs.119403 | hexosaminidase | 1.23 |
| | | | | | | | A (alpha | |
| | | | | | | | polypeptide) [S | |
| 445. | AACTTGATACG | 4.00 | 0.61 | 3.26 | 0.48 | Hs.119597 | stearoyl-CoA | 1.23 |
| | | | | | | | desaturase | |
| | | | | | | | (delta-9-desatur | |
| 446. | GGAATAAAAGT | 4.00 | 0.61 | 3.26 | 0.48 | Hs.12152 | APMCF1 protein | 1.23 |
| | | | | | | | [Swissprot: | |
| | | | | | | | sp\|Q9Y5M8;] | |
| 447. | AAACAGTAGTG | 4.00 | 0.61 | 3.26 | 0.48 | Hs.121849 | microtubule- | 1.23 |
| | | | | | | | associated | |
| | | | | | | | proteins 1A/1B li | |
| 448. | GAGGGAAGACC | 4.00 | 0.61 | 3.26 | 0.48 | Hs.123610 | ESTs | 1.23 |
| | | | | | | | [Swissprot: | |
| | | | | | | | none] | |
| 449. | TAATAAATAGA | 4.00 | 0.61 | 3.26 | 0.48 | Hs.123654 | PCF11p | 1.23 |
| | | | | | | | homolog | |
| | | | | | | | [Swissprot: | |
| | | | | | | | sp\|O94913;] | |
| 450. | TAGACAATGCT | 4.00 | 0.61 | 3.26 | 0.48 | Hs.12421 | hypothetical | 1.23 |
| | | | | | | | protein | |
| | | | | | | | [Swissprot: | |
| | | | | | | | sp\|Q92 | |
| 451. | GAATTAAACAA | 4.00 | 0.61 | 3.26 | 0.48 | Hs.124758 | ESTs | 1.23 |
| | | | | | | | [Swissprot: | |
| | | | | | | | none] | |
| 452. | TTTGGAAAAGC | 4.00 | 0.61 | 3.26 | 0.48 | Hs.12482 | glyceronephosphate | 1.23 |
| | | | | | | | O- | |
| | | | | | | | acyltransferase | |
| | | | | | | | [S | |
| 453. | TTTGTCTGTCT | 4.00 | 0.61 | 3.26 | 0.48 | Hs.125134 | pre-mRNA | 1.23 |
| | | | | | | | splicing SR | |
| | | | | | | | protein rA4 | |
| | | | | | | | [Swiss | |
| 454. | CACACTTGGAG | 4.00 | 0.61 | 3.26 | 0.48 | Hs.126063 | ESTs | 1.23 |
| | | | | | | | [Swissprot: | |
| | | | | | | | none] | |
| 455. | TGTTATTACTG | 4.00 | 0.61 | 3.26 | 0.48 | Hs.127294 | Homo sapiens | 1.23 |
| | | | | | | | cDNA: | |
| | | | | | | | FLJ21587 fis, | |
| | | | | | | | clone C | |
| 456. | TCCTTTTTCTC | 4.00 | 0.61 | 3.26 | 0.48 | Hs.1276 | BN51 (BHK21) | 1.23 |
| | | | | | | | temperature | |
| | | | | | | | sensitivity com | |
| 457. | TGAAAGGGAGA | 4.00 | 0.61 | 3.26 | 0.48 | Hs.128207 | polymerase | 1.23 |
| | | | | | | | (RNA) III (DNA | |
| | | | | | | | directed) (39k | |
| 458. | TGCCTTTAACC | 4.00 | 0.61 | 3.26 | 0.48 | Hs.12845 | hypothetical | 1.23 |
| | | | | | | | protein | |
| | | | | | | | MGC13159 | |
| | | | | | | | [Swisspro | |
| 459. | GATATGACAAG | 4.00 | 0.61 | 3.26 | 0.48 | Hs.128759 | sterile alpha and | 1.23 |
| | | | | | | | HEAT/Armadilio | |
| | | | | | | | motif p | |
| 460. | GCACCTTCTGG | 4.00 | 0.61 | 3.26 | 0.48 | Hs.132744 | hypothetical | 1.23 |
| | | | | | | | protein | |
| | | | | | | | [Swissprot: | |
| | | | | | | | sp\|Q96 | |
| 461. | CAGTAGACAGA | 4.00 | 0.61 | 3.26 | 0.48 | Hs.132881 | prefoldin 1 | 1.23 |
| | | | | | | | [Swissprot: | |
| | | | | | | | sp\|O60925; sp\|Q9 | |
| 462. | TTGTTTTCTGG | 4.00 | 0.61 | 3.26 | 0.48 | Hs.133463 | ESTs | 1.23 |
| | | | | | | | [Swissprot: | |
| | | | | | | | none] | |
| 463. | TAATCAAAAAA | 4.00 | 0.61 | 3.26 | 0.48 | Hs.135167 | Homo sapiens | 1.23 |
| | | | | | | | mRNA for | |
| | | | | | | | putative nuclear p | |
| 464. | AGAGTGCCTAT | 4.00 | 0.61 | 3.26 | 0.48 | Hs.137260 | hypothetical | 1.23 |
| | | | | | | | protein | |
| | | | | | | | FLJ23151 | |
| | | | | | | | [Swisspro | |
| 465. | GTGTCTGTCTC | 4.00 | 0.61 | 3.26 | 0.48 | Hs.137432 | ESTs | 1.23 |
| | | | | | | | [Swissprot: | |
| | | | | | | | none] | |
| 466. | GTGTATATGTA | 4.00 | 0.61 | 3.26 | 0.48 | Hs.138902 | ESTs, Weakly | 1.23 |
| | | | | | | | similar to | |
| | | | | | | | T12486 | |
| | | | | | | | hypotheti | |
| 467. | ATAAGGAAAAG | 4.00 | 0.61 | 3.26 | 0.48 | Hs.142150 | Kruppel-type | 1.23 |
| | | | | | | | zinc finger | |
| | | | | | | | (C2H2) [Swissp | |
| 468. | CAGCTTTTTCA | 4.00 | 0.61 | 3.26 | 0.48 | Hs.14394 | hypothetical | 1.23 |
| | | | | | | | protein | |
| | | | | | | | FLJ20157 | |
| | | | | | | | [Swisspro | |
| 469. | TTAAGAAGCCT | 4.00 | 0.61 | 3.26 | 0.48 | Hs.14791 | acyl-Coenzyme A | 1.23 |
| | | | | | | | dehydrogenase | |
| | | | | | | | family, me | |
| 470. | GTTTGTGAAAA | 4.00 | 0.61 | 3.26 | 0.48 | Hs.150627 | ESTs | 1.23 |
| | | | | | | | [Swissprot: | |
| | | | | | | | none] | |
| 471. | TACCTGTGGTC | 4.00 | 0.61 | 3.26 | 0.48 | Hs.150821 | ESTs, Weakly | 1.23 |
| | | | | | | | similar to I38022 | |
| | | | | | | | hypotheti | |
| 472. | TTTATTTGGCA | 4.00 | 0.61 | 3.26 | 0.48 | Hs.152931 | lamin B receptor | 1.23 |
| | | | | | | | [Swissprot: | |
| | | | | | | | sp\|Q14739; | |
| 473. | CAGCGCGCCCT | 4.00 | 0.61 | 3.26 | 0.48 | Hs.152932 | ESTs, Weakly | 1.23 |
| | | | | | | | similar to | |
| | | | | | | | TRHY_HUMAN | |
| | | | | | | | TRICH | |
| 474. | GCTAGTAGAGT | 4.00 | 0.61 | 3.26 | 0.48 | Hs.15299 | HMBA-inducible | 1.23 |
| | | | | | | | [Swissprot: | |
| | | | | | | | sp\|O94992;] | |
| 475. | TACCTATTTAC | 4.00 | 0.61 | 3.26 | 0.48 | Hs.153717 | ESTs | 1.23 |
| | | | | | | | [Swissprot: | |
| | | | | | | | none] | |
| 476. | ATGTAACTACT | 4.00 | 0.61 | 3.26 | 0.48 | Hs.153746 | hypothetical | 1.23 |
| | | | | | | | protein | |
| | | | | | | | FLJ22490 | |
| | | | | | | | [Swisspro | |
| 477. | CAACACCTGAG | 4.00 | 0.61 | 3.26 | 0.48 | Hs.1540 | nuclear matrix | 1.23 |
| | | | | | | | protein p84 | |
| | | | | | | | [Swissprot: | |
| 478. | AGAATAAATCT | 4.00 | 0.61 | 3.26 | 0.48 | Hs.154437 | phosphodiesterase | 1.23 |
| | | | | | | | 2A, cGMP- | |
| | | | | | | | stimulated [ | |
| 479. | TATTATATACA | 4.00 | 0.61 | 3.26 | 0.48 | Hs.154890 | fatty-acid- | 1.23 |
| | | | | | | | Coenzyme A | |
| | | | | | | | ligase, long- | |
| | | | | | | | chain | |
| 480. | ACCTAACTTTT | 4.00 | 0.61 | 3.26 | 0.48 | Hs.15519 | oxysterol-binding | 1.23 |
| | | | | | | | protein-like 2 | |
| | | | | | | | [Swiss | |
| 481. | TGCAAAGTACT | 4.00 | 0.61 | 3.26 | 0.48 | Hs.155478 | cyclin T2 | 1.23 |
| | | | | | | | [Swissprot: | |
| | | | | | | | sp\|O60583;] | |
| 482. | AGCTGGGATGG | 4.00 | 0.61 | 3.26 | 0.48 | Hs.15898 | 2,4-dienoyl CoA | 1.23 |
| | | | | | | | reductase 2, | |
| | | | | | | | peroxisomal | |
| 483. | ACAAGTACATT | 4.00 | 0.61 | 3.26 | 0.48 | Hs.160881 | Homo sapiens | 1.23 |
| | | | | | | | colon cancer | |
| | | | | | | | antigen NY-CO- | |
| 484. | AGGTGCGGGGG | 4.00 | 0.61 | 3.26 | 0.48 | Hs.165439 | arsA (bacterial) | 1.23 |
| | | | | | | | arsenite | |
| | | | | | | | transporter, A | |
| 485. | GCTAGGTATTT | 4.00 | 0.61 | 3.26 | 0.48 | Hs.165986 | testis derived | 1.23 |
| | | | | | | | transcript (3 LIM | |
| | | | | | | | domains | |
| 486. | CAAAAGCTTTG | 4.00 | 0.61 | 3.26 | 0.48 | Hs.165998 | PAI-1 mRNA- | 1.23 |
| | | | | | | | binding protein | |
| | | | | | | | [Swissprot: | |
| 487. | GTATTTAATAA | 4.00 | 0.61 | 3.26 | 0.48 | Hs.167031 | DKFZP566D133 | 1.23 |
| | | | | | | | protein | |
| | | | | | | | [Swissprot: | |
| | | | | | | | sp\|Q9Y | |
| 488. | CCTAGGGTTCC | 4.00 | 0.61 | 3.26 | 0.48 | Hs.167218 | BarH-like | 1.23 |
| | | | | | | | homeobox 2 | |
| | | | | | | | [Swissprot: | |
| | | | | | | | sp\|Q9U | |
| 489. | CTGCCCAGTGG | 4.00 | 0.61 | 3.26 | 0.48 | Hs.169793 | ribosomal | 1.23 |
| | | | | | | | protein L32 | |
| | | | | | | | [Swissprot: | |
| | | | | | | | sp\|O6 | |
| 490. | TTTGTTTTTGA | 4.00 | 0.61 | 3.26 | 0.48 | Hs.170088 | GLUT4 | 1.23 |
| | | | | | | | enhancer factor | |
| | | | | | | | [Swissprot: | |
| | | | | | | | sp\|O9 | |
| 491. | GAGGGGCACTG | 4.00 | 0.61 | 3.26 | 0.48 | Hs.170132 | hypothetical | 1.23 |
| | | | | | | | protein | |
| | | | | | | | FLJ22494 | |
| | | | | | | | [Swisspro | |
| 492. | AGACAGTCATT | 4.00 | 0.61 | 3.26 | 0.48 | Hs.17109 | integral | 1.23 |
| | | | | | | | membrane | |
| | | | | | | | protein 2A | |
| | | | | | | | [Swissprot | |
| 493. | CACTGAACTCT | 4.00 | 0.61 | 3.26 | 0.48 | Hs.172028 | a disintegrin and | 1.23 |
| | | | | | | | metalloproteinase | |
| | | | | | | | doma | |
| 494. | TGTGGGGAAAG | 4.00 | 0.61 | 3.26 | 0.48 | Hs.172280 | SWI/SNF | 1.23 |
| | | | | | | | related, matrix | |
| | | | | | | | associated, acti | |
| 495. | AGAAGCCGTGG | 4.00 | 0.61 | 3.26 | 0.48 | Hs.172631 | integrin, alpha M | 1.23 |
| | | | | | | | (complement | |
| | | | | | | | component | |
| 496. | TGCCTTGAAAG | 4.00 | 0.61 | 3.26 | 0.48 | Hs.173162 | neighbor of | 1.23 |
| | | | | | | | COX4 | |
| | | | | | | | [Swissprot: | |
| | | | | | | | sp\|O43402; | |
| 497. | GCCCCCCTGCC | 4.00 | 0.61 | 3.26 | 0.48 | Hs.173739 | hypothetical | 1.23 |
| | | | | | | | protein | |
| | | | | | | | FLJ10297 | |
| | | | | | | | [Swisspro | |
| 498. | CATAAATATTA | 4.00 | 0.61 | 3.26 | 0.48 | Hs.174151 | aldehyde | 1.23 |
| | | | | | | | oxidase 1 | |
| | | | | | | | [Swissprot: | |
| | | | | | | | sp\|Q0627 | |
| 499. | CTGCTGAGCCT | 4.00 | 0.61 | 3.26 | 0.48 | Hs.1742 | IQ motif | 1.23 |
| | | | | | | | containing | |
| | | | | | | | GTPase | |
| | | | | | | | activating pr | |
| 500. | CTGTGACATTT | 4.00 | 0.61 | 3.26 | 0.48 | Hs.177584 | 3-oxoacid CoA | 1.23 |
| | | | | | | | transferase | |
| | | | | | | | [Swissprot: s | |
| 501. | AAGGAAGATGG | 4.00 | 0.61 | 3.26 | 0.48 | Hs.180062 | proteasome | 1.23 |
| | | | | | | | (prosome, | |
| | | | | | | | macropain) | |
| | | | | | | | subunit, | |
| 502. | CTCGGCGAGCC | 4.00 | 0.61 | 3.26 | 0.48 | Hs.181077 | hypothetical | 1.23 |
| | | | | | | | protein | |
| | | | | | | | DKFZp586I021 | |
| | | | | | | | [Swis | |
| 503. | AAGATCAAGTC | 4.00 | 0.61 | 3.26 | 0.48 | Hs.182426 | ribosomal | 1.23 |
| | | | | | | | protein S2 | |
| | | | | | | | [Swissprot: | |
| | | | | | | | sp\|P15 | |
| 504. | AATATAAAACA | 4.00 | 0.61 | 3.26 | 0.48 | Hs.183434 | ATPase, H+ | 1.23 |
| | | | | | | | transporting, | |
| | | | | | | | lysosomal (vacu | |
| 505. | GCATTCGCAGT | 4.00 | 0.61 | 3.26 | 0.48 | Hs.183842 | ubiquitin B | 1.23 |
| | | | | | | | [Swissprot: | |
| | | | | | | | sp\|P02248; sp\|Q9 | |
| 506. | TAAGATGGCAA | 4.00 | 0.61 | 3.26 | 0.48 | Hs.183858 | transcriptional | 1.23 |
| | | | | | | | intermediary | |
| | | | | | | | factor 1 [ | |
| 507. | GTTTTATGAAG | 4.00 | 0.61 | 3.26 | 0.48 | Hs.184581 | Homo sapiens | 1.23 |
| | | | | | | | cDNA FLJ14821 | |
| | | | | | | | fis, clone OV | |
| 508. | GGTCTGGCCTG | 4.00 | 0.61 | 3.26 | 0.48 | Hs.184641 | fatty acid | 1.23 |
| | | | | | | | desaturase 2 | |
| | | | | | | | [Swiss prot: sp\| | |
| 509. | CACGACTGTTC | 4.00 | 0.61 | 3.26 | 0.48 | Hs.184779 | Homo sapiens | 1.23 |
| | | | | | | | mRNA; cDNA | |
| | | | | | | | DKFZp586B1922 | |
| | | | | | | | (f | |
| 510. | GTGAAACCTGG | 4.00 | 0.61 | 3.26 | 0.48 | Hs.1852 | acid | 1.23 |
| | | | | | | | phosphatase, | |
| | | | | | | | prostate | |
| | | | | | | | [Swissprot: | |
| 511. | CTGCTCACCTT | 4.00 | 0.61 | 3.26 | 0.48 | Hs.18627 | hypothetical | 1.23 |
| | | | | | | | protein | |
| | | | | | | | DKFZp564L242 | |
| | | | | | | | 3 [Swi | |
| 512. | TAAGAAAAGGC | 4.00 | 0.61 | 3.26 | 0.48 | Hs.18747 | POP7 | 1.23 |
| | | | | | | | (processing of | |
| | | | | | | | precursor, S. cerevi | |
| 513. | TCAGTGACCAG | 4.00 | 0.61 | 3.26 | 0.48 | Hs.1880 | hypothetical | 1.23 |
| | | | | | | | protein | |
| | | | | | | | MGC5363 | |
| | | | | | | | [Swissprot | |
| 514. | ACACAAGTTTA | 4.00 | 0.61 | 3.26 | 0.48 | Hs.193162 | Homo sapiens | 1.23 |
| | | | | | | | cDNA FLJ11983 | |
| | | | | | | | fis, clone HE | |
| 515. | ACAAGATTAAA | 4.00 | 0.61 | 3.26 | 0.48 | Hs.19339 | ESTs | 1.23 |
| | | | | | | | [Swissprot: | |
| | | | | | | | none] | |
| 516. | TACAAAAGTGG | 4.00 | 0.61 | 3.26 | 0.48 | Hs.194662 | calponin 3, | 1.23 |
| | | | | | | | acidic | |
| | | | | | | | [Swissprot: | |
| | | | | | | | sp\|Q1541 | |
| 517. | TTACCATTGGT | 4.00 | 0.61 | 3.26 | 0.48 | Hs.194718 | zinc finger | 1.23 |
| | | | | | | | protein 265 | |
| | | | | | | | [Swissprot: sp\| | |
| 518. | AAGGAGAGCCC | 4.00 | 0.61 | 3.26 | 0.48 | Hs.194816 | stomatin | 1.23 |
| | | | | | | | (EBP72)-like 1 | |
| | | | | | | | [Swissprot: sp\| | |
| 519. | ATTAAAAAAAA | 4.00 | 0.61 | 3.26 | 0.48 | Hs.198726 | cold shock | 1.23 |
| | | | | | | | domain protein | |
| | | | | | | | A [Swissprot: | |
| 520. | ATACATTCCAT | 4.00 | 0.61 | 3.26 | 0.48 | Hs.201366 | ESTs | 1.23 |
| | | | | | | | [Swissprot: | |
| | | | | | | | none] | |
| 521. | TGAAGATGGAG | 4.00 | 0.61 | 3.26 | 0.48 | Hs.202852 | ESTs | 1.23 |
| | | | | | | | [Swissprot: | |
| | | | | | | | none] | |
| 522. | CTGTTATAGGA | 4.00 | 0.61 | 3.26 | 0.48 | Hs.206521 | YME1 | 1.23 |
| | | | | | | | (S. cerevisiae)- | |
| | | | | | | | like 1 | |
| | | | | | | | [Swissprot: | |
| 523. | TAAAACCCTAT | 4.00 | 0.61 | 3.26 | 0.48 | Hs.211582 | myosin, light | 1.23 |
| | | | | | | | polypeptide | |
| | | | | | | | kinase [Swiss | |
| 524. | AGTCTTAATGT | 4.00 | 0.61 | 3.26 | 0.48 | Hs.211610 | CUG triplet | 1.23 |
| | | | | | | | repeat, RNA- | |
| | | | | | | | binding protein | |
| 525. | GGAGCACTCTC | 4.00 | 0.61 | 3.26 | 0.48 | Hs.22051 | hypothetical | 1.23 |
| | | | | | | | protein | |
| | | | | | | | MGC15548 | |
| | | | | | | | [Swisspro | |
| 526. | TGGATCATTCT | 4.00 | 0.61 | 3.26 | 0.48 | Hs.223394 | hypothetical | 1.23 |
| | | | | | | | protein | |
| | | | | | | | MGC2742 | |
| | | | | | | | [Swissprot | |
| 527. | GCCTTGGGTGA | 4.00 | 0.61 | 3.26 | 0.48 | Hs.2250 | leukemia | 1.23 |
| | | | | | | | inhibitory factor | |
| | | | | | | | (cholinergic | |
| 528. | ATATTACAGTG | 4.00 | 0.61 | 3.26 | 0.48 | Hs.226318 | CCR4-NOT | 1.23 |
| | | | | | | | transcription | |
| | | | | | | | complex, subunit | |
| 529. | TAGCTAATGCC | 4.00 | 0.61 | 3.26 | 0.48 | Hs.23236 | ESTs | 1.23 |
| | | | | | | | [Swissprot: | |
| | | | | | | | none] | |
| 530. | TTTATATCTTT | 4.00 | 0.61 | 3.26 | 0.48 | Hs.23360 | likely ortholog of | 1.23 |
| | | | | | | | yeast ARV1 | |
| | | | | | | | [Swisspro | |
| 531. | CTTATTGCCCT | 4.00 | 0.61 | 3.26 | 0.48 | Hs.234265 | DKFZP586G011 | 1.23 |
| | | | | | | | protein | |
| | | | | | | | [Swissprot: | |
| | | | | | | | sp\|Q9H | |
| 532. | CTGCCATTCTT | 4.00 | 0.61 | 3.26 | 0.48 | Hs.2384 | tumor protein | 1.23 |
| | | | | | | | D52 [Swissprot: | |
| | | | | | | | sp\|P55327 | |
| 533. | ATGTATAGGGC | 4.00 | 0.61 | 3.26 | 0.48 | Hs.238809 | ESTs | 1.23 |
| | | | | | | | [Swissprot: | |
| | | | | | | | none] | |
| 534. | ATTAACTGCTA | 4.00 | 0.61 | 3.26 | 0.48 | Hs.23964 | sin3-associated | 1.23 |
| | | | | | | | polypeptide, | |
| | | | | | | | 18 kD [Swis | |
| 535. | TTAATCTGGTA | 4.00 | 0.61 | 3.26 | 0.48 | Hs.24095 | ESTs | 1.23 |
| | | | | | | | [Swissprot: | |
| | | | | | | | none] | |
| 536. | TATCTAGCTGC | 4.00 | 0.61 | 3.26 | 0.48 | Hs.241545 | hypothetical | 1.23 |
| | | | | | | | protein | |
| | | | | | | | [Swissprot: | |
| | | | | | | | sp\|O95 | |
| 537. | AGTGCTTTGAG | 4.00 | 0.61 | 3.26 | 0.48 | Hs.24654 | Homo sapiens | 1.23 |
| | | | | | | | cDNA FLJ11640 | |
| | | | | | | | fis, clone HE | |
| 538. | TTTTTATCCAT | 4.00 | 0.61 | 3.26 | 0.48 | Hs.248572 | hypothetical | 1.23 |
| | | | | | | | protein | |
| | | | | | | | FLJ22965 | |
| | | | | | | | [Swisspro | |
| 539. | AGCTCTGGAAG | 4.00 | 0.61 | 3.26 | 0.48 | Hs.24994 | CGI-53 protein | 1.23 |
| | | | | | | | [Swissprot: | |
| | | | | | | | sp\|P10244; sp | |
| 540. | TGAACCAGGTG | 4.00 | 0.61 | 3.26 | 0.48 | Hs.25059 | A kinase (PRKA) | 1.23 |
| | | | | | | | anchor protein 8 | |
| | | | | | | | [Swiss | |
| 541. | CACTATATTTG | 4.00 | 0.61 | 3.26 | 0.48 | Hs.250666 | hairy | 1.23 |
| | | | | | | | (Drosophila)- | |
| | | | | | | | homolog | |
| | | | | | | | [Swissprot: | |
| 542. | TGTGCACAATA | 4.00 | 0.61 | 3.26 | 0.48 | Hs.251636 | ubiquitin specific | 1.23 |
| | | | | | | | protease 3 | |
| | | | | | | | [Swisspro | |
| 543. | CATACATTGAT | 4.00 | 0.61 | 3.26 | 0.48 | Hs.251830 | ESTs | 1.23 |
| | | | | | | | [Swissprot: | |
| | | | | | | | none] | |
| 544. | GCTGCCAAAAG | 4.00 | 0.61 | 3.26 | 0.48 | Hs.251946 | poly(A)-binding | 1.23 |
| | | | | | | | protein, | |
| | | | | | | | cytoplasmic 1-I | |
| 545. | AGTTTTGCTGT | 4.00 | 0.61 | 3.26 | 0.48 | Hs.252748 | KIAA1728 | 1.23 |
| | | | | | | | protein | |
| | | | | | | | [Swissprot: | |
| | | | | | | | sp\|Q9C0D5; | |
| 546. | TAATTGCTTAT | 4.00 | 0.61 | 3.26 | 0.48 | Hs.25882 | DKFZP586M182 | 1.23 |
| | | | | | | | 4 protein | |
| | | | | | | | [Swissprot: | |
| | | | | | | | sp\|Q9 | |
| 547. | ATGTGAGGGAG | 4.00 | 0.61 | 3.26 | 0.48 | Hs.266935 | tRNA | 1.23 |
| | | | | | | | selenocysteine | |
| | | | | | | | associated | |
| | | | | | | | protein | |
| 548. | ACATTGGTAAA | 4.00 | 0.61 | 3.26 | 0.48 | Hs.267993 | hypothetical | 1.23 |
| | | | | | | | protein | |
| | | | | | | | FLJ10143 | |
| | | | | | | | [Swisspro | |
| 549. | GGTGGTGTCTG | 4.00 | 0.61 | 3.26 | 0.48 | Hs.2704 | glutathione | 1.23 |
| | | | | | | | peroxidase 2 | |
| | | | | | | | (gastrointestin | |
| 550. | AAAACCTAAAT | 4.00 | 0.61 | 3.26 | 0.48 | Hs.27135 | B-cell receptor- | 1.23 |
| | | | | | | | associated | |
| | | | | | | | protein BAP29 | |
| 551. | GCCCTACAGAT | 4.00 | 0.61 | 3.26 | 0.48 | Hs.27135 | B-cell receptor- | 1.23 |
| | | | | | | | associated | |
| | | | | | | | protein BAP29 | |
| 552. | TAGTTAAGCCA | 4.00 | 0.61 | 3.26 | 0.48 | Hs.271926 | serologically | 1.23 |
| | | | | | | | defined colon | |
| | | | | | | | cancer antig | |
| 553. | AACTTAAAAAA | 4.00 | 0.61 | 3.26 | 0.48 | Hs.272458 | protein | 1.23 |
| | | | | | | | phosphatase 3 | |
| | | | | | | | (formerly 2B), | |
| | | | | | | | cat | |
| 554. | GTGCGCCGCTG | 4.00 | 0.61 | 3.26 | 0.48 | Hs.272586 | KIAA0943 | 1.23 |
| | | | | | | | protein | |
| | | | | | | | [Swissprot: | |
| | | | | | | | sp\|Q96K07; | |
| 555. | GAGGGTATACT | 4.00 | 0.61 | 3.26 | 0.48 | Hs.274184 | transcription | 1.23 |
| | | | | | | | factor binding to | |
| | | | | | | | IGHM enh | |
| 556. | GTTTAAGAATT | 4.00 | 0.61 | 3.26 | 0.48 | Hs.274412 | similar to yeast | 1.23 |
| | | | | | | | Upf3, variant A | |
| | | | | | | | [Swiss | |
| 557. | AGGCCACCCTG | 4.00 | 0.61 | 3.26 | 0.48 | Hs.277477 | major | 1.23 |
| | | | | | | | histocompatibility | |
| | | | | | | | complex, class | |
| 558. | GGGAAGAGTGA | 4.00 | 0.61 | 3.26 | 0.48 | Hs.279474 | Homo sapiens | 1.23 |
| | | | | | | | HSPC070 | |
| | | | | | | | protein | |
| | | | | | | | (HSPC070), | |
| 559. | CGGAGCCGGCT | 4.00 | 0.61 | 3.26 | 0.48 | Hs.279780 | NEDD8 ultimate | 1.23 |
| | | | | | | | buster-1 | |
| | | | | | | | [Swissprot: sp\| | |
| 560. | AATGAAAGGTT | 4.00 | 0.61 | 3.26 | 0.48 | Hs.279842 | HSPC157 | 1.23 |
| | | | | | | | protein | |
| | | | | | | | [Swissprot: | |
| | | | | | | | sp\|Q9P007;] | |
| 561. | CGGCTCAAGTC | 4.00 | 0.61 | 3.26 | 0.48 | Hs.279868 | SUMO-1 | 1.23 |
| | | | | | | | activating | |
| | | | | | | | enzyme subunit | |
| | | | | | | | 1 [Swi | |
| 562. | GTCTTCTTAAT | 4.00 | 0.61 | 3.26 | 0.48 | Hs.279884 | DNAJ domain- | 1.23 |
| | | | | | | | containing | |
| | | | | | | | [Swissprot: sp\|Q | |
| 563. | ACGTGAGTGCT | 4.00 | 0.61 | 3.26 | 0.48 | Hs.279932 | CGI-105 protein | 1.23 |
| | | | | | | | [Swissprot: | |
| | | | | | | | sp\|Q96GK7; s | |
| 564. | GCCAGCCTATG | 4.00 | 0.61 | 3.26 | 0.48 | Hs.283663 | ESTs | 1.23 |
| | | | | | | | [Swissprot: | |
| | | | | | | | none] | |
| 565. | TGTGAGTTATT | 4.00 | 0.61 | 3.26 | 0.48 | Hs.288036 | tRNA | 1.23 |
| | | | | | | | isopentenylpyrophosphate | |
| | | | | | | | transferas | |
| 566. | TGTGTTGAGAC | 4.00 | 0.61 | 3.26 | 0.48 | Hs.288036 | tRNA | 1.23 |
| | | | | | | | isopentenylpyrophosphate | |
| | | | | | | | transferas | |
| 567. | ACCATAATGTG | 4.00 | 0.61 | 3.26 | 0.48 | Hs.288151 | hypothetical | 1.23 |
| | | | | | | | protein | |
| | | | | | | | FLJ23445 | |
| | | | | | | | [Swisspro | |
| 568. | GTATCTATGCA | 4.00 | 0.61 | 3.26 | 0.48 | Hs.288462 | hypothetical | 1.23 |
| | | | | | | | protein | |
| | | | | | | | FLJ21511 | |
| | | | | | | | [Swisspro | |
| 569. | CTCCAATGTAT | 4.00 | 0.61 | 3.26 | 0.48 | Hs.288773 | zinc finger | 1.23 |
| | | | | | | | protein 294 | |
| | | | | | | | [Swissprot: sp\| | |
| 570. | AAGGTCTATTC | 4.00 | 0.61 | 3.26 | 0.48 | Hs.29041 | Homo sapiens | 1.23 |
| | | | | | | | cDNA FLJ14177 | |
| | | | | | | | fis, clone NT | |
| 571. | TTTGCAAATAA | 4.00 | 0.61 | 3.26 | 0.48 | Hs.293055 | ESTs | 1.23 |
| | | | | | | | [Swissprot: | |
| | | | | | | | none] | |
| 572. | GGTAGCTCAGG | 4.00 | 0.61 | 3.26 | 0.48 | Hs.293860 | spinster-like | 1.23 |
| | | | | | | | protein | |
| | | | | | | | [Swissprot: | |
| | | | | | | | sp\|Q9 | |
| 573. | CCTGAGTTGAA | 4.00 | 0.61 | 3.26 | 0.48 | Hs.294083 | clone FLB4739 | 1.23 |
| | | | | | | | [Swissprot: | |
| | | | | | | | sp\|Q9P1N5;] | |
| 574. | TCTCCCACACC | 4.00 | 0.61 | 3.26 | 0.48 | Hs.2961 | S100 calcium- | 1.23 |
| | | | | | | | binding protein | |
| | | | | | | | A3 [Swissp | |
| 575. | AAGACAGTGGG | 4.00 | 0.61 | 3.26 | 0.48 | Hs.296290 | ribosomal | 1.23 |
| | | | | | | | protein L37a | |
| | | | | | | | [Swissprot: sp\|P | |
| 576. | GAGGGCCTTCA | 4.00 | 0.61 | 3.26 | 0.48 | Hs.296356 | Homo sapiens | 1.23 |
| | | | | | | | mRNA; cDNA | |
| | | | | | | | DKFZp434M162 | |
| | | | | | | | (fr | |
| 577. | CAGTCAGGCTG | 4.00 | 0.61 | 3.26 | 0.48 | Hs.298476 | solute carrier | 1.23 |
| | | | | | | | family 26, | |
| | | | | | | | member 6 [Swi | |
| 578. | ATAAATTTGTG | 4.00 | 0.61 | 3.26 | 0.48 | Hs.307013 | keratin | 1.23 |
| | | | | | | | associated | |
| | | | | | | | protein 9.2 | |
| | | | | | | | [Swisspr | |
| 579. | TGGAATTGTTT | 4.00 | 0.61 | 3.26 | 0.48 | Hs.307023 | keratin | 1.23 |
| | | | | | | | associated | |
| | | | | | | | protein 4.3 | |
| | | | | | | | [Swisspr | |
| 580. | GGGGTGGCAGG | 4.00 | 0.61 | 3.26 | 0.48 | Hs.310419 | ESTs | 1.23 |
| | | | | | | | [Swissprot: | |
| | | | | | | | none] | |
| 581. | CAAGAGGCAGT | 4.00 | 0.61 | 3.26 | 0.48 | Hs.311594 | EST [Swissprot: | 1.23 |
| | | | | | | | none] | |
| 582. | CCATTGCAATC | 4.00 | 0.61 | 3.26 | 0.48 | Hs.312642 | ESTs, Weakly | 1.23 |
| | | | | | | | similar to | |
| | | | | | | | 2109260A B cell | |
| 583. | AGGGACTCCTC | 4.00 | 0.61 | 3.26 | 0.48 | Hs.31438 | KIAA0435 gene | 1.23 |
| | | | | | | | product | |
| | | | | | | | [Swissprot: | |
| | | | | | | | sp\|O4 | |
| 584. | ACAAACTAAAA | 4.00 | 0.61 | 3.26 | 0.48 | Hs.31524 | ESTs | 1.23 |
| | | | | | | | [Swissprot: | |
| | | | | | | | none] | |
| 585. | CAAATATCTTG | 4.00 | 0.61 | 3.26 | 0.48 | Hs.315482 | ESTs | 1.23 |
| | | | | | | | [Swissprot: | |
| | | | | | | | none] | |
| 586. | TGCGTGACAGA | 4.00 | 0.61 | 3.26 | 0.48 | Hs.31819 | HT014 | 1.23 |
| | | | | | | | [Swissprot: | |
| | | | | | | | sp\|Q9GZP4; sp\| | |
| | | | | | | | Q9NRI8;] | |
| 587. | GCACTCTAACC | 4.00 | 0.61 | 3.26 | 0.48 | Hs.318827 | EST [Swissprot: | 1.23 |
| | | | | | | | none] | |
| 588. | TTTAATTGTAA | 4.00 | 0.61 | 3.26 | 0.48 | Hs.32241 | ESTs | 1.23 |
| | | | | | | | [Swissprot: | |
| | | | | | | | none] | |
| 589. | AGGTCAAAAAA | 4.00 | 0.61 | 3.26 | 0.48 | Hs.323342 | actin related | 1.23 |
| | | | | | | | protein 2/3 | |
| | | | | | | | complex, subun | |
| 590. | ATCATTCCCTC | 4.00 | 0.61 | 3.26 | 0.48 | Hs.323401 | dpy-30-like | 1.23 |
| | | | | | | | protein | |
| | | | | | | | [Swissprot: | |
| | | | | | | | sp\|Q9C0 | |
| 591. | GTGAGTGTGTC | 4.00 | 0.61 | 3.26 | 0.48 | Hs.323537 | hypothetical | 1.23 |
| | | | | | | | protein | |
| | | | | | | | FLJ12953 | |
| | | | | | | | similar to | |
| 592. | AAAATAAATGT | 4.00 | 0.61 | 3.26 | 0.48 | Hs.324473 | mitogen- | 1.23 |
| | | | | | | | activated protein | |
| | | | | | | | kinase 1 [Swi | |
| 593. | CTCGAGGAGGA | 4.00 | 0.61 | 3.26 | 0.48 | Hs.3254 | mitochondrial | 1.23 |
| | | | | | | | ribosomal | |
| | | | | | | | protein L23 [Sw | |
| 594. | GTGGCACGCAT | 4.00 | 0.61 | 3.26 | 0.48 | Hs.326800 | Human EST | 1.23 |
| | | | | | | | clone 53125 | |
| | | | | | | | mariner | |
| | | | | | | | transposon | |
| 595. | TGAATAAAAAA | 4.00 | 0.61 | 3.26 | 0.48 | Hs.327213 | EST [Swissprot: | 1.23 |
| | | | | | | | none] | |
| 596. | GTCTGGAAGGA | 4.00 | 0.61 | 3.26 | 0.48 | Hs.32950 | keratin, hair, | 1.23 |
| | | | | | | | acidic, 3B | |
| | | | | | | | [Swissprot: sp | |
| 597. | GCAGAAAATTT | 4.00 | 0.61 | 3.26 | 0.48 | Hs.333555 | chromosome 2 | 1.23 |
| | | | | | | | open reading | |
| | | | | | | | frame 2 [Swis | |
| 598. | TACCCCATAAA | 4.00 | 0.61 | 3.26 | 0.48 | Hs.334544 | Homo sapiens, | 1.23 |
| | | | | | | | clone | |
| | | | | | | | IMAGE: 4098392, | |
| | | | | | | | mRNA, | |
| 599. | GCAAGCCCCAG | 4.00 | 0.61 | 3.26 | 0.48 | Hs.334895 | ribosomel | 1.23 |
| | | | | | | | protein L10a | |
| | | | | | | | [Swissprot: sp\|O | |
| 600. | GGCAAAGCCCC | 4.00 | 0.61 | 3.26 | 0.48 | Hs.334895 | ribosomal | 1.23 |
| | | | | | | | protein L10a | |
| | | | | | | | [Swissprot: sp\|O | |
| 601. | CAGATTTGCCT | 4.00 | 0.61 | 3.26 | 0.48 | Hs.33979 | CGI-02 protein | 1.23 |
| | | | | | | | [Swissprot: | |
| | | | | | | | sp\|Q96FE6; sp | |
| 602. | TTTTGGGCAGT | 4.00 | 0.61 | 3.26 | 0.48 | Hs.34045 | hypothetical | 1.23 |
| | | | | | | | protein | |
| | | | | | | | FLJ20764 | |
| | | | | | | | [Swisspro | |
| 603. | GGTAAAATTAT | 4.00 | 0.61 | 3.26 | 0.48 | Hs.340959 | Ts translation | 1.23 |
| | | | | | | | elongation | |
| | | | | | | | factor, mitoch | |
| 604. | AATTACCAAAG | 4.00 | 0.61 | 3.26 | 0.48 | Hs.343566 | KIAA0251 | 1.23 |
| | | | | | | | protein | |
| | | | | | | | [Swissprot: | |
| | | | | | | | sp\|O00236; | |
| 605. | ACTATTAGTGC | 4.00 | 0.61 | 3.26 | 0.48 | Hs.3436 | CDK2- | 1.23 |
| | | | | | | | associated | |
| | | | | | | | protein 1 | |
| | | | | | | | [Swissprot: s | |
| 606. | TTTTGTCAACA | 4.00 | 0.61 | 3.26 | 0.48 | Hs.343661 | tripartite motif- | 1.23 |
| | | | | | | | containing 7 | |
| | | | | | | | [Swisspro | |
| 607. | GCTGGAAATGT | 4.00 | 0.61 | 3.26 | 0.48 | Hs.343847 | EST [Swissprot: | 1.23 |
| | | | | | | | none] | |
| 608. | CACTTGCAGTA | 4.00 | 0.61 | 3.26 | 0.48 | Hs.343877 | hypothetical | 1.23 |
| | | | | | | | protein | |
| | | | | | | | FLJ20039 | |
| | | | | | | | [Swisspro | |
| 609. | TACATATCTGT | 4.00 | 0.61 | 3.26 | 0.48 | Hs.34497 | hypothetical | 1.23 |
| | | | | | | | protein | |
| | | | | | | | FLJ22116 | |
| | | | | | | | [Swisspro | |
| 610. | GCCACCACCAC | 4.00 | 0.61 | 3.26 | 0.48 | Hs.347326 | intercellular | 1.23 |
| | | | | | | | adhesion | |
| | | | | | | | molecule 2 | |
| | | | | | | | [Swis | |
| 611. | ATGGCACCAGT | 4.00 | 0.61 | 3.26 | 0.48 | Hs.347873 | EST [Swissprot: | 1.23 |
| | | | | | | | none] | |
| 612. | TTTGTTTTTAT | 4.00 | 0.61 | 3.26 | 0.48 | Hs.3622 | procollagen- | 1.23 |
| | | | | | | | proline, 2- | |
| | | | | | | | oxoglutarate 4-di | |
| 613. | TGGTTTTCTCC | 4.00 | 0.61 | 3.26 | 0.48 | Hs.37129 | sodium channel, | 1.23 |
| | | | | | | | nonvoltage- | |
| | | | | | | | gated 1, beta | |
| 614. | AATAAAGTTAT | 4.00 | 0.61 | 3.26 | 0.48 | Hs.38260 | ubiquitin specific | 1.23 |
| | | | | | | | protease 18 | |
| | | | | | | | [Swisspr | |
| 615. | CTGCTTTAGGA | 4.00 | 0.61 | 3.26 | 0.48 | Hs.40782 | ESTs | 1.23 |
| | | | | | | | [Swissprot: | |
| | | | | | | | none] | |
| 616. | CTGTAACTGAC | 4.00 | 0.61 | 3.26 | 0.48 | Hs.42792 | Homo sapiens, | 1.23 |
| | | | | | | | clone | |
| | | | | | | | IMAGE: 3899073, | |
| | | | | | | | mRNA, | |
| 617. | GTGAGACACTA | 4.00 | 0.61 | 3.26 | 0.48 | Hs.43436 | adenylate kinase | 1.23 |
| | | | | | | | 3 alpha like | |
| | | | | | | | [Swisspro | |
| 618. | AACCAGGTGGA | 4.00 | 0.61 | 3.26 | 0.48 | Hs.44095 | cyclin M3 | 1.23 |
| | | | | | | | [Swissprot: | |
| | | | | | | | sp\|Q9NRK4; sp\| | |
| | | | | | | | Q9NX | |
| 619. | CGGCCCATCTG | 4.00 | 0.61 | 3.26 | 0.48 | Hs.44175 | Sec15B protein | 1.23 |
| | | | | | | | [Swissprot: | |
| | | | | | | | sp\|Q9H8D6; sp | |
| 620. | TTTTCTTCGGA | 4.00 | 0.61 | 3.26 | 0.48 | Hs.45033 | lacrimal proline | 1.23 |
| | | | | | | | rich protein | |
| | | | | | | | [Swisspro | |
| 621. | TTCGTATTACA | 4.00 | 0.61 | 3.26 | 0.48 | Hs.46743 | McKusick- | 1.23 |
| | | | | | | | Kaufman | |
| | | | | | | | syndrome | |
| | | | | | | | [Swissprot: s | |
| 622. | CTGGTGTCTGG | 4.00 | 0.61 | 3.26 | 0.48 | Hs.4747 | dyskeratosis | 1.23 |
| | | | | | | | congenita 1, | |
| | | | | | | | dyskerin [Swl | |
| 623. | GCCCTAGCAAT | 4.00 | 0.61 | 3.26 | 0.48 | Hs.47986 | hypothetical | 1.23 |
| | | | | | | | protein | |
| | | | | | | | MGC10940 | |
| | | | | | | | [Swisspro | |
| 624. | ATTTTGCAGTG | 4.00 | 0.61 | 3.26 | 0.48 | Hs.48924 | armadillo repeat | 1.23 |
| | | | | | | | protein ALEX2 | |
| | | | | | | | [Swisspr | |
| 625. | GTCAGTGTTAC | 4.00 | 0.61 | 3.26 | 0.48 | Hs.48938 | hypothetical | 1.23 |
| | | | | | | | protein | |
| | | | | | | | FLJ21802 | |
| | | | | | | | [Swisspro | |
| 626. | CAAACTTTGAA | 4.00 | 0.61 | 3.26 | 0.48 | Hs.49391 | chromosome 21 | 1.23 |
| | | | | | | | open reading | |
| | | | | | | | frame 91 [Sw | |
| 627. | TTTGCCTGGGG | 4.00 | 0.61 | 3.26 | 0.48 | Hs.50186 | ESTs | 1.23 |
| | | | | | | | [Swissprot: | |
| | | | | | | | none] | |
| 628. | CATCTTTTTAT | 4.00 | 0.61 | 3.26 | 0.48 | Hs.5085 | dolichyl- | 1.23 |
| | | | | | | | phosphate | |
| | | | | | | | mannosyltransferase p | |
| 629. | TCATTTGCTCC | 4.00 | 0.61 | 3.26 | 0.48 | Hs.5169 | suppressor of | 1.23 |
| | | | | | | | G2 allele of | |
| | | | | | | | SKP1, S. cere | |
| 630. | TAGCAGACCCT | 4.00 | 0.61 | 3.26 | 0.48 | Hs.5241 | fatty acid binding | 1.23 |
| | | | | | | | protein 1, liver | |
| | | | | | | | [Sw | |
| 631. | GCTGATCTGTC | 4.00 | 0.61 | 3.26 | 0.48 | Hs.5308 | ubiquitin A-52 | 1.23 |
| | | | | | | | residue | |
| | | | | | | | ribosomal | |
| | | | | | | | protein | |
| 632. | TAATGCCTTGT | 4.00 | 0.61 | 3.26 | 0.48 | Hs.55118 | ESTs | 1.23 |
| | | | | | | | [Swissprot: | |
| | | | | | | | none] | |
| 633. | ACTTGAATTCT | 4.00 | 0.61 | 3.26 | 0.48 | Hs.552 | steroid-5-alpha- | 1.23 |
| | | | | | | | reductase, alpha | |
| | | | | | | | polypep | |
| 634. | TGTCAAAAAAA | 4.00 | 0.61 | 3.26 | 0.48 | Hs.55296 | HLA-B | 1.23 |
| | | | | | | | associated | |
| | | | | | | | transcript 1 | |
| | | | | | | | [Swisspro | |
| 635. | TGTTTGGTTTG | 4.00 | 0.61 | 3.26 | 0.48 | Hs.56205 | insulin induced | 1.23 |
| | | | | | | | gene 1 | |
| | | | | | | | [Swissprot: sp\|O | |
| 636. | GACCAACAGTG | 4.00 | 0.61 | 3.26 | 0.48 | Hs.58650 | melanoma | 1.23 |
| | | | | | | | antigen | |
| | | | | | | | recognized by T | |
| | | | | | | | cells 2 | |
| 637. | TTGGGTTGTTA | 4.00 | 0.61 | 3.26 | 0.48 | Hs.5990 | tetratricopeptide | 1.23 |
| | | | | | | | repeat domain 4 | |
| | | | | | | | [Swis | |
| 638. | CCCAAACCTTC | 4.00 | 0.61 | 3.26 | 0.48 | Hs.61184 | CGI-79 protein | 1.23 |
| | | | | | | | [Swissprot: | |
| | | | | | | | sp\|Q9Y388; sp | |
| 639. | AAGGATGAATT | 4.00 | 0.61 | 3.26 | 0.48 | Hs.61539 | ESTs | 1.23 |
| | | | | | | | [Swissprot: | |
| | | | | | | | none] | |
| 640. | TGTTTCAGGAT | 4.00 | 0.61 | 3.26 | 0.48 | Hs.6216 | Homo sapiens | 1.23 |
| | | | | | | | cDNA FLJ10471 | |
| | | | | | | | fis, clone NT | |
| 641. | GTTTGTTGGGA | 4.00 | 0.61 | 3.26 | 0.48 | Hs.64691 | KIAA0483 | 1.23 |
| | | | | | | | protein | |
| | | | | | | | [Swissprot: | |
| | | | | | | | sp\|O75070; | |
| 642. | GTTAAATAAGA | 4.00 | 0.61 | 3.26 | 0.48 | Hs.6529 | ESTs, Weakly | 1.23 |
| | | | | | | | similar to I78885 | |
| | | | | | | | serine/th | |
| 643. | CCCTATGTTTG | 4.00 | 0.61 | 3.26 | 0.48 | Hs.6557 | zinc finger | 1.23 |
| | | | | | | | protein 161 | |
| | | | | | | | [Swissprot: sp\| | |
| 644. | GTGATTGTTCA | 4.00 | 0.61 | 3.26 | 0.48 | Hs.6727 | Ras-GTPase | 1.23 |
| | | | | | | | activating | |
| | | | | | | | protein SH3 | |
| | | | | | | | domain | |
| 645. | GGGCAGAATAA | 4.00 | 0.61 | 3.26 | 0.48 | Hs.6727 | Ras-GTPase | 1.23 |
| | | | | | | | activating | |
| | | | | | | | protein SH3 | |
| | | | | | | | domain | |
| 646. | TCTTTGTCTAA | 4.00 | 0.61 | 3.26 | 0.48 | Hs.6838 | ras homolog | 1.23 |
| | | | | | | | gene family, | |
| | | | | | | | member E [Swis | |
| 647. | ACACAGCTCTG | 4.00 | 0.61 | 3.26 | 0.48 | Hs.69559 | KIAA1096 | 1.23 |
| | | | | | | | protein | |
| | | | | | | | [Swissprot: | |
| | | | | | | | sp\|Q9NSM8; | |
| 648. | GTCCCGGGCAT | 4.00 | 0.61 | 3.26 | 0.48 | Hs.7218 | similar to acetyl- | 1.23 |
| | | | | | | | coenzyme A | |
| | | | | | | | synthetase | |
| 649. | TTTTAATTGCT | 4.00 | 0.61 | 3.26 | 0.48 | Hs.72242 | KIAA1322 | 1.23 |
| | | | | | | | protein | |
| | | | | | | | [Swissprot: | |
| | | | | | | | sp\|Q9P2M4; | |
| 650. | CCTCAGCCTCC | 4.00 | 0.61 | 3.26 | 0.48 | Hs.7337 | hypothetical | 1.23 |
| | | | | | | | protein | |
| | | | | | | | FLJ10936 | |
| | | | | | | | [Swisspro | |
| 651. | AATATTTACCA | 4.00 | 0.61 | 3.26 | 0.48 | Hs.74313 | KIAA1265 | 1.23 |
| | | | | | | | protein | |
| | | | | | | | [Swissprot: | |
| | | | | | | | sp\|Q9ULF5; | |
| 652. | CAGGTCGCTAC | 4.00 | 0.61 | 3.26 | 0.48 | Hs.75066 | translin | 1.23 |
| | | | | | | | [Swissprot: | |
| | | | | | | | sp\|Q15631;] | |
| 653. | GGCACAATCAA | 4.00 | 0.61 | 3.26 | 0.48 | Hs.75981 | ubiquitin specific | 1.23 |
| | | | | | | | protease 14 | |
| | | | | | | | (tRNA-gua | |
| 654. | CCTAATGTGTT | 4.00 | 0.61 | 3.26 | 0.48 | Hs.76084 | hypothetical | 1.23 |
| | | | | | | | protein | |
| | | | | | | | MGC2721 | |
| | | | | | | | [Swissprot | |
| 655. | TTCTCTATTGG | 4.00 | 0.61 | 3.26 | 0.48 | Hs.76536 | transducin | 1.23 |
| | | | | | | | (beta)-like 1 | |
| | | | | | | | [Swissprot: sp | |
| 656. | GACACCGGATT | 4.00 | 0.61 | 3.26 | 0.48 | Hs.76780 | protein | 1.23 |
| | | | | | | | phosphatase 1, | |
| | | | | | | | regulatory (inhib | |
| 657. | GTGAAGGCAGG | 4.00 | 0.61 | 3.26 | 0.48 | HS.77039 | ribosomal | 1.23 |
| | | | | | | | protein S3A | |
| | | | | | | | [Swissprot: | |
| | | | | | | | sp\|P4 | |
| 658. | AGAAAAACAAA | 4.00 | 0.61 | 3.26 | 0.48 | Hs.77965 | peptidyl-prolyl | 1.23 |
| | | | | | | | isomerase G | |
| | | | | | | | (cyclophilin | |
| 659. | CATTTTAATAC | 4.00 | 0.61 | 3.26 | 0.48 | Hs.78305 | RAB2, member | 1.23 |
| | | | | | | | RAS oncogene | |
| | | | | | | | family [Swiss | |
| 660. | TGAAGGTGGAT | 4.00 | 0.61 | 3.26 | 0.48 | Hs.7840 | calcineurin | 1.23 |
| | | | | | | | binding protein 1 | |
| | | | | | | | [Swisspro | |
| 661. | CCTTTGTAAAA | 4.00 | 0.61 | 3.26 | 0.48 | Hs.78465 | v-jun avian | 1.23 |
| | | | | | | | sarcoma virus | |
| | | | | | | | 17 oncogene ho | |
| 662. | TAAAACTTACC | 4.00 | 0.61 | 3.26 | 0.48 | Hs.7857 | erythrocyte | 1.23 |
| | | | | | | | membrane | |
| | | | | | | | protein band | |
| | | | | | | | 4.1-li | |
| 663. | TATAAAGTAAA | 4.00 | 0.61 | 3.26 | 0.48 | Hs.7879 | interferon- | 1.23 |
| | | | | | | | related | |
| | | | | | | | developmental | |
| | | | | | | | regulat | |
| 664. | TAAGTCTATAT | 4.00 | 0.61 | 3.26 | 0.48 | Hs.78864 | Fc fragment of | 1.23 |
| | | | | | | | IgG. low affinity | |
| | | | | | | | IIa, re | |
| 665. | CTTTGCACTCT | 4.00 | 0.61 | 3.26 | 0.48 | Hs.78869 | transcription | 1.23 |
| | | | | | | | elongation factor | |
| | | | | | | | A (SII), | |
| 666. | AGTATGGAATG | 4.00 | 0.61 | 3.26 | 0.48 | Hs.78989 | alcohol | 1.23 |
| | | | | | | | dehydrogenase | |
| | | | | | | | 5 (class III), chi | |
| 667. | GTGGAGGTGCG | 4.00 | 0.61 | 3.26 | 0.48 | Hs.79093 | EBNA-2 co- | 1.23 |
| | | | | | | | activator | |
| | | | | | | | (100 kD) | |
| | | | | | | | [Swissprot: | |
| 668. | CATTTAAGTTT | 4.00 | 0.61 | 3.26 | 0.48 | Hs.79300 | ubiquitin- | 1.23 |
| | | | | | | | conjugating | |
| | | | | | | | enzyme E2 | |
| | | | | | | | variant | |
| 669. | AGACTAACACG | 4.00 | 0.61 | 3.26 | 0.48 | Hs.79358 | testis-specific | 1.23 |
| | | | | | | | kinase 1 | |
| | | | | | | | [Swissprot: sp | |
| 670. | GTACCTAGAGT | 4.00 | 0.61 | 3.26 | 0.48 | Hs.7940 | RAP1, GTP- | 1.23 |
| | | | | | | | GDP | |
| | | | | | | | dissociation | |
| | | | | | | | stimulator 1 | |
| 671. | GTGAAGTCTTC | 4.00 | 0.61 | 3.26 | 0.48 | Hs.79748 | solute carrier | 1.23 |
| | | | | | | | family 3 | |
| | | | | | | | (activators of d | |
| 672. | TTCAGTGCTAC | 4.00 | 0.61 | 3.26 | 0.48 | Hs.7988 | Homo sapiens, | 1.23 |
| | | | | | | | Similar to | |
| | | | | | | | hypothetical pr | |
| 673. | TAAAAAAAAAT | 4.00 | 0.61 | 3.26 | 0.48 | Hs.80612 | ubiquitin- | 1.23 |
| | | | | | | | conjugating | |
| | | | | | | | enzyme E2A | |
| | | | | | | | (RAD6 h | |
| 674. | GGGGCACCCGC | 4.00 | 0.61 | 3.26 | 0.48 | Hs.82124 | laminin, beta 1 | 1.23 |
| | | | | | | | [Swissprot: | |
| | | | | | | | sp\|P07942;] | |
| 675. | TAGGTCCCCTT | 4.00 | 0.61 | 3.26 | 0.48 | Hs.82985 | collagen, type V, | 1.23 |
| | | | | | | | alpha 2 | |
| | | | | | | | [Swissprot: s | |
| 676. | ACTATCTCTAG | 4.00 | 0.61 | 3.26 | 0.48 | Hs.86347 | hypothetical | 1.23 |
| | | | | | | | protein | |
| | | | | | | | [Swissprot: | |
| | | | | | | | sp\|Q96 | |
| 677. | CTCCGAGGCAG | 4.00 | 0.61 | 3.26 | 0.48 | Hs.86508 | ESTs | 1.23 |
| | | | | | | | [Swissprot: | |
| | | | | | | | none] | |
| 678. | GCAGCAGTGTC | 4.00 | 0.61 | 3.26 | 0.48 | Hs.86538 | ESTs | 1.23 |
| | | | | | | | [Swissprot: | |
| | | | | | | | none] | |
| 679. | CAAGCTTGGTC | 4.00 | 0.61 | 3.26 | 0.48 | Hs.86858 | ribosomal | 1.23 |
| | | | | | | | protein S6 | |
| | | | | | | | kinase, 70 kD, | |
| | | | | | | | polyp | |
| 680. | CTGAGCTGTAC | 4.00 | 0.61 | 3.26 | 0.48 | Hs.8737 | WD repeat | 1.23 |
| | | | | | | | domain 6 | |
| | | | | | | | [Swissprot: | |
| | | | | | | | sp\|Q9BU8 | |
| 681. | AAGTCATCTAT | 4.00 | 0.61 | 3.26 | 0.48 | Hs.8769 | brain cell, | 1.23 |
| | | | | | | | membrane | |
| | | | | | | | protein 1 | |
| | | | | | | | [Swisspro | |
| 682. | TGGGTTTTTAT | 4.00 | 0.61 | 3.26 | 0.48 | Hs.88143 | ESTs | 1.23 |
| | | | | | | | [Swissprot: | |
| | | | | | | | none] | |
| 683. | GGAATGAGAAG | 4.00 | 0.61 | 3.26 | 0.48 | Hs.90077 | TGFB-induced | 1.23 |
| | | | | | | | factor (TALE | |
| | | | | | | | family homeobo | |
| 684. | ATACCAGATAC | 4.00 | 0.61 | 3.26 | 0.48 | Hs.9071 | progesterone | 1.23 |
| | | | | | | | receptor | |
| | | | | | | | membrane | |
| | | | | | | | component | |
| 685. | TATTAGTCACA | 4.00 | 0.61 | 3.26 | 0.48 | Hs.9078 | immature colon | 1.23 |
| | | | | | | | carcinoma | |
| | | | | | | | transcript 1 [ | |
| 686. | ACATAAGATCA | 4.00 | 0.61 | 3.26 | 0.48 | Hs.93832 | putative | 1.23 |
| | | | | | | | membrane | |
| | | | | | | | protein | |
| | | | | | | | [Swissprot: s | |
| 687. | CCTCTTCAGGC | 4.00 | 0.61 | 3.26 | 0.48 | Hs.9614 | nucleophosmin | 1.23 |
| | | | | | | | (nucleolar | |
| | | | | | | | phosphoprotein | |
| 688. | GACAAGGACAG | 4.00 | 0.61 | 3.26 | 0.48 | Hs.96322 | hypothetical | 1.23 |
| | | | | | | | protein | |
| | | | | | | | FLJ23560 | |
| | | | | | | | [Swisspro | |
| 689. | CTGCAAATGAA | 4.00 | 0.61 | 3.26 | 0.48 | Hs.96918 | Homo sapiens | 1.23 |
| | | | | | | | cDNA: | |
| | | | | | | | FLJ21561 fis, | |
| | | | | | | | clone C | |
| 690. | CCAATGGCCAG | 4.00 | 0.61 | 3.26 | 0.48 | Hs.98135 | hypothetical | 1.23 |
| | | | | | | | protein | |
| | | | | | | | FLJ20559 | |
| | | | | | | | [Swisspro | |
| 691. | GGAGTGCAGCT | 4.00 | 0.61 | 3.26 | 0.48 | Hs.98491 | Homo sapiens | 1.23 |
| | | | | | | | cDNA FLJ20525 | |
| | | | | | | | fis, clone KA | |
| 692. | TGCACACACAC | 4.00 | 0.61 | 3.26 | 0.48 | Hs.99816 | beta-catenin- | 1.23 |
| | | | | | | | interacting | |
| | | | | | | | protein ICAT [ | |
| 693. | GCTTCCATCTT | 3.55 | 0.98 | 1.90 | 0.41 | Hs.55296 | HLA-B | 1.87 |
| | | | | | | | associated | |
| | | | | | | | transcript 1 | |
| | | | | | | | [Swisspro | |
| 694. | CTTCGAAACTC | −3.95 | 0.65 | −2.46 | 0.32 | Hs.51299 | NADH | 1.61 |
| | | | | | | | dehydrogenase | |
| | | | | | | | (ubiquinone) | |
| | | | | | | | flavopro | |
| 695. | TGTGGGAACCA | −4.93 | 0.89 | −2.46 | 0.32 | Hs.7750 | hypothetical | 2.00 |
| | | | | | | | protein | |
| | | | | | | | AL133206 | |
| | | | | | | | [Swisspro | |
| 696. | CATTAAAGGGT | 3.04 | 0.44 | 2.44 | 0.29 | Hs.105509 | CTL2 gene | 1.25 |
| | | | | | | | [Swissprot: | |
| | | | | | | | sp\|Q9NY68;] | |
| 697. | TTTGGAAAAAA | 3.04 | 0.44 | 2.44 | 0.29 | Hs.12482 | glyceronephosphate | 1.25 |
| | | | | | | | O- | |
| | | | | | | | acyltransferase | |
| | | | | | | | [S | |
| 698. | GGGGGAATTTT | 3.04 | 0.44 | 2.44 | 0.29 | Hs.129548 | heterogeneous | 1.25 |
| | | | | | | | nuclear | |
| | | | | | | | ribonucleoprotein | |
| 699. | TTTTGATCACT | 3.04 | 0.44 | 2.44 | 0.29 | Hs.129952 | KIAA0560 gene | 1.25 |
| | | | | | | | product | |
| | | | | | | | [Swissprot: | |
| | | | | | | | sp\|O6 | |
| 700. | AGATGTGTGGG | 3.04 | 0.44 | 2.44 | 0.29 | Hs.146812 | hydroxyacyl- | 1.25 |
| | | | | | | | Coenzyme A | |
| | | | | | | | dehydrogenase/ | |
| | | | | | | | 3-k | |
| 701. | GGTCCCCTACC | 3.04 | 0.44 | 2.44 | 0.29 | Hs.151761 | KIAA0100 gene | 1.25 |
| | | | | | | | product | |
| | | | | | | | [Swissprot: | |
| | | | | | | | sp\|Q1 | |
| 702. | TCATAAGCAAT | 3.04 | 0.44 | 2.44 | 0.29 | Hs.177486 | amyloid beta | 1.25 |
| | | | | | | | (A4) precursor | |
| | | | | | | | protein (pro | |
| 703. | ACAAATCCTTG | 3.04 | 0.44 | 2.44 | 0.29 | Hs.179661 | FK506-binding | 1.25 |
| | | | | | | | protein 1A | |
| | | | | | | | (12 kD) [Swissp | |
| 704. | TGTAGTATTTG | 3.04 | 0.44 | 2.44 | 0.29 | Hs.18842 | protein kinase C | 1.25 |
| | | | | | | | and casein | |
| | | | | | | | kinase subst | |
| 705. | AACCCGGGGGG | 3.04 | 0.44 | 2.44 | 0.29 | Hs.200720 | EST [Swissprot: | 1.25 |
| | | | | | | | none] | |
| 706. | TGCTAGATTGG | 3.04 | 0.44 | 2.44 | 0.29 | Hs.239663 | myeloid/lymphoid | 1.25 |
| | | | | | | | or mixed- | |
| | | | | | | | lineage leukem | |
| 707. | GAGTAAAAAAT | 3.04 | 0.44 | 2.44 | 0.29 | Hs.279789 | histone | 1.25 |
| | | | | | | | deacetylase 3 | |
| | | | | | | | [Swissprot: | |
| | | | | | | | sp\|O1 | |
| 708. | TGTTACCAAGA | 3.04 | 0.44 | 2.44 | 0.29 | Hs.289068 | Homo sapiens | 1.25 |
| | | | | | | | cDNA FLJ11918 | |
| | | | | | | | fis, clone HE | |
| 709. | GAGCCGCCTCT | 3.04 | 0.44 | 2.44 | 0.29 | Hs.30026 | HSPC182 | 1.25 |
| | | | | | | | protein | |
| | | | | | | | [Swissprot: | |
| | | | | | | | sp\|Q96JR5; s | |
| 710. | TGGTGACAGTT | 3.04 | 0.44 | 2.44 | 0.29 | Hs.301005 | histone H2A.F/Z | 1.25 |
| | | | | | | | variant | |
| | | | | | | | [Swissprot: non | |
| 711. | GAAGTTGCCTT | 3.04 | 0.44 | 2.44 | 0.29 | Hs.319252 | Homo sapiens, | 1.25 |
| | | | | | | | Similar to | |
| | | | | | | | KIAA0843 protei | |
| 712. | GCCAGAAGGGG | 3.04 | 0.44 | 2.44 | 0.29 | Hs.321775 | hypothetical | 1.25 |
| | | | | | | | protein | |
| | | | | | | | DKFZp434D1428 | |
| | | | | | | | [Swi | |
| 713. | CTGCAACCTAA | 3.04 | 0.44 | 2.44 | 0.29 | Hs.50785 | SEC22, vesicle | 1.25 |
| | | | | | | | trafficking | |
| | | | | | | | protein (S, c | |
| 714. | AATAAACGTGT | 3.04 | 0.44 | 2.44 | 0.29 | Hs.57304 | Ras-related | 1.25 |
| | | | | | | | GTP-binding | |
| | | | | | | | protein [Swissp | |
| 715. | GGACCAGGCTG | 3.04 | 0.44 | 2.44 | 0.29 | Hs.62771 | Homo sapiens | 1.25 |
| | | | | | | | mRNA; cDNA | |
| | | | | | | | DKFZp761E1423 | |
| | | | | | | | (f | |
| 716. | TATCAAAACAC | 3.04 | 0.44 | 2.44 | 0.29 | Hs.74420 | origin | 1.25 |
| | | | | | | | recognition | |
| | | | | | | | complex, subunit 3 | |
| | | | | | | | (y | |
| 717. | TCGAAGAACCG | 3.04 | 0.44 | 2.44 | 0.29 | Hs.76294 | CD63 antigen | 1.25 |
| | | | | | | | (melanoma 1 | |
| | | | | | | | antigen) [Swis | |
| 718. | CTCTTCGAGAA | 3.04 | 0.44 | 2.44 | 0.29 | Hs.76686 | glutathione | 1.25 |
| | | | | | | | peroxidase 1 | |
| | | | | | | | [Swissprot: sp | |
| 719. | GGAGACTTCCT | 3.04 | 0.44 | 2.44 | 0.29 | Hs.77840 | annexin A4 | 1.25 |
| | | | | | | | [Swissprot: | |
| | | | | | | | sp\|P09525; sp\|Q96 | |
| 720. | CAGGACAGTTT | 3.04 | 0.44 | 2.44 | 0.29 | Hs.78305 | RAB2, member | 1.25 |
| | | | | | | | RAS oncogene | |
| | | | | | | | family [Swiss | |
| 721. | CCAAGGACTCT | 3.04 | 0.44 | 2.44 | 0.29 | Hs.79058 | suppressor of Ty | 1.25 |
| | | | | | | | (S. cerevisiae) 4 | |
| | | | | | | | homolo | |
| 722. | TAGAATGCAAA | 3.04 | 0.44 | 2.44 | 0.29 | Hs.7946 | KIAA1288 | 1.25 |
| | | | | | | | protein | |
| | | | | | | | [Swissprot: | |
| | | | | | | | sp\|Q9H7T2; | |
| 723. | CAGACGCTCCG | 3.04 | 0.44 | 2.44 | 0.29 | Hs.83006 | mitochondrial | 1.25 |
| | | | | | | | ribosomal | |
| | | | | | | | protein S33 [Sw | |
| 724. | GTTTAAAAAGA | 3.04 | 0.44 | 2.44 | 0.29 | Hs.90005 | stathmin-like 2 | 1.25 |
| | | | | | | | [Swissprot: | |
| | | | | | | | sp\|Q93045;] | |
| 725. | TGGAACAGGAT | 3.04 | 0.44 | 2.44 | 0.29 | Hs.90077 | TGFB-induced | 1.25 |
| | | | | | | | factor (TALE | |
| | | | | | | | family homeobo | |
| 726. | TTCCTCCACCC | 3.04 | 0.44 | 2.44 | 0.29 | Hs.9061 | hypothetical | 1.25 |
| | | | | | | | protein | |
| | | | | | | | MGC2477 | |
| | | | | | | | [Swissprot | |
| 727. | ACCCACCTGCA | 3.04 | 0.44 | 2.44 | 0.29 | Hs.9100 | hypothetical | 1.25 |
| | | | | | | | gene supported | |
| | | | | | | | by AK023162 | |
| 728. | GTGCCTAGGGA | 3.04 | 0.76 | 1.63 | 0.27 | Hs.12854 | angiotensin II, | 1.87 |
| | | | | | | | type I receptor- | |
| | | | | | | | associat | |
| 729. | CCCCCAGATGA | 3.04 | 0.76 | 1.63 | 0.27 | Hs.25817 | BTB (POZ) | 1.87 |
| | | | | | | | domain | |
| | | | | | | | containing 2 | |
| | | | | | | | [Swisspro | |
| 730. | GTCTGGGGGAT | 3.04 | 0.76 | 1.63 | 0.27 | Hs.334333 | Homo sapiens | 1.87 |
| | | | | | | | cDNA: | |
| | | | | | | | FLJ22330 fis, | |
| | | | | | | | clone H | |
| 731. | TGGGAAGTGGG | 4.05 | 0.67 | 1.63 | 0.20 | Hs.112844 | maternally | 2.48 |
| | | | | | | | expressed 3 | |
| | | | | | | | [Swissprot: sp\|Q | |
| 732. | GCAGGAGGTGA | 4.05 | 0.67 | 1.63 | 0.20 | Hs.11441 | chromosome 1 | 2.48 |
| | | | | | | | open reading | |
| | | | | | | | frame 8 [Swis | |
| 733. | CCTTACCTACA | 4.05 | 0.67 | 1.63 | 0.20 | Hs.184542 | CGI-127 protein | 2.48 |
| | | | | | | | [Swissprot: | |
| | | | | | | | sp\|Q9Y3C9;] | |
| 734. | GCGAAACCCTA | 4.05 | 0.67 | 1.63 | 0.20 | Hs.270249 | ESTs, Weakly | 2.48 |
| | | | | | | | similar to | |
| | | | | | | | 2004399A | |
| | | | | | | | chromos | |
| 735. | GCCAGACACCC | 4.05 | 0.67 | 1.63 | 0.20 | Hs.3804 | DKFZP564C1940 | 2.48 |
| | | | | | | | protein | |
| | | | | | | | [Swissprot: | |
| | | | | | | | sp\|O9 | |
| 736. | AATGTTGTGCA | 4.05 | 0.67 | 1.63 | 0.20 | Hs.91546 | cytochrome | 2.48 |
| | | | | | | | P450 retinoid | |
| | | | | | | | metabolizing pr | |
| 737. | GTTGCAGATAA | 4.00 | 0.61 | 1.63 | 0.10 | Hs.100293 | O-linked N- | 2.45 |
| | | | | | | | acetylglucosamine | |
| | | | | | | | (GlcNAc) tr | |
| 738. | TGGTTGCGACA | 4.00 | 0.61 | 1.63 | 0.10 | Hs.101408 | branched chain | 2.45 |
| | | | | | | | aminotransferase | |
| | | | | | | | 2, mitoc | |
| 739. | CAAATATGGTT | 4.00 | 0.61 | 1.63 | 0.10 | Hs.10351 | KIAA0308 | 2.45 |
| | | | | | | | protein | |
| | | | | | | | [Swissprot: | |
| | | | | | | | sp\|O15025; | |
| 740. | GAGAAAAAAAA | 4.00 | 0.61 | 1.63 | 0.10 | Hs.10450 | Homo sapiens | 2.45 |
| | | | | | | | cDNA: | |
| | | | | | | | FLJ22063 fis, | |
| | | | | | | | clone H | |
| 741. | TTTGAAAACAA | 4.00 | 0.61 | 1.63 | 0.10 | Hs.107203 | hypothetical | 2.45 |
| | | | | | | | protein from | |
| | | | | | | | EUROIMAGE | |
| | | | | | | | 1759 | |
| 742. | TGTAACAATAA | 4.00 | 0.61 | 1.63 | 0.10 | Hs.12600 | N- | 2.45 |
| | | | | | | | ethylmaleimide- | |
| | | | | | | | sensitive factor | |
| | | | | | | | attach | |
| 743. | TGTTGTATTTG | 4.00 | 0.61 | 1.63 | 0.10 | Hs.128653 | hypothetical | 2.45 |
| | | | | | | | protein | |
| | | | | | | | DKFZp564F013 | |
| | | | | | | | [Swis | |
| 744. | GAAAGGTGGTT | 4.00 | 0.61 | 1.63 | 0.10 | Hs.14394 | hypothetical | 2.45 |
| | | | | | | | protein | |
| | | | | | | | FLJ20157 | |
| | | | | | | | [Swisspro | |
| 745. | GGATGTAGAGA | 4.00 | 0.61 | 1.63 | 0.10 | Hs.155485 | huntingtin | 2.45 |
| | | | | | | | interacting | |
| | | | | | | | protein 2 [Swiss | |
| 746. | CACTGTGTGTA | 4.00 | 0.61 | 1.63 | 0.10 | Hs.164207 | hypothetical | 2.45 |
| | | | | | | | protein | |
| | | | | | | | FLJ21172 | |
| | | | | | | | [Swisspro | |
| 747. | CCTCTGGAGGC | 4.00 | 0.61 | 1.63 | 0.10 | Hs.167246 | P450 | 2.45 |
| | | | | | | | (cytochrome) | |
| | | | | | | | oxidoreductase | |
| | | | | | | | [Swiss | |
| 748. | TATTCCTGTGA | 4.00 | 0.61 | 1.63 | 0.10 | Hs.168075 | karyopherin | 2.45 |
| | | | | | | | (importin) beta 2 | |
| | | | | | | | [Swisspro | |
| 749. | CAACTTTAGGG | 4.00 | 0.61 | 1.63 | 0.10 | Hs.170311 | heterogeneous | 2.45 |
| | | | | | | | nuclear | |
| | | | | | | | ribonucleoprotein | |
| 750. | CATTTACTCTA | 4.00 | 0.61 | 1.63 | 0.10 | Hs.17109 | integral | 2.45 |
| | | | | | | | membrane | |
| | | | | | | | protein 2A | |
| | | | | | | | [Swissprot | |
| 751. | GGGCTGGACGG | 4.00 | 0.61 | 1.63 | 0.10 | Hs.180338 | tumor necrosis | 2.45 |
| | | | | | | | factor receptor | |
| | | | | | | | superfaml | |
| 752. | AACTGGGTCTG | 4.00 | 0.61 | 1.63 | 0.10 | Hs.182215 | ADP-ribosylation | 2.45 |
| | | | | | | | factor-like 3 | |
| | | | | | | | [Swisspr | |
| 753. | TGCAGGTTTGT | 4.00 | 0.61 | 1.63 | 0.10 | Hs.183800 | Ran GTPase | 2.45 |
| | | | | | | | activating | |
| | | | | | | | protein 1 | |
| | | | | | | | [Swissp | |
| 754. | AGGAAAGCCAG | 4.00 | 0.61 | 1.63 | 0.10 | Hs.19012 | Rab9 effector | 2.45 |
| | | | | | | | p40 [Swissprot: | |
| | | | | | | | sp\|O00568 | |
| 755. | CTAAATCACTG | 4.00 | 0.61 | 1.63 | 0.10 | Hs.19180 | Homo sapiens | 2.45 |
| | | | | | | | mRNA; cDNA | |
| | | | | | | | DKFZp564E122 | |
| | | | | | | | (fr | |
| 756. | AAAAATAAAGA | 4.00 | 0.61 | 1.63 | 0.10 | Hs.19414 | ESTs, Weakly | 2.45 |
| | | | | | | | similar to | |
| | | | | | | | INI7_HUMAN | |
| | | | | | | | INTER | |
| 757. | GAAGCAATAAA | 4.00 | 0.61 | 1.63 | 0.10 | Hs.198253 | major | 2.45 |
| | | | | | | | histocompatibility | |
| | | | | | | | complex, class | |
| 758. | GTGAAACCCTT | 4.00 | 0.61 | 1.63 | 0.10 | Hs.206955 | ESTs, Weakly | 2.45 |
| | | | | | | | similar to | |
| | | | | | | | 2109260A B cell | |
| 759. | GAAATGTATGC | 4.00 | 0.61 | 1.63 | 0.10 | Hs.21610 | DKFZP434B203 | 2.45 |
| | | | | | | | protein | |
| | | | | | | | [Swissprot: | |
| | | | | | | | sp\|Q9U | |
| 760. | CCACGTGTCCG | 4.00 | 0.61 | 1.63 | 0.10 | Hs.239451 | Homo sapiens, | 2.45 |
| | | | | | | | Similar to zinc | |
| | | | | | | | finger pro | |
| 761. | TAACTCCAAAG | 4.00 | 0.61 | 1.63 | 0.10 | Hs.24743 | hypothetical | 2.45 |
| | | | | | | | protein | |
| | | | | | | | FLJ20171 | |
| | | | | | | | [Swisspro | |
| 762. | TGGAGAGAATA | 4.00 | 0.61 | 1.63 | 0.10 | Hs.249247 | heterogeneous | 2.45 |
| | | | | | | | nuclear | |
| | | | | | | | ribonucleoprotein | |
| 763. | CTTGAGTCACA | 4.00 | 0.61 | 1.63 | 0.10 | Hs.261023 | hypothetical | 2.45 |
| | | | | | | | protein | |
| | | | | | | | FLJ20958 | |
| | | | | | | | [Swisspro | |
| 764. | CAATCTGATGC | 4.00 | 0.61 | 1.63 | 0.10 | Hs.26176 | hypothetical | 2.45 |
| | | | | | | | protein | |
| | | | | | | | FLJ10261 | |
| | | | | | | | [Swisspro | |
| 765. | CAATAAAACTG | 4.00 | 0.61 | 1.63 | 0.10 | Hs.267905 | hypothetical | 2.45 |
| | | | | | | | protein | |
| | | | | | | | FLJ10422 | |
| | | | | | | | [Swisspro | |
| 766. | CTTGCAGTCCT | 4.00 | 0.61 | 1.63 | 0.10 | Hs.27018 | Ris [Swissprot: | 2.45 |
| | | | | | | | sp\|Q9NYN1;] | |
| 767. | AGCCGGATGCT | 4.00 | 0.61 | 1.63 | 0.10 | Hs.284232 | KIAA0720 | 2.45 |
| | | | | | | | protein | |
| | | | | | | | [Swissprot: | |
| | | | | | | | sp\|O94827; | |
| 768. | GGAGCTTGAGG | 4.00 | 0.61 | 1.63 | 0.10 | Hs.288316 | chromosome 6 | 2.45 |
| | | | | | | | open reading | |
| | | | | | | | frame 9 [Swis | |
| 769. | CTTTACCAAAA | 4.00 | 0.61 | 1.63 | 0.10 | Hs.289088 | heat shock 90 kD | 2.45 |
| | | | | | | | protein 1, alpha | |
| | | | | | | | [Swiss | |
| 770. | CCACCACACCC | 4.00 | 0.61 | 1.63 | 0.10 | Hs.291047 | ESTs | 2.45 |
| | | | | | | | [Swissprot: | |
| | | | | | | | none] | |
| 771. | CAGGGCTCGCG | 4.00 | 0.61 | 1.63 | 0.10 | Hs.29288 | hypothetical | 2.45 |
| | | | | | | | protein | |
| | | | | | | | FLJ21865 | |
| | | | | | | | [Swisspro | |
| 772. | AACCCAGAAGG | 4.00 | 0.61 | 1.63 | 0.10 | Hs.295749 | EST, Weakly | 2.45 |
| | | | | | | | similar to | |
| | | | | | | | ALU2_HUMAN | |
| | | | | | | | ALU SU | |
| 773. | TTCCAAAGCGA | 4.00 | 0.61 | 1.63 | 0.10 | Hs.299315 | collapsin | 2.45 |
| | | | | | | | response | |
| | | | | | | | mediator protein- | |
| | | | | | | | 5; C | |
| 774. | GAGCTCAAGAT | 4.00 | 0.61 | 1.63 | 0.10 | Hs.306327 | Homo sapiens | 2.45 |
| | | | | | | | mRNA; cDNA | |
| | | | | | | | DKFZp434A012 | |
| | | | | | | | (fr | |
| 775. | CTGGTTTAAAT | 4.00 | 0.61 | 1.63 | 0.10 | Hs.306867 | KIAA1228 | 2.45 |
| | | | | | | | protein | |
| | | | | | | | (Swissprot: | |
| | | | | | | | sp\|Q9BVG1; | |
| 776. | ATGGCTCATTC | 4.00 | 0.61 | 1.63 | 0.10 | Hs.320321 | EST [Swissprot: | 2.45 |
| | | | | | | | none] | |
| 777. | TTTCAATACCA | 4.00 | 0.61 | 1.63 | 0.10 | Hs.322710 | ESTs | 2.45 |
| | | | | | | | [Swissprot: | |
| | | | | | | | none] | |
| 778. | TCTCCACGAAG | 4.00 | 0.61 | 1.63 | 0.10 | Hs.323342 | actin related | 2.45 |
| | | | | | | | protein 2/3 | |
| | | | | | | | complex, subun | |
| 779. | GCACTTACAAA | 4.00 | 0.61 | 1.63 | 0.10 | Hs.326248 | Homo sapiens | 2.45 |
| | | | | | | | mRNA; cDNA | |
| | | | | | | | DKFZp564H2416 | |
| | | | | | | | (f | |
| 780. | AGCCATCACAC | 4.00 | 0.61 | 1.63 | 0.10 | Hs.335156 | EST [Swissprot: | 2.45 |
| | | | | | | | none] | |
| 781. | AACTAAAAAAC | 4.00 | 0.61 | 1.63 | 0.10 | Hs.340245 | hypothetical | 2.45 |
| | | | | | | | protein | |
| | | | | | | | PRO1197 | |
| | | | | | | | [Swissprot | |
| 782. | GGAGGATCACT | 4.00 | 0.61 | 1.63 | 0.10 | Hs.345050 | ESTs | 2.45 |
| | | | | | | | [Swissprot: | |
| | | | | | | | none] | |
| 783. | AAATTGTTCCA | 4.00 | 0.61 | 1.63 | 0.10 | Hs.346918 | proteasome | 2.45 |
| | | | | | | | (prosome, | |
| | | | | | | | macropain) | |
| | | | | | | | subunit. | |
| 784. | TTCTTTGGGAA | 4.00 | 0.61 | 1.63 | 0.10 | Hs.346945 | hypothetical | 2.45 |
| | | | | | | | gene MGC1127 | |
| | | | | | | | [Swissprot: s | |
| 785. | GTATACCTACC | 4.00 | 0.61 | 1.63 | 0.10 | Hs.37040 | platelet-derived | 2.45 |
| | | | | | | | growth factor | |
| | | | | | | | alpha pol | |
| 786. | GTGGGTCAGCT | 4.00 | 0.61 | 1.63 | 0.10 | Hs.38592 | hypothetical | 2.45 |
| | | | | | | | protein | |
| | | | | | | | FLJ23342 | |
| | | | | | | | [Swisspro | |
| 787. | GCAGAGATGGG | 4.00 | 0.61 | 1.63 | 0.10 | Hs.39850 | hypothetical | 2.45 |
| | | | | | | | protein | |
| | | | | | | | FUJ20517 | |
| | | | | | | | [Swisspro | |
| 788. | CTGGGCCATTG | 4.00 | 0.61 | 1.63 | 0.10 | Hs.4 | alcohol | 2.45 |
| | | | | | | | dehydrogenase | |
| | | | | | | | 1B (class I), beta | |
| 789. | GATCAAAACTG | 4.00 | 0.61 | 1.63 | 0.10 | Hs.41267 | chromosome 21 | 2.45 |
| | | | | | | | open reading | |
| | | | | | | | frame 7 [Swi | |
| 790. | GTGGTGGACGC | 4.00 | 0.61 | 1.63 | 0.10 | Hs.47193 | Homo sapiens | 2.45 |
| | | | | | | | cDNA: | |
| | | | | | | | FLJ21221 fis, | |
| | | | | | | | clone C | |
| 791. | ATTGTGCTACT | 4.00 | 0.61 | 1.63 | 0.10 | Hs.50628 | adaptor-related | 2.45 |
| | | | | | | | protein complex | |
| | | | | | | | 4, sigma | |
| 792. | TTCTCTTTCAA | 4.00 | 0.61 | 1.63 | 0.10 | Hs.5354 | hypothetical | 2.45 |
| | | | | | | | protein | |
| | | | | | | | FLJ12716 | |
| | | | | | | | [Swisspro | |
| 793. | GCCAAAGATGT | 4.00 | 0.61 | 1.63 | 0.10 | Hs.58636 | squamous cell | 2.45 |
| | | | | | | | carcinoma | |
| | | | | | | | antigen recogniz | |
| 794. | CTGGTCGTTGG | 4.00 | 0.61 | 1.63 | 0.10 | Hs.5985 | non-kinase | 2.45 |
| | | | | | | | Cdc42 effector | |
| | | | | | | | protein SPEC2 | |
| 795. | AGCACTGTACT | 4.00 | 0.61 | 1.63 | 0.10 | Hs.6375 | uncharacterized | 2.45 |
| | | | | | | | hypothalamus | |
| | | | | | | | protein HT0 | |
| 796. | ACAAGATATTT | 4.00 | 0.61 | 1.63 | 0.10 | Hs.74122 | caspase 4, | 2.45 |
| | | | | | | | apoptosis- | |
| | | | | | | | related cysteine | |
| | | | | | | | pr | |
| 797. | TACATTTGAAT | 4.00 | 0.61 | 1.63 | 0.10 | Hs.77876 | hypothetical | 2.45 |
| | | | | | | | gene | |
| | | | | | | | MGC19595 | |
| | | | | | | | [Swissprot: | |
| 798. | AGAAATCACTG | 4.00 | 0.61 | 1.63 | 0.10 | Hs.8110 | L-3-hydroxyacyl- | 2.45 |
| | | | | | | | Coenzyme A | |
| | | | | | | | dehydrogenase | |
| 799. | AGTCCTTGAAA | 4.00 | 0.61 | 1.63 | 0.10 | Hs.81665 | v-kit Hardy- | 2.45 |
| | | | | | | | Zuckerman 4 | |
| | | | | | | | feline sarcoma v | |
| 800. | TAAATGAAAAA | 4.00 | 0.61 | 1.63 | 0.10 | Hs.82120 | nuclear receptor | 2.45 |
| | | | | | | | subfamily 4, | |
| | | | | | | | group A, m | |
| 801. | TTAGTCTTCAG | 4.00 | 0.61 | 1.63 | 0.10 | Hs.82712 | fragile X mental | 2.45 |
| | | | | | | | retardation, | |
| | | | | | | | autosomal | |
| 802. | AGTGTGCGCTT | 4.00 | 0.61 | 1.63 | 0.10 | Hs.83086 | Homo sapiens | 2.45 |
| | | | | | | | GT212 mRNA | |
| | | | | | | | [Swissprot: non | |
| 803. | TCTTTCGTCTG | 4.00 | 0.61 | 1.63 | 0.10 | Hs.87138 | ESTs | 2.45 |
| | | | | | | | [Swissprot: | |
| | | | | | | | none] | |
| 804. | TAATCTTFACT | 4.00 | 0.61 | 1.63 | 0.10 | Hs.90744 | proteasome | 2.45 |
| | | | | | | | (prosome, | |
| | | | | | | | macropain) 26S | |
| | | | | | | | subu | |
| 805. | CTTTTGTCAGC | 4.00 | 0.61 | 1.63 | 0.10 | Hs.90858 | Homo sapiens | 2.45 |
| | | | | | | | clone 25023 | |
| | | | | | | | mRNA sequence | |
| 806. | TCACGCGCTCC | 4.00 | 0.61 | 1.63 | 0.10 | Hs.93231 | ESTs | 2.45 |
| | | | | | | | [Swissprot: | |
| | | | | | | | none] | |
| 807. | ACCAGGCAAGG | 4.00 | 0.61 | 1.63 | 0.10 | Hs.93871 | hypothetical | 2.45 |
| | | | | | | | protein | |
| | | | | | | | FLJ10783 | |
| | | | | | | | [Swisspro | |
| 808. | AGTTTGGGCTG | 4.00 | 0.61 | 1.63 | 0.10 | Hs.9911 | hypothetical | 2.45 |
| | | | | | | | protein | |
| | | | | | | | FLJ11773 | |
| | | | | | | | [Swisspro | |
| 809. | CCAAATGATGA | 4.00 | 0.61 | 1.63 | 0.10 | Hs.99519 | hypothetical | 2.45 |
| | | | | | | | protein | |
| | | | | | | | FLJ14007 | |
| | | | | | | | [Swisspro | |
| 810. | TCAAATGCATC | −3.95 | 0.65 | −1.23 | 0.07 | Hs.182447 | heterogeneous | 3.21 |
| | | | | | | | nuclear | |
| | | | | | | | ribonucleoprotein | |
| 811. | TTTCTGCACTT | −3.95 | 0.65 | −1.23 | 0.07 | Hs.306019 | Homo sapiens, | 3.21 |
| | | | | | | | Similar to | |
| | | | | | | | secretory carri | |
| 812. | CTAATAAATGC | −3.95 | 0.65 | −1.23 | 0.07 | Hs.43621 | hypothetical | 3.21 |
| | | | | | | | protein | |
| | | | | | | | MBC3205 | |
| | | | | | | | [Swissprot | |
| 813. | GTGGGGCTAGG | −3.95 | 0.65 | −1.23 | 0.07 | Hs.75180 | protein | 3.21 |
| | | | | | | | phosphatase 5, | |
| | | | | | | | catalytic subunit | |
| 814. | ACAGTCTTGCC | −3.95 | 0.65 | −1.23 | 0.07 | Hs.77665 | KIAA0102 gene | 3.21 |
| | | | | | | | product | |
| | | | | | | | [Swissprot: | |
| | | | | | | | sp\|Q1 | |
| 815. | CCTGTAAAGCC | −3.95 | 0.65 | −1.23 | 0.07 | Hs.9691 | Homo sapiens | 3.21 |
| | | | | | | | cDNA: | |
| | | | | | | | FLJ23249 fis, | |
| | | | | | | | clone C | |
| 816. | GCGGGAGGGCT | −4.93 | 0.89 | −1.23 | 0.07 | Hs.154162 | ADP-ribosylation | 4.01 |
| | | | | | | | factor-like 2 | |
| | | | | | | | [Swisspr | |
| 817. | ATCCCTCAGTG | −4.93 | 0.89 | −1.23 | 0.07 | Hs.181243 | activating | 4.01 |
| | | | | | | | transcription | |
| | | | | | | | factor 4 (tax-r | |
| 818. | ACTCAGAAGAG | −4.93 | 0.89 | −1.23 | 0.07 | Hs.198272 | NADH | 4.01 |
| | | | | | | | dehydrogenase | |
| | | | | | | | (ubiquinone) 1 | |
| | | | | | | | beta s | |
| 819. | TTCTCTCAACT | −4.93 | 0.89 | −1.23 | 0.07 | Hs.27445 | unknown | 4.01 |
| | | | | | | | [Swissprot: | |
| | | | | | | | sp\|Q9NZZ4; sp\|Q9UL33 | |
| 820. | GATATCAGTCT | −4.93 | 0.89 | −1.23 | 0.07 | Hs.66394 | ring finger | 4.01 |
| | | | | | | | protein 4 | |
| | | | | | | | [Swissprot: | |
| | | | | | | | sp\|P7 | |
| 821. | TGTTCCCTTTG | 3.04 | 0.44 | 1.22 | 0.05 | Hs.118630 | MAX-interacting | 2.49 |
| | | | | | | | protein 1 | |
| | | | | | | | [Swissprot: s | |
| 822. | GATCAATCAGT | 3.04 | 0.44 | 1.22 | 0.05 | Hs.16530 | small inducible | 2.49 |
| | | | | | | | cytokine | |
| | | | | | | | subfamily A (Cy | |
| 823. | TGTCCTGGTTC | 3.04 | 0.44 | 1.22 | 0.05 | Hs.179665 | cyclin- | 2.49 |
| | | | | | | | dependent | |
| | | | | | | | kinase inhibitor | |
| | | | | | | | 1A (p2 | |
| 824. | AAGGGGCCTTT | 3.04 | 0.44 | 1.22 | 0.05 | Hs.26208 | collagen, type | 2.49 |
| | | | | | | | XVI, alpha 1 | |
| | | | | | | | [Swissprot: | |
| 825. | TCTTCTTTCAG | 3.04 | 0.44 | 1.22 | 0.05 | Hs.287830 | Homo sapiens | 2.49 |
| | | | | | | | mRNA; cDNA | |
| | | | | | | | DKFZp434E1515 | |
| | | | | | | | (f | |
| 826. | ATCATAGCTCA | 3.04 | 0.44 | 1.22 | 0.05 | Hs.309821 | EST [Swissprot: | 2.49 |
| | | | | | | | none] | |
| 827. | GATGAACACTG | 3.04 | 0.44 | 1.22 | 0.05 | Hs.32826 | CGI-130 protein | 2.49 |
| | | | | | | | [Swissprot: | |
| | | | | | | | sp\|Q9BTT2; s | |
| 828. | TGCAAAAAAAA | 3.04 | 0.44 | 1.22 | 0.05 | Hs.328801 | ESTs | 2.49 |
| | | | | | | | [Swissprot: | |
| | | | | | | | none] | |
| 829. | GGGGCTGTGGC | 3.04 | 0.44 | 1.22 | 0.05 | Hs.331 | general | 2.49 |
| | | | | | | | transcription | |
| | | | | | | | factor IIIC, polyp | |
| 830. | TTGAATAGTGA | 3.04 | 0.44 | 1.22 | 0.05 | Hs.38516 | Homo sapiens, | 2.49 |
| | | | | | | | clone | |
| | | | | | | | MGC: 15887 | |
| | | | | | | | IMAGE: 3530 | |
| 831. | TGACCACCCTT | 3.04 | 0.44 | 1.22 | 0.05 | Hs.42390 | nasopharyngeal | 2.49 |
| | | | | | | | carcinoma | |
| | | | | | | | susceptibility | |
| 832. | CTTAATCTTGT | 3.04 | 0.44 | 1.22 | 0.05 | Hs.75462 | BTG family, | 2.49 |
| | | | | | | | member 2 | |
| | | | | | | | [Swissprot: | |
| | | | | | | | sp\|P78 | |
| 833. | GCTGTTCATTG | 3.04 | 0.44 | 1.22 | 0.05 | Hs.77306 | survival of motor | 2.49 |
| | | | | | | | neuron 2, | |
| | | | | | | | centromeric | |
| 834. | TTCTGGCTGCG | 4.05 | 0.67 | 1.09 | 0.02 | Hs.119251 | ubiquinol- | 3.72 |
| | | | | | | | cytochrome c | |
| | | | | | | | reductase core | |
| | | | | | | | pr | |
| 835. | CACTCGTGTGA | 4.05 | 0.67 | 1.09 | 0.02 | Hs.146409 | cell division | 3.72 |
| | | | | | | | cycle 42 (GTP- | |
| | | | | | | | binding prot | |
| 836. | CGCCTATAATC | 4.05 | 0.67 | 1.09 | 0.02 | Hs.194110 | hypothetical | 3.72 |
| | | | | | | | protein | |
| | | | | | | | PRO2730 | |
| | | | | | | | [Swissprot | |
| 837. | TCTCCAGGAAC | 4.05 | 0.67 | 1.09 | 0.02 | Hs.237924 | CGI-69 protein | 3.72 |
| | | | | | | | [Swissprot: | |
| | | | | | | | sp\|Q9BZJ4;] | |

TABLE 11

| | | Scalp/ | | Scalp/ | | | |
|---|---|---|---|---|---|---|---|
| No. | Tag sequence | Face | Sign. | Breast | Sign. | Annotation | Description |
| 838. | GCGAAACCCCA | −2.14 | 0.91 | −8.39 | 8.72 | Hs.287478 | Homo sapiens cDNA |
| | | | | | | | FLJ12009 fis, clone HE |
| 839. | CCTGTGATCCC | −2.96 | 0.42 | −28.24 | 6.89 | Hs.347176 | Homo sapiens mRNA; |
| | | | | | | | cDNA DKFZp586H0718 (f [symbol] |
| 840. | CCTGTAATTCC | −2.96 | 0.73 | −15.35 | 6.66 | Hs.317508 | ESTs [Swissprot: none] |
| 841. | CCCAACGCGCT | −2.30 | 0.63 | −10.23 | 5.92 | Hs.347939 | hemoglobin, alpha 2 |
| | | | | | | | [Swissprot: sp\|P019 |
| 842. | CCTGCAATCCC | −1.97 | 0.33 | −13.50 | 5.71 | Hs.3280 | caspase 6, apoptosis- |
| | | | | | | | related cysteine pr |
| 843. | TCTGTAATCCC | −1.97 | 0.45 | −9.41 | 5.31 | Hs.142 | sulfotransferase family, |
| | | | | | | | cytosolic, 1A, |
| 844. | CCACTGCATTC | −2.22 | 0.73 | −7.06 | 4.71 | Hs.347796 | ESTs [Swissprot: none] |
| 845. | AATCTAGTTCT | −2.96 | 0.73 | −10.44 | 4.16 | Hs.251440 | EST, Highly similar to |
| | | | | | | | A48118 major epid |
| 846. | GAGAATGACAG | 2.37 | 1.14 | 22.81 | 3.58 | Hs.99376 | ESTs [Swissprot: none] |
| 847. | GTGAAACTCCG | −1.97 | 0.45 | −6.96 | 3.56 | Hs.285737 | Homo sapiens cDNA: |
| | | | | | | | FLJ20895 fis, clone A |
| 848. | GTGAAACCCTA | −1.97 | 0.33 | −9.21 | 3.56 | Hs.326711 | Homo sapiens, clone |
| | | | | | | | MGC: 12257 IMAGE: 3950 |
| 849. | CTGAGACAAAG | 1.91 | 0.94 | 13.85 | 3.40 | Hs.101025 | basic transcription factor 3 |
| | | | | | | | [Swissprot |
| 850. | TGGGTGAAAAA | 2.79 | 1.14 | 17.92 | 2.80 | Hs.148725 | ESTs [Swissprot: none] |
| 851. | TAATGGTAACT | 2.03 | 0.90 | 11.40 | 2.69 | Hs.181028 | cytochrome c oxidase |
| | | | | | | | subunit Va [Swissp |
| 852. | TGCAGCACGAG | −1.97 | 0.33 | −7.37 | 2.67 | Hs.110309 | major histocompatibility |
| | | | | | | | complex, class |
| 853. | GTGAAATCCCG | −1.97 | 0.33 | −7.37 | 2.67 | Hs.344010 | Homo sapiens cDNA: |
| | | | | | | | FLJ23128 fis, clone L |
| 854. | TAAATGAATAA | 2.53 | 0.95 | 16.29 | 2.55 | Hs.332404 | CDA02 protein |
| | | | | | | | [Swissprot: |
| | | | | | | | sp\|Q96EW9; sp\| |
| 855. | TTTCTGTATGT | 2.03 | 0.70 | 16.29 | 2.55 | Hs.180877 | H3 histone, family 3B |
| | | | | | | | (H3.3B) [Swisspro |
| 856. | TTCCCTTCTTC | 2.03 | 1.19 | 4.07 | 2.32 | Hs.814 | major histocompatibility |
| | | | | | | | complex, class |
| 857. | GCGTCGGTGCA | 2.43 | 1.04 | 9.77 | 2.23 | Hs.155597 | (Manual assignment) |
| | | | | | | | Adipsin, minor tag [ |
| 858. | TGGAGAGCAAC | 2.43 | 1.04 | 9.77 | 2.23 | Hs.4113 | S-adenosylhomocysteine |
| | | | | | | | hydrolase-like 1 |
| 859. | GAGAACCGTAG | 2.70 | 0.86 | 13.03 | 2.03 | Hs.105547 | neural proliferation, |
| | | | | | | | differentiation an |
| 860. | TCGTAACGAGG | 2.03 | 0.59 | 13.03 | 2.03 | Hs.11197 | hypothetical protein |
| | | | | | | | FLJ14987 [Swisspro |
| 861. | ACCTGTATCCC | −2.96 | 1.28 | −3.99 | 2.02 | Hs.182241 | interferon induced |
| | | | | | | | transmembrane protein |
| 862. | ATGGAGACTTC | 2.79 | 1.14 | 8.96 | 2.00 | Hs.239760 | citrate synthase |
| | | | | | | | [Swissprot: sp\|O75390; |
| 863. | TTTACAAAGAG | 2.23 | 0.87 | 8.96 | 2.00 | Hs.75360 | carboxypeptidase E |
| | | | | | | | [Swissprot: sp\|P1687 |
| 864. | CCTGTAGTCCT | −1.97 | 0.33 | −5.52 | 1.81 | Hs.179657 | plasminogen activator, |
| | | | | | | | urokinase recepto |
| 865. | TTGTCGATGGG | 2.53 | 0.95 | 8.15 | 1.77 | Hs.55505 | hypothetical protein |
| | | | | | | | FLJ20442 [Swisspro |
| 866. | ATAGAGGCAAT | 2.37 | 0.66 | 11.40 | 1.77 | Hs.173714 | MORF-related gene X |
| | | | | | | | [Swissprot: sp\|Q150 |
| 867. | TGTTCTCCATT | 2.37 | 0.66 | 11.40 | 1.77 | Hs.182255 | non-histone chromosome |
| | | | | | | | protein 2 (S. cer |
| 868. | CAGACCATTGT | 2.37 | 0.66 | 11.40 | 1.77 | Hs.211612 | SEC24 (S. cerevisiae) |
| | | | | | | | related gene famil |
| 869. | GCTTATAGTCA | 2.37 | 0.66 | 11.40 | 1.77 | Hs.256697 | histidine triad nucleotide- |
| | | | | | | | binding prote |
| 870. | TCAATAAAGAA | 1.91 | 0.94 | 3.46 | 1.77 | Hs.79322 | glutaminyl-tRNA |
| | | | | | | | synthetase [Swissprot: |
| 871. | CACACAGTTTT | −2.96 | 0.42 | −8.59 | 1.75 | Hs.204354 | ras homolog gene family, |
| | | | | | | | member B [Swis |
| 872. | GGAGCCATTCT | 2.03 | 0.80 | 4.89 | 1.66 | Hs.272630 | ATPase, H+ transporting |
| | | | | | | | lysosomal (vacuo |
| 873. | TGAATGGCCTA | 2.28 | 0.77 | 7.33 | 1.55 | Hs.20597 | host cell factor homolog |
| | | | | | | | [Swissprot: sp |
| 874. | AGCCTTTGTTG | −2.47 | 0.53 | −4.91 | 1.54 | Hs.9930 | serine (or cysteine) |
| | | | | | | | proteinase inhibito |
| 875. | ATGGCGATCTA | 2.03 | 0.48 | 9.77 | 1.51 | Hs.180450 | ribosomal protein S24 |
| | | | | | | | [Swissprot: sp\|P1 |
| 876. | GTATTGGCCTT | 2.03 | 0.48 | 9.77 | 1.51 | Hs.28757 | transmembrane 9 |
| | | | | | | | superfamily member 2 [S [symbol] |
| 877. | TGGCCAATAAA | 2.03 | 0.48 | 9.77 | 1.51 | Hs.57988 | hypothetical protein |
| | | | | | | | FLJ22357 similar to |
| 878. | TGACTTTTCTG | 2.03 | 0.48 | 9.77 | 1.51 | Hs.62112 | zinc finger protein 207 |
| | | | | | | | [Swissprot: sp\| |
| 879. | AGTAAACCATC | 2.03 | 0.48 | 9.77 | 1.51 | Hs.80285 | Homo sapiens mRNA; |
| | | | | | | | cDNA DKFZp586C1723 (f [symbol] |
| 880. | TAACATTAAAG | 2.03 | 0.48 | 9.77 | 1.51 | Hs.84981 | X-ray repair |
| | | | | | | | complementing defective |
| | | | | | | | rep |
| 881. | AGCCTGCAGAA | −2.96 | 0.42 | −7.37 | 1.45 | Hs.10927 | hypothetical protein |
| | | | | | | | R33729_1 [Swisspro |
| 882. | GGGAAACCCCA | −2.96 | 0.42 | −7.37 | 1.45 | Hs.306865 | Homo sapiens cDNA: |
| | | | | | | | FLJ23041 fis, clone L |
| 883. | TTTATTGAAAA | 2.70 | 0.86 | 6.52 | 1.33 | Hs.43910 | CD164 antigen, |
| | | | | | | | sialomucin [Swissprot: s |
| 884. | CGTGGGGCTGC | 2.03 | 0.59 | 6.52 | 1.33 | Hs.298023 | aquaporin 5 [Swissprot: |
| | | | | | | | none] |
| 885. | GGAGAGACAGG | 2.03 | 0.59 | 6.52 | 1.33 | Hs.46366 | hypothetical protein |
| | | | | | | | [Swissprot: sp\|Q96 |

TABLE 12

| | | | | | | | | Quotlent |
|---|---|---|---|---|---|---|---|---|
| | | | | | | | | (scalp/ |
| | | | | | | | | face)/ |
| | | Scalp/ | | Scalp/ | | | | (scalp/ |
| No. | Tag sequence | face | Sign. | breast | Sign. | Annotation | Description | breast) |
| 886. | ATCTCGGCTCA | −2.96 | 0.73 | −4.30 | 1.27 | Hs.327030 | EST, Weakly | 0.69 |
| | | | | | | | similar to | |
| | | | | | | | S65657 alpha- | |
| | | | | | | | 1C-a | |
| 887. | CTAAGTAGAGT | 2.53 | 0.55 | 8.15 | 1.25 | Hs.111301 | matrix | 0.31 |
| | | | | | | | metalloproteinase | |
| | | | | | | | 2 (gelatinase A [symbol] | |
| 888. | TCTGGCAGTAG | 2.53 | 0.55 | 8.15 | 1.25 | Hs.113503 | karyopherin | 0.31 |
| | | | | | | | (importin) beta 3 | |
| | | | | | | | [Swisspro | |
| 889. | ATGGCGGCGAT | 2.53 | 0.55 | 8.15 | 1.25 | Hs.165590 | ribosomal | 0.31 |
| | | | | | | | protein S13 | |
| | | | | | | | [Swissprot: | |
| | | | | | | | sp\|Q0 | |
| 890. | AATGCTGGCAA | 2.53 | 0.55 | 8.15 | 1.25 | Hs.181195 | DnaJ (Hsp40) | 0.31 |
| | | | | | | | homolog, | |
| | | | | | | | subfamily B, | |
| | | | | | | | membe | |
| 891. | CAATTGTAAAT | 2.53 | 0.55 | 8.15 | 1.25 | Hs.18792 | thioredoxin-like, | 0.31 |
| | | | | | | | 32 kD | |
| | | | | | | | [Swissprot: sp\|O [symbol] | |
| 892. | TGGTAACTGGC | 2.53 | 0.55 | 8.15 | 1.25 | Hs.19385 | CGI-58 protein | 0.31 |
| | | | | | | | [Swissprot: | |
| | | | | | | | sp\|Q9Y369;] | |
| 893. | TTCTCTACAAG | 2.53 | 0.55 | 8.15 | 1.25 | Hs.20157 | hypothetical | 0.31 |
| | | | | | | | protein | |
| | | | | | | | FLJ13660 | |
| | | | | | | | similar to | |
| 894. | TGTGAATAAAG | 2.53 | 0.55 | 8.15 | 1.25 | Hs.22412 | hypothetical | 0.31 |
| | | | | | | | protein | |
| | | | | | | | MGC3035 | |
| | | | | | | | [Swissprot | |
| 895. | TAAGGACGAGA | 2.53 | 0.55 | 8.15 | 1.25 | Hs.238707 | hypothetical | 0.31 |
| | | | | | | | protein | |
| | | | | | | | FLJ22457 | |
| | | | | | | | [Swisspro | |
| 896. | TAATGACAATA | 2.53 | 0.55 | 8.15 | 1.25 | Hs.239069 | four and a half | 0.31 |
| | | | | | | | LIM domains 1 | |
| | | | | | | | [Swisspro | |
| 897. | ATAACTTTGAG | 2.53 | 0.55 | 8.15 | 1.25 | Hs.307011 | keratin | 0.31 |
| | | | | | | | associated | |
| | | | | | | | protein 9.8 | |
| | | | | | | | [Swisspr | |
| 898. | CTATTCACTGT | 2.53 | 0.55 | 8.15 | 1.25 | Hs.42959 | KIAA1012 | 0.31 |
| | | | | | | | protein | |
| | | | | | | | [Swissprot: | |
| | | | | | | | sp\|Q9H0L2; | |
| 899. | CTTAAATCTGG | 2.53 | 0.55 | 8.15 | 1.25 | Hs.94 | DnaJ (Hsp40) | 0.31 |
| | | | | | | | homolog, | |
| | | | | | | | subfamily A, | |
| | | | | | | | membe | |
| 900. | GTTCTGGTTTA | −1.97 | 0.45 | −3.27 | 1.16 | Hs.241336 | ATPase inhibitor | 0.60 |
| | | | | | | | precursor | |
| | | | | | | | [Swissprot: | |
| 901. | GTGAAACACCG | −2.96 | 0.42 | −6.14 | 1.15 | Hs.297962 | Homo sapiens, | 0.48 |
| | | | | | | | clone | |
| | | | | | | | IMAGE: 3346445, | |
| | | | | | | | mRNA | |
| 902. | TTTCAGGGGAG | 2.23 | 0.87 | 2.99 | 1.06 | Hs.118126 | protective | 0.75 |
| | | | | | | | protein for beta- | |
| | | | | | | | galactosidas | |
| 903. | ATGTACTCTGG | −1.97 | 0.33 | −3.68 | 1.01 | Hs.75432 | IMP (inosine | 0.54 |
| | | | | | | | monophosphate) | |
| | | | | | | | dehydrogenas | |
| 904. | TTTGTGTTGTA | 2.03 | 0.35 | 6.52 | 0.99 | Hs.101302 | collagen, type | 0.31 |
| | | | | | | | XII. alpha 1 | |
| | | | | | | | [Swissprot: | |
| 905. | GGAAGCTAAGT | 2.03 | 0.35 | 6.52 | 0.99 | Hs.136348 | osteoblast | 0.31 |
| | | | | | | | specific factor 2 | |
| | | | | | | | (fasciclin | |
| 906. | CCCATTCCTCG | 2.03 | 0.35 | 6.52 | 0.99 | Hs.152151 | plakophilin 4 | 0.31 |
| | | | | | | | [Swissprot: | |
| | | | | | | | sp\|O95645; sp\| | |
| 907. | GATAGGTCGGG | 2.03 | 0.35 | 6.52 | 0.99 | Hs.154721 | aconitase 1, | 0.31 |
| | | | | | | | soluble | |
| | | | | | | | [Swissprot: | |
| | | | | | | | sp\|P21 | |
| 908. | GCACAAGTTCT | 2.03 | 0.35 | 6.52 | 0.99 | Hs.155106 | receptor | 0.31 |
| | | | | | | | (calcitonin) | |
| | | | | | | | activity | |
| | | | | | | | modifying | |
| 909. | GTTTGTCAATG | 2.03 | 0.35 | 6.52 | 0.99 | Hs.163565 | ESTs | 0.31 |
| | | | | | | | [Swissprot: | |
| | | | | | | | none] | |
| 910. | CGCACACACAT | 2.03 | 0.35 | 6.52 | 0.99 | Hs.172690 | diacylglycerol | 0.31 |
| | | | | | | | kinase, alpha | |
| | | | | | | | (80 kD) [Sw | |
| 911. | CAGTTCCATAA | 2.03 | 0.35 | 6.52 | 0.99 | Hs.180145 | HSPC030 | 0.31 |
| | | | | | | | protein | |
| | | | | | | | [Swissprot: | |
| | | | | | | | sp\|Q9P085; s | |
| 912. | GATGGCTGCCT | 2.03 | 0.35 | 6.52 | 0.99 | Hs.18104 | hypothetical | 0.31 |
| | | | | | | | protein | |
| | | | | | | | FLJ11274 | |
| | | | | | | | [Swisspro | |
| 913. | AGACTAAGGTT | 2.03 | 0.35 | 6.52 | 0.99 | Hs.22635 | ESTs | 0.31 |
| | | | | | | | [Swissprot: | |
| | | | | | | | none] | |
| 914. | GCCTTATGTAT | 2.03 | 0.35 | 6.52 | 0.99 | Hs.250697 | ras-like protein | 0.31 |
| | | | | | | | [Swissprot: | |
| | | | | | | | sp\|P17081; | |
| 915. | ATGGAATGCTA | 2.03 | 0.35 | 6.52 | 0.99 | Hs.268551 | receptor- | 0.31 |
| | | | | | | | interacting | |
| | | | | | | | serine-threonine | |
| | | | | | | | ki | |
| 916. | CTGTTTGTTCA | 2.03 | 0.35 | 6.52 | 0.99 | Hs.288965 | Homo sapiens | 0.31 |
| | | | | | | | cDNA: | |
| | | | | | | | FLJ22300 fis, | |
| | | | | | | | clone H | |
| 917. | GATGTGTGCTT | 2.03 | 0.35 | 6.52 | 0.99 | Hs.301005 | histone H2A.F/Z | 0.31 |
| | | | | | | | variant | |
| | | | | | | | [Swissprot: non | |
| 918. | CCACAGTAGAT | 2.03 | 0.35 | 6.52 | 0.99 | Hs.62112 | zinc finger | 0.31 |
| | | | | | | | protein 207 | |
| | | | | | | | [Swissprot: sp\| | |
| 919. | AAACCTGGGAA | 2.03 | 0.35 | 6.52 | 0.99 | Hs.63788 | propionyl | 0.31 |
| | | | | | | | Coenzyme A | |
| | | | | | | | carboxylase, | |
| | | | | | | | beta p | |
| 920. | CTGTAACATAT | 2.03 | 0.35 | 6.52 | 0.99 | Hs.75790 | phosphatidylinositol | 0.31 |
| | | | | | | | glycan, class | |
| | | | | | | | C [S | |
| 921. | TTTTGGATGTA | 2.03 | 0.35 | 6.52 | 0.99 | Hs.75875 | ubiquitin- | 0.31 |
| | | | | | | | conjugating | |
| | | | | | | | enzyme E2 | |
| | | | | | | | variant | |
| 922. | CTGGGCCTGAA | 2.03 | 0.35 | 6.52 | 0.99 | Hs.76507 | LPS-induced | 0.31 |
| | | | | | | | TNF-alpha factor | |
| | | | | | | | [Swissprot | |
| 923. | TATATCAGTGT | 2.03 | 0.35 | 6.52 | 0.99 | Hs.90336 | ATPase, H+ | 0.31 |
| | | | | | | | transporting, | |
| | | | | | | | lysosomal (vacu [symbol] | |
| 924. | GGTGAGGGAGG | 2.03 | 0.35 | 6.52 | 0.99 | Hs.9071 | progesterone | 0.31 |
| | | | | | | | receptor | |
| | | | | | | | membrane | |
| | | | | | | | component | |
| 925. | CAGTGTATATA | 2.03 | 0.48 | 4.89 | 0.89 | Hs.108725 | HSPC040 | 0.42 |
| | | | | | | | protein | |
| | | | | | | | [Swissprot: | |
| | | | | | | | sp\|Q9HB67; s | |
| 926. | GGGAAGGCACT | 2.03 | 0.48 | 4.89 | 0.89 | Hs.13144 | HSPC160 | 0.42 |
| | | | | | | | protein | |
| | | | | | | | [Swissprot: | |
| | | | | | | | sp\|Q9P004;] | |
| 927. | ACTGATGCAAG | 2.03 | 0.48 | 4.89 | 0.89 | Hs.161049 | ESTs | 0.42 |
| | | | | | | | [Swissprot: | |
| | | | | | | | none] | |
| 928. | GCAGTGCCACT | 2.03 | 0.48 | 4.89 | 0.89 | Hs.22972 | hypothetical | 0.42 |
| | | | | | | | protein | |
| | | | | | | | FLJ13352 | |
| | | | | | | | [Swisspro | |
| 929. | GTGCCTAGGAG | 2.03 | 0.48 | 4.89 | 0.89 | Hs.25999 | hypothetical | 0.42 |
| | | | | | | | protein | |
| | | | | | | | FLJ22195 | |
| | | | | | | | [Swisspro | |
| 930. | CGCTTTTGTAG | 2.03 | 0.48 | 4.89 | 0.89 | Hs.5297 | DKFZP564A2416 | 0.42 |
| | | | | | | | protein | |
| | | | | | | | [Swissprot: | |
| | | | | | | | sp\|Q9 | |
| 931. | GAGAAGACTTC | 2.03 | 0.48 | 4.89 | 0.89 | Hs.86978 | prolyl | 0.42 |
| | | | | | | | endopeptidase | |
| | | | | | | | [Swissprot: | |
| | | | | | | | sp\|P48 | |
| 932. | ACCTTGTGCCC | 2.03 | 0.48 | 4.89 | 0.89 | Hs.878 | sorbitol | 0.42 |
| | | | | | | | dehydrogenase | |
| | | | | | | | [Swissprot: sp\|Q [symbol] | |
| 933. | GTGCTGGAGAA | 2.70 | 0.86 | 3.26 | 0.88 | Hs.53125 | small nuclear | 0.83 |
| | | | | | | | ribonucleoprotein | |
| | | | | | | | D2 polyp | |
| 934. | AGCCCTCCCTG | 2.03 | 0.59 | 3.26 | 0.88 | Hs.74111 | RNA-binding | 0.62 |
| | | | | | | | protein | |
| | | | | | | | (autoantigenic) | |
| | | | | | | | [Sw | |
| 935. | GGTGGGGAGAT | −2.96 | 0.42 | −4.91 | 0.86 | Hs.157236 | membrane | 0.60 |
| | | | | | | | protein of | |
| | | | | | | | cholinergic | |
| | | | | | | | synaptic | |
| 936. | GGCAACGTGGT | −2.96 | 0.42 | −4.91 | 0.86 | Hs.300954 | Huntingtin | 0.60 |
| | | | | | | | interacting | |
| | | | | | | | protein K [Swiss | |
| 937. | TGTTTGTGTGT | −2.96 | 0.42 | −4.91 | 0.86 | Hs.343214 | Homo sapiens, | 0.60 |
| | | | | | | | clone | |
| | | | | | | | MGC: 19762 | |
| | | | | | | | IMAGE: 3636 | |
| 938. | AGGATGACCAG | −2.96 | 0.42 | −4.91 | 0.86 | Hs.69554 | hypothetical | 0.60 |
| | | | | | | | protein | |
| | | | | | | | FLJ20552 | |
| | | | | | | | [Swisspro | |
| 939. | AAGCTGAGTGG | −2.96 | 0.42 | −4.91 | 0.86 | Hs.79024 | heterogeneous | 0.60 |
| | | | | | | | nuclear | |
| | | | | | | | ribonucleoprotein | |
| 940. | AAAGTCTAGAA | −1.97 | 0.75 | −2.05 | 0.79 | Hs.82932 | cyclin D1 | 0.96 |
| | | | | | | | (PRAD1: | |
| | | | | | | | parathyroid | |
| | | | | | | | adenomatos | |
| 941. | CCCATAATCCC | 2.23 | 0.87 | 2.24 | 0.76 | Hs.111256 | arachidonate 15- | 1.00 |
| | | | | | | | lipoxygenase, | |
| | | | | | | | second typ | |
| 942. | CGGATAACCAG | −2.47 | 0.53 | −3.07 | 0.76 | Hs.343258 | proliferation- | 0.80 |
| | | | | | | | associated 2G4, | |
| | | | | | | | 38 kD [Swi | |
| 943. | TGGTACACGTA | 2.37 | 1.14 | 1.90 | 0.71 | Hs.279574 | CGI-39 protein; | 1.25 |
| | | | | | | | cell death- | |
| | | | | | | | regulatory pr | |
| 944. | GGGTCAAAAGG | 2.03 | 0.90 | 1.90 | 0.71 | Hs.181307 | H3 histone, | 1.07 |
| | | | | | | | family 3A | |
| | | | | | | | [Swissprot: | |
| | | | | | | | sp\|P0 | |
| 945. | GTATTCCCCTT | −1.97 | 0.45 | −2.46 | 0.70 | Hs.117176 | poly(A)-binding | 0.80 |
| | | | | | | | protein, nuclear | |
| | | | | | | | 1 [Swi | |
| 946. | GATCAGGCCAG | −1.97 | 0.45 | −2.46 | 0.70 | Hs.119571 | collagen, type | 0.80 |
| | | | | | | | III, alpha 1 | |
| | | | | | | | (Ehlers-Danl | |
| 947. | AGGCAGGACGG | 2.53 | 0.55 | 4.07 | 0.68 | Hs.133081 | ESTs, Weakly | 0.62 |
| | | | | | | | similar to | |
| | | | | | | | T08700 | |
| | | | | | | | hypotheti | |
| 948. | AAATTTTAAAA | 2.53 | 0.55 | 4.07 | 0.68 | Hs.142442 | HP1-BP74 | 0.62 |
| | | | | | | | [Swissprot: | |
| | | | | | | | sp\|Q9UHY0;] | |
| 949. | TTTTAGCAGGA | 2.53 | 0.55 | 4.07 | 0.68 | Hs.146393 | homocysteine- | 0.62 |
| | | | | | | | inducible, | |
| | | | | | | | endoplasmic reti [symbol] | |
| 950. | GTCACAACCTG | 2.53 | 0.55 | 4.07 | 0.68 | Hs.159608 | aldehyde | 0.62 |
| | | | | | | | dehydrogenase | |
| | | | | | | | 3 family, | |
| | | | | | | | member | |
| 951. | AAAGCAAACCA | 2.53 | 0.55 | 4.07 | 0.68 | Hs.2331 | E2F transcription | 0.62 |
| | | | | | | | factor 5, p130- | |
| | | | | | | | binding | |
| 952. | GAGAATCTGCT | 2.53 | 0.55 | 4.07 | 0.68 | Hs.23960 | cyclin B1 | 0.62 |
| | | | | | | | [Swissprot: | |
| | | | | | | | sp\|P14635; sp\|Q9BP | |
| 953. | CACCGCTGCAG | 2.53 | 0.55 | 4.07 | 0.68 | Hs.275215 | hydroxysteroid | 0.62 |
| | | | | | | | (11-beta) | |
| | | | | | | | dehydrogenase 1 | |
| 954. | CAGGGTGGGTG | 2.53 | 0.55 | 4.07 | 0.68 | Hs.278222 | Homo sapiens | 0.62 |
| | | | | | | | cDNA FLJ14885 | |
| | | | | | | | fis, clone PL | |
| 955. | CAGTTCTTGAT | 2.53 | 0.55 | 4.07 | 0.68 | Hs.284217 | serologically | 0.62 |
| | | | | | | | defined colon | |
| | | | | | | | cancer antig | |
| 956. | TTGGTGTGCTG | 2.53 | 0.55 | 4.07 | 0.68 | Hs.288552 | hypothetical | 0.62 |
| | | | | | | | protein | |
| | | | | | | | FLJ23558 | |
| | | | | | | | [Swisspro | |
| 957. | CCATTTTTACC | 2.53 | 0.55 | 4.07 | 0.68 | Hs.59271 | U2(RNU2) small | 0.62 |
| | | | | | | | nuclear RNA | |
| | | | | | | | auxillary fac | |
| 958. | CAGATAACATA | 2.53 | 0.55 | 4.07 | 0.68 | Hs.75187 | translocase of | 0.62 |
| | | | | | | | outer | |
| | | | | | | | mitochondrial | |
| | | | | | | | membr | |
| 959. | CGATGGTCCCC | 2.53 | 0.55 | 4.07 | 0.68 | Hs.7771 | B-cell associated | 0.62 |
| | | | | | | | protein | |
| | | | | | | | [Swissprot: s | |
| 960. | CTCATATGTTA | 2.53 | 0.55 | 4.07 | 0.68 | Hs.8939 | Yes-associated | 0.62 |
| | | | | | | | protein 1, 65 kDa | |
| | | | | | | | [Swiss | |
| 961. | CAGCAGAAGCA | −2.22 | 0.73 | −2.15 | 0.66 | Hs.323806 | small EDRK-rich | 1.03 |
| | | | | | | | factor 2 | |
| | | | | | | | [Swissprot: sp | |
| 962. | GCCCTTTCTCT | −1.97 | 0.56 | −2.15 | 0.66 | Hs.7835 | endocytic | 0.92 |
| | | | | | | | receptor | |
| | | | | | | | (macrophage | |
| | | | | | | | mannose r | |
| 963. | CTCAACCCCCC | −1.97 | 0.56 | −2.15 | 0.66 | Hs.89137 | low density | 0.92 |
| | | | | | | | lipoprotein- | |
| | | | | | | | related protein | |
| 964. | AAGTGATTCTG | −2.96 | 0.42 | −3.68 | 0.58 | Hs.180677 | zinc finger | 0.80 |
| | | | | | | | protein 162 | |
| | | | | | | | [Swissprot: sp\| | |
| 965. | GGGCCCAGGGG | −2.96 | 0.42 | −3.68 | 0.58 | Hs.3803 | reticulon 2 | 0.80 |
| | | | | | | | [Swissprot: | |
| | | | | | | | sp\|O75298; sp\|Q9 | |
| 966. | TAGTTGTAGGG | −2.96 | 0.42 | −3.68 | 0.58 | Hs.5324 | hypothetical | 0.80 |
| | | | | | | | protein | |
| | | | | | | | [Swissprot: | |
| | | | | | | | sp\|O95 | |
| 967. | AATTTGCAACA | −2.96 | 0.42 | −3.68 | 0.58 | Hs.75258 | H2A histone | 0.80 |
| | | | | | | | family, member | |
| | | | | | | | Y [Swissprot | |
| 968. | CAGAGACGTGG | −2.96 | 0.42 | −3.68 | 0.58 | Hs.76111 | dystroglycan 1 | 0.80 |
| | | | | | | | (dystrophin- | |
| | | | | | | | associated gl | |
| 969. | GCAGCTAATTT | −2.96 | 0.42 | −3.68 | 0.58 | Hs.8207 | GK001 protein | 0.80 |
| | | | | | | | [Swissprot: | |
| | | | | | | | sp\|Q96A33; sp\| | |
| 970. | TTTTGAAGCAG | 2.03 | 0.59 | 2.17 | 0.57 | Hs.80464 | hepatitis B virus | 0.94 |
| | | | | | | | x-interacting | |
| | | | | | | | protein | |
| 971. | GACCAGAAAAA | −2.96 | 0.73 | −2.46 | 0.52 | Hs.180714 | cytochrome c | 1.20 |
| | | | | | | | oxidase subunit | |
| | | | | | | | VIa polypep | |
| 972. | CCAAGGATTGG | −2.96 | 0.73 | −2.46 | 0.52 | Hs.9003 | hypothetical | 1.20 |
| | | | | | | | protein | |
| | | | | | | | FLJ13868 | |
| | | | | | | | [Swisspro | |
| 973. | TTTCTTAAAGG | 2.03 | 0.48 | 2.44 | 0.52 | Hs.197114 | serine/arginine | 0.83 |
| | | | | | | | repetitive matrix | |
| | | | | | | | 2 [Sw | |
| 974. | CGGCACCTTAA | 2.03 | 0.48 | 2.44 | 0.52 | Hs.209100 | DKFZP434C171 | 0.83 |
| | | | | | | | protein | |
| | | | | | | | [Swissprot: | |
| | | | | | | | sp\|Q9H | |
| 975. | AGCAGGGCTCC | −1.97 | 0.33 | −2.46 | 0.52 | Hs.278027 | LIM domain | 0.80 |
| | | | | | | | kinase 2 | |
| | | | | | | | [Swissprot: | |
| | | | | | | | sp\|O003 | |
| 976. | ATTATCCAGGG | −2.96 | 1.01 | −2.05 | 0.48 | Hs.301404 | RNA binding | 1.44 |
| | | | | | | | motif protein 3 | |
| | | | | | | | [Swissprot: | |
| 977. | GCTCCCAGACT | −2.63 | 0.82 | −2.05 | 0.48 | Hs.5097 | synaptogyrin 2 | 1.28 |
| | | | | | | | [Swissprot: | |
| | | | | | | | sp\|O43760;] | |
| 978. | TTAAAAAAAAA | −2.30 | 0.63 | −2.05 | 0.48 | Hs.19054 | hypothetical | 1.12 |
| | | | | | | | protein | |
| | | | | | | | PRO2521 | |
| | | | | | | | [Swissprot | |
| 979. | TGTTTGAATTC | 2.03 | 0.21 | 3.26 | 0.48 | Hs.103422 | Homo sapiens | 0.62 |
| | | | | | | | mRNA; cDNA | |
| | | | | | | | DKFZp434F1622 | |
| | | | | | | | (f | |
| 980. | AAACTTGCTCT | 2.03 | 0.21 | 3.26 | 0.48 | Hs.104117 | cytochrome | 0.62 |
| | | | | | | | P450, subfamily | |
| | | | | | | | IIIA (niphedl | |
| 981. | ATGGCAGAGAC | 2.03 | 0.21 | 3.26 | 0.48 | Hs.104335 | hypothetical | 0.62 |
| | | | | | | | protein | |
| | | | | | | | IMAGE3510317 | |
| | | | | | | | [Swis | |
| 982. | GTTCATAGGTC | 2.03 | 0.21 | 3.26 | 0.48 | Hs.107394 | secretory protein | 0.62 |
| | | | | | | | SEC8 | |
| | | | | | | | [Swissprot: sp\|Q [symbol] | |
| 983. | TGTTTGTACAT | 2.03 | 0.21 | 3.26 | 0.48 | Hs.107526 | UDP- | 0.62 |
| | | | | | | | Gal: betaGlcNAc | |
| | | | | | | | beta 1,4- | |
| | | | | | | | galactosylt | |
| 984. | ACAACTCCTGC | 2.03 | 0.21 | 3.26 | 0.48 | Hs.108447 | spinocerebellar | 0.62 |
| | | | | | | | ataxia 7 | |
| | | | | | | | (olivopontocere | |
| 985. | CTTAATAAAAG | 2.03 | 0.21 | 3.26 | 0.48 | Hs.108548 | PABP- | 0.62 |
| | | | | | | | interacting | |
| | | | | | | | protein 2 | |
| | | | | | | | [Swissprot: | |
| 986. | GCAGAGAAAAA | 2.03 | 0.21 | 3.26 | 0.48 | Hs.109606 | coronin, actin- | 0.62 |
| | | | | | | | binding protein, | |
| | | | | | | | 1A [Swi | |
| 987. | ACTTTTAATGA | 2.03 | 0.21 | 3.26 | 0.48 | Hs.111911 | ESTs, Weakly | 0.62 |
| | | | | | | | similar to | |
| | | | | | | | MUC2_HUMAN | |
| | | | | | | | MUCIN | |
| 988. | GCGACGGCCGT | 2.03 | 0.21 | 3.26 | 0.48 | Hs.112318 | 6.2 kd protein | 0.62 |
| | | | | | | | [Swissprot: | |
| | | | | | | | sp\|Q9P0U1;] | |
| 989. | AATAGGTCCAC | 2.03 | 0.21 | 3.26 | 0.48 | Hs.113029 | ribosomal | 0.62 |
| | | | | | | | protein S25 | |
| | | | | | | | [Swissprot: | |
| | | | | | | | sp\|P2 | |
| 990. | CCTGCCAAACT | 2.03 | 0.21 | 3.26 | 0.48 | Hs.114055 | ESTs | 0.62 |
| | | | | | | | [Swissprot: | |
| | | | | | | | none] | |
| 991. | TATGCCCTATC | 2.03 | 0.21 | 3.26 | 0.48 | Hs.115740 | KIAA0210 gene | 0.62 |
| | | | | | | | product | |
| | | | | | | | [Swissprot: | |
| | | | | | | | sp\|Q9 | |
| 992. | AGCAGTGACGG | 2.03 | 0.21 | 3.26 | 0.48 | Hs.116651 | epithelial V-like | 0.62 |
| | | | | | | | antigen 1 | |
| | | | | | | | [Swissprot: | |
| 993. | GACCCTGGGGA | 2.03 | 0.21 | 3.26 | 0.48 | Hs.116708 | ESTs, Weakly | 0.62 |
| | | | | | | | similar to | |
| | | | | | | | Y063_HUMAN | |
| | | | | | | | HYPOT | |
| 994. | TCGTTTCCTTC | 2.03 | 0.21 | 3.26 | 0.48 | Hs.11806 | 7- | 0.62 |
| | | | | | | | dehydrocholesterol | |
| | | | | | | | reductase | |
| | | | | | | | [Swisspr | |
| 995. | AAGCCTTAAAA | 2.03 | 0.21 | 3.26 | 0.48 | Hs.11861 | thyroid hormone | 0.62 |
| | | | | | | | receptor- | |
| | | | | | | | associated prot | |
| 996. | GACTGTTAATG | 2.03 | 0.21 | 3.26 | 0.48 | Hs.118684 | stromal cell- | 0.62 |
| | | | | | | | derived factor 2 | |
| | | | | | | | [Swisspro | |
| 997. | AGTGTGATACT | 2.03 | 0.21 | 3.26 | 0.48 | Hs.118820 | Homo sapiens, | 0.62 |
| | | | | | | | clone | |
| | | | | | | | IMAGE: 3357862, | |
| | | | | | | | mRNA, | |
| 998. | TTTAAAAGAGC | 2.03 | 0.21 | 3.26 | 0.48 | Hs.119 | Wilms' tumour 1- | 0.62 |
| | | | | | | | associating | |
| | | | | | | | protein [Sw | |
| 999. | TGTAATGTAAC | 2.03 | 0.21 | 3.26 | 0.48 | Hs.12229 | Homo sapiens | 0.62 |
| | | | | | | | cDNA FLJ11324 | |
| | | | | | | | fis, clone PL | |
| 1000 | GCAGCAAGTAA | 2.03 | 0.21 | 3.26 | 0.48 | Hs.122546 | hypothetical | 0.62 |
| | | | | | | | protein | |
| | | | | | | | FLJ23017 | |
| | | | | | | | [Swisspro | |
| 1001 | CTGCACCATCT | 2.03 | 0.21 | 3.26 | 0.48 | Hs.12259 | KIAA0630 | 0.62 |
| | | | | | | | protein | |
| | | | | | | | [Swissprot: | |
| | | | | | | | sp\|O75125; | |
| 1002 | CTGCTAATAAA | 2.03 | 0.21 | 3.26 | 0.48 | Hs.122854 | ESTs | 0.62 |
| | | | | | | | [Swissprot: | |
| | | | | | | | none] | |
| 1003 | ATATGTCTCTG | 2.03 | 0.21 | 3.26 | 0.48 | Hs.125276 | ESTs | 0.62 |
| | | | | | | | [Swissprot: | |
| | | | | | | | none] | |
| 1004 | TCAATCAGTGA | 2.03 | 0.21 | 3.26 | 0.48 | Hs.127270 | KIAA1545 | 0.62 |
| | | | | | | | protein | |
| | | | | | | | [Swissprot: | |
| | | | | | | | sp\|Q9HCM7; | |
| 1005 | ATGCTAGATTT | 2.03 | 0.21 | 3.26 | 0.48 | Hs.129548 | heterogeneous | 0.62 |
| | | | | | | | nuclear | |
| | | | | | | | ribonucleoprotein | |
| 1006 | GTGGTCAGTGG | 2.03 | 0.35 | 3.26 | 0.48 | Hs.132792 | serologically | 0.62 |
| | | | | | | | defined colon | |
| | | | | | | | cancer antig | |
| 1007 | AAGCCTTATAT | 2.03 | 0.21 | 3.26 | 0.48 | Hs.134190 | ESTs | 0.62 |
| | | | | | | | [Swissprot: | |
| | | | | | | | none] | |
| 1008 | AACTCTGGACC | 2.03 | 0.21 | 3.26 | 0.48 | Hs.134406 | hypothetical | 0.62 |
| | | | | | | | protein | |
| | | | | | | | FLJ20511 | |
| | | | | | | | [Swisspro | |
| 1009 | AGGAAGACTGA | 2.03 | 0.21 | 3.26 | 0.48 | Hs.135643 | ESTs | 0.62 |
| | | | | | | | [Swissprot: | |
| | | | | | | | none] | |
| 1010 | TTAAATCGTGA | 2.03 | 0.21 | 3.26 | 0.48 | Hs.13880 | CGI-143 protein | 0.62 |
| | | | | | | | [Swissprot: | |
| | | | | | | | sp\|Q9BUA7; s | |
| 1011 | GGCAGTTAACC | 2.03 | 0.21 | 3.26 | 0.48 | Hs.1390 | proteasome | 0.62 |
| | | | | | | | (prosome, | |
| | | | | | | | macropain) | |
| | | | | | | | subunit, | |
| 1012 | GTGAATAAACA | 2.03 | 0.21 | 3.26 | 0.48 | Hs.143601 | hypothetical | 0.62 |
| | | | | | | | protein hCLA-iso | |
| | | | | | | | [Swisspro | |
| 1013 | TGTCACCTGAA | 2.03 | 0.21 | 3.26 | 0.48 | Hs.145949 | cytokeratin type | 0.62 |
| | | | | | | | II [Swissprot: | |
| | | | | | | | sp\|O956 | |
| 1014 | TTGAAATAATA | 2.03 | 0.21 | 3.26 | 0.48 | Hs.146663 | ESTs | 0.62 |
| | | | | | | | [Swissprot: | |
| | | | | | | | none] | |
| 1015 | TTCTCTCCAAC | 2.03 | 0.35 | 3.26 | 0.48 | Hs.15087 | chromosome 1 | 0.62 |
| | | | | | | | open reading | |
| | | | | | | | frame 16 [Swi | |
| 1016 | TACAGTTCAGT | 2.03 | 0.21 | 3.26 | 0.48 | Hs.151498 | ESTS | 0.62 |
| | | | | | | | [Swissprot: | |
| | | | | | | | none] | |
| 1017 | TGTTAAGTTCT | 2.03 | 0.21 | 3.26 | 0.48 | Hs.151573 | cryptochrome 1 | 0.62 |
| | | | | | | | (photolyase-like) | |
| | | | | | | | [Swiss | |
| 1018 | GGTTATTTATG | 2.03 | 0.21 | 3.26 | 0.48 | Hs.152944 | loss of | 0.62 |
| | | | | | | | heterozygosity, | |
| | | | | | | | 11, | |
| | | | | | | | chromosomal | |
| 1019 | GGTCTACATAT | 2.03 | 0.21 | 3.26 | 0.48 | Hs.154574 | ESTs | 0.62 |
| | | | | | | | [Swissprot: | |
| | | | | | | | none] | |
| 1020 | GAACAGAAAAA | 2.03 | 0.21 | 3.26 | 0.48 | Hs.156276 | KIAA0783 gene | 0.62 |
| | | | | | | | product | |
| | | | | | | | [Swissprot: | |
| | | | | | | | sp\|O9 | |
| 1021 | GGTTTTTCCCT | 2.03 | 0.21 | 3.26 | 0.48 | Hs.157103 | hypothetical | 0.62 |
| | | | | | | | protein | |
| | | | | | | | FLJ12644 | |
| | | | | | | | [Swisspro | |
| 1022 | AGGAGGGATAA | 2.03 | 0.21 | 3.26 | 0.48 | Hs.16258 | Homo sapiens, | 0.62 |
| | | | | | | | Similar to | |
| | | | | | | | RAB24, member R | |
| 1023 | GAAGGATTGGG | 2.03 | 0.21 | 3.26 | 0.48 | Hs.16475 | Human DNA | 0.62 |
| | | | | | | | sequence from | |
| | | | | | | | clone RP5- | |
| | | | | | | | 852M4 | |
| 1024 | TGTTCATTTAT | 2.03 | 0.21 | 3.26 | 0.48 | Hs.166891 | regulatory factor | 0.62 |
| | | | | | | | X, 5 (influences | |
| | | | | | | | HLA c | |
| 1025 | TAGTAGATTAA | 2.03 | 0.21 | 3.26 | 0.48 | Hs.167011 | Homo sapiens | 0.62 |
| | | | | | | | cDNA: | |
| | | | | | | | FLJ21362 fis, | |
| | | | | | | | clone C | |
| 1026 | TTACTGGGTTT | 2.03 | 0.21 | 3.26 | 0.48 | Hs.170263 | tumor protein | 0.62 |
| | | | | | | | p53-binding | |
| | | | | | | | protein, 1 [S | |
| 1027 | GGATGCATTAG | 2.03 | 0.21 | 3.26 | 0.48 | Hs.172635 | Homo sapiens | 0.62 |
| | | | | | | | cDNA: | |
| | | | | | | | FLJ21367 fis, | |
| | | | | | | | clone C | |
| 1028 | GGGCCCCCAAA | 2.03 | 0.21 | 3.26 | 0.48 | Hs.172635 | Homo sapiens | 0.62 |
| | | | | | | | cDNA: | |
| | | | | | | | FLJ21367 fis, | |
| | | | | | | | clone C | |
| 1029 | TTGTAAATAGG | 2.03 | 0.21 | 3.26 | 0.48 | Hs.172647 | golgi | 0.62 |
| | | | | | | | autoantigen, | |
| | | | | | | | golgin subfamily | |
| | | | | | | | a, 1 | |
| 1030 | AAATTAAAGTC | 2.03 | 0.21 | 3.26 | 0.48 | Hs.172766 | MAP/microtubule | 0.62 |
| | | | | | | | affinity- | |
| | | | | | | | regulating kina | |
| 1031 | GAGTTTGTGTT | 2.03 | 0.21 | 3.26 | 0.48 | Hs.173042 | KIAA1143 | 0.62 |
| | | | | | | | protein | |
| | | | | | | | [Swissprot: | |
| | | | | | | | sp\|Q96HJ8; | |
| 1032 | GTGGTAGTACC | 2.03 | 0.21 | 3.26 | 0.48 | Hs.175563 | Homo sapiens | 0.62 |
| | | | | | | | mRNA; cDNA | |
| | | | | | | | DKFZp564N076 | |
| | | | | | | | 3 (f | |
| 1033 | TGGATGTACTT | 2.03 | 0.21 | 3.26 | 0.48 | Hs.178761 | 26S | 0.62 |
| | | | | | | | proteasome- | |
| | | | | | | | associated pad1 | |
| | | | | | | | homolog | |
| 1034 | TATCCCCAAAT | 2.03 | 0.21 | 3.26 | 0.48 | Hs.180841 | tumor necrosis | 0.62 |
| | | | | | | | factor receptor | |
| | | | | | | | superfami | |
| 1035 | GCTCTGTTCAT | 2.03 | 0.21 | 3.26 | 0.48 | Hs.18192 | serine/arginine | 0.62 |
| | | | | | | | repetitive matrix | |
| | | | | | | | 1 [Sw | |
| 1036 | TCATCATATTT | 2.03 | 0.21 | 3.26 | 0.48 | Hs.182470 | PTD010 protein | 0.62 |
| | | | | | | | [Swissprot: | |
| | | | | | | | sp\|Q9H3K2; s | |
| 1037 | ATTGCTAAGTG | 2.03 | 0.21 | 3.26 | 0.48 | Hs.182470 | PTD010 protein | 0.62 |
| | | | | | | | [Swissprot: | |
| | | | | | | | sp\|Q9H3K2; s | |
| 1038 | CTTTTTCGTAT | 2.03 | 0.21 | 3.26 | 0.48 | Hs.182538 | phospholipid | 0.62 |
| | | | | | | | scramblase 4 | |
| | | | | | | | [Swissprot: s | |
| 1039 | AGTAGTCTGCA | 2.03 | 0.21 | 3.26 | 0.48 | Hs.182695 | mitochondrial | 0.62 |
| | | | | | | | ribosomal | |
| | | | | | | | protein 63 [Swi | |
| 1040 | GGGCTGGGGGT | 2.03 | 0.21 | 3.26 | 0.48 | Hs.183698 | ribosomal | 0.62 |
| | | | | | | | protein L29 | |
| | | | | | | | [Swissprot: | |
| | | | | | | | sp\|P4 | |
| 1041 | AACTGTACTAC | 2.03 | 0.21 | 3.26 | 0.48 | Hs.184050 | v-Ki-ras2 Kirsten | 0.62 |
| | | | | | | | rat sarcoma 2 | |
| | | | | | | | viral on | |
| 1042 | GGAGATGGAGC | 2.03 | 0.21 | 3.26 | 0.48 | Hs.188757 | Homo sapiens, | 0.62 |
| | | | | | | | clone | |
| | | | | | | | MGC: 5564, | |
| | | | | | | | mRNA, comp | |
| 1043 | GTTAATCTGGA | 2.03 | 0.21 | 3.26 | 0.48 | Hs.189834 | DKFZP566E104 | 0.62 |
| | | | | | | | protein | |
| | | | | | | | [Swissprot: | |
| | | | | | | | sp\|Q9U | |
| 1044 | TTTGCCTGTTA | 2.03 | 0.21 | 3.26 | 0.48 | Hs.19575 | CGI-11 protein | 0.62 |
| | | | | | | | [Swissprot: | |
| | | | | | | | sp\|Q9H3E3; sp | |
| 1045 | AATGAGAAGGT | 2.03 | 0.21 | 3.26 | 0.48 | Hs.198248 | UDP- | 0.62 |
| | | | | | | | Gal: betaGlcNAc | |
| | | | | | | | beta 1,4- | |
| | | | | | | | galactosylt | |
| 1046 | CAGATTTCTGT | 2.03 | 0.21 | 3.26 | 0.48 | Hs.198891 | serine/threonine- | 0.62 |
| | | | | | | | protein kinase | |
| | | | | | | | PRP4 hom | |
| 1047 | ATACAGTTTGG | 2.03 | 0.21 | 3.26 | 0.48 | Hs.199061 | p300/CBP- | 0.62 |
| | | | | | | | associated factor | |
| | | | | | | | [Swissprot: | |
| 1048 | CCTTCTCACTC | 2.03 | 0.21 | 3.26 | 0.48 | Hs.20013 | GCIP-interacting | 0.62 |
| | | | | | | | protein p29 | |
| | | | | | | | [Swissprot | |
| 1049 | TGTAGCCTATG | 2.03 | 0.21 | 3.26 | 0.48 | Hs.201675 | RNA binding | 0.62 |
| | | | | | | | motif protein 5 | |
| | | | | | | | [Swissprot: | |
| 1050 | GCCTGTTTGGG | 2.03 | 0.21 | 3.26 | 0.48 | Hs.2056 | UDP | 0.62 |
| | | | | | | | glycosyltransferase | |
| | | | | | | | 1 family, | |
| | | | | | | | polype | |
| 1051 | GCACTGGGGCA | 2.03 | 0.21 | 3.26 | 0.48 | Hs.206259 | Homo sapiens | 0.62 |
| | | | | | | | mRNA for | |
| | | | | | | | KIAA1190 | |
| | | | | | | | protein, | |
| 1052 | GGAACTCTGTT | 2.03 | 0.21 | 3.26 | 0.48 | Hs.209473 | hypothetical | 0.62 |
| | | | | | | | protein | |
| | | | | | | | FLJ10520 | |
| | | | | | | | [Swisspro | |
| 1053 | TCAGAGTAATC | 2.03 | 0.21 | 3.26 | 0.48 | Hs.211579 | melanoma cell | 0.62 |
| | | | | | | | adhesion | |
| | | | | | | | molecule | |
| | | | | | | | [Swissp | |
| 1054 | TTGGCCAGGGT | 2.03 | 0.35 | 3.26 | 0.48 | Hs.213010 | EST [Swissprot: | 0.62 |
| | | | | | | | none] | |
| 1055 | TGACCTATTTC | 2.03 | 0.21 | 3.26 | 0.48 | Hs.214646 | KIAA0447 gene | 0.62 |
| | | | | | | | product | |
| | | | | | | | [Swissprot: | |
| | | | | | | | sp\|O7 | |
| 1056 | GTACTGTAAGA | 2.03 | 0.21 | 3.26 | 0.48 | Hs.21610 | DKFZP434B203 | 0.62 |
| | | | | | | | protein | |
| | | | | | | | [Swissprot: | |
| | | | | | | | sp\|Q9U | |
| 1057 | GAATTTCCCAG | 2.03 | 0.21 | 3.26 | 0.48 | Hs.2253 | complement | 0.62 |
| | | | | | | | component 2 | |
| | | | | | | | [Swissprot: sp\|O | |
| 1058 | TAAATAGAATT | 2.03 | 0.21 | 3.26 | 0.48 | Hs.22826 | tropomodulin 3 | 0.62 |
| | | | | | | | (ubiquitous) | |
| | | | | | | | [Swissprot: | |
| 1059 | TCAAGCAATCA | 2.03 | 0.21 | 3.26 | 0.48 | Hs.23388 | hypothetical | 0.62 |
| | | | | | | | protein | |
| | | | | | | | DKFZp434F031 | |
| | | | | | | | 8 [Swi | |
| 1060 | TGACCGGCGAG | 2.03 | 0.21 | 3.26 | 0.48 | Hs.23410 | translocase of | 0.62 |
| | | | | | | | inner | |
| | | | | | | | mitochondrial | |
| | | | | | | | membr | |
| 1061 | GCACCAAAAAA | 2.03 | 0.35 | 3.26 | 0.48 | Hs.23585 | KIAA1078 | 0.62 |
| | | | | | | | protein | |
| | | | | | | | [Swissprot: | |
| | | | | | | | sp\|Q96FB3; | |
| 1062 | GCCTGTTAAAA | 2.03 | 0.21 | 3.26 | 0.48 | Hs.239681 | hypothetical | 0.62 |
| | | | | | | | protein | |
| | | | | | | | FLJ20275 | |
| | | | | | | | [Swisspro | |
| 1063 | AATGTAGTTTT | 2.03 | 0.21 | 3.26 | 0.48 | Hs.24340 | centaurin, beta 2 | 0.62 |
| | | | | | | | [Swissprot: | |
| | | | | | | | sp\|Q15057 | |
| 1064 | GATAATGATTT | 2.03 | 0.21 | 3.26 | 0.48 | Hs.247118 | phosphatidylinositol | 0.62 |
| | | | | | | | glycan, class | |
| | | | | | | | B [S | |
| 1065 | GAAGAGCCATC | 2.03 | 0.21 | 3.26 | 0.48 | Hs.247935 | keratin | 0.62 |
| | | | | | | | associated | |
| | | | | | | | protein 1.3 | |
| | | | | | | | [Swisspr | |
| 1066 | TTAAAGATGCA | 2.03 | 0.21 | 3.26 | 0.48 | Hs.248 | mitogen- | 0.62 |
| | | | | | | | activated protein | |
| | | | | | | | kinase kinase | |
| 1067 | CTCACATTTGA | 2.03 | 0.21 | 3.26 | 0.48 | Hs.250646 | baculoviral IAP | 0.62 |
| | | | | | | | repeat- | |
| | | | | | | | containing 6 | |
| | | | | | | | [Sw | |
| 1068 | AAATGGCTTGA | 2.03 | 0.21 | 3.26 | 0.48 | Hs.25155 | neuroepithelial | 0.62 |
| | | | | | | | cell transforming | |
| | | | | | | | gene 1 | |
| 1069 | GTGGCTTCCCT | 2.03 | 0.21 | 3.26 | 0.48 | Hs.25648 | tumor necrosis | 0.62 |
| | | | | | | | factor receptor | |
| | | | | | | | superfami | |
| 1070 | GTACGCATTCC | 2.03 | 0.21 | 3.26 | 0.48 | Hs.258561 | general | 0.62 |
| | | | | | | | transcription | |
| | | | | | | | factor IIB [Swiss | |
| 1071 | TTTGAGACCTG | 2.03 | 0.21 | 3.26 | 0.48 | Hs.2707 | G1 to S phase | 0.62 |
| | | | | | | | transition 1 | |
| | | | | | | | [Swissprot: | |
| 1072 | TTCCATAATTA | 2.03 | 0.21 | 3.26 | 0.48 | Hs.27207 | KIAA0982 | 0.62 |
| | | | | | | | protein | |
| | | | | | | | [Swissprot: | |
| | | | | | | | sp\|Q9Y2I8; | |
| 1073 | CTGTGAGTTCG | 2.03 | 0.21 | 3.26 | 0.48 | Hs.272100 | chromosome 11 | 0.62 |
| | | | | | | | open reading | |
| | | | | | | | frame 21 [Sw | |
| 1074 | TCGTTACGCAG | 2.03 | 0.21 | 3.26 | 0.48 | Hs.2730 | heterogeneous | 0.62 |
| | | | | | | | nuclear | |
| | | | | | | | ribonucleoprotein | |
| 1075 | CAGGGGCTTAT | 2.03 | 0.21 | 3.26 | 0.48 | Hs.273387 | hypothetical | 0.62 |
| | | | | | | | protein | |
| | | | | | | | FLJ22559 | |
| | | | | | | | [Swisspro | |
| 1076 | CTGGGGGAGGG | 2.03 | 0.21 | 3.26 | 0.48 | Hs.274122 | erythrocyte | 0.62 |
| | | | | | | | membrane | |
| | | | | | | | protein band 4.9 | |
| | | | | | | | (d | |
| 1077 | CATTTTCCAGA | 2.03 | 0.21 | 3.26 | 0.48 | Hs.27475 | Homo sapiens | 0.62 |
| | | | | | | | cDNA FLJ12749 | |
| | | | | | | | fis, clone NT | |
| 1078 | TTGGTCAAACA | 2.03 | 0.21 | 3.26 | 0.48 | Hs.277445 | diacylglycerol | 0.62 |
| | | | | | | | kinase, zeta | |
| | | | | | | | (104 kD) [Sw | |
| 1079 | GCCAGCTGACG | 2.03 | 0.21 | 3.26 | 0.48 | Hs.27769 | ESTs, Weakly | 0.62 |
| | | | | | | | similar to | |
| | | | | | | | MCAT_HUMAN | |
| | | | | | | | MITOC | |
| 1080 | ATATGTATATT | 2.03 | 0.35 | 3.26 | 0.48 | Hs.278270 | unactive | 0.62 |
| | | | | | | | progesterone | |
| | | | | | | | receptor, 23 kD [ | |
| 1081 | TCTAGAATTTA | 2.03 | 0.21 | 3.26 | 0.48 | Hs.279591 | Homo sapiens | 0.62 |
| | | | | | | | clone 25056 | |
| | | | | | | | mRNA sequence | |
| 1082 | GAGGTTTTCTG | 2.03 | 0.21 | 3.26 | 0.48 | Hs.279639 | Homo sapiens | 0.62 |
| | | | | | | | mRNA; cDNA | |
| | | | | | | | DKFZp586M2022 | |
| | | | | | | | (f | |
| 1083 | CGGGGTTCTTG | 2.03 | 0.21 | 3.26 | 0.48 | Hs.28166 | cofactor required | 0.62 |
| | | | | | | | for Sp1 | |
| | | | | | | | transcriptiona | |
| 1084 | GAGATGGCTGG | 2.03 | 0.21 | 3.26 | 0.48 | Hs.285318 | Homo sapiens, | 0.62 |
| | | | | | | | Similar to | |
| | | | | | | | KIAA0626 gene p | |
| 1085 | TCTAAAGGTCA | 2.03 | 0.21 | 3.26 | 0.48 | Hs.286 | ribosomal | 0.62 |
| | | | | | | | protein L4 | |
| | | | | | | | [Swissprot; | |
| | | | | | | | sp\|P36 | |
| 1086 | TGTATGAATTG | 2.03 | 0.21 | 3.26 | 0.48 | Hs.28777 | H2A histone | 0.62 |
| | | | | | | | family, member | |
| | | | | | | | L [Swissprot | |
| 1087 | AATGTACCTGG | 2.03 | 0.21 | 3.26 | 0.48 | Hs.287921 | cAMP | 0.62 |
| | | | | | | | responsive | |
| | | | | | | | element binding | |
| | | | | | | | protein | |
| 1088 | TCTAGTCACTG | 2.03 | 0.21 | 3.26 | 0.48 | Hs.288087 | ESTs | 0.62 |
| | | | | | | | [Swissprot: | |
| | | | | | | | none] | |
| 1089 | GTTAAAGTTTA | 2.03 | 0.21 | 3.26 | 0.48 | Hs.288193 | hypothetical | 0.62 |
| | | | | | | | protein | |
| | | | | | | | MGC12217 | |
| | | | | | | | [Swisspro | |
| 1090 | TTTTTCCTAAG | 2.03 | 0.21 | 3.26 | 0.48 | Hs.29106 | mitogen- | 0.62 |
| | | | | | | | activated protein | |
| | | | | | | | kinase phospha | |
| 1091 | ACGTTTAAGGC | 2.03 | 0.21 | 3.26 | 0.48 | Hs.296381 | growth factor | 0.62 |
| | | | | | | | receptor-bound | |
| | | | | | | | protein 2 | |
| 1092 | CCCATTCATAG | 2.03 | 0.21 | 3.26 | 0.48 | Hs.29716 | hypothetical | 0.62 |
| | | | | | | | protein | |
| | | | | | | | FLJ10980 | |
| | | | | | | | [Swisspro | |
| 1093 | CTGGGTAACTG | 2.03 | 0.21 | 3.26 | 0.48 | Hs.300642 | serologically | 0.62 |
| | | | | | | | defined colon | |
| | | | | | | | cancer antig | |
| 1094 | TAAGAAGCCCC | 2.03 | 0.35 | 3.26 | 0.48 | Hs.301198 | roundabout | 0.62 |
| | | | | | | | (axon guidance | |
| | | | | | | | receptor, Dros | |
| 1095 | AAAGCTACTAG | 2.03 | 0.21 | 3.26 | 0.48 | Hs.31034 | peroxisomal | 0.62 |
| | | | | | | | biogenesis | |
| | | | | | | | factor 11A [Swis | |
| 1096 | TAGATAGAGTC | 2.03 | 0.21 | 3.26 | 0.48 | Hs.320831 | Home sapiens | 0.62 |
| | | | | | | | cDNA FLJ14597 | |
| | | | | | | | fis, clone NT | |
| 1097 | TACATAGAATT | 2.03 | 0.21 | 3.26 | 0.48 | Hs.323511 | Home sapiens | 0.62 |
| | | | | | | | cDNA: | |
| | | | | | | | FLJ23176 fis, | |
| | | | | | | | clone L | |
| 1098 | TATACCTGTGT | 2.03 | 0.21 | 3.26 | 0.48 | Hs.323748 | Homo sapiens | 0.62 |
| | | | | | | | clone | |
| | | | | | | | CDABP0086 | |
| | | | | | | | mRNA sequen | |
| 1099 | CTGCTGCTACT | 2.03 | 0.21 | 3.26 | 0.48 | Hs.325443 | breast cell | 0.62 |
| | | | | | | | glutaminase | |
| | | | | | | | [Swissprot: sp\| | |
| 1100 | AAGGAACTTGT | 2.03 | 0.21 | 3.26 | 0.48 | Hs.325825 | Home sapiens | 0.62 |
| | | | | | | | cDNA FLJ20848 | |
| | | | | | | | fis, clone AD | |
| 1101 | GGGAAAAAAAA | 2.03 | 0.21 | 3.26 | 0.48 | Hs.329726 | EST [Swissprot: | 0.62 |
| | | | | | | | none] | |
| 1102 | GTGCCCAGTCA | 2.03 | 0.21 | 3.26 | 0.48 | Hs.33010 | KIAA0633 | 0.62 |
| | | | | | | | protein | |
| | | | | | | | [Swissprot: | |
| | | | | | | | sp\|O75128; | |
| 1103 | CAGGGAGTGTG | 2.03 | 0.21 | 3.26 | 0.48 | Hs.332382 | hypothetical | 0.62 |
| | | | | | | | protein | |
| | | | | | | | MGC13007 | |
| | | | | | | | [Swisspro | |
| 1104 | GGGAGAACCCC | 2.03 | 0.21 | 3.26 | 0.48 | Hs.333189 | ESTs | 0.62 |
| | | | | | | | [Swissprot: | |
| | | | | | | | none] | |
| 1105 | AAAAGACCCGA | 2.03 | 0.21 | 3.26 | 0.48 | Hs.3343 | phosphoglycerate | 0.62 |
| | | | | | | | dehydrogenase | |
| | | | | | | | [Swisspr | |
| 1106 | GAGTGACTATC | 2.03 | 0.21 | 3.26 | 0.48 | Hs.334466 | hypothetical | 0.62 |
| | | | | | | | protein | |
| | | | | | | | [Swissprot: | |
| | | | | | | | sp\|Q9B | |
| 1107 | GCGAAAAAAAA | 2.03 | 0.21 | 3.26 | 0.48 | Hs.334725 | similar to | 0.62 |
| | | | | | | | putative | |
| | | | | | | | transmembrane | |
| | | | | | | | protei | |
| 1108 | AAAGTGAAGAA | 2.03 | 0.21 | 3.26 | 0.48 | Hs.334812 | hypothetical | 0.62 |
| | | | | | | | protein | |
| | | | | | | | DKFZp586K0717 | |
| | | | | | | | [Swi | |
| 1109 | GTGGCCAAAGT | 2.03 | 0.21 | 3.26 | 0.48 | Hs.334895 | ribosomal | 0.62 |
| | | | | | | | protein L10a | |
| | | | | | | | [Swissprot: sp\|O [symbol] | |
| 1110 | GACCCCTGTCA | 2.03 | 0.35 | 3.26 | 0.48 | Hs.336561 | KIAA1742 | 0.62 |
| | | | | | | | protein | |
| | | | | | | | [Swissprot: | |
| | | | | | | | sp\|Q9C0C1; | |
| 1111 | AAAAGACAAAT | 2.03 | 0.21 | 3.26 | 0.48 | Hs.343411 | DEAD/H (Asp- | 0.62 |
| | | | | | | | Glu-Ala-Asp/His) | |
| | | | | | | | box polypep | |
| 1112 | AAGAGGCTGAG | 2.03 | 0.21 | 3.26 | 0.48 | Hs.348424 | Homo sapiens, | 0.62 |
| | | | | | | | DKFZP434B103 | |
| | | | | | | | protein, clon | |
| 1113 | TATATATAGAG | 2.03 | 0.21 | 3.26 | 0.48 | Hs.34853 | inhibitor of DNA | 0.62 |
| | | | | | | | binding 4, | |
| | | | | | | | dominant neg | |
| 1114 | TGAAGAAAGGA | 2.03 | 0.21 | 3.26 | 0.48 | Hs.3577 | succinate | 0.62 |
| | | | | | | | dehydrogenase | |
| | | | | | | | complex, subunit | |
| 1115 | TTTATTTTAAT | 2.03 | 0.21 | 3.26 | 0.48 | Hs.37040 | platelet-derived | 0.62 |
| | | | | | | | growth factor | |
| | | | | | | | alpha pol | |
| 1116 | TTGTTTAAAGG | 2.03 | 0.21 | 3.26 | 0.48 | Hs.3838 | serum-inducible | 0.62 |
| | | | | | | | kinase | |
| | | | | | | | [Swissprot: sp\|Q [symbol] | |
| 1117 | ATCCCTTCCCG | 2.03 | 0.21 | 3.26 | 0.48 | Hs.3847 | peanut | 0.62 |
| | | | | | | | (Drosophila)-like | |
| | | | | | | | 1 [Swissprot: | |
| 1118 | AAGATCCTACG | 2.03 | 0.21 | 3.26 | 0.48 | Hs.42514 | hypothetical | 0.62 |
| | | | | | | | protein F25965 | |
| | | | | | | | [Swissprot: | |
| 1119 | TGGAACTGAGT | 2.03 | 0.35 | 3.26 | 0.48 | Hs.43899 | Homo sapiens | 0.62 |
| | | | | | | | mRNA; cDNA | |
| | | | | | | | DKFZp434C1714 | |
| | | | | | | | (f | |
| 1120 | GATGCATATAG | 2.03 | 0.21 | 3.26 | 0.48 | Hs.43910 | CD164 antigen, | 0.62 |
| | | | | | | | sialomucin | |
| | | | | | | | [Swissprot: s | |
| 1121 | TTTGGAAAGAG | 2.03 | 0.21 | 3.26 | 0.48 | Hs.44425 | ESTs, Weakly | 0.62 |
| | | | | | | | similar to | |
| | | | | | | | Z195_HUMAN | |
| | | | | | | | ZINC | |
| 1122 | AGTATCAGCAG | 2.03 | 0.21 | 3.26 | 0.48 | Hs.47546 | Homo sapiens | 0.62 |
| | | | | | | | cDNA FLJ11458 | |
| | | | | | | | fis, clone HE | |
| 1123 | CTCCTGAAAAA | 2.03 | 0.21 | 3.26 | 0.48 | Hs.4890 | ubiquitin- | 0.62 |
| | | | | | | | conjugating | |
| | | | | | | | enzyme E2E 3 | |
| | | | | | | | (homo | |
| 1124 | TGTGAGCCCTT | 2.03 | 0.21 | 3.26 | 0.48 | Hs.4997 | hypothetical | 0.62 |
| | | | | | | | protein | |
| | | | | | | | FLJ10482 | |
| | | | | | | | [Swisspro | |
| 1125 | CATTGTCTTCA | 2.03 | 0.21 | 3.26 | 0.48 | Hs.5054 | glutaredoxin 2 | 0.62 |
| | | | | | | | [Swissprot: | |
| | | | | | | | sp\|Q96JC0; sp | |
| 1126 | GGAAGAGGGTG | 2.03 | 0.21 | 3.26 | 0.48 | Hs.5331 | Homo sapiens | 0.62 |
| | | | | | | | cDNA: | |
| | | | | | | | FLJ22361 fis, | |
| | | | | | | | clone H | |
| 1127 | GACCAACTGGG | 2.03 | 0.21 | 3.26 | 0.48 | Hs.55067 | hypothetical | 0.62 |
| | | | | | | | protein | |
| | | | | | | | MGC15437 | |
| | | | | | | | [Swisspro | |
| 1128 | AGAATTTGAAT | 2.03 | 0.21 | 3.26 | 0.48 | Hs.5518 | Homo sapiens | 0.62 |
| | | | | | | | mRNA; cDNA | |
| | | | | | | | DKFZp566J2146 | |
| | | | | | | | (f | |
| 1129 | AGACCTGTAAT | 2.03 | 0.21 | 3.26 | 0.48 | Hs.57419 | CCCTC-binding | 0.62 |
| | | | | | | | factor (zinc | |
| | | | | | | | finger protei | |
| 1130 | AAATAATTGTG | 2.03 | 0.21 | 3.26 | 0.48 | Hs.57435 | solute carrier | 0.62 |
| | | | | | | | family 11 | |
| | | | | | | | (proton-coupled | |
| 1131 | GAACTGGATTT | 2.03 | 0.21 | 3.26 | 0.48 | Hs.57698 | NAD(P) | 0.62 |
| | | | | | | | dependent | |
| | | | | | | | steroid | |
| | | | | | | | dehydrogenase-I | |
| 1132 | CTCCTTAAAAC | 2.03 | 0.21 | 3.26 | 0.48 | Hs.59563 | hypothetical | 0.62 |
| | | | | | | | protein | |
| | | | | | | | FLJ00007 | |
| | | | | | | | [Swisspro | |
| 1133 | TCACAAACTTC | 2.03 | 0.21 | 3.26 | 0.48 | Hs.61828 | amyloid beta | 0.62 |
| | | | | | | | precursor | |
| | | | | | | | protein-binding p | |
| 1134 | TTACTTCAACT | 2.03 | 0.21 | 3.26 | 0.48 | Hs.6236 | Homo sapiens | 0.62 |
| | | | | | | | cDNA: | |
| | | | | | | | FLJ21487 fis, | |
| | | | | | | | clone C | |
| 1135 | TATAATAAAAA | 2.03 | 0.21 | 3.26 | 0.48 | Hs.64 | succinate | 0.62 |
| | | | | | | | dehydrogenase | |
| | | | | | | | complex, subunit | |
| 1136 | TCAGTGAACTG | 2.03 | 0.35 | 3.26 | 0.48 | Hs.6657 | hypothetical | 0.62 |
| | | | | | | | protein | |
| | | | | | | | bK1048E9.5 | |
| | | | | | | | [Swissp | |
| 1137 | GTCCGGTGGTT | 2.03 | 0.21 | 3.26 | 0.48 | Hs.6684 | KIAA0476 gene | 0.62 |
| | | | | | | | product | |
| | | | | | | | [Swissprot: | |
| | | | | | | | sp\|O7 | |
| 1138 | ATGCAGAGATT | 2.03 | 0.35 | 3.26 | 0.48 | Hs.7137 | clones 23667 | 0.62 |
| | | | | | | | and 23775 zinc | |
| | | | | | | | finger prote | |
| 1139 | CGTCCCGGAGC | 2.03 | 0.21 | 3.26 | 0.48 | Hs.7345 | MAD1 (mitotic | 0.62 |
| | | | | | | | arrest deficient, | |
| | | | | | | | yeast, h | |
| 1140 | GACTGGAAAAA | 2.03 | 0.21 | 3.26 | 0.48 | Hs.743 | Fc fragment of | 0.62 |
| | | | | | | | IgE, high affinity | |
| | | | | | | | I, rec | |
| 1141 | GACTGGACTCT | 2.03 | 0.21 | 3.26 | 0.48 | Hs.743 | Fc fragment of | 0.62 |
| | | | | | | | IgE, high affinity | |
| | | | | | | | I, rec | |
| 1142 | CAGTGATTCCA | 2.03 | 0.35 | 3.26 | 0.48 | Hs.75056 | adaptor-related | 0.62 |
| | | | | | | | protein complex | |
| | | | | | | | 3, delta | |
| 1143 | CTCTGAGAGAA | 2.03 | 0.21 | 3.26 | 0.48 | Hs.75113 | general | 0.62 |
| | | | | | | | transcription | |
| | | | | | | | factor IIIA [Swis | |
| 1144 | TACCCAGCAAA | 2.03 | 0.21 | 3.26 | 0.48 | Hs.75372 | N- | 0.62 |
| | | | | | | | acetylgalactosaminidase, | |
| | | | | | | | alpha- | |
| | | | | | | | [Swis | |
| 1145 | TTCCTTTTTAC | 2.03 | 0.21 | 3.26 | 0.48 | Hs.75682 | autoantigen | 0.62 |
| | | | | | | | [Swissprot: | |
| | | | | | | | sp\|Q13025; sp\|Q1 | |
| 1146 | CGGTCTTATGT | 2.03 | 0.21 | 3.26 | 0.48 | Hs.75842 | dual-specificity | 0.62 |
| | | | | | | | tyrosine-(Y)- | |
| | | | | | | | phosphoryl | |
| 1147 | TAATGTGTACA | 2.03 | 0.21 | 3.26 | 0.48 | Hs.76917 | F-box only | 0.62 |
| | | | | | | | protein 8 | |
| | | | | | | | [Swissprot: | |
| | | | | | | | sp\|Q9N | |
| 1148 | GTGAAGGCAGA | 2.03 | 0.21 | 3.26 | 0.48 | Hs.77039 | ribosomal | 0.62 |
| | | | | | | | protein S3A | |
| | | | | | | | [Swissprot: | |
| | | | | | | | sp\|P4 | |
| 1149 | CAATTTAAGTG | 2.03 | 0.35 | 3.26 | 0.48 | Hs.77324 | eukaryotic | 0.62 |
| | | | | | | | translation | |
| | | | | | | | termination facto | |
| 1150 | TAATTTGATTT | 2.03 | 0.21 | 3.26 | 0.48 | Hs.78563 | ubiquitin- | 0.62 |
| | | | | | | | conjugating | |
| | | | | | | | enzyme E2G 1 | |
| | | | | | | | (homo | |
| 1151 | ATTAAAGTCAG | 2.03 | 0.21 | 3.26 | 0.48 | Hs.78748 | KIAA0237 gene | 0.62 |
| | | | | | | | product | |
| | | | | | | | [Swissprot: | |
| | | | | | | | sp\|Q9 | |
| 1152 | ATGAAAAAAAA | 2.03 | 0.21 | 3.26 | 0.48 | Hs.78793 | protein kinase C, | 0.62 |
| | | | | | | | zeta [Swissprot: | |
| | | | | | | | sp\|Q | |
| 1153 | AGAAGAACGAG | 2.03 | 0.21 | 3.26 | 0.48 | Hs.79064 | deoxyhypusine | 0.62 |
| | | | | | | | synthase | |
| | | | | | | | [Swissprot: sp\|P [symbol] | |
| 1154 | GGAAGCCCACC | 2.03 | 0.21 | 3.26 | 0.48 | Hs.79265 | suppression of | 0.62 |
| | | | | | | | tumorigenicity 5 | |
| | | | | | | | [Swissp | |
| 1155 | TGAACAGCATA | 2.03 | 0.21 | 3.26 | 0.48 | Hs.79306 | eukaryotic | 0.62 |
| | | | | | | | translation | |
| | | | | | | | initiation factor | |
| 1156 | GCCTGTGCTGG | 2.03 | 0.21 | 3.26 | 0.48 | Hs.79391 | huntingtin | 0.62 |
| | | | | | | | (Huntington | |
| | | | | | | | disease) | |
| | | | | | | | [Swissp | |
| 1157 | AATTAATTGTA | 2.03 | 0.21 | 3.26 | 0.48 | Hs.7943 | RPB5-mediating | 0.62 |
| | | | | | | | protein | |
| | | | | | | | [Swissprot: sp\|O [symbol] | |
| 1158 | TAAATAATACA | 2.03 | 0.21 | 3.26 | 0.48 | Hs.79768 | KIAA0111 gene | 0.62 |
| | | | | | | | product | |
| | | | | | | | [Swissprot: | |
| | | | | | | | sp\|P3 | |
| 1159 | GGCACTTATGA | 2.03 | 0.21 | 3.26 | 0.48 | Hs.7978 | DKFZP434C131 | 0.62 |
| | | | | | | | protein | |
| | | | | | | | [Swissprot: | |
| | | | | | | | sp\|Q9N | |
| 1160 | TAAATTATTTC | 2.03 | 0.21 | 3.26 | 0.48 | Hs.79877 | myotubularin | 0.62 |
| | | | | | | | related protein 6 | |
| | | | | | | | [Swisspr | |
| 1161 | GGTGGCCCGGG | 2.03 | 0.21 | 3.26 | 0.48 | Hs.8039 | hypothetical | 0.62 |
| | | | | | | | protein | |
| | | | | | | | DKFZp566M1046 | |
| | | | | | | | [Swl | |
| 1162 | GTATAATAGCC | 2.03 | 0.21 | 3.26 | 0.48 | Hs.80506 | small nuclear | 0.62 |
| | | | | | | | ribonucleoprotein | |
| | | | | | | | polypept | |
| 1163 | TTATCAAAAAA | 2.03 | 0.21 | 3.26 | 0.48 | Hs.81452 | fatty-acid- | 0.62 |
| | | | | | | | Coenzyme A | |
| | | | | | | | ligase, long- | |
| | | | | | | | chain | |
| 1164 | TACATTCACCT | 2.03 | 0.21 | 3.26 | 0.48 | Hs.82043 | D123 gene | 0.62 |
| | | | | | | | product | |
| | | | | | | | [Swissprot: | |
| | | | | | | | sp\|O75794 | |
| 1165 | TGAAGGTTTTT | 2.03 | 0.21 | 3.26 | 0.48 | Hs.82240 | syntaxin 3A | 0.62 |
| | | | | | | | [Swissprot: | |
| | | | | | | | sp\|Q13277;] | |
| 1166 | TAAATGTTGAT | 2.03 | 0.21 | 3.26 | 0.48 | Hs.83572 | hypothetical | 0.62 |
| | | | | | | | protein | |
| | | | | | | | MGC14433 | |
| | | | | | | | [Swisspro | |
| 1167 | AAGGTAACTAA | 2.03 | 0.21 | 3.26 | 0.48 | Hs.83765 | dihydrofolate | 0.62 |
| | | | | | | | reductase | |
| | | | | | | | [Swissprot: sp\| | |
| 1168 | GGAAACTGATG | 2.03 | 0.21 | 3.26 | 0.48 | Hs.83916 | NADH | 0.62 |
| | | | | | | | dehydrogenase | |
| | | | | | | | (ubiquinone) 1 | |
| | | | | | | | alpha | |
| 1169 | ACATTGGTTAA | 2.03 | 0.21 | 3.26 | 0.48 | Hs.84087 | KIAA0143 | 0.62 |
| | | | | | | | protein | |
| | | | | | | | [Swissprot: | |
| | | | | | | | sp\|Q14156; | |
| 1170 | AGGTGCCTCGG | 2.03 | 0.21 | 3.26 | 0.48 | Hs.84285 | ubiquitin- | 0.62 |
| | | | | | | | conjugating | |
| | | | | | | | enzyme E2I | |
| | | | | | | | (homolo | |
| 1171 | GTAGAGCTTGA | 2.03 | 0.21 | 3.26 | 0.48 | Hs.8694 | hypothetical | 0.62 |
| | | | | | | | protein from | |
| | | | | | | | EUROIMAGE | |
| | | | | | | | 1034 | |
| 1172 | GGGCTGTGGAG | 2.03 | 0.21 | 3.26 | 0.48 | Hs.9071 | progesterone | 0.62 |
| | | | | | | | receptor | |
| | | | | | | | membrane | |
| | | | | | | | component | |
| 1173 | CACAGAATGCT | 2.03 | 0.21 | 3.26 | 0.48 | Hs.91773 | protein | 0.62 |
| | | | | | | | phosphatase 2 | |
| | | | | | | | (formerly 2A), | |
| | | | | | | | cat | |
| 1174 | CTAATTTAACT | 2.03 | 0.35 | 3.26 | 0.48 | Hs.9194 | putative | 0.62 |
| | | | | | | | glialblastoma | |
| | | | | | | | cell differentiat | |
| 1175 | AAGGCAAAGCT | 2.03 | 0.21 | 3.26 | 0.48 | Hs.93748 | Homo sapiens | 0.62 |
| | | | | | | | cDNA FLJ14844 | |
| | | | | | | | fis, clone PL | |
| 1176 | TTAGCACTGTG | 2.03 | 0.21 | 3.26 | 0.48 | Hs.94308 | RAB35, member | 0.62 |
| | | | | | | | RAS oncogene | |
| | | | | | | | family [Swis | |
| 1177 | TTGGGAATCCC | 2.03 | 0.21 | 3.26 | 0.48 | Hs.9547 | hypothetical | 0.62 |
| | | | | | | | protein | |
| | | | | | | | FLJ10916 | |
| | | | | | | | [Swisspro | |
| 1178 | AATTTGTGAAG | 2.03 | 0.21 | 3.26 | 0.48 | Hs.9599 | solute carrier | 0.62 |
| | | | | | | | family 25, | |
| | | | | | | | member 13 (cit | |
| 1179 | CTTCTCCAAAA | 2.03 | 0.21 | 3.26 | 0.48 | Hs.99949 | prolactin- | 0.62 |
| | | | | | | | induced protein | |
| | | | | | | | [Swissprot: s | |
| 1180 | TACCAAGACCC | 2.28 | 0.77 | 1.83 | 0.47 | Hs.3059 | coatomer protein | 1.25 |
| | | | | | | | complex, subunit | |
| | | | | | | | beta | |
| 1181 | GGATGTGAAAG | −1.97 | 0.56 | −1.84 | 0.46 | Hs.177543 | antigen | 1.07 |
| | | | | | | | Identified by | |
| | | | | | | | monoclonal | |
| | | | | | | | antibod | |
| 1182 | GGTAGCCTGGG | 2.37 | 0.66 | 1.90 | 0.41 | Hs.108327 | damage-specific | 1.25 |
| | | | | | | | DNA binding | |
| | | | | | | | protein 1 (1 | |
| 1183 | GGGACGAGTGA | 2.37 | 0.66 | 1.90 | 0.41 | Hs.3337 | transmembrane | 1.25 |
| | | | | | | | 4 superfamily | |
| | | | | | | | member 1 [S | |
| 1184 | CGTGGGACACT | 2.03 | 0.90 | 1.43 | 0.35 | Hs.110196 | NICE-1 protein | 1.42 |
| | | | | | | | [Swissprot: | |
| | | | | | | | sp\|Q9UGL9;] | |
| 1185 | TGTATTGTACA | 2.53 | 0.55 | 2.04 | 0.35 | Hs.118562 | Link guanine | 1.24 |
| | | | | | | | nucleotide | |
| | | | | | | | exchange factor | |
| 1186 | CTCACTTCTTA | 2.53 | 0.55 | 2.04 | 0.35 | Hs.165998 | PAI-1 mRNA- | 1.24 |
| | | | | | | | binding protein | |
| | | | | | | | [Swissprot: | |
| 1187 | GTGAGACCTCA | 2.53 | 0.55 | 2.04 | 0.35 | Hs.268541 | novel SH2- | 1.24 |
| | | | | | | | containing | |
| | | | | | | | protein 1 | |
| | | | | | | | [Swisspr | |
| 1188 | CAGTTACTTAG | 2.53 | 0.55 | 2.04 | 0.35 | Hs.279920 | tyrosine 3- | 1.24 |
| | | | | | | | monooxygenase/ | |
| | | | | | | | tryptophan 5-mo | |
| 1189 | CTCTGGGATAG | 2.53 | 0.55 | 2.04 | 0.35 | Hs.334437 | hypothetical | 1.24 |
| | | | | | | | protein | |
| | | | | | | | MGC4248 | |
| | | | | | | | [Swissprot | |
| 1190 | TCCTAGCCTGT | 2.53 | 0.55 | 2.04 | 0.35 | Hs.74711 | DnaJ (Hsp40) | 1.24 |
| | | | | | | | homolog, | |
| | | | | | | | subfamily C, | |
| | | | | | | | membe | |
| 1191 | AGCTCTGCTGC | −2.96 | 0.42 | −2.46 | 0.32 | Hs.102402 | Mad4 homolog | 1.20 |
| | | | | | | | [Swissprot: | |
| | | | | | | | sp\|Q14582; sp\|Q | |
| 1192 | AACCAAAAAAA | −2.96 | 0.42 | −2.46 | 0.32 | Hs.155410 | isocitrate | 1.20 |
| | | | | | | | dehydrogenase | |
| | | | | | | | 3 (NAD+) beta | |
| 1193 | AAGCGCTCTCG | −2.96 | 0.42 | −2.46 | 0.32 | Hs.168913 | serine/threonine | 1.20 |
| | | | | | | | kinase 24 | |
| | | | | | | | (Ste20, yeast | |
| 1194 | AGATAACACAG | −2.96 | 0.42 | −2.46 | 0.32 | Hs.194369 | arginine- | 1.20 |
| | | | | | | | glutamic acid | |
| | | | | | | | dipeptide (RE) re [symbol] | |
| 1195 | GACACGAACAA | −2.96 | 0.42 | −2.46 | 0.32 | Hs.25829 | ras-related | 1.20 |
| | | | | | | | protein | |
| | | | | | | | [Swissprot: | |
| | | | | | | | sp\|Q9HC | |
| 1196 | GTGGAACCCCG | −2.96 | 0.42 | −2.46 | 0.32 | Hs.270796 | ESTs | 1.20 |
| | | | | | | | [Swissprot: | |
| | | | | | | | none] | |
| 1197 | GTGCCCTGTTG | −2.96 | 0.42 | −2.46 | 0.32 | Hs.278411 | NCK-associated | 1.20 |
| | | | | | | | protein 1 | |
| | | | | | | | [Swissprot: sp | |
| 1198 | CGATCAGTTTG | −2.96 | 0.42 | −2.46 | 0.32 | Hs.34906 | ESTs, Weakly | 1.20 |
| | | | | | | | similar to | |
| | | | | | | | T17210 | |
| | | | | | | | hypotheti | |
| 1199 | GGGGAAGGGCA | −2.96 | 0.42 | −2.46 | 0.32 | Hs.65377 | Homo sapiens, | 1.20 |
| | | | | | | | clone | |
| | | | | | | | MGC: 16377 | |
| | | | | | | | IMAGE: 3936 | |
| 1200 | TCCGTGGTTGG | −2.96 | 0.42 | −2.46 | 0.32 | Hs.79516 | brain abundant, | 1.20 |
| | | | | | | | membrane | |
| | | | | | | | attached signal | |
| 1201 | TGGCCCCCGCC | −2.96 | 0.42 | −2.46 | 0.32 | Hs.93649 | upstream | 1.20 |
| | | | | | | | transcription | |
| | | | | | | | factor 2, c-fos i | |
| 1202 | CAGTTGGTTGT | −2.47 | 0.53 | −1.84 | 0.30 | Hs.155218 | E1B-55 kDa- | 1.34 |
| | | | | | | | associated | |
| | | | | | | | protein 5 | |
| | | | | | | | [Swisspr | |
| 1203 | TCCGCGAGAAG | −2.47 | 0.53 | −1.84 | 0.30 | Hs.343586 | zinc finger | 1.34 |
| | | | | | | | protein | |
| | | | | | | | homologous to | |
| | | | | | | | Zfp-36 | |
| 1204 | AAAAAAAAAAG | −1.97 | 0.33 | −1.84 | 0.30 | Hs.180842 | ribosomal | 1.07 |
| | | | | | | | protein L13 | |
| | | | | | | | [Swissprot: | |
| | | | | | | | sp\|P2 | |
| 1205 | AAGATAATGCC | 2.03 | 0.48 | 1.63 | 0.27 | Hs.102696 | MCT-1 protein | 1.25 |
| | | | | | | | [Swissprot: | |
| | | | | | | | sp\|Q9ULC4;] | |
| 1206 | TCACTGCACTC | 2.03 | 0.70 | 1.36 | 0.23 | Hs.288232 | Homo sapiens | 1.49 |
| | | | | | | | cDNA: | |
| | | | | | | | FLJ22642 fis, | |
| | | | | | | | clone H | |
| 1207 | GCACCTAGTGC | 2.03 | 0.35 | 1.63 | 0.20 | Hs.1287 | zinc finger | 1.25 |
| | | | | | | | protein 173 | |
| | | | | | | | [Swissprot: sp\| | |
| 1208 | GAGAGGGCAGA | 2.03 | 0.35 | 1.63 | 0.20 | Hs.26412 | ring finger | 1.25 |
| | | | | | | | protein 26 | |
| | | | | | | | [Swissprot: sp\|Q | |
| 1209 | TTAAAGGCCGG | 2.03 | 0.35 | 1.63 | 0.20 | Hs.79086 | mitochondrial | 1.25 |
| | | | | | | | ribosomal | |
| | | | | | | | protein L3 [Swi | |
| 1210 | GAATCGGTTAT | 2.53 | 0.55 | 1.36 | 0.14 | Hs.80595 | NADH | 1.86 |
| | | | | | | | dehydrogenase | |
| | | | | | | | (ubiquinone) Fe—S | |
| | | | | | | | pro | |
| 1211 | GGGCCAATAAA | −1.97 | 0.56 | −1.23 | 0.12 | Hs.78605 | DKFZP566I1024 | 1.60 |
| | | | | | | | protein | |
| | | | | | | | [Swissprot: | |
| | | | | | | | sp\|Q9 | |
| 1212 | TCAAATGCAAA | 2.03 | 0.21 | 1.63 | 0.10 | Hs.116875 | KIAA0156 gene | 1.25 |
| | | | | | | | product | |
| | | | | | | | [Swissprot: | |
| | | | | | | | sp\|Q1 | |
| 1213 | TCAATAAAGGA | 2.03 | 0.21 | 1.63 | 0.10 | Hs.118797 | ubiquitin- | 1.25 |
| | | | | | | | conjugating | |
| | | | | | | | enzyme E2D 3 | |
| | | | | | | | (homo | |
| 1214 | CAGAATAATGT | 2.03 | 0.21 | 1.63 | 0.10 | Hs.125031 | choline/ethanola | 1.25 |
| | | | | | | | minephosphotransferase | |
| 1215 | CCTGGCCAAAA | 2.03 | 0.21 | 1.63 | 0.10 | Hs.126824 | EST [Swissprot: | 1.25 |
| | | | | | | | none] | |
| 1216 | ACAATGTAGGA | 2.03 | 0.21 | 1.63 | 0.10 | Hs.137415 | Homo sapiens | 1.25 |
| | | | | | | | BAC clone | |
| | | | | | | | RP11-294L11 | |
| | | | | | | | from | |
| 1217 | AAACACCAAAT | 2.03 | 0.21 | 1.63 | 0.10 | Hs.146388 | microtubule- | 1.25 |
| | | | | | | | associated | |
| | | | | | | | protein 7 [Swiss | |
| 1218 | TGAAAGTCCTG | 2.03 | 0.21 | 1.63 | 0.10 | Hs.152707 | glioblastoma | 1.25 |
| | | | | | | | amplified | |
| | | | | | | | sequence | |
| | | | | | | | [Swissp | |
| 1219 | ATAATTGACTA | 2.03 | 0.21 | 1.63 | 0.10 | Hs.15591 | COP9 subunit 6 | 1.25 |
| | | | | | | | (MOV34 | |
| | | | | | | | homolog, 34 kD) [ | |
| 1220 | TACCATCCATA | 2.03 | 0.21 | 1.63 | 0.10 | Hs.169476 | glyceraldehyde- | 1.25 |
| | | | | | | | 3-phosphate | |
| | | | | | | | dehydrogenase | |
| 1221 | GTGTAGTTGAG | 2.03 | 0.21 | 1.63 | 0.10 | Hs.171811 | adenylate kinase | 1.25 |
| | | | | | | | 2 [Swissprot: | |
| | | | | | | | sp\|P5481 | |
| 1222 | TGACTGTCACG | 2.03 | 0.21 | 1.63 | 0.10 | Hs.177776 | hypothetical | 1.25 |
| | | | | | | | protein | |
| | | | | | | | MGC4276 | |
| | | | | | | | similar to | |
| 1223 | GGACATTTTTC | 2.03 | 0.21 | 1.63 | 0.10 | Hs.183593 | zinc finger | 1.25 |
| | | | | | | | protein 24 (KOX | |
| | | | | | | | 17) [Swissp | |
| 1224 | TCTCTCTGCCT | 2.03 | 0.21 | 1.63 | 0.10 | Hs.184987 | ESTs | 1.25 |
| | | | | | | | [Swissprot: | |
| | | | | | | | none] | |
| 1225 | ACTACAGCCAT | 2.03 | 0.21 | 1.63 | 0.10 | Hs.186961 | ubiquitin specific | 1.25 |
| | | | | | | | protease 25 | |
| | | | | | | | [Swisspr | |
| 1226 | TTGGACAAGAA | 2.03 | 0.21 | 1.63 | 0.10 | Hs.189902 | ESTs | 1.25 |
| | | | | | | | [Swissprot: | |
| | | | | | | | none] | |
| 1227 | GGGATTAAAGC | 2.03 | 0.21 | 1.63 | 0.10 | Hs.211579 | melanoma cell | 1.25 |
| | | | | | | | adhesion | |
| | | | | | | | molecule | |
| | | | | | | | [Swissp | |
| 1228 | CTTTTTGCCAC | 2.03 | 0.21 | 1.63 | 0.10 | Hs.240165 | ESTs | 1.25 |
| | | | | | | | [Swissprot: | |
| | | | | | | | none] | |
| 1229 | CACTATGTAAA | 2.03 | 0.21 | 1.63 | 0.10 | Hs.24143 | Wiskott-Aldrich | 1.25 |
| | | | | | | | syndrome | |
| | | | | | | | protein interac | |
| 1230 | TGCCTCCCATC | 2.03 | 0.21 | 1.63 | 0.10 | Hs.2437 | eukaryotic | 1.25 |
| | | | | | | | translation | |
| | | | | | | | initiation factor | |
| 1231 | ATAATCTGAAG | 2.03 | 0.21 | 1.63 | 0.10 | Hs.2441 | KIAA0022 gene | 1.25 |
| | | | | | | | product | |
| | | | | | | | [Swissprot: | |
| | | | | | | | sp\|Q1 | |
| 1232 | GTCAAAAAAAA | 2.03 | 0.21 | 1.63 | 0.10 | Hs.271699 | polymerase | 1.25 |
| | | | | | | | (DNA directed) | |
| | | | | | | | iota [Swisspr | |
| 1233 | GAATGTCCTTT | 2.03 | 0.21 | 1.63 | 0.10 | Hs.282093 | hypothetical | 1.25 |
| | | | | | | | protein | |
| | | | | | | | FLJ21918 | |
| | | | | | | | [Swisspro | |
| 1234 | GTTTCAGTTAC | 2.03 | 0.21 | 1.63 | 0.10 | Hs.285673 | hypothetical | 1.25 |
| | | | | | | | protein | |
| | | | | | | | FLJ20950 | |
| | | | | | | | [Swisspro | |
| 1235 | TTCATTAAAAA | 2.03 | 0.21 | 1.63 | 0.10 | Hs.287797 | integrin, beta 1 | 1.25 |
| | | | | | | | (fibronectin | |
| | | | | | | | receptor, | |
| 1236 | CACAGTATTTG | 2.03 | 0.21 | 1.63 | 0.10 | Hs.295923 | seven in | 1.25 |
| | | | | | | | absentia | |
| | | | | | | | (Drosophila) | |
| | | | | | | | homolog 1 | |
| 1237 | TCGTCCTAGAA | 2.03 | 0.21 | 1.63 | 0.10 | Hs.296406 | KIAA0685 gene | 1.25 |
| | | | | | | | product | |
| | | | | | | | [Swissprot: | |
| | | | | | | | sp\|O7 | |
| 1238 | ATTAGCAGAGT | 2.03 | 0.21 | 1.63 | 0.10 | Hs.297939 | cathepsin B | 1.25 |
| | | | | | | | [Swissprot: | |
| | | | | | | | sp\|P07858; sp\|Q9 | |
| 1239 | GTGAAACACCA | 2.03 | 0.21 | 1.63 | 0.10 | Hs.303974 | EST [Swissprot: | 1.25 |
| | | | | | | | none] | |
| 1240 | TCTGTAATCTC | 2.03 | 0.21 | 1.63 | 0.10 | Hs.311355 | EST [Swissprot: | 1.25 |
| | | | | | | | none] | |
| 1241 | GTGAAACCCTC | 2.03 | 0.21 | 1.63 | 0.10 | Hs.311523 | ESTs, Weakly | 1.25 |
| | | | | | | | similar to | |
| | | | | | | | Z195_HUMAN | |
| | | | | | | | ZINC | |
| 1242 | CTCCATTGCCA | 2.03 | 0.21 | 1.63 | 0.10 | Hs.31869 | sialoadhesin | 1.25 |
| | | | | | | | [Swissprot: | |
| | | | | | | | sp\|O43550; sp\|O | |
| 1243 | TGCCATATAAG | 2.03 | 0.21 | 1.63 | 0.10 | Hs.32271 | hypothetical | 1.25 |
| | | | | | | | protein | |
| | | | | | | | FLJ10846 | |
| | | | | | | | [Swisspro | |
| 1244 | TGGCTGTTAAT | 2.03 | 0.21 | 1.63 | 0.10 | Hs.331584 | cytokine-like | 1.25 |
| | | | | | | | nuclear factor n- | |
| | | | | | | | pac [Swi | |
| 1245 | CCTGTCAATGT | 2.03 | 0.21 | 1.63 | 0.10 | Hs.334334 | transcription | 1.25 |
| | | | | | | | factor AP-2 | |
| | | | | | | | alpha (activat | |
| 1246 | GTGGCGGACGC | 2.03 | 0.21 | 1.63 | 0.10 | Hs.335121 | EST [Swissprot: | 1.25 |
| | | | | | | | none] | |
| 1247 | CGAATGTCCTT | 2.03 | 0.21 | 1.63 | 0.10 | Hs.335952 | keratin 6B | 1.25 |
| | | | | | | | [Swissprot: | |
| | | | | | | | sp\|P48669;] | |
| 1248 | AGAACAGACCA | 2.03 | 0.21 | 1.63 | 0.10 | Hs.339352 | brother of CDO | 1.25 |
| | | | | | | | [Swissprot: | |
| | | | | | | | sp\|Q9BWV1;] | |
| 1249 | GTGTTAGCGCA | 2.03 | 0.21 | 1.63 | 0.10 | Hs.339696 | ribosomal | 1.25 |
| | | | | | | | protein S12 | |
| | | | | | | | [Swissprot: | |
| | | | | | | | sp\|P2 | |
| 1250 | TGCATCTGTGC | 2.03 | 0.21 | 1.63 | 0.10 | Hs.348390 | DVS27-related | 1.25 |
| | | | | | | | protein | |
| | | | | | | | [Swissprot: | |
| | | | | | | | sp\|O9 | |
| 1251 | CCAGTTTGTAT | 2.03 | 0.21 | 1.63 | 0.10 | Hs.42331 | ephrin-A4 | 1.25 |
| | | | | | | | [Swissprot: | |
| | | | | | | | sp\|P52798;] | |
| 1252 | TTTATTGAATT | 2.03 | 0.21 | 1.63 | 0.10 | Hs.43910 | CD164 antigen, | 1.25 |
| | | | | | | | sialomucin | |
| | | | | | | | [Swissprot: s | |
| 1253 | TGTGAAGATTA | 2.03 | 0.21 | 1.63 | 0.10 | Hs.44829 | ESTs, Weakly | 1.25 |
| | | | | | | | similar to | |
| | | | | | | | 2004399A | |
| | | | | | | | chromos | |
| 1254 | CTGGTGGTGCC | 2.03 | 0.21 | 1.63 | 0.10 | Hs.49427 | Gem-interacting | 1.25 |
| | | | | | | | protein | |
| | | | | | | | [Swissprot: sp\| | |
| 1255 | CCTTTCTTTAT | 2.03 | 0.21 | 1.63 | 0.10 | Hs.50535 | Homo sapiens, | 1.25 |
| | | | | | | | Similar to | |
| | | | | | | | hypothetical pr | |
| 1256 | TCTGTGCTGTC | 2.03 | 0.21 | 1.63 | 0.10 | Hs.5452 | ubiquitin specific | 1.25 |
| | | | | | | | protease 20 | |
| | | | | | | | [Swisspr | |
| 1257 | CCAGTGGCTCA | 2.03 | 0.21 | 1.63 | 0.10 | Hs.5753 | inositol(myo)- | 1.25 |
| | | | | | | | 1(or 4)- | |
| | | | | | | | monophosphatase 2 | |
| 1258 | AGGCCTGGGCC | 2.03 | 0.21 | 1.63 | 0.10 | Hs.6163 | PTEN induced | 1.25 |
| | | | | | | | putative kinase 1 | |
| | | | | | | | [Swisspr | |
| 1259 | TACAGACATAC | 2.03 | 0.21 | 1.63 | 0.10 | Hs.63984 | cadherin 13, H- | 1.25 |
| | | | | | | | cadherin (heart) | |
| | | | | | | | [Swissp | |
| 1260 | TGCCCTGTTCC | 2.03 | 0.21 | 1.63 | 0.10 | Hs.6651 | Homo sapiens | 1.25 |
| | | | | | | | clone 23645 | |
| | | | | | | | mRNA sequence | |
| 1261 | ATGACCTGAAG | 2.03 | 0.21 | 1.63 | 0.10 | Hs.74050 | follicular | 1.25 |
| | | | | | | | lymphoma | |
| | | | | | | | variant | |
| | | | | | | | translocatio | |
| 1262 | TTTAAAAAAAA | 2.03 | 0.21 | 1.63 | 0.10 | Hs.74088 | early growth | 1.25 |
| | | | | | | | response 3 | |
| | | | | | | | [Swissprot: sp\| | |
| 1263 | AATATTTAGTG | 2.03 | 0.21 | 1.63 | 0.10 | Hs.75133 | transcription | 1.25 |
| | | | | | | | factor 6-like 1 | |
| | | | | | | | (mitochond | |
| 1264 | TGCCCAGCAAA | 2.03 | 0.21 | 1.63 | 0.10 | Hs.76297 | G protein- | 1.25 |
| | | | | | | | coupled receptor | |
| | | | | | | | kinase 6 [Sw | |
| 1265 | ATTGATGACGG | 2.03 | 0.21 | 1.63 | 0.10 | Hs.7733 | tetratricopeptide | 1.25 |
| | | | | | | | repeat domain 1 | |
| | | | | | | | [Swis | |
| 1266 | GTGTTGAGAGA | 2.03 | 0.21 | 1.63 | 0.10 | Hs.77502 | methionine | 1.25 |
| | | | | | | | adenosyltransferase | |
| | | | | | | | II, alpha | |
| 1267 | GAGTAGAGGCC | 2.03 | 0.21 | 1.63 | 0.10 | Hs.77813 | sphingomyelin | 1.25 |
| | | | | | | | phosphodiesterase | |
| | | | | | | | 1, acid | |
| 1268 | TAACATTGGTG | 2.03 | 0.21 | 1.63 | 0.10 | Hs.79306 | eukaryotic | 1.25 |
| | | | | | | | translation | |
| | | | | | | | initiation factor | |
| 1269 | GAACCACAGGA | 2.03 | 0.21 | 1.63 | 0.10 | Hs.80042 | dolichyl-P- | 1.25 |
| | | | | | | | Glc: Man9GlcNA | |
| | | | | | | | c2-PP-dolichylgl | |
| 1270 | CCCTCCTCTCC | 2.03 | 0.21 | 1.63 | 0.10 | Hs.83173 | cyclin D3 | 1.25 |
| | | | | | | | [Swissprot: | |
| | | | | | | | sp\|P30281; sp\|Q96F | |
| 1271 | TAGCAATCAGA | 2.03 | 0.21 | 1.63 | 0.10 | Hs.83795 | interferon | 1.25 |
| | | | | | | | regulatory factor | |
| | | | | | | | 2 [Swisspr | |
| 1272 | TGTCTGTGTGT | 2.03 | 0.21 | 1.63 | 0.10 | Hs.93739 | ESTs | 1.25 |
| | | | | | | | [Swissprot: | |
| | | | | | | | none] | |
| 1273 | TGGCCAAAAAA | 2.03 | 0.21 | 1.63 | 0.10 | Hs.95044 | ESTs, Weakly | 1.25 |
| | | | | | | | similar to I38022 | |
| | | | | | | | hypotheti | |
| 1274 | GACAAAAAGTC | 2.03 | 0.21 | 1.63 | 0.10 | Hs.9683 | DnaJ (Hsp40) | 1.25 |
| | | | | | | | homolog, | |
| | | | | | | | subfamily C, | |
| | | | | | | | membe | |
| 1275 | GAAAAGCTCCT | 2.03 | 0.21 | 1.63 | 0.10 | Hs.99843 | DKFZP586N072 | 1.25 |
| | | | | | | | 1 protein | |
| | | | | | | | [Swissprot: | |
| | | | | | | | sp\|Q9 | |
| 1276 | TGTAAGTCTGC | −2.47 | 0.53 | −1.23 | 0.09 | Hs.119537 | GAP-associated | 2.01 |
| | | | | | | | tyrosine | |
| | | | | | | | phosphoprotein p | |
| 1277 | TGCTAAAAAAA | −2.47 | 0.53 | −1.23 | 0.09 | Hs.146550 | myosin, heavy | 2.01 |
| | | | | | | | polypeptide 9, | |
| | | | | | | | non-muscle | |
| 1278 | TTAATCCTAAA | −2.47 | 0.53 | −1.23 | 0.09 | Hs.150741 | 2′,3′-cyclic | 2.01 |
| | | | | | | | nucleotide 3′ | |
| | | | | | | | phosphodieste | |
| 1279 | TGCGGAGGCCC | −2.47 | 0.53 | −1.23 | 0.09 | Hs.25723 | Sjogren's | 2.01 |
| | | | | | | | syndrome/sclero | |
| | | | | | | | derma autoantig | |
| 1280 | GAAAAGCCTTC | −2.47 | 0.53 | −1.23 | 0.09 | Hs.78619 | gamma-glutamyl | 2.01 |
| | | | | | | | hydrolase | |
| | | | | | | | (conjugase, fol | |
| 1281 | GGAGGCAGGTG | −1.97 | 0.33 | −1.23 | 0.09 | Hs.206713 | UDP- | 1.60 |
| | | | | | | | Gal: betaGlcNAc | |
| | | | | | | | beta 1,4- | |
| | | | | | | | galactosylt | |
| 1282 | GCTAAAAAAAA | −1.97 | 0.33 | −1.23 | 0.09 | Hs.256697 | histidine triad | 1.60 |
| | | | | | | | nucleotide- | |
| | | | | | | | binding prote | |
| 1283 | TATTTATTCCT | −1.97 | 0.33 | −1.23 | 0.09 | Hs.274464 | diaphorase | 1.60 |
| | | | | | | | (NADH) | |
| | | | | | | | (cytochrome b-5 | |
| | | | | | | | reduct | |
| 1284 | CTTTCTTTGAG | −1.97 | 0.33 | −1.23 | 0.09 | Hs.4909 | dickkopf | 1.60 |
| | | | | | | | (Xenopus laevis) | |
| | | | | | | | homolog 3 [Sw | |
| 1285 | TAAATAATTTC | −2.96 | 0.42 | −1.23 | 0.07 | Hs.1197 | heat shock 10 kD | 2.41 |
| | | | | | | | protein 1 | |
| | | | | | | | (chaperonin 10 | |
| 1286 | CAGATTGTGAA | −2.96 | 0.42 | −1.23 | 0.07 | Hs.142653 | ret finger protein | 2.41 |
| | | | | | | | [Swissprot: | |
| | | | | | | | sp\|P1437 | |
| 1287 | ATGGCCATAGA | −2.96 | 0.42 | −1.23 | 0.07 | Hs.155206 | serine/threonine | 2.41 |
| | | | | | | | kinase 25 | |
| | | | | | | | (Ste20, yeast | |
| 1288 | TTTTTGATAAA | −2.96 | 0.42 | −1.23 | 0.07 | Hs.181165 | eukaryotic | 2.41 |
| | | | | | | | translation | |
| | | | | | | | elongation factor | |
| 1289 | TGGGGTGGAGT | −2.96 | 0.42 | −1.23 | 0.07 | Hs.26403 | glutathione | 2.41 |
| | | | | | | | transferase zeta | |
| | | | | | | | 1 (maleylac | |
| 1290 | GAGTTATGTTG | −2.96 | 0.42 | −1.23 | 0.07 | Hs.279915 | translocase of | 2.41 |
| | | | | | | | inner | |
| | | | | | | | mitochondrial | |
| | | | | | | | membr | |
| 1291 | ACCTCTGGCTT | −2.96 | 0.42 | −1.23 | 0.07 | Hs.283844 | similar to rat | 2.41 |
| | | | | | | | tricarboxylate | |
| | | | | | | | carrier-II | |
| 1292 | GCGGAGAGAGG | −2.96 | 0.42 | −1.23 | 0.07 | Hs.286 | ribosomal | 2.41 |
| | | | | | | | protein L4 | |
| | | | | | | | [Swissprot: | |
| | | | | | | | sp\|P36 | |
| 1293 | AATACTTAAAT | −2.96 | 0.42 | −1.23 | 0.07 | Hs.288908 | Homo sapiens | 2.41 |
| | | | | | | | cDNA: | |
| | | | | | | | FLJ21913 fis, | |
| | | | | | | | clone H | |
| 1294 | GCAAAGAAAAA | −2.96 | 0.42 | −1.23 | 0.07 | Hs.3844 | LIM domain only | 2.41 |
| | | | | | | | 4 [Swissprot: | |
| | | | | | | | sp\|O00158 | |
| 1295 | ACGGCTCCGAG | −2.96 | 0.42 | −1.23 | 0.07 | Hs.48563 | Homo sapiens, | 2.41 |
| | | | | | | | Similar to RIKEN | |
| | | | | | | | cDNA 1110 | |
| 1296 | CTCCTGAAGGC | −2.96 | 0.42 | −1.23 | 0.07 | Hs.4890 | ubiquitin- | 2.41 |
| | | | | | | | conjugating | |
| | | | | | | | enzyme E2E 3 | |
| | | | | | | | (homo | |
| 1297 | ACAGCGTCTGC | −2.96 | 0.42 | −1.23 | 0.07 | Hs.63128 | KIAA1292 | 2.41 |
| | | | | | | | protein | |
| | | | | | | | [Swissprot: | |
| | | | | | | | sp\|Q96GI9; | |
| 1298 | GCCACTACCCC | −2.96 | 0.42 | −1.23 | 0.07 | Hs.71475 | acid cluster | 2.41 |
| | | | | | | | protein 33 | |
| | | | | | | | [Swissprot: sp\| | |
| 1299 | AGGGGATTCCC | −2.96 | 0.42 | −1.23 | 0.07 | Hs.75412 | arginine-rich, | 2.41 |
| | | | | | | | mutated in early | |
| | | | | | | | stage tu | |
| 1300 | TAGGACCCTGC | −2.96 | 0.42 | −1.23 | 0.07 | Hs.76873 | hyaluronoglucos | 2.41 |
| | | | | | | | aminidase 2 | |
| | | | | | | | [Swissprot: | |
| 1301 | GAGCGGCCTCT | −2.96 | 0.42 | −1.23 | 0.07 | Hs.77868 | ORF [Swissprot: | 2.41 |
| | | | | | | | sp\|Q04323; sp\|Q9BV93; | |
| | | | | | | | sp\| | |
| 1302 | GCCCCAGGTAG | −2.96 | 0.42 | −1.23 | 0.07 | Hs.78466 | proteasome | 2.41 |
| | | | | | | | (prosome, | |
| | | | | | | | macropain) 26S | |
| | | | | | | | subu | |
| 1303 | TAAGCATTAAA | −2.96 | 0.42 | −1.23 | 0.07 | Hs.8180 | syndecan | 2.41 |
| | | | | | | | binding protein | |
| | | | | | | | (syntenin) [Sw | |
| 1304 | ACAAAATAAAA | −2.96 | 0.42 | −1.23 | 0.07 | Hs.83469 | nuclear factor | 2.41 |
| | | | | | | | (erythroid- | |
| | | | | | | | derived 2)-lik | |
| 1305 | GGCTGCCCTGG | −2.96 | 0.42 | −1.23 | 0.07 | Hs.90259 | ESTs, Weakly | 2.41 |
| | | | | | | | similar to | |
| | | | | | | | CA13_HUMAN | |
| | | | | | | | COLLA | |
| 1306 | TTGAGCCAGCC | −2.96 | 0.42 | −1.23 | 0.07 | Hs.91142 | KH-type splicing | 2.41 |
| | | | | | | | regulatory | |
| | | | | | | | protein (FUS | |
| 1307 | CCATTCTCCTG | 2.70 | 0.86 | 1.09 | 0.04 | Hs.306907 | hypothetical | 2.48 |
| | | | | | | | protein | |
| | | | | | | | FLJ23519 | |
| | | | | | | | [Swisspro | |
| 1308 | CTCATCTGCTG | 2.70 | 0.86 | 1.09 | 0.04 | Hs.82109 | syndecan 1 | 2.48 |
| | | | | | | | [Swissprot: | |
| | | | | | | | sp\|P18827; sp\|Q96 | |
| 1309 | AAACCAGGGCC | 2.03 | 0.35 | 1.09 | 0.02 | Hs.279836 | HSPC166 | 1.86 |
| | | | | | | | protein | |
| | | | | | | | [Swissprot: | |
| | | | | | | | sp\|Q96FI2; s | |
| 1310 | TGGACCCCCCG | 2.03 | 0.35 | 1.09 | 0.02 | Hs.300071 | hypothetical | 1.86 |
| | | | | | | | protein | |
| | | | | | | | MGC: 5244, | |
| | | | | | | | [Swisspr | |
| 1311 | TTTCAGTGGGT | 2.03 | 0.35 | 1.09 | 0.02 | Hs.31218 | secretory carrier | 1.86 |
| | | | | | | | membrane | |
| | | | | | | | protein 1 [S | |
| 1312 | GCTTAACCTGG | −1.97 | 0.75 | −1.02 | 0.00 | Hs.77508 | glutamate | 1.93 |
| | | | | | | | dehydrogenase | |
| | | | | | | | 1 [Swissprot: s | |
| 1313 | ACTTACCTGCT | −2.08 | 1.18 | −1.09 | 0.00 | Hs.174031 | cytochrome c | 1.91 |
| | | | | | | | oxidase subunit | |
| | | | | | | | VIb [Swiss | |

What is claimed is:

1. An in vitro method for identifying genetically encoded molecules which are differentially expressed in hairy skin when compared to hairless skin, comprising:
a) providing a first sample of human hairy skin comprising genetically encoded molecules which are transcribed and optionally translated in said hairy skin;
b) providing a second sample of human hairless skin comprising genetically encoded molecules which are transcribed and, optionally translated in said hairless skin; and
c) subjecting said first and second samples to a serial analysis of gene expression (SAGE), thereby identifying genetically encoded molecules which are differentially expressed in hairy as compared to hairless skin.

2. The method as claimed in claim 1, wherein said first sample is obtained from hairy scalp and said second sample is obtained from hairless facial skin.

3. The method of claim 1, wherein said genetically encoded molecules are selected from the group consisting of at least one mRNA molecule, at least one protein or polypeptide, or fragments thereof,

4. The method of claim 3, wherein said encoded molecule is translated and is selected from the group consisting of a protein, a polypeptide or a fragment of either, said molecule being characterized and optionally quantitated by at least one method selected from the group consisting of
i. one- or two-dimensional gel electrophoresis
ii. affinity chromatography
iii. protein-protein complexation in solution
iv. mass spectrometry, especially matrix-assisted laser desorption ionization (MALDI) and,
v. use of protein chips.

5. The method of claim 3, wherein said encoded molecule is an mRNA molecule or fragment thereof, said molecule being characterized and optionally quantitated by at least one method selected from the group consisting of
i. Northern blots
ii. reverse transcriptase polymerase chain reaction (RT-PCR),
iii. RNase protection experiments,
iv. dot blots,
v. cDNA sequencing,
vi. clone hybridization,
vii. differential display,
viii. subtractive hybridization,
ix. cDNA fragment fingerprinting,
x. total gene expression analysis (TOGA),
xi. serial analysis of gene expression (SAGE), and
xii. massively parallel signature sequencing (MPSS®) and,
xiii. use of nucleic acid chips.

6. An in vitro method for determining the homeostasis of hairy skin in humans, said method comprising:
a) performing the method of claim 1, thereby identifying a population of hairy skin associated genetically encoded molecules
b) obtaining a hairy skin sample from a patient and determining the presence and optionally the amount of hairy skin associated genetically encoded molecules present in the sample and
c) designating the sample of b) as
i) healthy or homeostasis-undergoing hairy skin if it comprises molecules which are expressed at higher levels in hairy skin relative to hairless skin, or
ii) diseased or homeostasis-impaired hairy skin if it comprises molecules which are expressed at higher levels in hairless skin relative to hairy skin.

7. The method of claim 6, wherein said genetically encoded molecules are selected from the group consisting of at least one mRNA molecule, at least one protein or polypeptide, or fragments thereof

8. The method as claimed in claim 6, comprising:
a) determining the presence and optionally the amount of at least one of the genetically encoded molecules or fragments thereof in said patient sample, said molecule having a UniGene accession number set forth in column 7 of Tables 11 and 12;
b) comparing the results from a) with the expression quotients set forth in column 3 and column 5 in Tables 11 and 12, and
c) designating the patient sample as healthy hairy skin if it comprises molecules which are expressed at levels at least 1.9-fold higher in healthy hairy skin when compared to hairless skin, or designating the patient sample as diseased or homeostasis-impaired hairy skin if it comprises molecules which are expressed at levels at least 1.9-fold higher in hairless skin when compared to hairy skin.

9. The method as claimed in claim 6, comprising
a) determining the presence and optionally the amount of at least one of the genetically encoded molecules or fragments thereof in said patient sample, said molecule having a UniGene accession number set forth in column 7 of Tables 9 and 10,
b) comparing the results from a) with the expression quotients set forth in column 3 and column 5 in Tables 9 and 10, and
c) designating the patient sample as healthy hairy skin if it comprises molecules which are expressed at levels at least 3-fold higher in healthy hairy skin when compared to hairless skin, or designating the patient sample as diseased or homeostasis-impaired hairy skin if it comprises molecules which are expressed at levels at least 3-fold higher in hairless skin when compared to hairy skin.

10. The method as claimed in claim 6, comprising
a) determining the presence and optionally the amount of at least one of the genetically encoded molecules or fragments thereof in said patient sample, said molecule having a UniGene accession number set forth in column 7 of Tables 7 and 8,
b) comparing the results from a) with the expression quotients set forth in column 3 and column 5 in Tables 7 and 8, and
c) designating the patient sample as healthy hairy skin if it comprises molecules which are expressed at levels at least 5-fold higher in healthy hairy skin when compared to hairless skin, or designating the patient sample as diseased or homeostasis-impaired hairy skin if it comprises molecules which are expressed at levels at least 5-fold higher in hairless skin when compared to hairy skin.

11. The method as claimed in claim 6, comprising
a) determining the presence and optionally the amount of at least one of the genetically encoded molecules or fragments thereof in said patient sample, said molecule having a UniGene accession number set forth in column 7 of Table 6,
b) comparing the results from a) with the expression quotients set forth in column 3 and column 5 in Table 6, and
c) designating the patient sample as healthy hairy skin if it comprises molecules which are expressed at levels at least 1.9-fold higher in healthy hairy skin when compared to hairless skin, or designating the patient sample as diseased or homeostasis-impaired hairy skin if it comprises molecules which are expressed at levels at least 1.9-fold higher in hairless skin when compared to hairy skin.

12. The method as claimed in claim 6, comprising
a) determining the presence and optionally the amount of at least one of the genetically encoded molecules or fragments thereof in said patient sample, said molecule having a UniGene accession number set forth in column 7 of Table 5,
b) comparing the results from a) with the expression quotients set forth in column 3 and column 5 in Table 5, and
c) designating the patient sample as healthy hairy skin if it comprises molecules which are expressed at levels at least 3-fold higher in healthy hairy skin when compared to hairless skin, or designating the patient sample as diseased or homeostasis-impaired hairy skin if it comprises molecules which are expressed at levels at least 3-fold higher in hairless skin when compared to hairy skin.

13. The method as claimed in claim 6, comprising
a) determining the presence and optionally the amount of at least one of the genetically encoded molecules or fragments thereof in said patient sample, said molecule having a UniGene accession number set forth in column 7 of Table 4,
b) comparing the results from a) with the expression quotients set forth in column 3 and column 5 in Table 4, and
c) designating the patient sample as healthy hairy skin if it comprises molecules which are expressed at levels at least 5-fold higher in healthy hairy skin when compared to hairless skin, or designating the patient sample as diseased or homeostasis-impaired hairy skin if it comprises molecules which are expressed at levels at least 5-fold higher in hairless skin when compared to hairy skin.

14. The method as claimed in claim 6, comprising
a) determining the presence and optionally the amount of at least one of the genetically encoded molecules or fragments thereof in said patient sample, said molecule having a UniGene accession number set forth in column 7 of Table 3,
b) comparing the results from a) with the expression quotients set forth in column 3 and column 5 in Table 3, and
c) designating the patient sample as healthy hairy skin if it comprises molecules which are expressed at levels at least 10-fold higher in healthy hairy skin when compared to hairless skin, or designating the patient sample as diseased or homeostasis-impaired hairy skin if it comprises molecules which are expressed at levels at least 10-fold higher in hairless skin when compared to hairy skin.

15. The method of claim 6, wherein said encoded molecule is translated and is selected from the group consisting of a protein, a polypeptide or a fragment of either, said molecule being characterized and optionally quantitated by at least one method selected from the group consisting of
i. one- or two-dimensional gel electrophoresis
ii. affinity chromatography
iii. protein-protein complexation in solution
iv. mass spectrometry, especially matrix-assisted laser desorption ionization (MALDI) and,
v. use of protein chips.

16. The method of claim 6, wherein said encoded molecule is an mRNA molecule or fragment thereof, said molecule being characterized and optionally quantitated by at least one method selected from the group consisting of
i. Northern blots
ii. reverse transcriptase polymerase chain reaction (RT-PCR),
iii. RNase protection experiments,
iv. dot blots,
v. cDNA sequencing,
vi. clone hybridization,
vii. differential display,
viii. subtractive hybridization,
ix. cDNA fragment fingerprinting,
x. total gene expression analysis (TOGA),
xi. serial analysis of gene expression (SAGE), and
xii. massively parallel signature sequencing (MPSS®) and,
xiii. use of nucleic acid chip.

17. An in vitro method for determining the homeostasis of hairy skin in humans, comprising:
a) obtaining a sample of genetically encoded molecules or fragments thereof from hairy human skin,
b) quantifying at least two of the genetically encoded hairy skin associated molecules or fragments thereof present in the sample, said hairy skin associated molecules or fragments thereof being identified by the method of claim 1,
c) determining the expression ratios of the at least two genetically encoded hairy skin associated molecules, thereby generating an expression quotient,
d) comparing the expression quotients from c) with the expression quotients provided in at least one table selected from the group consisting of Table 3, Table 4, Table 5, Table 6, Table 7, Table 8, Table 9, Table 10, Table 11 and Table 12 and
e) designating the patient sample as healthy hairy skin if the expression ratios of the sample correspond to the expression ratios in present in hairy skin, or designating the sample as diseased skin if the expression ratios of the patient sample correspond to the expression ratios of hairless skin.

18. The method of claim 17, wherein said encoded molecule is translated and is selected from the group consisting of a protein, a polypeptide or a fragment of either, said molecule being characterized and optionally quantitated by at least one method selected from the group consisting of
i. one- or two-dimensional gel electrophoresis
ii. affinity chromatography
iii. protein-protein complexation in solution
iv. mass spectrometry, especially matrix-assisted laser desorption ionization (MALDI) and,
v. use of protein chips.

19. The method of claim 17, wherein said encoded molecule is an mRNA molecule or fragment thereof, said molecule being characterized and optionally quantitated by at least one method selected from the group consisting of
i. Northern blots
ii. reverse transcriptase polymerase chain reaction (RT-PCR),
iii. RNase protection experiments,
iv. dot blots,
v. cDNA sequencing,
vi. clone hybridization,
vii. differential display,
viii. subtractive hybridization,
ix. cDNA fragment fingerprinting,
x. total gene expression analysis (TOGA),
xi. serial analysis of gene expression (SAGE), and
xii. massively parallel signature sequencing (MPSS®) and,
xiii. use of nucleic acid chips.

20. The method as claimed in claim 1, comprising detecting and optionally quantitating from about 1 to about 5000 genetically encoded molecules which are defined by their UniGene accession number in column 7 in a Table selected from the group consisting of Tables 3 to 12.

21. A test kit for determining the homeostasis of hairy skin in humans in vitro, said test kit comprising reagents suitable for carrying out the method of claim 1.

22. The test kit of claim 21 further comprising reagents suitable for performing at least one of the methods of claim 4.

23. The test kit of claim 21, further comprising reagents suitable for performing at least one of the methods of claim 5.

24. A biochip for determining the homeostasis of hairy skin in humans in vitro, including
i. a solid, i.e. rigid or flexible support and
ii. probes immobilized thereon which are capable of specific binding to at least one of the proteins, mRNA molecules or fragments thereof defined by their UniGene accession number in column 7 in a Table selected from the group of Tables 3 to 12.

25. The biochip of claim 24, comprising probes having at least one sequence selected from the group consisting of SEQ ID NOS: 2, 4, 9, 12, 14, 16, 22, 25, 29, 31, 35, 36, 38, 39, 40, 42, 43, 44, 46, 59, 62, 63, 65, 67, 68, 69, and 74.

26. The biochip as claimed in claim 24, including from about 1 to about 5000, different probes.

27. The biochip as claimed in claim 24, wherein said probes are selected from the group consisting of DNA probes, RNA probes, and PNA probes.

28. The biochip as claimed in claim 24, including probes with a length of about 10 to about 1000 nucleotides.

29. The biochip as claimed in claim 24, wherein said probes are selected from the group consisting of peptide, protein and antibody probes.

30. A test method for determining the efficacy of cosmetic or pharmaceutical active ingredients for disorders or impairments of homeostasis of hairy skin in vitro, said method comprising
a) obtaining a hairy skin sample and determining the status of said skin;
b) contacting said skin sample with an active ingredient for disorders or impairments of homeostasis of hairy skin at least once,
c) determining the skin status of hairy skin following step b) and
d) comparing the skin status results from a) and c), agents which improve said skin status being efficacious for disorders or impairments of homeostasis of hairy skin.

31. The method of claim 30, further comprising formulating said efficacious agent into a cosmetic or pharmaceutically acceptable carrier.

32. A test kit for determining the efficacy of cosmetic or pharmaceutical active ingredients for disorders or impairments of homeostasis of hairy skin, including reagents suitable for performing the method of claim 30.

33. The test kit of claim 32, comprising a biochip.


SEQUENCE LISTING (1335 sequences)

SEQ ID NO: 1 - DNA, 11 bp, Homo Sapiens
gtttgcaagtg

SEQ ID NO: 2 - DNA, 11 bp, Homo Sapiens
atgcgaaaggc

SEQ ID NO: 3 - DNA, 11 bp, Homo Sapiens
cttatggttga

SEQ ID NO: 4 - DNA, 11 bp, Homo Sapiens
ttggggaaaca

SEQ ID NO: 5 - DNA, 11 bp, Homo Sapiens
ggccgtgctgc

SEQ ID NO: 6 - DNA, 11 bp, Homo Sapiens
tgcgcctttat

SEQ ID NO: 7 - DNA, 11 bp, Homo Sapiens
tcattgtaatg

SEQ ID NO: 8 - DNA, 11 bp, Homo Sapiens
aggaacacaaa

SEQ ID NO: 9 - DNA, 11 bp, Homo Sapiens
tagttgcaaat

SEQ ID NO: 10 - DNA, 11 bp, Homo Sapiens
tttggggctgg

SEQ ID NO: 11 - DNA, 11 bp, Homo Sapiens
aatggaatgga

SEQ ID NO: 12 - DNA, 11 bp, Homo Sapiens
ctcttcaggag

SEQ ID NO: 13 - DNA, 11 bp, Homo Sapiens
ggaggccgagc

SEQ ID NO: 14 - DNA, 11 bp, Homo Sapiens
ttttaacaaaa

SEQ ID NO: 15 - DNA, 11 bp, Homo Sapiens
ggcaggatgat

SEQ ID NO: 16 - DNA, 11 bp, Homo Sapiens
tgactgtatta

SEQ ID NO: 17 - DNA, 11 bp, Homo Sapiens
tgaaaagctta

SEQ ID NO: 18 - DNA, 11 bp, Homo Sapiens
aagcacaaaaa

SEQ ID NO: 19 - DNA, 11 bp, Homo Sapiens
cctaaggctaa

SEQ ID NO: 20 - DNA, 11 bp, Homo Sapiens
ggagtctaact

SEQ ID NO: 21 - DNA, 11 bp, Homo Sapiens
caggaggaaag

SEQ ID NO: 22 - DNA, 11 bp, Homo Sapiens
ttttactcaca

SEQ ID NO: 23 - DNA, 11 bp, Homo Sapiens
ggtgaagacaa

SEQ ID NO: 24 - DNA, 11 bp, Homo Sapiens
tgtgctaatat

SEQ ID NO: 25 - DNA, 11 bp, Homo Sapiens
ccactcctcca

SEQ ID NO: 26 - DNA, 11 bp, Homo Sapiens
caccattcagt

SEQ ID NO: 27 - DNA, 11 bp, Homo Sapiens
gaggacccaac

SEQ ID NO: 28 - DNA, 11 bp, Homo Sapiens
ctgcctcctta

SEQ ID NO: 29 - DNA, 11 bp, Homo Sapiens
ttctctccaca

SEQ ID NO: 30 - DNA, 11 bp, Homo Sapiens
gggcttactgt

SEQ ID NO: 31 - DNA, 11 bp, Homo Sapiens
atttagcaagc

SEQ ID NO: 32 - DNA, 11 bp, Homo Sapiens
gtagacttgtc

SEQ ID NO: 33 - DNA, 11 bp, Homo Sapiens
cagaagaggct

SEQ ID NO: 34 - DNA, 11 bp, Homo Sapiens
cggttactgtg

SEQ ID NO: 35 - DNA, 11 bp, Homo Sapiens
acattccaagt

SEQ ID NO: 36 - DNA, 11 bp, Homo Sapiens
cctctttgcat

SEQ ID NO: 37 - DNA, 11 bp, Homo Sapiens
ggaagatgaac

SEQ ID NO: 38 - DNA, 11 bp, Homo Sapiens
cagggagcgcc

SEQ ID NO: 39 - DNA, 11 bp, Homo Sapiens
cccccacctaa

SEQ ID NO: 40 - DNA, 11 bp, Homo Sapiens
ttgttattgcc

SEQ ID NO: 41 - DNA, 11 bp, Homo Sapiens
gctgaacgcgt

SEQ ID NO: 42 - DNA, 11 bp, Homo Sapiens
aactgcttcaa

SEQ ID NO: 43 - DNA, 11 bp, Homo Sapiens
cgcaccattgc

SEQ ID NO: 44 - DNA, 11 bp, Homo Sapiens
tggcctctctg

SEQ ID NO: 45 - DNA, 11 bp, Homo Sapiens
tggctgtgtgg

SEQ ID NO: 46 - DNA, 11 bp, Homo Sapiens
gtgctgattct

SEQ ID NO: 47 - DNA, 11 bp, Homo Sapiens
tgctttgggat

SEQ ID NO: 48 - DNA, 11 bp, Homo Sapiens
actgggtctat

SEQ ID NO: 49 - DNA, 11 bp, Homo Sapiens
aaggtaatatg

SEQ ID NO: 50 - DNA, 11 bp, Homo Sapiens
gggcccttcct

SEQ ID NO: 51 - DNA, 11 bp, Homo Sapiens
aaggcaattta

SEQ ID NO: 52 - DNA, 11 bp, Homo Sapiens
tgtgggtgctg

SEQ ID NO: 53 - DNA, 11 bp, Homo Sapiens
gctgccctggg

SEQ ID NO: 54 - DNA, 11 bp, Homo Sapiens
cgcaccattgc

SEQ ID NO: 55 - DNA, 11 bp, Homo Sapiens
tggcctctctg

SEQ ID NO: 56 - DNA, 11 bp, Homo Sapiens
tggctgtgtgg

SEQ ID NO: 57 - DNA, 11 bp, Homo Sapiens
gtgcgctgagc

SEQ ID NO: 58 - DNA, 11 bp, Homo Sapiens
agaaccttaaa

SEQ ID NO: 59 - DNA, 11 bp, Homo Sapiens
cagtttgtaca

SEQ ID NO: 60 - DNA, 11 bp, Homo Sapiens
ggtgtctgtgg

SEQ ID NO: 61 - DNA, 11 bp, Homo Sapiens
cagttctctgt

SEQ ID NO: 62 - DNA, 11 bp, Homo Sapiens
ttgatgtacag

SEQ ID NO: 63 - DNA, 11 bp, Homo Sapiens
tacatcagtaa

SEQ ID NO: 64 - DNA, 11 bp, Homo Sapiens
ggaggctgagg

SEQ ID NO: 65 - DNA, 11 bp, Homo Sapiens
taaccaatcag

SEQ ID NO: 66 - DNA, 11 bp, Homo Sapiens
gactctggtgc

SEQ ID NO: 67 - DNA, 11 bp, Homo Sapiens
gatctcttggg

SEQ ID NO: 68 - DNA, 11 bp, Homo Sapiens
gaaaacaaagt

SEQ ID NO: 69 - DNA, 11 bp, Homo Sapiens
tttggttttcc

SEQ ID NO: 70 - DNA, 11 bp, Homo Sapiens
gcaaaaaaaaa

SEQ ID NO: 71 - DNA, 11 bp, Homo Sapiens
gagagctaact

SEQ ID NO: 72 - DNA, 11 bp, Homo Sapiens
acattcttttt

SEQ ID NO: 73 - DNA, 11 bp, Homo Sapiens
cttcttgcccc

SEQ ID NO: 74 - DNA, 11 bp, Homo Sapiens
tcaccttaggt

SEQ ID NO: 75 - DNA, 11 bp, Homo Sapiens
ggaggtggagg

SEQ ID NO: 76 - DNA, 11 bp, Homo Sapiens
gggggtgggtg

SEQ ID NO: 77 - DNA, 11 bp, Homo Sapiens
ggctatgttaa

SEQ ID NO: 78 - DNA, 11 bp, Homo Sapiens
ctagattccct

SEQ ID NO: 79 - DNA, 11 bp, Homo Sapiens
aggtggacagg

SEQ ID NO: 80 - DNA, 11 bp, Homo Sapiens
gcatagtgttt

SEQ ID NO: 81 - DNA, 11 bp, Homo Sapiens
gatgtgaaaag

SEQ ID NO: 82 - DNA, 11 bp, Homo Sapiens
aggtctaaggc

SEQ ID NO: 83 - DNA, 11 bp, Homo Sapiens
gtctcatttga

SEQ ID NO: 84 - DNA, 11 bp, Homo Sapiens
tctggggaaat

SEQ ID NO: 85 - DNA, 11 bp, Homo Sapiens
ttgtctgcttt

SEQ ID NO: 86 - DNA, 11 bp, Homo Sapiens
gttcttgtatc

SEQ ID NO: 87 - DNA, 11 bp, Homo Sapiens
cttctggataa

SEQ ID NO: 88 - DNA, 11 bp, Homo Sapiens
aaaataaacaa

SEQ ID NO: 89 - DNA, 11 bp, Homo Sapiens
ttacttccccc

SEQ ID NO: 90 - DNA, 11 bp, Homo Sapiens
ctttttgtgcc

SEQ ID NO: 91 - DNA, 11 bp, Homo Sapiens
tgggtctgaac

SEQ ID NO: 92 - DNA, 11 bp, Homo Sapiens
tgccaattaag

SEQ ID NO: 93 - DNA, 11 bp, Homo Sapiens
aagggggcaag

SEQ ID NO: 94 - DNA, 11 bp, Homo Sapiens
tctgcaaagga

SEQ ID NO: 95 - DNA, 11 bp, Homo Sapiens
acgaaaccccg

SEQ ID NO: 96 - DNA, 11 bp, Homo Sapiens
catcctgctgc

SEQ ID NO: 97 - DNA, 11 bp, Homo Sapiens
ctgttgctggg

SEQ ID NO: 98 - DNA, 11 bp, Homo Sapiens
gacaaagagag

SEQ ID NO: 99 - DNA, 11 bp, Homo Sapiens
tggatgtacat

SEQ ID NO: 100 - DNA, 11 bp, Homo Sapiens
tgtgagggaag

SEQ ID NO: 101 - DNA, 11 bp, Homo Sapiens
atgttcaggct

SEQ ID NO: 102 - DNA, 11 bp, Homo Sapiens
tatgagataga

SEQ ID NO: 103 - DNA, 11 bp, Homo Sapiens
taccaaaataa

SEQ ID NO: 104 - DNA, 11 bp, Homo Sapiens
tgctgcccccc

SEQ ID NO: 105 - DNA, 11 bp, Homo Sapiens
ttttattggaa

SEQ ID NO: 106 - DNA, 11 bp, Homo Sapiens
ctcggtgatgt

SEQ ID NO: 107 - DNA, 11 bp, Homo Sapiens
caaataaacct

SEQ ID NO: 108 - DNA, 11 bp, Homo Sapiens
gcaaatcagat

SEQ ID NO: 109 - DNA, 11 bp, Homo Sapiens
taactaacaaa

SEQ ID NO: 110 - DNA, 11 bp, Homo Sapiens
gtggcctgtgc

SEQ ID NO: 111 - DNA, 11 bp, Homo Sapiens
ggggctggggt

SEQ ID NO: 112 - DNA, 11 bp, Homo Sapiens
taataaagaac

SEQ ID NO: 113 - DNA, 11 bp, Homo Sapiens
cttttttccta

SEQ ID NO: 114 - DNA, 11 bp, Homo Sapiens
ggggtggggcc

SEQ ID NO: 115 - DNA, 11 bp, Homo Sapiens
aaactctatat

SEQ ID NO: 116 - DNA, 11 bp, Homo Sapiens
gcccagtgata

SEQ ID NO: 117 - DNA, 11 bp, Homo Sapiens
ctaaggaccaa

SEQ ID NO: 118 - DNA, 11 bp, Homo Sapiens
cacgaagatgc

SEQ ID NO: 119 - DNA, 11 bp, Homo Sapiens
gtatttaacat

SEQ ID NO: 120 - DNA, 11 bp, Homo Sapiens
gactgaatgta

SEQ ID NO: 121 - DNA, 11 bp, Homo Sapiens
tacagatcaca

SEQ ID NO: 122 - DNA, 11 bp, Homo Sapiens
ccctatcacaa

SEQ ID NO: 123 - DNA, 11 bp, Homo Sapiens
gacataaatcc

SEQ ID NO: 124 - DNA, 11 bp, Homo Sapiens
tgcttcatctg

SEQ ID NO: 125 - DNA, 11 bp, Homo Sapiens
cagtgttgggg

SEQ ID NO: 126 - DNA, 11 bp, Homo Sapiens
aagtttccaat

SEQ ID NO: 127 - DNA, 11 bp, Homo Sapiens
ttaaatagcac

SEQ ID NO: 128 - DNA, 11 bp, Homo Sapiens
tttttcactta

SEQ ID NO: 129 - DNA, 11 bp, Homo Sapiens
cctatggcttt

SEQ ID NO: 130 - DNA, 11 bp, Homo Sapiens
gcattgatgtg

SEQ ID NO: 131 - DNA, 11 bp, Homo Sapiens
gacgtccagag

SEQ ID NO: 132 - DNA, 11 bp, Homo Sapiens
gttgtaaataa

SEQ ID NO: 133 - DNA, 11 bp, Homo Sapiens
ctgatttgtac

SEQ ID NO: 134 - DNA, 11 bp, Homo Sapiens
ccctgctcctc

SEQ ID NO: 135 - DNA, 11 bp, Homo Sapiens
tggcgtggccg

SEQ ID NO: 136 - DNA, 11 bp, Homo Sapiens
ctggatctggg

SEQ ID NO: 137 - DNA, 11 bp, Homo Sapiens
cagatcgaaaa

SEQ ID NO: 138 - DNA, 11 bp, Homo Sapiens
caggttgtgag

SEQ ID NO: 139 - DNA, 11 bp, Homo Sapiens
aaataaaaggt

SEQ ID NO: 140 - DNA, 11 bp, Homo Sapiens
gttaccgagga

SEQ ID NO: 141 - DNA, 11 bp, Homo Sapiens
atttcaaaaaa

SEQ ID NO: 142 - DNA, 11 bp, Homo Sapiens
gttatggctgg

SEQ ID NO: 143 - DNA, 11 bp, Homo Sapiens
ttccctgtgag

SEQ ID NO: 144 - DNA, 11 bp, Homo Sapiens
gaagatgaata

SEQ ID NO: 145 - DNA, 11 bp, Homo Sapiens
actactaaata

SEQ ID NO: 146 - DNA, 11 bp, Homo Sapiens
gagaaaaagtg

SEQ ID NO: 147 - DNA, 11 bp, Homo Sapiens
ggcgggtcggg

SEQ ID NO: 148 - DNA, 11 bp, Homo Sapiens
aaaccagaggt

SEQ ID NO: 149 - DNA, 11 bp, Homo Sapiens
gccaggttgcc

SEQ ID NO: 150 - DNA, 11 bp, Homo Sapiens
gctggagttgc

SEQ ID NO: 151 - DNA, 11 bp, Homo Sapiens
acaggtgttta

SEQ ID NO: 152 - DNA, 11 bp, Homo Sapiens
tttcagattgg

SEQ ID NO: 153 - DNA, 11 bp, Homo Sapiens
tgtgacactga

SEQ ID NO: 154 - DNA, 11 bp, Homo Sapiens
aagttggtgct

SEQ ID NO: 155 - DNA, 11 bp, Homo Sapiens
ctttatgtgat

SEQ ID NO: 156 - DNA, 11 bp, Homo Sapiens
ggttgtctaat

SEQ ID NO: 157 - DNA, 11 bp, Homo Sapiens
ctggagtgcag

SEQ ID NO: 158 - DNA, 11 bp, Homo Sapiens
attatcctaat

SEQ ID NO: 159 - DNA, 11 bp, Homo Sapiens
gcaataagtgt

SEQ ID NO: 160 - DNA, 11 bp, Homo Sapiens
ttgtaataaaa

SEQ ID NO: 161 - DNA, 11 bp, Homo Sapiens
gaaagcatacc

SEQ ID NO: 162 - DNA, 11 bp, Homo Sapiens
aaggagattat

SEQ ID NO: 163 - DNA, 11 bp, Homo Sapiens
gctgtataatt

SEQ ID NO: 164 - DNA, 11 bp, Homo Sapiens
gttggataggg

SEQ ID NO: 165 - DNA, 11 bp, Homo Sapiens
cagaaatatat

SEQ ID NO: 166 - DNA, 11 bp, Homo Sapiens
gaggtgaaggg

SEQ ID NO: 167 - DNA, 11 bp, Homo Sapiens
ttactctttct

SEQ ID NO: 168 - DNA, 11 bp, Homo Sapiens
taaactgtaaa

SEQ ID NO: 169 - DNA, 11 bp, Homo Sapiens
cgtttaatcat

SEQ ID NO: 170 - DNA, 11 bp, Homo Sapiens
gtcataagagg

SEQ ID NO: 171 - DNA, 11 bp, Homo Sapiens
ctgaactcttc

SEQ ID NO: 172 - DNA, 11 bp, Homo Sapiens
caggctgtaga

SEQ ID NO: 173 - DNA, 11 bp, Homo Sapiens
ttctggtatta

SEQ ID NO: 174 - DNA, 11 bp, Homo Sapiens
agggtgggggt

SEQ ID NO: 175 - DNA, 11 bp, Homo Sapiens
tgaagtgcttg

SEQ ID NO: 176 - DNA, 11 bp, Homo Sapiens
ttaatagtggg

SEQ ID NO: 177 - DNA, 11 bp, Homo Sapiens
attctgtcata

SEQ ID NO: 178 - DNA, 11 bp, Homo Sapiens
aaatatgtgtt

SEQ ID NO: 179 - DNA, 11 bp, Homo Sapiens
tgcactccttc

SEQ ID NO: 180 - DNA, 11 bp, Homo Sapiens
agccctggctg

SEQ ID NO: 181 - DNA, 11 bp, Homo Sapiens
agctccttgag

SEQ ID NO: 182 - DNA, 11 bp, Homo Sapiens
tgacttgtaat

SEQ ID NO: 183 - DNA, 11 bp, Homo Sapiens
tatccaagtaa

SEQ ID NO: 184 - DNA, 11 bp, Homo Sapiens
agtccccaacc

SEQ ID NO: 185 - DNA, 11 bp, Homo Sapiens
gaaacagacgg

SEQ ID NO: 186 - DNA, 11 bp, Homo Sapiens
gagagcctcag

SEQ ID NO: 187 - DNA, 11 bp, Homo Sapiens
tgtactactta

SEQ ID NO: 188 - DNA, 11 bp, Homo Sapiens
gtgggaccatt

SEQ ID NO: 189 - DNA, 11 bp, Homo Sapiens
gttcttctgtt

SEQ ID NO: 190 - DNA, 11 bp, Homo Sapiens
actgaaggcgc

SEQ ID NO: 191 - DNA, 11 bp, Homo Sapiens
ggtttgattac

SEQ ID NO: 192 - DNA, 11 bp, Homo Sapiens
ccgtagtgcct

SEQ ID NO: 193 - DNA, 11 bp, Homo Sapiens
tggggattacc

SEQ ID NO: 194 - DNA, 11 bp, Homo Sapiens
cgatattcccc

SEQ ID NO: 195 - DNA, 11 bp, Homo Sapiens
ttggtgcttgg

SEQ ID NO: 196 - DNA, 11 bp, Homo Sapiens
gtccctgcctt

SEQ ID NO: 197 - DNA, 11 bp, Homo Sapiens
tattctcaata

SEQ ID NO: 198 - DNA, 11 bp, Homo Sapiens
tgtggcaaagt

SEQ ID NO: 199 - DNA, 11 bp, Homo Sapiens
ccccatactac

SEQ ID NO: 200 - DNA, 11 bp, Homo Sapiens
gtgttgggggt

SEQ ID NO: 201 - DNA, 11 bp, Homo Sapiens
gccagctgaca

SEQ ID NO: 202 - DNA, 11 bp, Homo Sapiens
ctgaggcctgg

SEQ ID NO: 203 - DNA, 11 bp, Homo Sapiens
ggcgcctcctt

SEQ ID NO: 204 - DNA, 11 bp, Homo Sapiens
aagaagcaaga

SEQ ID NO: 205 - DNA, 11 bp, Homo Sapiens
gcggggtaccc

SEQ ID NO: 206 - DNA, 11 bp, Homo Sapiens
attttgtcgtg

SEQ ID NO: 207 - DNA, 11 bp, Homo Sapiens
tggcttattaa

SEQ ID NO: 208 - DNA, 11 bp, Homo Sapiens
gcacttcaaac

SEQ ID NO: 209 - DNA, 11 bp, Homo Sapiens
tatatggatgt

SEQ ID NO: 210 - DNA, 11 bp, Homo Sapiens
tcagtttggag

SEQ ID NO: 211 - DNA, 11 bp, Homo Sapiens
aaaataaagct

SEQ ID NO: 212 - DNA, 11 bp, Homo Sapiens
cacaaaatctc

SEQ ID NO: 213 - DNA, 11 bp, Homo Sapiens
ctgaaaaaaaa

SEQ ID NO: 214 - DNA, 11 bp, Homo Sapiens
aatggaaggtg

SEQ ID NO: 215 - DNA, 11 bp, Homo Sapiens
gctcattaaag

SEQ ID NO: 216 - DNA, 11 bp, Homo Sapiens
atggcctcctc

SEQ ID NO: 217 - DNA, 11 bp, Homo Sapiens
ttttctgctgg

SEQ ID NO: 218 - DNA, 11 bp, Homo Sapiens
atttgtgtgta

SEQ ID NO: 219 - DNA, 11 bp, Homo Sapiens
tgtgtgtgtgt

SEQ ID NO: 220 - DNA, 11 bp, Homo Sapiens
tctgttaataa

SEQ ID NO: 221 - DNA, 11 bp, Homo Sapiens
aacaactggct

SEQ ID NO: 222 - DNA, 11 bp, Homo Sapiens
tgtcagagatg

SEQ ID NO: 223 - DNA, 11 bp, Homo Sapiens
tgggactccag

SEQ ID NO: 224 - DNA, 11 bp, Homo Sapiens
gaaactagatc

SEQ ID NO: 225 - DNA, 11 bp, Homo Sapiens
gtggcgcgcac

SEQ ID NO: 226 - DNA, 11 bp, Homo Sapiens
tgggctctgaa

SEQ ID NO: 227 - DNA, 11 bp, Homo Sapiens
taccaagccag

SEQ ID NO: 228 - DNA, 11 bp, Homo Sapiens
ctgaaaaccac

SEQ ID NO: 229 - DNA, 11 bp, Homo Sapiens
aattgccactg

SEQ ID NO: 230 - DNA, 11 bp, Homo Sapiens
ttcagggcttc

SEQ ID NO: 231 - DNA, 11 bp, Homo Sapiens
agctttccaat

SEQ ID NO: 232 - DNA, 11 bp, Homo Sapiens
ttgggagtgag

SEQ ID NO: 233 - DNA, 11 bp, Homo Sapiens
tggttctatat

SEQ ID NO: 234 - DNA, 11 bp, Homo Sapiens
ggtgtgcacct

SEQ ID NO: 235 - DNA, 11 bp, Homo Sapiens
agtggctgccc

SEQ ID NO: 236 - DNA, 11 bp, Homo Sapiens
cagggcgggtt

SEQ ID NO: 237 - DNA, 11 bp, Homo Sapiens
taaacattgtc

SEQ ID NO: 238 - DNA, 11 bp, Homo Sapiens
gcagctgacgg

SEQ ID NO: 239 - DNA, 11 bp, Homo Sapiens
aataatcctgg

SEQ ID NO: 240 - DNA, 11 bp, Homo Sapiens
actgcaccact

SEQ ID NO: 241 - DNA, 11 bp, Homo Sapiens
tggctgcatag

SEQ ID NO: 242 - DNA, 11 bp, Homo Sapiens
ggcttgctgac

SEQ ID NO: 243 - DNA, 11 bp, Homo Sapiens
tttctggaggt

SEQ ID NO: 244 - DNA, 11 bp, Homo Sapiens
ttgggaggctg

SEQ ID NO: 245 - DNA, 11 bp, Homo Sapiens
aagatattctc

SEQ ID NO: 246 - DNA, 11 bp, Homo Sapiens
ccttgtccagc

SEQ ID NO: 247 - DNA, 11 bp, Homo Sapiens
gcttttattca

SEQ ID NO: 248 - DNA, 11 bp, Homo Sapiens
tttgttaattc

SEQ ID NO: 249 - DNA, 11 bp, Homo Sapiens
ggggcaacagc

SEQ ID NO: 250 - DNA, 11 bp, Homo Sapiens
ctggcgccgat

SEQ ID NO: 251 - DNA, 11 bp, Homo Sapiens
catcgttacat

SEQ ID NO: 252 - DNA, 11 bp, Homo Sapiens
ttgtccagagg

SEQ ID NO: 253 - DNA, 11 bp, Homo Sapiens
gacaatgccag

SEQ ID NO: 254 - DNA, 11 bp, Homo Sapiens
tacattctgtg

SEQ ID NO: 255 - DNA, 11 bp, Homo Sapiens
atagacataaa

SEQ ID NO: 256 - DNA, 11 bp, Homo Sapiens
gtggatggact

SEQ ID NO: 257 - DNA, 11 bp, Homo Sapiens
ccttccaaatt

SEQ ID NO: 258 - DNA, 11 bp, Homo Sapiens
acaaaaaaaaa

SEQ ID NO: 259 - DNA, 11 bp, Homo Sapiens
caagcaaaata

SEQ ID NO: 260 - DNA, 11 bp, Homo Sapiens
tatttcaattg

SEQ ID NO: 261 - DNA, 11 bp, Homo Sapiens
tcatagccttg

SEQ ID NO: 262 - DNA, 11 bp, Homo Sapiens
ttggtggaggt

SEQ ID NO: 263 - DNA, 11 bp, Homo Sapiens
tcttgaacagc

SEQ ID NO: 264 - DNA, 11 bp, Homo Sapiens
tcagacaaaag

SEQ ID NO: 265 - DNA, 11 bp, Homo Sapiens
tacccccaaac

SEQ ID NO: 266 - DNA, 11 bp, Homo Sapiens
gacccttttgg

SEQ ID NO: 267 - DNA, 11 bp, Homo Sapiens
ttggcattgtc

SEQ ID NO: 268 - DNA, 11 bp, Homo Sapiens
tgaaagtaaca

SEQ ID NO: 269 - DNA, 11 bp, Homo Sapiens
gaggagtgggt

SEQ ID NO: 270 - DNA, 11 bp, Homo Sapiens
gcaagacccca

SEQ ID NO: 271 - DNA, 11 bp, Homo Sapiens
tgcttgacaag

SEQ ID NO: 272 - DNA, 11 bp, Homo Sapiens
tgtggtggtgt

SEQ ID NO: 273 - DNA, 11 bp, Homo Sapiens
aagccttgctg

SEQ ID NO: 274 - DNA, 11 bp, Homo Sapiens
cacgcaatgct

SEQ ID NO: 275 - DNA, 11 bp, Homo Sapiens
gctaacccctg

SEQ ID NO: 276 - DNA, 11 bp, Homo Sapiens
tgtggcgtata

SEQ ID NO: 277 - DNA, 11 bp, Homo Sapiens
gactcttcagt

SEQ ID NO: 278 - DNA, 11 bp, Homo Sapiens
gatgaactgaa

SEQ ID NO: 279 - DNA, 11 bp, Homo Sapiens
ctgccaagttg

SEQ ID NO: 280 - DNA, 11 bp, Homo Sapiens
aatgaacaata

SEQ ID NO: 281 - DNA, 11 bp, Homo Sapiens
gttgtctttga

SEQ ID NO: 282 - DNA, 11 bp, Homo Sapiens
gattgaacctc

SEQ ID NO: 283 - DNA, 11 bp, Homo Sapiens
agctcttggag

SEQ ID NO: 284 - DNA, 11 bp, Homo Sapiens
tgatgtggaat

SEQ ID NO: 285 - DNA, 11 bp, Homo Sapiens
tcacaaaagag

SEQ ID NO: 286 - DNA, 11 bp, Homo Sapiens
tagatgtgatt

SEQ ID NO: 287 - DNA, 11 bp, Homo Sapiens
gcatctgttta

SEQ ID NO: 288 - DNA, 11 bp, Homo Sapiens
gctctgtaagc

SEQ ID NO: 289 - DNA, 11 bp, Homo Sapiens
tgaagttatac

SEQ ID NO: 290 - DNA, 11 bp, Homo Sapiens
ttatggatctc

SEQ ID NO: 291 - DNA, 11 bp, Homo Sapiens
gtgagacccca

SEQ ID NO: 292 - DNA, 11 bp, Homo Sapiens
ccagtgcactc

SEQ ID NO: 293 - DNA, 11 bp, Homo Sapiens
ttgtaaaagga

SEQ ID NO: 294 - DNA, 11 bp, Homo Sapiens
taagtttaatt

SEQ ID NO: 295 - DNA, 11 bp, Homo Sapiens
caaaatcagga

SEQ ID NO: 296 - DNA, 11 bp, Homo Sapiens
atgtgtaacga

SEQ ID NO: 297 - DNA, 11 bp, Homo Sapiens
gcagtcgcttg

SEQ ID NO: 298 - DNA, 11 bp, Homo Sapiens
gggcagaatat

SEQ ID NO: 299 - DNA, 11 bp, Homo Sapiens
cagttccctcc

SEQ ID NO: 300 - DNA, 11 bp, Homo Sapiens
tcgggtgtggg

SEQ ID NO: 301 - DNA, 11 bp, Homo Sapiens
ttaaaatacag

SEQ ID NO: 302 - DNA, 11 bp, Homo Sapiens
ctcagaacact

SEQ ID NO: 303 - DNA, 11 bp, Homo Sapiens
cctgcaccaac

SEQ ID NO: 304 - DNA, 11 bp, Homo Sapiens
ttgtttgtaaa

SEQ ID NO: 305 - DNA, 11 bp, Homo Sapiens
tgtttatattg

SEQ ID NO: 306 - DNA, 11 bp, Homo Sapiens
gcacctcctag

SEQ ID NO: 307 - DNA, 11 bp, Homo Sapiens
taaatctgaag

SEQ ID NO: 308 - DNA, 11 bp, Homo Sapiens
cataaccttcc

SEQ ID NO: 309 - DNA, 11 bp, Homo Sapiens
gggggggtctc

SEQ ID NO: 310 - DNA, 11 bp, Homo Sapiens
tgtctggttgt

SEQ ID NO: 311 - DNA, 11 bp, Homo Sapiens
tctggtctggg

SEQ ID NO: 312 - DNA, 11 bp, Homo Sapiens
agtcagctgga

SEQ ID NO: 313 - DNA, 11 bp, Homo Sapiens
aaatgacttat

SEQ ID NO: 314 - DNA, 11 bp, Homo Sapiens
tagagtgtaaa

SEQ ID NO: 315 - DNA, 11 bp, Homo Sapiens
tcagtgtgtga

SEQ ID NO: 316 - DNA, 11 bp, Homo Sapiens
acacctctaaa

SEQ ID NO: 317 - DNA, 11 bp, Homo Sapiens
gaggctcaatc

SEQ ID NO: 318 - DNA, 11 bp, Homo Sapiens
ctgaactgtga

SEQ ID NO: 319 - DNA, 11 bp, Homo Sapiens
cacctagcata

SEQ ID NO: 320 - DNA, 11 bp, Homo Sapiens
aagttttctaa

SEQ ID NO: 321 - DNA, 11 bp, Homo Sapiens
ctaaaaggaga

SEQ ID NO: 322 - DNA, 11 bp, Homo Sapiens
attcctttaat

SEQ ID NO: 323 - DNA, 11 bp, Homo Sapiens
gcttaatgtgt

SEQ ID NO: 324 - DNA, 11 bp, Homo Sapiens
ctatggttgca

SEQ ID NO: 325 - DNA, 11 bp, Homo Sapiens
cttttcatcat

SEQ ID NO: 326 - DNA, 11 bp, Homo Sapiens
gtctttctggg

SEQ ID NO: 327 - DNA, 11 bp, Homo Sapiens
tgagttttaca

SEQ ID NO: 328 - DNA, 11 bp, Homo Sapiens
tctacaaatat

SEQ ID NO: 329 - DNA, 11 bp, Homo Sapiens
ctgagttaggt

SEQ ID NO: 330 - DNA, 11 bp, Homo Sapiens
ggaaggggagg

SEQ ID NO: 331 - DNA, 11 bp, Homo Sapiens
gagatgtctga

SEQ ID NO: 332 - DNA, 11 bp, Homo Sapiens
gttacaaacta

SEQ ID NO: 333 - DNA, 11 bp, Homo Sapiens
tctggtttgtc

SEQ ID NO: 334 - DNA, 11 bp, Homo Sapiens
ttctaatcatt

SEQ ID NO: 335 - DNA, 11 bp, Homo Sapiens
tagtttcaact

SEQ ID NO: 336 - DNA, 11 bp, Homo Sapiens
gaggttcttcc

SEQ ID NO: 337 - DNA, 11 bp, Homo Sapiens
agggctgcagg

SEQ ID NO: 338 - DNA, 11 bp, Homo Sapiens
gtgtaataaga

SEQ ID NO: 339 - DNA, 11 bp, Homo Sapiens
ggatacaacct

SEQ ID NO: 340 - DNA, 11 bp, Homo Sapiens
aagaatcaaaa

SEQ ID NO: 341 - DNA, 11 bp, Homo Sapiens
tgagcctcgtg

SEQ ID NO: 342 - DNA, 11 bp, Homo Sapiens
actgtttgttt

SEQ ID NO: 343 - DNA, 11 bp, Homo Sapiens
ccactgcagtc

SEQ ID NO: 344 - DNA, 11 bp, Homo Sapiens
ctgctatggtc

SEQ ID NO: 345 - DNA, 11 bp, Homo Sapiens
tgaggcctctc

SEQ ID NO: 346 - DNA, 11 bp, Homo Sapiens
gctgtaggggc

SEQ ID NO: 347 - DNA, 11 bp, Homo Sapiens
ttggcaacatt

SEQ ID NO: 348 - DNA, 11 bp, Homo Sapiens
atagtagcttc

SEQ ID NO: 349 - DNA, 11 bp, Homo Sapiens
gggctacgtcc

SEQ ID NO: 350 - DNA, 11 bp, Homo Sapiens
gaaagccccta

SEQ ID NO: 351 - DNA, 11 bp, Homo Sapiens
acccagcgggc

SEQ ID NO: 352 - DNA, 11 bp, Homo Sapiens
agagctcacta

SEQ ID NO: 353 - DNA, 11 bp, Homo Sapiens
cttctagggag

SEQ ID NO: 354 - DNA, 11 bp, Homo Sapiens
cctaagggaga

SEQ ID NO: 355 - DNA, 11 bp, Homo Sapiens
actaactgtgt

SEQ ID NO: 356 - DNA, 11 bp, Homo Sapiens
tgtaagaaaag

SEQ ID NO: 357 - DNA, 11 bp, Homo Sapiens
ttaaatcccat

SEQ ID NO: 358 - DNA, 11 bp, Homo Sapiens
taaacttctga

SEQ ID NO: 359 - DNA, 11 bp, Homo Sapiens
caagtaactag

SEQ ID NO: 360 - DNA, 11 bp, Homo Sapiens
acatagaccga

SEQ ID NO: 361 - DNA, 11 bp, Homo Sapiens
agctgtctctt

SEQ ID NO: 362 - DNA, 11 bp, Homo Sapiens
tgggagaagtg

SEQ ID NO: 363 - DNA, 11 bp, Homo Sapiens
ccattgcattc

SEQ ID NO: 364 - DNA, 11 bp, Homo Sapiens
tatagacctca

SEQ ID NO: 365 - DNA, 11 bp, Homo Sapiens
tgccctcagga

SEQ ID NO: 366 - DNA, 11 bp, Homo Sapiens
gtctttaggaa

SEQ ID NO: 367 - DNA, 11 bp, Homo Sapiens
gaaaaaaatgg

SEQ ID NO: 368 - DNA, 11 bp, Homo Sapiens
ggagagtacac

SEQ ID NO: 369 - DNA, 11 bp, Homo Sapiens
aattgaaaagg

SEQ ID NO: 370 - DNA, 11 bp, Homo Sapiens
ttgtcctctgg

SEQ ID NO: 371 - DNA, 11 bp, Homo Sapiens
ctgctaagatg

SEQ ID NO: 372 - DNA, 11 bp, Homo Sapiens
cagaactagac

SEQ ID NO: 373 - DNA, 11 bp, Homo Sapiens
ttttgatgaga

SEQ ID NO: 374 - DNA, 11 bp, Homo Sapiens
gctagtctgtg

SEQ ID NO: 375 - DNA, 11 bp, Homo Sapiens
ggcacagaagg

SEQ ID NO: 376 - DNA, 11 bp, Homo Sapiens
tttttcttaaa

SEQ ID NO: 377 - DNA, 11 bp, Homo Sapiens
gccaaagtgtt

SEQ ID NO: 378 - DNA, 11 bp, Homo Sapiens
tatgaacaaaa

SEQ ID NO: 379 - DNA, 11 bp, Homo Sapiens
cattcaccata

SEQ ID NO: 380 - DNA, 11 bp, Homo Sapiens
ataggtcagaa

SEQ ID NO: 381 - DNA, 11 bp, Homo Sapiens
aagaactaaaa

SEQ ID NO: 382 - DNA, 11 bp, Homo Sapiens
tgatattaggg

SEQ ID NO: 383 - DNA, 11 bp, Homo Sapiens
gtttccccaga

SEQ ID NO: 384 - DNA, 11 bp, Homo Sapiens
cagataagttt

SEQ ID NO: 385 - DNA, 11 bp, Homo Sapiens
taaaaggaggt

SEQ ID NO: 386 - DNA, 11 bp, Homo Sapiens
caatggagctt

SEQ ID NO: 387 - DNA, 11 bp, Homo Sapiens
tatgtatgttg

SEQ ID NO: 388 - DNA, 11 bp, Homo Sapiens
tccagaataaa

SEQ ID NO: 389 - DNA, 11 bp, Homo Sapiens
tttatctgctg

SEQ ID NO: 390 - DNA, 11 bp, Homo Sapiens
ccaataaaaga

SEQ ID NO: 391 - DNA, 11 bp, Homo Sapiens
agccgggcttt

SEQ ID NO: 392 - DNA, 11 bp, Homo Sapiens
caaaactgttt

SEQ ID NO: 393 - DNA, 11 bp, Homo Sapiens
tatttttactg

SEQ ID NO: 394 - DNA, 11 bp, Homo Sapiens
atacatactgt

SEQ ID NO: 395 - DNA, 11 bp, Homo Sapiens
gccaacaacga

SEQ ID NO: 396 - DNA, 11 bp, Homo Sapiens
tgtttatccta

SEQ ID NO: 397 - DNA, 11 bp, Homo Sapiens
cgaaggctgta

SEQ ID NO: 398 - DNA, 11 bp, Homo Sapiens
gccattataag

SEQ ID NO: 399 - DNA, 11 bp, Homo Sapiens
atgtttgccct

SEQ ID NO: 400 - DNA, 11 bp, Homo Sapiens
tatgcttagta

SEQ ID NO: 401 - DNA, 11 bp, Homo Sapiens
ttttacagtac

SEQ ID NO: 402 - DNA, 11 bp, Homo Sapiens
tctgtaaaaaa

SEQ ID NO: 403 - DNA, 11 bp, Homo Sapiens
gaaaaaataaa

SEQ ID NO: 404 - DNA, 11 bp, Homo Sapiens
tttgcctggat

SEQ ID NO: 405 - DNA, 11 bp, Homo Sapiens
ttactgtgtaa

SEQ ID NO: 406 - DNA, 11 bp, Homo Sapiens
aataaaggcta

SEQ ID NO: 407 - DNA, 11 bp, Homo Sapiens
ccattgtactc

SEQ ID NO: 408 - DNA, 11 bp, Homo Sapiens
aaactcgagca

SEQ ID NO: 409 - DNA, 11 bp, Homo Sapiens
gtgactgccac

SEQ ID NO: 410 - DNA, 11 bp, Homo Sapiens
gggcaagccag

SEQ ID NO: 411 - DNA, 11 bp, Homo Sapiens
atactgtcagt

SEQ ID NO: 412 - DNA, 11 bp, Homo Sapiens
atgggggtgat

SEQ ID NO: 413 - DNA, 11 bp, Homo Sapiens
gaaaaaatgtt

SEQ ID NO: 414 - DNA, 11 bp, Homo Sapiens
gaataaatgtt

SEQ ID NO: 415 - DNA, 11 bp, Homo Sapiens
gtaggagctgg

SEQ ID NO: 416 - DNA, 11 bp, Homo Sapiens
gtgtatctttt

SEQ ID NO: 417 - DNA, 11 bp, Homo Sapiens
cttttgttctg

SEQ ID NO: 418 - DNA, 11 bp, Homo Sapiens
gggcttggtat

SEQ ID NO: 419 - DNA, 11 bp, Homo Sapiens
cacaccattgt

SEQ ID NO: 420 - DNA, 11 bp, Homo Sapiens
gagtgagtgag

SEQ ID NO: 421 - DNA, 11 bp, Homo Sapiens
ttgctatgaaa

SEQ ID NO: 422 - DNA, 11 bp, Homo Sapiens
agaagtactga

SEQ ID NO: 423 - DNA, 11 bp, Homo Sapiens
gcttaatgttt

SEQ ID NO: 424 - DNA, 11 bp, Homo Sapiens
tgactgctgct

SEQ ID NO: 425 - DNA, 11 bp, Homo Sapiens
tctcaagaagc

SEQ ID NO: 426 - DNA, 11 bp, Homo Sapiens
ctcccggaaat

SEQ ID NO: 427 - DNA, 11 bp, Homo Sapiens
tgtgagggcat

SEQ ID NO: 428 - DNA, 11 bp, Homo Sapiens
gtgaatgtatg

SEQ ID NO: 429 - DNA, 11 bp, Homo Sapiens
aatttagagca

SEQ ID NO: 430 - DNA, 11 bp, Homo Sapiens
cctgttctcct

SEQ ID NO: 431 - DNA, 11 bp, Homo Sapiens
aagttgtgtgc

SEQ ID NO: 432 - DNA, 11 bp, Homo Sapiens
ttgcagagggg

SEQ ID NO: 433 - DNA, 11 bp, Homo Sapiens
ttggcagtatt

SEQ ID NO: 434 - DNA, 11 bp, Homo Sapiens
tttgttaaaaa

SEQ ID NO: 435 - DNA, 11 bp, Homo Sapiens
acccactgtgc

SEQ ID NO: 436 - DNA, 11 bp, Homo Sapiens
ttgtttctact

SEQ ID NO: 437 - DNA, 11 bp, Homo Sapiens
aagactatgtt

SEQ ID NO: 438 - DNA, 11 bp, Homo Sapiens
tcaaatgtgaa

SEQ ID NO: 439 - DNA, 11 bp, Homo Sapiens
tgaatgaatgg

SEQ ID NO: 440 - DNA, 11 bp, Homo Sapiens
ttttgtgttgg

SEQ ID NO: 441 - DNA, 11 bp, Homo Sapiens
gactcccagga

SEQ ID NO: 442 - DNA, 11 bp, Homo Sapiens
gaccgcaggag

SEQ ID NO: 443 - DNA, 11 bp, Homo Sapiens
gctttgggatg

SEQ ID NO: 444 - DNA, 11 bp, Homo Sapiens
agcacattctt

SEQ ID NO: 445 - DNA, 11 bp, Homo Sapiens
aacttgatacg

SEQ ID NO: 446 - DNA, 11 bp, Homo Sapiens
ggaataaaagt

SEQ ID NO: 447 - DNA, 11 bp, Homo Sapiens
aaacagtagtg

SEQ ID NO: 448 - DNA, 11 bp, Homo Sapiens
gagggaagacc

SEQ ID NO: 449 - DNA, 11 bp, Homo Sapiens
taataaataga

SEQ ID NO: 450 - DNA, 11 bp, Homo Sapiens
tagacaatgct

SEQ ID NO: 451 - DNA, 11 bp, Homo Sapiens
gaattaaacaa

SEQ ID NO: 452 - DNA, 11 bp, Homo Sapiens
tttggaaaagc

SEQ ID NO: 453 - DNA, 11 bp, Homo Sapiens
tttgtctgtct

SEQ ID NO: 454 - DNA, 11 bp, Homo Sapiens
cacacttggag

SEQ ID NO: 455 - DNA, 11 bp, Homo Sapiens
tgttattactg

SEQ ID NO: 456 - DNA, 11 bp, Homo Sapiens
tcctttttctc

SEQ ID NO: 457 - DNA, 11 bp, Homo Sapiens
tgaaagggaga

SEQ ID NO: 458 - DNA, 11 bp, Homo Sapiens
tgcctttaacc

SEQ ID NO: 459 - DNA, 11 bp, Homo Sapiens
gatatgacaag

SEQ ID NO: 460 - DNA, 11 bp, Homo Sapiens
gcaccttctgg

SEQ ID NO: 461 - DNA, 11 bp, Homo Sapiens
cagtagacaga

SEQ ID NO: 462 - DNA, 11 bp, Homo Sapiens
ttgttttctgg

SEQ ID NO: 463 - DNA, 11 bp, Homo Sapiens
taatcaaaaaa

SEQ ID NO: 464 - DNA, 11 bp, Homo Sapiens
agagtgcctat

SEQ ID NO: 465 - DNA, 11 bp, Homo Sapiens
gtgtctgtctc

SEQ ID NO: 466 - DNA, 11 bp, Homo Sapiens
gtgtatatgta

SEQ ID NO: 467 - DNA, 11 bp, Homo Sapiens
ataaggaaaag

SEQ ID NO: 468 - DNA, 11 bp, Homo Sapiens
cagctttttca

SEQ ID NO: 469 - DNA, 11 bp, Homo Sapiens
ttaagaagcct

SEQ ID NO: 470 - DNA, 11 bp, Homo Sapiens
gtttgtgaaaa

SEQ ID NO: 471 - DNA, 11 bp, Homo Sapiens
tacctgtggtc

SEQ ID NO: 472 - DNA, 11 bp, Homo Sapiens
tttatttggca

SEQ ID NO: 473 - DNA, 11 bp, Homo Sapiens
cagcgcgccct

SEQ ID NO: 474 - DNA, 11 bp, Homo Sapiens
gctagtagagt

SEQ ID NO: 475 - DNA, 11 bp, Homo Sapiens
tacctatttac

SEQ ID NO: 476 - DNA, 11 bp, Homo Sapiens
atgtaactact

SEQ ID NO: 477 - DNA, 11 bp, Homo Sapiens
caacacctgag

SEQ ID NO: 478 - DNA, 11 bp, Homo Sapiens
agaataaatct

SEQ ID NO: 479 - DNA, 11 bp, Homo Sapiens
tattatataca

SEQ ID NO: 480 - DNA, 11 bp, Homo Sapiens
acctaactttt

SEQ ID NO: 481 - DNA, 11 bp, Homo Sapiens
tgcaaagtact

SEQ ID NO: 482 - DNA, 11 bp, Homo Sapiens
agctgggatgg

SEQ ID NO: 483 - DNA, 11 bp, Homo Sapiens
acaagtacatt

SEQ ID NO: 484 - DNA, 11 bp, Homo Sapiens
aggtgcggggg

SEQ ID NO: 485 - DNA, 11 bp, Homo Sapiens
gctaggtattt

SEQ ID NO: 486 - DNA, 11 bp, Homo Sapiens
caaaagctttg

SEQ ID NO: 487 - DNA, 11 bp, Homo Sapiens
gtatttaataa

SEQ ID NO: 488 - DNA, 11 bp, Homo Sapiens
cctagggttcc

SEQ ID NO: 489 - DNA, 11 bp, Homo Sapiens
ctgcccagtgg

SEQ ID NO: 490 - DNA, 11 bp, Homo Sapiens
tttgtttttga

SEQ ID NO: 491 - DNA, 11 bp, Homo Sapiens
gaggggcactg

SEQ ID NO: 492 - DNA, 11 bp, Homo Sapiens
agacagtcatt

SEQ ID NO: 493 - DNA, 11 bp, Homo Sapiens
cactgaactct

SEQ ID NO: 494 - DNA, 11 bp, Homo Sapiens
tgtggggaaag

SEQ ID NO: 495 - DNA, 11 bp, Homo Sapiens
agaagccgtgg

SEQ ID NO: 496 - DNA, 11 bp, Homo Sapiens
tgccttgaaag

SEQ ID NO: 497 - DNA, 11 bp, Homo Sapiens
gcccccctgcc

SEQ ID NO: 498 - DNA, 11 bp, Homo Sapiens
cataaatatta

SEQ ID NO: 499 - DNA, 11 bp, Homo Sapiens
ctgctgagcct

SEQ ID NO: 500 - DNA, 11 bp, Homo Sapiens
ctgtgacattt

SEQ ID NO: 501 - DNA, 11 bp, Homo Sapiens
aaggaagatgg

SEQ ID NO: 502 - DNA, 11 bp, Homo Sapiens
ctcggcgagcc

SEQ ID NO: 503 - DNA, 11 bp, Homo Sapiens
aagatcaagtc

SEQ ID NO: 504 - DNA, 11 bp, Homo Sapiens
aatataaaaca

SEQ ID NO: 505 - DNA, 11 bp, Homo Sapiens
gcattcgcagt

SEQ ID NO: 506 - DNA, 11 bp, Homo Sapiens
taagatggcaa

SEQ ID NO: 507 - DNA, 11 bp, Homo Sapiens
gttttatgaag

SEQ ID NO: 508 - DNA, 11 bp, Homo Sapiens
ggtctggcctg

SEQ ID NO: 509 - DNA, 11 bp, Homo Sapiens
cacgactgttc

SEQ ID NO: 510 - DNA, 11 bp, Homo Sapiens
gtgaaacctgg

SEQ ID NO: 511 - DNA, 11 bp, Homo Sapiens
ctgctcacctt

SEQ ID NO: 512 - DNA, 11 bp, Homo Sapiens
taagaaaaggc

SEQ ID NO: 513 - DNA, 11 bp, Homo Sapiens
tcagtgaccag

SEQ ID NO: 514 - DNA, 11 bp, Homo Sapiens
acacaagttta

SEQ ID NO: 515 - DNA, 11 bp, Homo Sapiens
acaagattaaa

SEQ ID NO: 516 - DNA, 11 bp, Homo Sapiens
tacaaaagtgg

SEQ ID NO: 517 - DNA, 11 bp, Homo Sapiens
ttaccattggt

SEQ ID NO: 518 - DNA, 11 bp, Homo Sapiens
aaggagagccc

SEQ ID NO: 519 - DNA, 11 bp, Homo Sapiens
attaaaaaaaa

SEQ ID NO: 520 - DNA, 11 bp, Homo Sapiens
atacattccat

SEQ ID NO: 521 - DNA, 11 bp, Homo Sapiens
tgaagatggag

SEQ ID NO: 522 - DNA, 11 bp, Homo Sapiens
ctgttatagga

SEQ ID NO: 523 - DNA, 11 bp, Homo Sapiens
taaaaccctat

SEQ ID NO: 524 - DNA, 11 bp, Homo Sapiens
agtcttaatgt

SEQ ID NO: 525 - DNA, 11 bp, Homo Sapiens
ggagcactctc

SEQ ID NO: 526 - DNA, 11 bp, Homo Sapiens
tggatcattct

SEQ ID NO: 527 - DNA, 11 bp, Homo Sapiens
gccttgggtga

SEQ ID NO: 528 - DNA, 11 bp, Homo Sapiens
atattacagtg

SEQ ID NO: 529 - DNA, 11 bp, Homo Sapiens
tagctaatgcc

SEQ ID NO: 530 - DNA, 11 bp, Homo Sapiens
tttatatcttt

SEQ ID NO: 531 - DNA, 11 bp, Homo Sapiens
cttattgccct

SEQ ID NO: 532 - DNA, 11 bp, Homo Sapiens
ctgccattctt

SEQ ID NO: 533 - DNA, 11 bp, Homo Sapiens
atgtatagggc

SEQ ID NO: 534 - DNA, 11 bp, Homo Sapiens
attaactgcta

SEQ ID NO: 535 - DNA, 11 bp, Homo Sapiens
ttaatctggta

SEQ ID NO: 536 - DNA, 11 bp, Homo Sapiens
tatctagctgc

SEQ ID NO: 537 - DNA, 11 bp, Homo Sapiens
agtgctttgag

SEQ ID NO: 538 - DNA, 11 bp, Homo Sapiens
tttttatccat

SEQ ID NO: 539 - DNA, 11 bp, Homo Sapiens
agctctggaag

SEQ ID NO: 540 - DNA, 11 bp, Homo Sapiens
tgaaccaggtg

SEQ ID NO: 541 - DNA, 11 bp, Homo Sapiens
cactatatttg

SEQ ID NO: 542 - DNA, 11 bp, Homo Sapiens
tgtgcacaata

SEQ ID NO: 543 - DNA, 11 bp, Homo Sapiens
catacattgat

SEQ ID NO: 544 - DNA, 11 bp, Homo Sapiens
gctgccaaaag

SEQ ID NO: 545 - DNA, 11 bp, Homo Sapiens
agttttgctgt

SEQ ID NO: 546 - DNA, 11 bp, Homo Sapiens
taattgcttat

SEQ ID NO: 547 - DNA, 11 bp, Homo Sapiens
atgtgagggag

SEQ ID NO: 548 - DNA, 11 bp, Homo Sapiens
acattggtaaa

SEQ ID NO: 549 - DNA, 11 bp, Homo Sapiens
ggtggtgtctg

SEQ ID NO: 550 - DNA, 11 bp, Homo Sapiens
aaaacctaaat

SEQ ID NO: 551 - DNA, 11 bp, Homo Sapiens
gccctacagat

SEQ ID NO: 552 - DNA, 11 bp, Homo Sapiens
tagttaagcca

SEQ ID NO: 553 - DNA, 11 bp, Homo Sapiens
aacttaaaaaa

SEQ ID NO: 554 - DNA, 11 bp, Homo Sapiens
gtgcgccgctg

SEQ ID NO: 555 - DNA, 11 bp, Homo Sapiens
gagggtatact

SEQ ID NO: 556 - DNA, 11 bp, Homo Sapiens
gtttaagaatt

SEQ ID NO: 557 - DNA, 11 bp, Homo Sapiens
aggccaccctg

SEQ ID NO: 558 - DNA, 11 bp, Homo Sapiens
gggaagagtga

SEQ ID NO: 559 - DNA, 11 bp, Homo Sapiens
cggagccggct

SEQ ID NO: 560 - DNA, 11 bp, Homo Sapiens
aatgaaaggtt

SEQ ID NO: 561 - DNA, 11 bp, Homo Sapiens
cggctcaagtc

SEQ ID NO: 562 - DNA, 11 bp, Homo Sapiens
gtcttcttaat

SEQ ID NO: 563 - DNA, 11 bp, Homo Sapiens
acgtgagtgct

SEQ ID NO: 564 - DNA, 11 bp, Homo Sapiens
gccagcctatg

SEQ ID NO: 565 - DNA, 11 bp, Homo Sapiens
tgtgagttatt

SEQ ID NO: 566 - DNA, 11 bp, Homo Sapiens
tgtgttgagac

SEQ ID NO: 567 - DNA, 11 bp, Homo Sapiens
accataatgtg

SEQ ID NO: 568 - DNA, 11 bp, Homo Sapiens
gtatctatgca

SEQ ID NO: 569 - DNA, 11 bp, Homo Sapiens
ctccaatgtat

SEQ ID NO: 570 - DNA, 11 bp, Homo Sapiens
aaggtctattc

SEQ ID NO: 571 - DNA, 11 bp, Homo Sapiens
tttgcaaataa

SEQ ID NO: 572 - DNA, 11 bp, Homo Sapiens
ggtagctcagg

SEQ ID NO: 573 - DNA, 11 bp, Homo Sapiens
cctgagttgaa

SEQ ID NO: 574 - DNA, 11 bp, Homo Sapiens
tctcccacacc

SEQ ID NO: 575 - DNA, 11 bp, Homo Sapiens
aagacagtggg

SEQ ID NO: 576 - DNA, 11 bp, Homo Sapiens
gagggccttca

SEQ ID NO: 577 - DNA, 11 bp, Homo Sapiens
cagtcaggctg

SEQ ID NO: 578 - DNA, 11 bp, Homo Sapiens
ataaatttgtg

SEQ ID NO: 579 - DNA, 11 bp, Homo Sapiens
tggaattgttt

SEQ ID NO: 580 - DNA, 11 bp, Homo Sapiens
ggggtggcagg

SEQ ID NO: 581 - DNA, 11 bp, Homo Sapiens
caagaggcagt

SEQ ID NO: 582 - DNA, 11 bp, Homo Sapiens
ccattgcaatc

SEQ ID NO: 583 - DNA, 11 bp, Homo Sapiens
agggactcctc

SEQ ID NO: 584 - DNA, 11 bp, Homo Sapiens
acaaactaaaa

SEQ ID NO: 585 - DNA, 11 bp, Homo Sapiens
caaatatcttg

SEQ ID NO: 586 - DNA, 11 bp, Homo Sapiens
tgcgtgacaga

SEQ ID NO: 587 - DNA, 11 bp, Homo Sapiens
gcactctaacc

SEQ ID NO: 588 - DNA, 11 bp, Homo Sapiens
tttaattgtaa

SEQ ID NO: 589 - DNA, 11 bp, Homo Sapiens
aggtcaaaaaa

SEQ ID NO: 590 - DNA, 11 bp, Homo Sapiens
atcattccctc

SEQ ID NO: 591 - DNA, 11 bp, Homo Sapiens
gtgagtgtgtc

SEQ ID NO: 592 - DNA, 11 bp, Homo Sapiens
aaaataaatgt

SEQ ID NO: 593 - DNA, 11 bp, Homo Sapiens
ctcgaggagga

SEQ ID NO: 594 - DNA, 11 bp, Homo Sapiens
gtggcacgcat

SEQ ID NO: 595 - DNA, 11 bp, Homo Sapiens
tgaataaaaaa

SEQ ID NO: 596 - DNA, 11 bp, Homo Sapiens
gtctggaagga

SEQ ID NO: 597 - DNA, 11 bp, Homo Sapiens
gcagaaaattt

SEQ ID NO: 598 - DNA, 11 bp, Homo Sapiens
taccccataaa

SEQ ID NO: 599 - DNA, 11 bp, Homo Sapiens
gcaagccccag

SEQ ID NO: 600 - DNA, 11 bp, Homo Sapiens
ggcaaagcccc

SEQ ID NO: 601 - DNA, 11 bp, Homo Sapiens
cagatttgcct

SEQ ID NO: 602 - DNA, 11 bp, Homo Sapiens
ttttgggcagt

SEQ ID NO: 603 - DNA, 11 bp, Homo Sapiens
ggtaaaattat

SEQ ID NO: 604 - DNA, 11 bp, Homo Sapiens
aattaccaaag

SEQ ID NO: 605 - DNA, 11 bp, Homo Sapiens
actattagtgc

SEQ ID NO: 606 - DNA, 11 bp, Homo Sapiens
ttttgtcaaca

SEQ ID NO: 607 - DNA, 11 bp, Homo Sapiens
gctggaaatgt

SEQ ID NO: 608 - DNA, 11 bp, Homo Sapiens
cacttgcagta

SEQ ID NO: 609 - DNA, 11 bp, Homo Sapiens
tacatatctgt

SEQ ID NO: 610 - DNA, 11 bp, Homo Sapiens
gccaccaccac

SEQ ID NO: 611 - DNA, 11 bp, Homo Sapiens
atggcaccagt

SEQ ID NO: 612 - DNA, 11 bp, Homo Sapiens
tttgtttttat

SEQ ID NO: 613 - DNA, 11 bp, Homo Sapiens
tggttttctcc

SEQ ID NO: 614 - DNA, 11 bp, Homo Sapiens
aataaagttat

SEQ ID NO: 615 - DNA, 11 bp, Homo Sapiens
ctgctttagga

SEQ ID NO: 616 - DNA, 11 bp, Homo Sapiens
ctgtaactgac

SEQ ID NO: 617 - DNA, 11 bp, Homo Sapiens
gtgagacacta

SEQ ID NO: 618 - DNA, 11 bp, Homo Sapiens
aaccaggtgga

SEQ ID NO: 619 - DNA, 11 bp, Homo Sapiens
cggcccatctg

SEQ ID NO: 620 - DNA, 11 bp, Homo Sapiens
ttttcttcgga

SEQ ID NO: 621 - DNA, 11 bp, Homo Sapiens
ttcgtattaca

SEQ ID NO: 622 - DNA, 11 bp, Homo Sapiens
ctggtgtctgg

SEQ ID NO: 623 - DNA, 11 bp, Homo Sapiens
gccctagcaat

SEQ ID NO: 624 - DNA, 11 bp, Homo Sapiens
attttgcagtg

SEQ ID NO: 625 - DNA, 11 bp, Homo Sapiens
gtcagtgttac

SEQ ID NO: 626 - DNA, 11 bp, Homo Sapiens
caaactttgaa

SEQ ID NO: 627 - DNA, 11 bp, Homo Sapiens
tttgcctgggg

SEQ ID NO: 628 - DNA, 11 bp, Homo Sapiens
catctttttat

SEQ ID NO: 629 - DNA, 11 bp, Homo Sapiens
tcatttgctcc

SEQ ID NO: 630 - DNA, 11 bp, Homo Sapiens
tagcagaccct

SEQ ID NO: 631 - DNA, 11 bp, Homo Sapiens
gctgatctgtc

SEQ ID NO: 632 - DNA, 11 bp, Homo Sapiens
taatgccttgt

SEQ ID NO: 633 - DNA, 11 bp, Homo Sapiens
acttgaattct

SEQ ID NO: 634 - DNA, 11 bp, Homo Sapiens
tgtcaaaaaaa

SEQ ID NO: 635 - DNA, 11 bp, Homo Sapiens
tgtttggtttg

SEQ ID NO: 636 - DNA, 11 bp, Homo Sapiens
gaccaacagtg

SEQ ID NO: 637 - DNA, 11 bp, Homo Sapiens
ttgggttgtta

SEQ ID NO: 638 - DNA, 11 bp, Homo Sapiens
cccaaaccttc

SEQ ID NO: 639 - DNA, 11 bp, Homo Sapiens
aaggatgaatt

SEQ ID NO: 640 - DNA, 11 bp, Homo Sapiens
tgtttcaggat

SEQ ID NO: 641 - DNA, 11 bp, Homo Sapiens
gtttgttggga

SEQ ID NO: 642 - DNA, 11 bp, Homo Sapiens
gttaaataaga

SEQ ID NO: 643 - DNA, 11 bp, Homo Sapiens
ccctatgtttg

SEQ ID NO: 644 - DNA, 11 bp, Homo Sapiens
gtgattgttca

SEQ ID NO: 645 - DNA, 11 bp, Homo Sapiens
gggcagaataa

SEQ ID NO: 646 - DNA, 11 bp, Homo Sapiens
tctttgtctaa

SEQ ID NO: 647 - DNA, 11 bp, Homo Sapiens
acacagctctg

SEQ ID NO: 648 - DNA, 11 bp, Homo Sapiens
gtcccgggcat

SEQ ID NO: 649 - DNA, 11 bp, Homo Sapiens
ttttaattgct

SEQ ID NO: 650 - DNA, 11 bp, Homo Sapiens
cctcagcctcc

SEQ ID NO: 651 - DNA, 11 bp, Homo Sapiens
aatatttacca

SEQ ID NO: 652 - DNA, 11 bp, Homo Sapiens
caggtcgctac

SEQ ID NO: 653 - DNA, 11 bp, Homo Sapiens
ggcacaatcaa

SEQ ID NO: 654 - DNA, 11 bp, Homo Sapiens
cctaatgtgtt

SEQ ID NO: 655 - DNA, 11 bp, Homo Sapiens
ttctctattgg

SEQ ID NO: 656 - DNA, 11 bp, Homo Sapiens
gacaccggatt

SEQ ID NO: 657 - DNA, 11 bp, Homo Sapiens
gtgaaggcagg

SEQ ID NO: 658 - DNA, 11 bp, Homo Sapiens
agaaaaacaaa

SEQ ID NO: 659 - DNA, 11 bp, Homo Sapiens
cattttaatac

SEQ ID NO: 660 - DNA, 11 bp, Homo Sapiens
tgaaggtggat

SEQ ID NO: 661 - DNA, 11 bp, Homo Sapiens
cctttgtaaaa

SEQ ID NO: 662 - DNA, 11 bp, Homo Sapiens
taaaacttacc

SEQ ID NO: 663 - DNA, 11 bp, Homo Sapiens
tataaagtaaa

SEQ ID NO: 664 - DNA, 11 bp, Homo Sapiens
taagtctatat

SEQ ID NO: 665 - DNA, 11 bp, Homo Sapiens
ctttgcactct

SEQ ID NO: 666 - DNA, 11 bp, Homo Sapiens
agtatggaatg

SEQ ID NO: 667 - DNA, 11 bp, Homo Sapiens
gtggaggtgcg

SEQ ID NO: 668 - DNA, 11 bp, Homo Sapiens
catttaagttt

SEQ ID NO: 669 - DNA, 11 bp, Homo Sapiens
agactaacacg

SEQ ID NO: 670 - DNA, 11 bp, Homo Sapiens
gtacctagagt

SEQ ID NO: 671 - DNA, 11 bp, Homo Sapiens
gtgaagtcttc

SEQ ID NO: 672 - DNA, 11 bp, Homo Sapiens
ttcagtgctac

SEQ ID NO: 673 - DNA, 11 bp, Homo Sapiens
taaaaaaaaat

SEQ ID NO: 674 - DNA, 11 bp, Homo Sapiens
ggggcacccgc

SEQ ID NO: 675 - DNA, 11 bp, Homo Sapiens
taggtcccctt

SEQ ID NO: 676 - DNA, 11 bp, Homo Sapiens
actatctctag

SEQ ID NO: 677 - DNA, 11 bp, Homo Sapiens
ctccgaggcag

SEQ ID NO: 678 - DNA, 11 bp, Homo Sapiens
gcagcagtgtc

SEQ ID NO: 679 - DNA, 11 bp, Homo Sapiens
caagcttggtc

SEQ ID NO: 680 - DNA, 11 bp, Homo Sapiens
ctgagctgtac

SEQ ID NO: 681 - DNA, 11 bp, Homo Sapiens
aagtcatctat

SEQ ID NO: 682 - DNA, 11 bp, Homo Sapiens
tgggtttttat

SEQ ID NO: 683 - DNA, 11 bp, Homo Sapiens
ggaatgagaag

SEQ ID NO: 684 - DNA, 11 bp, Homo Sapiens
ataccagatac

SEQ ID NO: 685 - DNA, 11 bp, Homo Sapiens
tattagtcaca

SEQ ID NO: 686 - DNA, 11 bp, Homo Sapiens
acataagatca

SEQ ID NO: 687 - DNA, 11 bp, Homo Sapiens
cctcttcaggc

SEQ ID NO: 688 - DNA, 11 bp, Homo Sapiens
gacaaggacag

SEQ ID NO: 689 - DNA, 11 bp, Homo Sapiens
ctgcaaatgaa

SEQ ID NO: 690 - DNA, 11 bp, Homo Sapiens
ccaatggccag

SEQ ID NO: 691 - DNA, 11 bp, Homo Sapiens
ggagtgcagct

SEQ ID NO: 692 - DNA, 11 bp, Homo Sapiens
tgcacacacac

SEQ ID NO: 693 - DNA, 11 bp, Homo Sapiens
gcttccatctt

SEQ ID NO: 694 - DNA, 11 bp, Homo Sapiens
cttcgaaactc

SEQ ID NO: 695 - DNA, 11 bp, Homo Sapiens
tgtgggaacca

SEQ ID NO: 696 - DNA, 11 bp, Homo Sapiens
cattaaagggt

SEQ ID NO: 697 - DNA, 11 bp, Homo Sapiens
tttggaaaaaa

SEQ ID NO: 698 - DNA, 11 bp, Homo Sapiens
gggggaatttt

SEQ ID NO: 699 - DNA, 11 bp, Homo Sapiens
ttttgatcact

SEQ ID NO: 700 - DNA, 11 bp, Homo Sapiens
agatgtgtggg

SEQ ID NO: 701 - DNA, 11 bp, Homo Sapiens
ggtcccctacc

SEQ ID NO: 702 - DNA, 11 bp, Homo Sapiens
tcataagcaat

SEQ ID NO: 703 - DNA, 11 bp, Homo Sapiens
acaaatccttg

SEQ ID NO: 704 - DNA, 11 bp, Homo Sapiens
tgtagtatttg

SEQ ID NO: 705 - DNA, 11 bp, Homo Sapiens
aacccgggggg

SEQ ID NO: 706 - DNA, 11 bp, Homo Sapiens
tgctagattgg

SEQ ID NO: 707 - DNA, 11 bp, Homo Sapiens
gagtaaaaaat

SEQ ID NO: 708 - DNA, 11 bp, Homo Sapiens
tgttaccaaga

SEQ ID NO: 709 - DNA, 11 bp, Homo Sapiens
gagccgcctct

SEQ ID NO: 710 - DNA, 11 bp, Homo Sapiens
tggtgacagtt

SEQ ID NO: 711 - DNA, 11 bp, Homo Sapiens
gaagttgcctt

SEQ ID NO: 712 - DNA, 11 bp, Homo Sapiens
gccagaagggg

SEQ ID NO: 713 - DNA, 11 bp, Homo Sapiens
ctgcaacctaa

SEQ ID NO: 714 - DNA, 11 bp, Homo Sapiens
aataaacgtgt

SEQ ID NO: 715 - DNA, 11 bp, Homo Sapiens
ggaccaggctg

SEQ ID NO: 716 - DNA, 11 bp, Homo Sapiens
tatcaaaacac

SEQ ID NO: 717 - DNA, 11 bp, Homo Sapiens
tcgaagaaccg

SEQ ID NO: 718 - DNA, 11 bp, Homo Sapiens
ctcttcgagaa

SEQ ID NO: 719 - DNA, 11 bp, Homo Sapiens
ggagacttcct

SEQ ID NO: 720 - DNA, 11 bp, Homo Sapiens
caggacagttt

SEQ ID NO: 721 - DNA, 11 bp, Homo Sapiens
ccaaggactct

SEQ ID NO: 722 - DNA, 11 bp, Homo Sapiens
tagaatgcaaa

SEQ ID NO: 723 - DNA, 11 bp, Homo Sapiens
cagacgctccg

SEQ ID NO: 724 - DNA, 11 bp, Homo Sapiens
gtttaaaaaga

SEQ ID NO: 725 - DNA, 11 bp, Homo Sapiens
tggaacaggat

SEQ ID NO: 726 - DNA, 11 bp, Homo Sapiens
ttcctccaccc

SEQ ID NO: 727 - DNA, 11 bp, Homo Sapiens
acccacctgca

SEQ ID NO: 728 - DNA, 11 bp, Homo Sapiens
gtgcctaggga

SEQ ID NO: 729 - DNA, 11 bp, Homo Sapiens
cccccagatga

SEQ ID NO: 730 - DNA, 11 bp, Homo Sapiens
gtctgggggat

SEQ ID NO: 731 - DNA, 11 bp, Homo Sapiens
tgggaagtggg

SEQ ID NO: 732 - DNA, 11 bp, Homo Sapiens
gcaggaggtga

SEQ ID NO: 733 - DNA, 11 bp, Homo Sapiens
ccttacctaca

SEQ ID NO: 734 - DNA, 11 bp, Homo Sapiens
gcgaaacccta

SEQ ID NO: 735 - DNA, 11 bp, Homo Sapiens
gccagacaccc

SEQ ID NO: 736 - DNA, 11 bp, Homo Sapiens
aatgttgtgca

SEQ ID NO: 737 - DNA, 11 bp, Homo Sapiens
gttgcagataa

SEQ ID NO: 738 - DNA, 11 bp, Homo Sapiens
tggttgcgaca

SEQ ID NO: 739 - DNA, 11 bp, Homo Sapiens
caaatatggtt

SEQ ID NO: 740 - DNA, 11 bp, Homo Sapiens
gagaaaaaaaa

SEQ ID NO: 741 - DNA, 11 bp, Homo Sapiens
tttgaaaacaa

SEQ ID NO: 742 - DNA, 11 bp, Homo Sapiens
tgtaacaataa

SEQ ID NO: 743 - DNA, 11 bp, Homo Sapiens
tgttgtatttg

SEQ ID NO: 744 - DNA, 11 bp, Homo Sapiens
gaaaggtggtt

SEQ ID NO: 745 - DNA, 11 bp, Homo Sapiens
ggatgtagaga

SEQ ID NO: 746 - DNA, 11 bp, Homo Sapiens
cactgtgtgta

SEQ ID NO: 747 - DNA, 11 bp, Homo Sapiens
cctctggaggc

SEQ ID NO: 748 - DNA, 11 bp, Homo Sapiens
tattcctgtga

SEQ ID NO: 749 - DNA, 11 bp, Homo Sapiens
caactttaggg

SEQ ID NO: 750 - DNA, 11 bp, Homo Sapiens
catttactcta

SEQ ID NO: 751 - DNA, 11 bp, Homo Sapiens
gggctggacgg

SEQ ID NO: 752 - DNA, 11 bp, Homo Sapiens
aactgggtctg

SEQ ID NO: 753 - DNA, 11 bp, Homo Sapiens
tgcaggtttgt

SEQ ID NO: 754 - DNA, 11 bp, Homo Sapiens
aggaaagccag

SEQ ID NO: 755 - DNA, 11 bp, Homo Sapiens
ctaaatcactg

SEQ ID NO: 756 - DNA, 11 bp, Homo Sapiens
aaaaataaaga

SEQ ID NO: 757 - DNA, 11 bp, Homo Sapiens
gaagcaataaa

SEQ ID NO: 758 - DNA, 11 bp, Homo Sapiens
gtgaaaccctt

SEQ ID NO: 759 - DNA, 11 bp, Homo Sapiens
gaaatgtatgc

SEQ ID NO: 760 - DNA, 11 bp, Homo Sapiens
ccacgtgtccg

SEQ ID NO: 761 - DNA, 11 bp, Homo Sapiens
taactccaaag

SEQ ID NO: 762 - DNA, 11 bp, Homo Sapiens
tggagagaata

SEQ ID NO: 763 - DNA, 11 bp, Homo Sapiens
cttgagtcaca

SEQ ID NO: 764 - DNA, 11 bp, Homo Sapiens
caatctgatgc

SEQ ID NO: 765 - DNA, 11 bp, Homo Sapiens
caataaaactg

SEQ ID NO: 766 - DNA, 11 bp, Homo Sapiens
cttgcagtcct

SEQ ID NO: 767 - DNA, 11 bp, Homo Sapiens
agccggatgct

SEQ ID NO: 768 - DNA, 11 bp, Homo Sapiens
ggagcttgagg

SEQ ID NO: 769 - DNA, 11 bp, Homo Sapiens
ctttaccaaaa

SEQ ID NO: 770 - DNA, 11 bp, Homo Sapiens
ccaccacaccc

SEQ ID NO: 771 - DNA, 11 bp, Homo Sapiens
cagggctcgcg

SEQ ID NO: 772 - DNA, 11 bp, Homo Sapiens
aacccagaagg

SEQ ID NO: 773 - DNA, 11 bp, Homo Sapiens
ttccaaagcga

SEQ ID NO: 774 - DNA, 11 bp, Homo Sapiens
gagctcaagat

SEQ ID NO: 775 - DNA, 11 bp, Homo Sapiens
ctggtttaaat

SEQ ID NO: 776 - DNA, 11 bp, Homo Sapiens
atggctcattc

SEQ ID NO: 777 - DNA, 11 bp, Homo Sapiens
tttcaatacca

SEQ ID NO: 778 - DNA, 11 bp, Homo Sapiens
tctccacgaag

SEQ ID NO: 779 - DNA, 11 bp, Homo Sapiens
gcacttacaaa

SEQ ID NO: 780 - DNA, 11 bp, Homo Sapiens
agccatcacac

SEQ ID NO: 781 - DNA, 11 bp, Homo Sapiens
aactaaaaaac

SEQ ID NO: 782 - DNA, 11 bp, Homo Sapiens
ggaggatcact

SEQ ID NO: 783 - DNA, 11 bp, Homo Sapiens
aaattgttcca

SEQ ID NO: 784 - DNA, 11 bp, Homo Sapiens
ttctttgggaa

SEQ ID NO: 785 - DNA, 11 bp, Homo Sapiens
gtatacctacc

SEQ ID NO: 786 - DNA, 11 bp, Homo Sapiens
gtgggtcagct

SEQ ID NO: 787 - DNA, 11 bp, Homo Sapiens
gcagagatggg

SEQ ID NO: 788 - DNA, 11 bp, Homo Sapiens
ctgggccattg

SEQ ID NO: 789 - DNA, 11 bp, Homo Sapiens
gatcaaaactg

SEQ ID NO: 790 - DNA, 11 bp, Homo Sapiens
gtggtggacgc

SEQ ID NO: 791 - DNA, 11 bp, Homo Sapiens
attgtgctact

SEQ ID NO: 792 - DNA, 11 bp, Homo Sapiens
ttctctttcaa

SEQ ID NO: 793 - DNA, 11 bp, Homo Sapiens
gccaaagatgt

SEQ ID NO: 794 - DNA, 11 bp, Homo Sapiens
ctggtcgttgg

SEQ ID NO: 795 - DNA, 11 bp, Homo Sapiens
agcactgtact

SEQ ID NO: 796 - DNA, 11 bp, Homo Sapiens
acaagatattt

SEQ ID NO: 797 - DNA, 11 bp, Homo Sapiens
tacatttgaat

SEQ ID NO: 798 - DNA, 11 bp, Homo Sapiens
agaaatcactg

SEQ ID NO: 799 - DNA, 11 bp, Homo Sapiens
agtccttgaaa

SEQ ID NO: 800 - DNA, 11 bp, Homo Sapiens
taaatgaaaaa

SEQ ID NO: 801 - DNA, 11 bp, Homo Sapiens
ttagtcttcag

SEQ ID NO: 802 - DNA, 11 bp, Homo Sapiens
agtgtgcgctt

SEQ ID NO: 803 - DNA, 11 bp, Homo Sapiens
tctttcgtctg

SEQ ID NO: 804 - DNA, 11 bp, Homo Sapiens
taatctttact

SEQ ID NO: 805 - DNA, 11 bp, Homo Sapiens
cttttgtcagc

SEQ ID NO: 806 - DNA, 11 bp, Homo Sapiens
tcacgcgctcc

SEQ ID NO: 807 - DNA, 11 bp, Homo Sapiens
accaggcaagg

SEQ ID NO: 808 - DNA, 11 bp, Homo Sapiens
agtttgggctg

SEQ ID NO: 809 - DNA, 11 bp, Homo Sapiens
ccaaatgatga

SEQ ID NO: 810 - DNA, 11 bp, Homo Sapiens
tcaaatgcatc

SEQ ID NO: 811 - DNA, 11 bp, Homo Sapiens
tttctgcactt

SEQ ID NO: 812 - DNA, 11 bp, Homo Sapiens
ctaataaatgc

SEQ ID NO: 813 - DNA, 11 bp, Homo Sapiens
gtggggctagg

SEQ ID NO: 814 - DNA, 11 bp, Homo Sapiens
acagtcttgcc

SEQ ID NO: 815 - DNA, 11 bp, Homo Sapiens
cctgtaaagcc

SEQ ID NO: 816 - DNA, 11 bp, Homo Sapiens
gcgggagggct

SEQ ID NO: 817 - DNA, 11 bp, Homo Sapiens
atccctcagtg

SEQ ID NO: 818 - DNA, 11 bp, Homo Sapiens
actcagaagag

SEQ ID NO: 819 - DNA, 11 bp, Homo Sapiens
ttctctcaact

SEQ ID NO: 820 - DNA, 11 bp, Homo Sapiens
gatatcagtct

SEQ ID NO: 821 - DNA, 11 bp, Homo Sapiens
tgttccctttg

SEQ ID NO: 822 - DNA, 11 bp, Homo Sapiens
gatcaatcagt

SEQ ID NO: 823 - DNA, 11 bp, Homo Sapiens
tgtcctggttc

SEQ ID NO: 824 - DNA, 11 bp, Homo Sapiens
aaggggccttt

SEQ ID NO: 825 - DNA, 11 bp, Homo Sapiens
tcttctttcag

SEQ ID NO: 826 - DNA, 11 bp, Homo Sapiens
atcatagctca

SEQ ID NO: 827 - DNA, 11 bp, Homo Sapiens
gatgaacactg

SEQ ID NO: 828 - DNA, 11 bp, Homo Sapiens
tgcaaaaaaaa

SEQ ID NO: 829 - DNA, 11 bp, Homo Sapiens
ggggctgtggc

SEQ ID NO: 830 - DNA, 11 bp, Homo Sapiens
ttgaatagtga

SEQ ID NO: 831 - DNA, 11 bp, Homo Sapiens
tgaccaccctt

SEQ ID NO: 832 - DNA, 11 bp, Homo Sapiens
cttaatcttgt

SEQ ID NO: 833 - DNA, 11 bp, Homo Sapiens
gctgttcattg

SEQ ID NO: 834 - DNA, 11 bp, Homo Sapiens
ttctggctgcg

SEQ ID NO: 835 - DNA, 11 bp, Homo Sapiens
cactcgtgtga

SEQ ID NO: 836 - DNA, 11 bp, Homo Sapiens
cgcctataatc

SEQ ID NO: 837 - DNA, 11 bp, Homo Sapiens
tctccaggaac

SEQ ID NO: 838 - DNA, 11 bp, Homo Sapiens
gcgaaacccca

SEQ ID NO: 839 - DNA, 11 bp, Homo Sapiens
cctgtgatccc

SEQ ID NO: 840 - DNA, 11 bp, Homo Sapiens
cctgtaattcc

SEQ ID NO: 841 - DNA, 11 bp, Homo Sapiens
cccaacgcgct

SEQ ID NO: 842 - DNA, 11 bp, Homo Sapiens
cctgcaatccc

SEQ ID NO: 843 - DNA, 11 bp, Homo Sapiens
tctgtaatccc

SEQ ID NO: 844 - DNA, 11 bp, Homo Sapiens
ccactgcattc

SEQ ID NO: 845 - DNA, 11 bp, Homo Sapiens
aatctagttct

SEQ ID NO: 846 - DNA, 11 bp, Homo Sapiens
gagaatgacag

SEQ ID NO: 847 - DNA, 11 bp, Homo Sapiens
gtgaaactccg

SEQ ID NO: 848 - DNA, 11 bp, Homo Sapiens
gtgaaacccta

SEQ ID NO: 849 - DNA, 11 bp, Homo Sapiens
ctgagacaaag

SEQ ID NO: 850 - DNA, 11 bp, Homo Sapiens
tgggtgaaaaa

SEQ ID NO: 851 - DNA, 11 bp, Homo Sapiens
taatggtaact

SEQ ID NO: 852 - DNA, 11 bp, Homo Sapiens
tgcagcacgag

SEQ ID NO: 853 - DNA, 11 bp, Homo Sapiens
gtgaaatcccg

SEQ ID NO: 854 - DNA, 11 bp, Homo Sapiens
taaatgaataa

SEQ ID NO: 855 - DNA, 11 bp, Homo Sapiens
tttctgtatgt

SEQ ID NO: 856 - DNA, 11 bp, Homo Sapiens
ttcccttcttc

SEQ ID NO: 857 - DNA, 11 bp, Homo Sapiens
gcgtcggtgca

SEQ ID NO: 858 - DNA, 11 bp, Homo Sapiens
tggagagcaac

SEQ ID NO: 859 - DNA, 11 bp, Homo Sapiens
gagaaccgtag

SEQ ID NO: 860 - DNA, 11 bp, Homo Sapiens
tcgtaacgagg

SEQ ID NO: 861 - DNA, 11 bp, Homo Sapiens
acctgtatccc

SEQ ID NO: 862 - DNA, 11 bp, Homo Sapiens
atggagacttc

SEQ ID NO: 863 - DNA, 11 bp, Homo Sapiens
tttacaaagag

SEQ ID NO: 864 - DNA, 11 bp, Homo Sapiens
cctgtagtcct

SEQ ID NO: 865 - DNA, 11 bp, Homo Sapiens
ttgtcgatggg

SEQ ID NO: 866 - DNA, 11 bp, Homo Sapiens
atagaggcaat

SEQ ID NO: 867 - DNA, 11 bp, Homo Sapiens
tgttctccatt

SEQ ID NO: 868 - DNA, 11 bp, Homo Sapiens
cagaccattgt

SEQ ID NO: 869 - DNA, 11 bp, Homo Sapiens
gcttatagtca

SEQ ID NO: 870 - DNA, 11 bp, Homo Sapiens
tcaataaagaa

SEQ ID NO: 871 - DNA, 11 bp, Homo Sapiens
cacacagtttt

SEQ ID NO: 872 - DNA, 11 bp, Homo Sapiens
ggagccattct

SEQ ID NO: 873 - DNA, 11 bp, Homo Sapiens
tgaatggccta

SEQ ID NO: 874 - DNA, 11 bp, Homo Sapiens
agcctttgttg

SEQ ID NO: 875 - DNA, 11 bp, Homo Sapiens
atggcgatcta

SEQ ID NO: 876 - DNA, 11 bp, Homo Sapiens
gtattggcctt

SEQ ID NO: 877 - DNA, 11 bp, Homo Sapiens
tggccaataaa

SEQ ID NO: 878 - DNA, 11 bp, Homo Sapiens
tgacttttctg

SEQ ID NO: 879 - DNA, 11 bp, Homo Sapiens
agtaaaccatc

SEQ ID NO: 880 - DNA, 11 bp, Homo Sapiens
taacattaaag

SEQ ID NO: 881 - DNA, 11 bp, Homo Sapiens
agcctgcagaa

SEQ ID NO: 882 - DNA, 11 bp, Homo Sapiens
gggaaacccca

SEQ ID NO: 883 - DNA, 11 bp, Homo Sapiens
tttattgaaaa

SEQ ID NO: 884 - DNA, 11 bp, Homo Sapiens
cgtggggctgc

SEQ ID NO: 885 - DNA, 11 bp, Homo Sapiens
ggagagacagg

SEQ ID NO: 886 - DNA, 11 bp, Homo Sapiens
atctcggctca

SEQ ID NO: 887 - DNA, 11 bp, Homo Sapiens
ctaagtagagt

SEQ ID NO: 888 - DNA, 11 bp, Homo Sapiens
tctggcagtag

SEQ ID NO: 889 - DNA, 11 bp, Homo Sapiens
atggcggcgat

SEQ ID NO: 890 - DNA, 11 bp, Homo Sapiens
aatgctggcaa

SEQ ID NO: 891 - DNA, 11 bp, Homo Sapiens
caattgtaaat

SEQ ID NO: 892 - DNA, 11 bp, Homo Sapiens
tggtaactggc

SEQ ID NO: 893 - DNA, 11 bp, Homo Sapiens
ttctctacaag

SEQ ID NO: 894 - DNA, 11 bp, Homo Sapiens
tgtgaataaag

SEQ ID NO: 895 - DNA, 11 bp, Homo Sapiens
taaggacgaga

SEQ ID NO: 896 - DNA, 11 bp, Homo Sapiens
taatgacaata

SEQ ID NO: 897 - DNA, 11 bp, Homo Sapiens
ataactttgag

SEQ ID NO: 898 - DNA, 11 bp, Homo Sapiens
ctattcactgt

SEQ ID NO: 899 - DNA, 11 bp, Homo Sapiens
cttaaatctgg

SEQ ID NO: 900 - DNA, 11 bp, Homo Sapiens
gttctggttta

SEQ ID NO: 901 - DNA, 11 bp, Homo Sapiens
gtgaaacaccg

SEQ ID NO: 902 - DNA, 11 bp, Homo Sapiens
tttcaggggag

SEQ ID NO: 903 - DNA, 11 bp, Homo Sapiens
atgtactctgg

SEQ ID NO: 904 - DNA, 11 bp, Homo Sapiens
tttgtgttgta

SEQ ID NO: 905 - DNA, 11 bp, Homo Sapiens
ggaagctaagt

SEQ ID NO: 906 - DNA, 11 bp, Homo Sapiens
cccattcctcg

SEQ ID NO: 907 - DNA, 11 bp, Homo Sapiens
gataggtcggg

SEQ ID NO: 908 - DNA, 11 bp, Homo Sapiens
gcacaagttct

SEQ ID NO: 909 - DNA, 11 bp, Homo Sapiens
gtttgtcaatg

SEQ ID NO: 910 - DNA, 11 bp, Homo Sapiens
cgcacacacat

SEQ ID NO: 911 - DNA, 11 bp, Homo Sapiens
cagttccataa

SEQ ID NO: 912 - DNA, 11 bp, Homo Sapiens
gatggctgcct

SEQ ID NO: 913 - DNA, 11 bp, Homo Sapiens
agactaaggtt

SEQ ID NO: 914 - DNA, 11 bp, Homo Sapiens
gccttatgtat

SEQ ID NO: 915 - DNA, 11 bp, Homo Sapiens
atggaatgcta

SEQ ID NO: 916 - DNA, 11 bp, Homo Sapiens
ctgtttgttca

SEQ ID NO: 917 - DNA, 11 bp, Homo Sapiens
gatgtgtgctt

SEQ ID NO: 918 - DNA, 11 bp, Homo Sapiens
ccacagtagat

SEQ ID NO: 919 - DNA, 11 bp, Homo Sapiens
aaacctgggaa

SEQ ID NO: 920 - DNA, 11 bp, Homo Sapiens
ctgtaacatat

SEQ ID NO: 921 - DNA, 11 bp, Homo Sapiens
ttttggatgta

SEQ ID NO: 922 - DNA, 11 bp, Homo Sapiens
ctgggcctgaa

SEQ ID NO: 923 - DNA, 11 bp, Homo Sapiens
tatatcagtgt

SEQ ID NO: 924 - DNA, 11 bp, Homo Sapiens
ggtgagggagg

SEQ ID NO: 925 - DNA, 11 bp, Homo Sapiens
cagtgtatata

SEQ ID NO: 926 - DNA, 11 bp, Homo Sapiens
gggaaggcact

SEQ ID NO: 927 - DNA, 11 bp, Homo Sapiens
actgatgcaag

SEQ ID NO: 928 - DNA, 11 bp, Homo Sapiens
gcagtgccact

SEQ ID NO: 929 - DNA, 11 bp, Homo Sapiens
gtgcctaggag

SEQ ID NO: 930 - DNA, 11 bp, Homo Sapiens
cgcttttgtag

SEQ ID NO: 931 - DNA, 11 bp, Homo Sapiens
gagaagacttc

SEQ ID NO: 932 - DNA, 11 bp, Homo Sapiens
accttgtgccc

SEQ ID NO: 933 - DNA, 11 bp, Homo Sapiens
gtgctggagaa

SEQ ID NO: 934 - DNA, 11 bp, Homo Sapiens
agccctccctg

SEQ ID NO: 935 - DNA, 11 bp, Homo Sapiens
ggtggggagat

SEQ ID NO: 936 - DNA, 11 bp, Homo Sapiens
ggcaacgtggt

SEQ ID NO: 937 - DNA, 11 bp, Homo Sapiens
tgtttgtgtgt

SEQ ID NO: 938 - DNA, 11 bp, Homo Sapiens
aggatgaccag

SEQ ID NO: 939 - DNA, 11 bp, Homo Sapiens
aagctgagtgg

SEQ ID NO: 940 - DNA, 11 bp, Homo Sapiens
aaagtctagaa

SEQ ID NO: 941 - DNA, 11 bp, Homo Sapiens
cccataatccc

SEQ ID NO: 942 - DNA, 11 bp, Homo Sapiens
cggataaccag

SEQ ID NO: 943 - DNA, 11 bp, Homo Sapiens
tggtacacgta

SEQ ID NO: 944 - DNA, 11 bp, Homo Sapiens
gggtcaaaagg

SEQ ID NO: 945 - DNA, 11 bp, Homo Sapiens
gtattcccctt

SEQ ID NO: 946 - DNA, 11 bp, Homo Sapiens
gatcaggccag

SEQ ID NO: 947 - DNA, 11 bp, Homo Sapiens
aggcaggacgg

SEQ ID NO: 948 - DNA, 11 bp, Homo Sapiens
aaattttaaaa

SEQ ID NO: 949 - DNA, 11 bp, Homo Sapiens
ttttagcagga

SEQ ID NO: 950 - DNA, 11 bp, Homo Sapiens
gtcacaacctg

SEQ ID NO: 951 - DNA, 11 bp, Homo Sapiens
aaagcaaacca

SEQ ID NO: 952 - DNA, 11 bp, Homo Sapiens
gagaatctgct

SEQ ID NO: 953 - DNA, 11 bp, Homo Sapiens
caccgctgcag

SEQ ID NO: 954 - DNA, 11 bp, Homo Sapiens
cagggtgggtg

SEQ ID NO: 955 - DNA, 11 bp, Homo Sapiens
cagttcttgat

SEQ ID NO: 956 - DNA, 11 bp, Homo Sapiens
ttggtgtgctg

SEQ ID NO: 957 - DNA, 11 bp, Homo Sapiens
ccatttttacc

SEQ ID NO: 958 - DNA, 11 bp, Homo Sapiens
cagataacata

SEQ ID NO: 959 - DNA, 11 bp, Homo Sapiens
cgatggtcccc

SEQ ID NO: 960 - DNA, 11 bp, Homo Sapiens
ctcatatgtta

SEQ ID NO: 961 - DNA, 11 bp, Homo Sapiens
cagcagaagca

SEQ ID NO: 962 - DNA, 11 bp, Homo Sapiens
gccctttctct

SEQ ID NO: 963 - DNA, 11 bp, Homo Sapiens
ctcaacccccc

SEQ ID NO: 964 - DNA, 11 bp, Homo Sapiens
aagtgattctg

SEQ ID NO: 965 - DNA, 11 bp, Homo Sapiens
gggcccagggg

SEQ ID NO: 966 - DNA, 11 bp, Homo Sapiens
tagttgtaggg

SEQ ID NO: 967 - DNA, 11 bp, Homo Sapiens
aatttgcaaca

SEQ ID NO: 968 - DNA, 11 bp, Homo Sapiens
cagagacgtgg

SEQ ID NO: 969 - DNA, 11 bp, Homo Sapiens
gcagctaattt

SEQ ID NO: 970 - DNA, 11 bp, Homo Sapiens
ttttgaagcag

SEQ ID NO: 971 - DNA, 11 bp, Homo Sapiens
gaccagaaaaa

SEQ ID NO: 972 - DNA, 11 bp, Homo Sapiens
ccaaggattgg

SEQ ID NO: 973 - DNA, 11 bp, Homo Sapiens
tttcttaaagg

SEQ ID NO: 974 - DNA, 11 bp, Homo Sapiens
cggcaccttaa

SEQ ID NO: 975 - DNA, 11 bp, Homo Sapiens
agcagggctcc

SEQ ID NO: 976 - DNA, 11 bp, Homo Sapiens
attatccaggg

SEQ ID NO: 977 - DNA, 11 bp, Homo Sapiens
gctcccagact

SEQ ID NO: 978 - DNA, 11 bp, Homo Sapiens
ttaaaaaaaaa

SEQ ID NO: 979 - DNA, 11 bp, Homo Sapiens
tgtttgaattc

SEQ ID NO: 980 - DNA, 11 bp, Homo Sapiens
aaacttgctct

SEQ ID NO: 981 - DNA, 11 bp, Homo Sapiens
atggcagagac

SEQ ID NO: 982 - DNA, 11 bp, Homo Sapiens
gttcataggtc

SEQ ID NO: 983 - DNA, 11 bp, Homo Sapiens
tgtttgtacat

SEQ ID NO: 984 - DNA, 11 bp, Homo Sapiens
acaactcctgc

SEQ ID NO: 985 - DNA, 11 bp, Homo Sapiens
cttaataaaag

SEQ ID NO: 986 - DNA, 11 bp, Homo Sapiens
gcagagaaaaa

SEQ ID NO: 987 - DNA, 11 bp, Homo Sapiens
acttttaatga

SEQ ID NO: 988 - DNA, 11 bp, Homo Sapiens
gcgacggccgt

SEQ ID NO: 989 - DNA, 11 bp, Homo Sapiens
aataggtccac

SEQ ID NO: 990 - DNA, 11 bp, Homo Sapiens
cctgccaaact

SEQ ID NO: 991 - DNA, 11 bp, Homo Sapiens
tatgccctatc

SEQ ID NO: 992 - DNA, 11 bp, Homo Sapiens
agcagtgacgg

SEQ ID NO: 993 - DNA, 11 bp, Homo Sapiens
gaccctgggga

SEQ ID NO: 994 - DNA, 11 bp, Homo Sapiens
tcgtttccttc

SEQ ID NO: 995 - DNA, 11 bp, Homo Sapiens
aagccttaaaa

SEQ ID NO: 996 - DNA, 11 bp, Homo Sapiens
gactgttaatg

SEQ ID NO: 997 - DNA, 11 bp, Homo Sapiens
agtgtgatact

SEQ ID NO: 998 - DNA, 11 bp, Homo Sapiens
tttaaaagagc

SEQ ID NO: 999 - DNA, 11 bp, Homo Sapiens
tgtaatgtaac

SEQ ID NO: 1000 - DNA, 11 bp, Homo Sapiens
gcagcaagtaa

SEQ ID NO: 1001 - DNA, 11 bp, Homo Sapiens
ctgcaccatct

SEQ ID NO: 1002 - DNA, 11 bp, Homo Sapiens
ctgctaataaa

SEQ ID NO: 1003 - DNA, 11 bp, Homo Sapiens
atatgtctctg

SEQ ID NO: 1004 - DNA, 11 bp, Homo Sapiens
tcaatcagtga

SEQ ID NO: 1005 - DNA, 11 bp, Homo Sapiens
atgctagattt

SEQ ID NO: 1006 - DNA, 11 bp, Homo Sapiens
gtggtcagtgg

SEQ ID NO: 1007 - DNA, 11 bp, Homo Sapiens
aagccttatat

SEQ ID NO: 1008 - DNA, 11 bp, Homo Sapiens
aactctggacc

SEQ ID NO: 1009 - DNA, 11 bp, Homo Sapiens
aggaagactga

SEQ ID NO: 1010 - DNA, 11 bp, Homo Sapiens
ttaaatcgtga

SEQ ID NO: 1011 - DNA, 11 bp, Homo Sapiens
ggcagttaacc

SEQ ID NO: 1012 - DNA, 11 bp, Homo Sapiens
gtgaataaaca

SEQ ID NO: 1013 - DNA, 11 bp, Homo Sapiens
tgtcacctgaa

SEQ ID NO: 1014 - DNA, 11 bp, Homo Sapiens
ttgaaataata

SEQ ID NO: 1015 - DNA, 11 bp, Homo Sapiens
ttctctccaac

SEQ ID NO: 1016 - DNA, 11 bp, Homo Sapiens
tacagttcagt

SEQ ID NO: 1017 - DNA, 11 bp, Homo Sapiens
tgttaagttct

SEQ ID NO: 1018 - DNA, 11 bp, Homo Sapiens
ggttatttatg

SEQ ID NO: 1019 - DNA, 11 bp, Homo Sapiens
ggtctacatat

SEQ ID NO: 1020 - DNA, 11 bp, Homo Sapiens
gaacagaaaaa

SEQ ID NO: 1021 - DNA, 11 bp, Homo Sapiens
ggtttttccct

SEQ ID NO: 1022 - DNA, 11 bp, Homo Sapiens
aggagggataa

SEQ ID NO: 1023 - DNA, 11 bp, Homo Sapiens
gaaggattggg

SEQ ID NO: 1024 - DNA, 11 bp, Homo Sapiens
tgttcatttat

SEQ ID NO: 1025 - DNA, 11 bp, Homo Sapiens
tagtagattaa

SEQ ID NO: 1026 - DNA, 11 bp, Homo Sapiens
ttactgggttt

SEQ ID NO: 1027 - DNA, 11 bp, Homo Sapiens
ggatgcattag

SEQ ID NO: 1028 - DNA, 11 bp, Homo Sapiens
gggcccccaaa

SEQ ID NO: 1029 - DNA, 11 bp, Homo Sapiens
ttgtaaatagg

SEQ ID NO: 1030 - DNA, 11 bp, Homo Sapiens
aaattaaagtc

SEQ ID NO: 1031 - DNA, 11 bp, Homo Sapiens
gagtttgtgtt

SEQ ID NO: 1032 - DNA, 11 bp, Homo Sapiens
gtggtagtacc

SEQ ID NO: 1033 - DNA, 11 bp, Homo Sapiens
tggatgtactt

SEQ ID NO: 1034 - DNA, 11 bp, Homo Sapiens
tatccccaaat

SEQ ID NO: 1035 - DNA, 11 bp, Homo Sapiens
gctctgttcat

SEQ ID NO: 1036 - DNA, 11 bp, Homo Sapiens
tcatcatattt

SEQ ID NO: 1037 - DNA, 11 bp, Homo Sapiens
attgctaagtg

SEQ ID NO: 1038 - DNA, 11 bp, Homo Sapiens
ctttttcgtat

SEQ ID NO: 1039 - DNA, 11 bp, Homo Sapiens
agtagtctgca

SEQ ID NO: 1040 - DNA, 11 bp, Homo Sapiens
gggctgggggt

SEQ ID NO: 1041 - DNA, 11 bp, Homo Sapiens
aactgtactac

SEQ ID NO: 1042 - DNA, 11 bp, Homo Sapiens
ggagatggagc

SEQ ID NO: 1043 - DNA, 11 bp, Homo Sapiens
gttaatctgga

SEQ ID NO: 1044 - DNA, 11 bp, Homo Sapiens
tttgcctgtta

SEQ ID NO: 1045 - DNA, 11 bp, Homo Sapiens
aatgagaaggt

SEQ ID NO: 1046 - DNA, 11 bp, Homo Sapiens
cagatttctgt

SEQ ID NO: 1047 - DNA, 11 bp, Homo Sapiens
atacagtttgg

SEQ ID NO: 1048 - DNA, 11 bp, Homo Sapiens
ccttctcactc

SEQ ID NO: 1049 - DNA, 11 bp, Homo Sapiens
tgtagcctatg

SEQ ID NO: 1050 - DNA, 11 bp, Homo Sapiens
gcctgtttggg

SEQ ID NO: 1051 - DNA, 11 bp, Homo Sapiens
gcactggggca

SEQ ID NO: 1052 - DNA, 11 bp, Homo Sapiens
ggaactctgtt

SEQ ID NO: 1053 - DNA, 11 bp, Homo Sapiens
tcagagtaatc

SEQ ID NO: 1054 - DNA, 11 bp, Homo Sapiens
ttggccagggt

SEQ ID NO: 1055 - DNA, 11 bp, Homo Sapiens
tgacctatttc

SEQ ID NO: 1056 - DNA, 11 bp, Homo Sapiens
gtactgtaaga

SEQ ID NO: 1057 - DNA, 11 bp, Homo Sapiens
gaatttcccag

SEQ ID NO: 1058 - DNA, 11 bp, Homo Sapiens
taaatagaatt

SEQ ID NO: 1059 - DNA, 11 bp, Homo Sapiens
tcaagcaatca

SEQ ID NO: 1060 - DNA, 11 bp, Homo Sapiens
tgaccggcgag

SEQ ID NO: 1061 - DNA, 11 bp, Homo Sapiens
gcaccaaaaaa

SEQ ID NO: 1062 - DNA, 11 bp, Homo Sapiens
gcctgttaaaa

SEQ ID NO: 1063 - DNA, 11 bp, Homo Sapiens
aatgtagtttt

SEQ ID NO: 1064 - DNA, 11 bp, Homo Sapiens
gataatgattt

SEQ ID NO: 1065 - DNA, 11 bp, Homo Sapiens
gaagagccatc

SEQ ID NO: 1066 - DNA, 11 bp, Homo Sapiens
ttaaagatgca

SEQ ID NO: 1067 - DNA, 11 bp, Homo Sapiens
ctcacatttga

SEQ ID NO: 1068 - DNA, 11 bp, Homo Sapiens
aaatggcttga

SEQ ID NO: 1069 - DNA, 11 bp, Homo Sapiens
gtggcttccct

SEQ ID NO: 1070 - DNA, 11 bp, Homo Sapiens
gtacgcattcc

SEQ ID NO: 1071 - DNA, 11 bp, Homo Sapiens
tttgagacctg

SEQ ID NO: 1072 - DNA, 11 bp, Homo Sapiens
ttccataatta

SEQ ID NO: 1073 - DNA, 11 bp, Homo Sapiens
ctgtgagttcg

SEQ ID NO: 1074 - DNA, 11 bp, Homo Sapiens
tcgttacgcag

SEQ ID NO: 1075 - DNA, 11 bp, Homo Sapiens
caggggcttat

SEQ ID NO: 1076 - DNA, 11 bp, Homo Sapiens
ctgggggaggg

SEQ ID NO: 1077 - DNA, 11 bp, Homo Sapiens
cattttccaga

SEQ ID NO: 1078 - DNA, 11 bp, Homo Sapiens
ttggtcaaaca

SEQ ID NO: 1079 - DNA, 11 bp, Homo Sapiens
gccagctgacg

SEQ ID NO: 1080 - DNA, 11 bp, Homo Sapiens
atatgtatatt

SEQ ID NO: 1081 - DNA, 11 bp, Homo Sapiens
tctagaattta

SEQ ID NO: 1082 - DNA, 11 bp, Homo Sapiens
gaggttttctg

SEQ ID NO: 1083 - DNA, 11 bp, Homo Sapiens
cggggttcttg

SEQ ID NO: 1084 - DNA, 11 bp, Homo Sapiens
gagatggctgg

SEQ ID NO: 1085 - DNA, 11 bp, Homo Sapiens
tctaaaggtca

SEQ ID NO: 1086 - DNA, 11 bp, Homo Sapiens
tgtatgaattg

SEQ ID NO: 1087 - DNA, 11 bp, Homo Sapiens
aatgtacctgg

SEQ ID NO: 1088 - DNA, 11 bp, Homo Sapiens
tctagtcactg

SEQ ID NO: 1089 - DNA, 11 bp, Homo Sapiens
gttaaagttta

SEQ ID NO: 1090 - DNA, 11 bp, Homo Sapiens
tttttcctaag

SEQ ID NO: 1091 - DNA, 11 bp, Homo Sapiens
acgtttaaggc

SEQ ID NO: 1092 - DNA, 11 bp, Homo Sapiens
cccattcatag

SEQ ID NO: 1093 - DNA, 11 bp, Homo Sapiens
ctgggtaactg

SEQ ID NO: 1094 - DNA, 11 bp, Homo Sapiens
taagaagcccc

SEQ ID NO: 1095 - DNA, 11 bp, Homo Sapiens
aaagctactag

SEQ ID NO: 1096 - DNA, 11 bp, Homo Sapiens
tagatagagtc

SEQ ID NO: 1097 - DNA, 11 bp, Homo Sapiens
tacatagaatt

SEQ ID NO: 1098 - DNA, 11 bp, Homo Sapiens
tatacctgtgt

SEQ ID NO: 1099 - DNA, 11 bp, Homo Sapiens
ctgctgctact

SEQ ID NO: 1100 - DNA, 11 bp, Homo Sapiens
aaggaacttgt

SEQ ID NO: 1101 - DNA, 11 bp, Homo Sapiens
gggaaaaaaaa

SEQ ID NO: 1102 - DNA, 11 bp, Homo Sapiens
gtgcccagtca

SEQ ID NO: 1103 - DNA, 11 bp, Homo Sapiens
cagggagtgtg

SEQ ID NO: 1104 - DNA, 11 bp, Homo Sapiens
gggagaacccc

SEQ ID NO: 1105 - DNA, 11 bp, Homo Sapiens
aaaagacccga

SEQ ID NO: 1106 - DNA, 11 bp, Homo Sapiens
gagtgactatc

SEQ ID NO: 1107 - DNA, 11 bp, Homo Sapiens
gcgaaaaaaaa

SEQ ID NO: 1108 - DNA, 11 bp, Homo Sapiens
aaagtgaagaa

SEQ ID NO: 1109 - DNA, 11 bp, Homo Sapiens
gtggccaaagt

SEQ ID NO: 1110 - DNA, 11 bp, Homo Sapiens
gacccctgtca

SEQ ID NO: 1111 - DNA, 11 bp, Homo Sapiens
aaaagacaaat

SEQ ID NO: 1112 - DNA, 11 bp, Homo Sapiens
aagaggctgag

SEQ ID NO: 1113 - DNA, 11 bp, Homo Sapiens
tatatatagag

SEQ ID NO: 1114 - DNA, 11 bp, Homo Sapiens
tgaagaaagga

SEQ ID NO: 1115 - DNA, 11 bp, Homo Sapiens
tttattttaat

SEQ ID NO: 1116 - DNA, 11 bp, Homo Sapiens
ttgtttaaagg

SEQ ID NO: 1117 - DNA, 11 bp, Homo Sapiens
atcccttcccg

SEQ ID NO: 1118 - DNA, 11 bp, Homo Sapiens
aagatcctacg

SEQ ID NO: 1119 - DNA, 11 bp, Homo Sapiens
tggaactgagt

SEQ ID NO: 1120 - DNA, 11 bp, Homo Sapiens
gatgcatatag

SEQ ID NO: 1121 - DNA, 11 bp, Homo Sapiens
tttggaaagag

SEQ ID NO: 1122 - DNA, 11 bp, Homo Sapiens
agtatcagcag

SEQ ID NO: 1123 - DNA, 11 bp, Homo Sapiens
ctcctgaaaaa

SEQ ID NO: 1124 - DNA, 11 bp, Homo Sapiens
tgtgagccctt

SEQ ID NO: 1125 - DNA, 11 bp, Homo Sapiens
cattgtcttca

SEQ ID NO: 1126 - DNA, 11 bp, Homo Sapiens
ggaagagggtg

SEQ ID NO: 1127 - DNA, 11 bp, Homo Sapiens
gaccaactggg

SEQ ID NO: 1128 - DNA, 11 bp, Homo Sapiens
agaatttgaat

SEQ ID NO: 1129 - DNA, 11 bp, Homo Sapiens
agacctgtaat

SEQ ID NO: 1130 - DNA, 11 bp, Homo Sapiens
aaataattgtg

SEQ ID NO: 1131 - DNA, 11 bp, Homo Sapiens
gaactggattt

SEQ ID NO: 1132 - DNA, 11 bp, Homo Sapiens
ctccttaaaac

SEQ ID NO: 1133 - DNA, 11 bp, Homo Sapiens
tcacaaacttc

SEQ ID NO: 1134 - DNA, 11 bp, Homo Sapiens
ttacttcaact

SEQ ID NO: 1135 - DNA, 11 bp, Homo Sapiens
tataataaaaa

SEQ ID NO: 1136 - DNA, 11 bp, Homo Sapiens
tcagtgaactg

SEQ ID NO: 1137 - DNA, 11 bp, Homo Sapiens
gtccggtggtt

SEQ ID NO: 1138 - DNA, 11 bp, Homo Sapiens
atgcagagatt

SEQ ID NO: 1139 - DNA, 11 bp, Homo Sapiens
cgtcccggagc

SEQ ID NO: 1140 - DNA, 11 bp, Homo Sapiens
gactggaaaaa

SEQ ID NO: 1141 - DNA, 11 bp, Homo Sapiens
gactggactct

SEQ ID NO: 1142 - DNA, 11 bp, Homo Sapiens
cagtgattcca

SEQ ID NO: 1143 - DNA, 11 bp, Homo Sapiens
ctctgagagaa

SEQ ID NO: 1144 - DNA, 11 bp, Homo Sapiens
tacccagcaaa

SEQ ID NO: 1145 - DNA, 11 bp, Homo Sapiens
ttcctttttac

SEQ ID NO: 1146 - DNA, 11 bp, Homo Sapiens
cggtcttatgt

SEQ ID NO: 1147 - DNA, 11 bp, Homo Sapiens
taatgtgtaca

SEQ ID NO: 1148 - DNA, 11 bp, Homo Sapiens
gtgaaggcaga

SEQ ID NO: 1149 - DNA, 11 bp, Homo Sapiens
caatttaagtg

SEQ ID NO: 1150 - DNA, 11 bp, Homo Sapiens
taatttgattt

SEQ ID NO: 1151 - DNA, 11 bp, Homo Sapiens
attaaagtcag

SEQ ID NO: 1152 - DNA, 11 bp, Homo Sapiens
atgaaaaaaaa

SEQ ID NO: 1153 - DNA, 11 bp, Homo Sapiens
agaagaacgag

SEQ ID NO: 1154 - DNA, 11 bp, Homo Sapiens
ggaagcccacc

SEQ ID NO: 1155 - DNA, 11 bp, Homo Sapiens
tgaacagcata

SEQ ID NO: 1156 - DNA, 11 bp, Homo Sapiens
gcctgtgctgg

SEQ ID NO: 1157 - DNA, 11 bp, Homo Sapiens
aattaattgta

SEQ ID NO: 1158 - DNA, 11 bp, Homo Sapiens
taaataataca

SEQ ID NO: 1159 - DNA, 11 bp, Homo Sapiens
ggcacttatga

SEQ ID NO: 1160 - DNA, 11 bp, Homo Sapiens
taaattatttc

SEQ ID NO: 1161 - DNA, 11 bp, Homo Sapiens
ggtggcccggg

SEQ ID NO: 1162 - DNA, 11 bp, Homo Sapiens
gtataatagcc

SEQ ID NO: 1163 - DNA, 11 bp, Homo Sapiens
ttatcaaaaaa

SEQ ID NO: 1164 - DNA, 11 bp, Homo Sapiens
tacattcacct

SEQ ID NO: 1165 - DNA, 11 bp, Homo Sapiens
tgaaggttttt

SEQ ID NO: 1166 - DNA, 11 bp, Homo Sapiens
taaatgttgat

SEQ ID NO: 1167 - DNA, 11 bp, Homo Sapiens
aaggtaactaa

SEQ ID NO: 1168 - DNA, 11 bp, Homo Sapiens
ggaaactgatg

SEQ ID NO: 1169 - DNA, 11 bp, Homo Sapiens
acattggttaa

SEQ ID NO: 1170 - DNA, 11 bp, Homo Sapiens
aggtgcctcgg

SEQ ID NO: 1171 - DNA, 11 bp, Homo Sapiens
gtagagcttga

SEQ ID NO: 1172 - DNA, 11 bp, Homo Sapiens
gggctgtggag

SEQ ID NO: 1173 - DNA, 11 bp, Homo Sapiens
cacagaatgct

SEQ ID NO: 1174 - DNA, 11 bp, Homo Sapiens
ctaatttaact

SEQ ID NO: 1175 - DNA, 11 bp, Homo Sapiens
aaggcaaagct

SEQ ID NO: 1176 - DNA, 11 bp, Homo Sapiens
ttagcactgtg

SEQ ID NO: 1177 - DNA, 11 bp, Homo Sapiens
ttgggaatccc

SEQ ID NO: 1178 - DNA, 11 bp, Homo Sapiens
aatttgtgaag

SEQ ID NO: 1179 - DNA, 11 bp, Homo Sapiens
cttctccaaaa

SEQ ID NO: 1180 - DNA, 11 bp, Homo Sapiens
taccaagaccc

SEQ ID NO: 1181 - DNA, 11 bp, Homo Sapiens
ggatgtgaaag

SEQ ID NO: 1182 - DNA, 11 bp, Homo Sapiens
ggtagcctggg

SEQ ID NO: 1183 - DNA, 11 bp, Homo Sapiens
gggacgagtga

SEQ ID NO: 1184 - DNA, 11 bp, Homo Sapiens
cgtgggacact

SEQ ID NO: 1185 - DNA, 11 bp, Homo Sapiens
tgtattgtaca

SEQ ID NO: 1186 - DNA, 11 bp, Homo Sapiens
ctcacttctta

SEQ ID NO: 1187 - DNA, 11 bp, Homo Sapiens
gtgagacctca

SEQ ID NO: 1188 - DNA, 11 bp, Homo Sapiens
cagttacttag

SEQ ID NO: 1189 - DNA, 11 bp, Homo Sapiens
ctctgggatag

SEQ ID NO: 1190 - DNA, 11 bp, Homo Sapiens
tcctagcctgt

SEQ ID NO: 1191 - DNA, 11 bp, Homo Sapiens
agctctgctgc

SEQ ID NO: 1192 - DNA, 11 bp, Homo Sapiens
aaccaaaaaaa

SEQ ID NO: 1193 - DNA, 11 bp, Homo Sapiens
aagcgctctcg

SEQ ID NO: 1194 - DNA, 11 bp, Homo Sapiens
agataacacag

SEQ ID NO: 1195 - DNA, 11 bp, Homo Sapiens
gacacgaacaa

SEQ ID NO: 1196 - DNA, 11 bp, Homo Sapiens
gtggaaccccg

SEQ ID NO: 1197 - DNA, 11 bp, Homo Sapiens
gtgccctgttg

SEQ ID NO: 1198 - DNA, 11 bp, Homo Sapiens
cgatcagtttg

SEQ ID NO: 1199 - DNA, 11 bp, Homo Sapiens
ggggaagggca

SEQ ID NO: 1200 - DNA, 11 bp, Homo Sapiens
tccgtggttgg

SEQ ID NO: 1201 - DNA, 11 bp, Homo Sapiens
tggcccccgcc

SEQ ID NO: 1202 - DNA, 11 bp, Homo Sapiens
cagttggttgt

SEQ ID NO: 1203 - DNA, 11 bp, Homo Sapiens
tccgcgagaag

SEQ ID NO: 1204 - DNA, 11 bp, Homo Sapiens
aaaaaaaaaag

SEQ ID NO: 1205 - DNA, 11 bp, Homo Sapiens
aagataatgcc

SEQ ID NO: 1206 - DNA, 11 bp, Homo Sapiens
tcactgcactc

SEQ ID NO: 1207 - DNA, 11 bp, Homo Sapiens
gcacctagtgc

SEQ ID NO: 1208 - DNA, 11 bp, Homo Sapiens
gagagggcaga

SEQ ID NO: 1209 - DNA, 11 bp, Homo Sapiens
ttaaaggccgg

SEQ ID NO: 1210 - DNA, 11 bp, Homo Sapiens
gaatcggttat

SEQ ID NO: 1211 - DNA, 11 bp, Homo Sapiens
gggccaataaa

SEQ ID NO: 1212 - DNA, 11 bp, Homo Sapiens
tcaaatgcaaa

SEQ ID NO: 1213 - DNA, 11 bp, Homo Sapiens
tcaataaagga

SEQ ID NO: 1214 - DNA, 11 bp, Homo Sapiens
cagaataatgt

SEQ ID NO: 1215 - DNA, 11 bp, Homo Sapiens
cctggccaaaa

SEQ ID NO: 1216 - DNA, 11 bp, Homo Sapiens
acaatgtagga

SEQ ID NO: 1217 - DNA, 11 bp, Homo Sapiens
aaacaccaaat

SEQ ID NO: 1218 - DNA, 11 bp, Homo Sapiens
tgaaagtcctg

SEQ ID NO: 1219 - DNA, 11 bp, Homo Sapiens
ataattgacta

SEQ ID NO: 1220 - DNA, 11 bp, Homo Sapiens
taccatccata

SEQ ID NO: 1221 - DNA, 11 bp, Homo Sapiens
gtgtagttgag

SEQ ID NO: 1222 - DNA, 11 bp, Homo Sapiens
tgactgtcacg

SEQ ID NO: 1223 - DNA, 11 bp, Homo Sapiens
ggacatttttc

SEQ ID NO: 1224 - DNA, 11 bp, Homo Sapiens
tctctctgcct

SEQ ID NO: 1225 - DNA, 11 bp, Homo Sapiens
actacagccat

SEQ ID NO: 1226 - DNA, 11 bp, Homo Sapiens
ttggacaagaa

SEQ ID NO: 1227 - DNA, 11 bp, Homo Sapiens
gggattaaagc

SEQ ID NO: 1228 - DNA, 11 bp, Homo Sapiens
ctttttgccac

SEQ ID NO: 1229 - DNA, 11 bp, Homo Sapiens
cactatgtaaa

SEQ ID NO: 1230 - DNA, 11 bp, Homo Sapiens
tgcctcccatc

SEQ ID NO: 1231 - DNA, 11 bp, Homo Sapiens
ataatctgaag

SEQ ID NO: 1232 - DNA, 11 bp, Homo Sapiens
gtcaaaaaaaa

SEQ ID NO: 1233 - DNA, 11 bp, Homo Sapiens
gaatgtccttt

SEQ ID NO: 1234 - DNA, 11 bp, Homo Sapiens
gtttcagttac

SEQ ID NO: 1235 - DNA, 11 bp, Homo Sapiens
ttcattaaaaa

SEQ ID NO: 1236 - DNA, 11 bp, Homo Sapiens
cacagtatttg

SEQ ID NO: 1237 - DNA, 11 bp, Homo Sapiens
tcgtcctagaa

SEQ ID NO: 1238 - DNA, 11 bp, Homo Sapiens
attagcagagt

SEQ ID NO: 1239 - DNA, 11 bp, Homo Sapiens
gtgaaacacca

SEQ ID NO: 1240 - DNA, 11 bp, Homo Sapiens
tctgtaatctc

SEQ ID NO: 1241 - DNA, 11 bp, Homo Sapiens
gtgaaaccctc

SEQ ID NO: 1242 - DNA, 11 bp, Homo Sapiens
ctccattgcca

SEQ ID NO: 1243 - DNA, 11 bp, Homo Sapiens
tgccatataag

SEQ ID NO: 1244 - DNA, 11 bp, Homo Sapiens
tggctgttaat

SEQ ID NO: 1245 - DNA, 11 bp, Homo Sapiens
cctgtcaatgt

SEQ ID NO: 1246 - DNA, 11 bp, Homo Sapiens
gtggcggacgc

SEQ ID NO: 1247 - DNA, 11 bp, Homo Sapiens
cgaatgtcctt

SEQ ID NO: 1248 - DNA, 11 bp, Homo Sapiens
agaacagacca

SEQ ID NO: 1249 - DNA, 11 bp, Homo Sapiens
gtgttagcgca

SEQ ID NO: 1250 - DNA, 11 bp, Homo Sapiens
tgcatctgtgc

SEQ ID NO: 1251 - DNA, 11 bp, Homo Sapiens
ccagtttgtat

SEQ ID NO: 1252 - DNA, 11 bp, Homo Sapiens
tttattgaatt

SEQ ID NO: 1253 - DNA, 11 bp, Homo Sapiens
tgtgaagatta

SEQ ID NO: 1254 - DNA, 11 bp, Homo Sapiens
ctggtggtgcc

SEQ ID NO: 1255 - DNA, 11 bp, Homo Sapiens
cctttctttat

SEQ ID NO: 1256 - DNA, 11 bp, Homo Sapiens
tctgtgctgtc

SEQ ID NO: 1257 - DNA, 11 bp, Homo Sapiens
ccagtggctca

SEQ ID NO: 1258 - DNA, 11 bp, Homo Sapiens
aggcctgggcc

SEQ ID NO: 1259 - DNA, 11 bp, Homo Sapiens
tacagacatac

SEQ ID NO: 1260 - DNA, 11 bp, Homo Sapiens
tgccctgttcc

SEQ ID NO: 1261 - DNA, 11 bp, Homo Sapiens
atgacctgaag

SEQ ID NO: 1262 - DNA, 11 bp, Homo Sapiens
tttaaaaaaaa

SEQ ID NO: 1263 - DNA, 11 bp, Homo Sapiens
aatatttagtg

SEQ ID NO: 1264 - DNA, 11 bp, Homo Sapiens
tgcccagcaaa

SEQ ID NO: 1265 - DNA, 11 bp, Homo Sapiens
attgatgacgg

SEQ ID NO: 1266 - DNA, 11 bp, Homo Sapiens
gtgttgagaga

SEQ ID NO: 1267 - DNA, 11 bp, Homo Sapiens
gagtagaggcc

SEQ ID NO: 1268 - DNA, 11 bp, Homo Sapiens
taacattggtg

SEQ ID NO: 1269 - DNA, 11 bp, Homo Sapiens
gaaccacagga

SEQ ID NO: 1270 - DNA, 11 bp, Homo Sapiens
ccctcctctcc

SEQ ID NO: 1271 - DNA, 11 bp, Homo Sapiens
tagcaatcaga

SEQ ID NO: 1272 - DNA, 11 bp, Homo Sapiens
tgtctgtgtgt

SEQ ID NO: 1273 - DNA, 11 bp, Homo Sapiens
tggccaaaaaa

SEQ ID NO: 1274 - DNA, 11 bp, Homo Sapiens
gacaaaaagtc

SEQ ID NO: 1275 - DNA, 11 bp, Homo Sapiens
gaaaagctcct

SEQ ID NO: 1276 - DNA, 11 bp, Homo Sapiens
tgtaagtctgc

SEQ ID NO: 1277 - DNA, 11 bp, Homo Sapiens
tgctaaaaaaa

SEQ ID NO: 1278 - DNA, 11 bp, Homo Sapiens
ttaatcctaaa

SEQ ID NO: 1279 - DNA, 11 bp, Homo Sapiens
tgcggaggccc

SEQ ID NO: 1280 - DNA, 11 bp, Homo Sapiens
gaaaagccttc

SEQ ID NO: 1281 - DNA, 11 bp, Homo Sapiens
ggaggcaggtg

SEQ ID NO: 1282 - DNA, 11 bp, Homo Sapiens
gctaaaaaaaa

SEQ ID NO: 1283 - DNA, 11 bp, Homo Sapiens
tatttattcct

SEQ ID NO: 1284 - DNA, 11 bp, Homo Sapiens
ctttctttgag

SEQ ID NO: 1285 - DNA, 11 bp, Homo Sapiens
taaataatttc

SEQ ID NO: 1286 - DNA, 11 bp, Homo Sapiens
cagattgtgaa

SEQ ID NO: 1287 - DNA, 11 bp, Homo Sapiens
atggccataga

SEQ ID NO: 1288 - DNA, 11 bp, Homo Sapiens
tttttgataaa

SEQ ID NO: 1289 - DNA, 11 bp, Homo Sapiens
tggggtggagt

SEQ ID NO: 1290 - DNA, 11 bp, Homo Sapiens
gagttatgttg

SEQ ID NO: 1291 - DNA, 11 bp, Homo Sapiens
acctctggctt

SEQ ID NO: 1292 - DNA, 11 bp, Homo Sapiens
gcggagagagg

SEQ ID NO: 1293 - DNA, 11 bp, Homo Sapiens
aatacttaaat

SEQ ID NO: 1294 - DNA, 11 bp, Homo Sapiens
gcaaagaaaaa

SEQ ID NO: 1295 - DNA, 11 bp, Homo Sapiens
acggctccgag

SEQ ID NO: 1296 - DNA, 11 bp, Homo Sapiens
ctcctgaaggc

SEQ ID NO: 1297 - DNA, 11 bp, Homo Sapiens
acagcgtctgc

SEQ ID NO: 1298 - DNA, 11 bp, Homo Sapiens
gccactacccc

SEQ ID NO: 1299 - DNA, 11 bp, Homo Sapiens
aggggattccc

SEQ ID NO: 1300 - DNA, 11 bp, Homo Sapiens
taggaccctgc

SEQ ID NO: 1301 - DNA, 11 bp, Homo Sapiens
gagcggcctct

SEQ ID NO: 1302 - DNA, 11 bp, Homo Sapiens
gccccaggtag

SEQ ID NO: 1303 - DNA, 11 bp, Homo Sapiens
taagcattaaa

SEQ ID NO: 1304 - DNA, 11 bp, Homo Sapiens
acaaaataaaa

SEQ ID NO: 1305 - DNA, 11 bp, Homo Sapiens
ggctgccctgg

SEQ ID NO: 1306 - DNA, 11 bp, Homo Sapiens
ttgagccagcc

SEQ ID NO: 1307 - DNA, 11 bp, Homo Sapiens
ccattctcctg

SEQ ID NO: 1308 - DNA, 11 bp, Homo Sapiens
ctcatctgctg

SEQ ID NO: 1309 - DNA, 11 bp, Homo Sapiens
aaaccagggcc

SEQ ID NO: 1310 - DNA, 11 bp, Homo Sapiens
tggaccccccg

SEQ ID NO: 1311 - DNA, 11 bp, Homo Sapiens
tttcagtgggt

SEQ ID NO: 1312 - DNA, 11 bp, Homo Sapiens
gcttaacctgg

SEQ ID NO: 1313 - DNA, 11 bp, Homo Sapiens
acttacctgct

SEQ ID NO: 1314 - DNA, 11 bp, Homo Sapiens
  Tab. 1
gctggagttgc

SEQ ID NO: 1315 - DNA, 11 bp, Homo Sapiens
  Tab. 1
gtctggaagga

SEQ ID NO: 1316 - DNA, 11 bp, Homo Sapiens
  Tab. 1
tgcttttgcct

SEQ ID NO: 1317 - DNA, 11 bp, Homo Sapiens
  Tab. 1
aaagcacaagt

SEQ ID NO: 1318 - DNA, 11 bp, Homo Sapiens
  Tab. 1
cgaatgtcctt

SEQ ID NO: 1319 - DNA, 11 bp, Homo Sapiens
  Tab. 1
aaagcatccct

SEQ ID NO: 1320 - DNA, 11 bp, Homo Sapiens
  Tab. 1
ttgggggtgta

SEQ ID NO: 1321 - DNA, 11 bp, Homo Sapiens
  Tab. 1
aaaaggttatg

SEQ ID NO: 1322 - DNA, 11 bp, Homo Sapiens
  Tab. 1
cttttttccta

SEQ ID NO: 1323 - DNA, 11 bp, Homo Sapiens
  Tab. 1
acgttaaagac

SEQ ID NO: 1324 - DNA, 11 bp, Homo Sapiens
  Tab. 1
gtggagggcac

SEQ ID NO: 1325 - DNA, 11 bp, Homo Sapiens
  Tab. 2
gaaaacaaagt

SEQ ID NO: 1326 - DNA, 11 bp, Homo Sapiens
  Tab. 2
cctctttgcat

SEQ ID NO: 1327 - DNA, 11 bp, Homo Sapiens
  Tab. 2
aaaataaacct

SEQ ID NO: 1328 - DNA, 11 bp, Homo Sapiens
  Tab. 2
gagagctaact

SEQ ID NO: 1329 - DNA, 11 bp, Homo Sapiens
  Tab. 2
agaaaaaaaaa

SEQ ID NO: 1330 - DNA, 11 bp, Homo Sapiens
  Tab. 2
ggcttctaaca

SEQ ID NO: 1331 - DNA, 11 bp, Homo Sapiens
  Tab. 2
catatcattaa

SEQ ID NO: 1332 - DNA, 11 bp, Homo Sapiens
  Tab. 2
acatttcaaag

SEQ ID NO: 1333 - DNA, 11 bp, Homo Sapiens
  Tab. 2
gcccctgctga

SEQ ID NO: 1334 - DNA, 11 bp, Homo Sapiens
  Tab. 2
gatgtgcacga

SEQ ID NO: 1335 - DNA, 11 bp, Homo Sapiens
  Tab. 2
taataaagaat